(19) United States
(12) Patent Application Publication
(10) Pub. No.: US 2024/0173432 A1
(43) Pub. Date: May 30, 2024

(54) Compositions and Methods for Treatment of Myotonic Dystrophy Type 1 with CRISPR/SluCas9

(71) Applicant: Vertex Pharmaceuticals Incorporated, Boston, MA (US)

(72) Inventors: Guoxiang Ruan, Boston, MA (US); Jianming Liu, Boston, MA (US); Tudor Fulga, Boston, MA (US); Mehmet Fatih Bolukbasi, Arlington, MA (US); Eric Gunnar Anderson, Cambridge, MA (US); Lingjun Rao, Boston, MA (US); Norzehan Abdul-Manan, Boston, MA (US); Matthias Heidenreich, Boston, MA (US); Gregoriy Aleksandrovich Dokshin, Boston, MA (US); Jesper Gromada, Boston, MA (US)

(73) Assignee: Vertex Pharmaceuticals Incorporated, Boston, MA (US)

(21) Appl. No.: 18/456,288

(22) Filed: Aug. 25, 2023

Related U.S. Application Data
(63) Continuation of application No. PCT/US22/17854, filed on Feb. 25, 2022.
(60) Provisional application No. 63/154,444, filed on Feb. 26, 2021.
(60) Provisional application No. 63/179,859, filed on Apr. 26, 2021.
(60) Provisional application No. 63/276,002, filed on Nov. 5, 2021.
(60) Provisional application No. 63/306,902, filed on Feb. 4, 2022.

(51) Int. Cl. A61K 48/00; A61K 31/506; A61K 31/5377; A61P 19/00; C12N 9/22; C12N 15/11

(52) U.S. Cl. CPC A61K 48/005; A61K 31/506; A61K 31/5377; A61P 19/00; C12N 9/22; C12N 15/11; C12N 2310/20

(57) ABSTRACT

Compositions and methods for treating Myotonic Dystrophy Type 1 (DM1) are encompassed.

This application is a bypass continuation of PCT/US2022/017854 filed Feb. 25, 2022, which claims the benefit of priority to U.S. Provisional Application No. 63/154,444, filed Feb. 26, 2021; U.S. Provisional Patent Application No. 63/179,859, filed Apr. 26, 2021; U.S. Provisional Application No. 63/276,002, filed Nov. 5, 2021; and U.S. Provisional Patent Application No. 63/306,902, filed Feb. 4, 2022; all of which are incorporated by reference in their entirety.

The instant application contains a Sequence Listing which has been submitted electronically in XML format and is hereby incorporated by reference in its entirety. Said XML copy, created on Aug. 17, 2023, is named 2023-08-17_01245-0027-00US_ST26 and is 798,961 bytes in size.

### INTRODUCTION AND SUMMARY

Myotonic Dystrophy Type 1 (DM1) is an autosomal dominant muscle disorder caused by the expansion of CTG repeats in the 3′ untranslated region (UTR) of human DMPK gene, which leads to RNA foci and mis-splicing of genes important for muscle function. The disorder affects skeletal and smooth muscle as well as the eye, heart, endocrine system, and central nervous system, and causes muscle weakness, wasting, physical disablement, and shortened lifespan.

CRISPR-based genome editing can provide sequence-specific cleavage of genomic DNA using a Cas9 and a guide RNA. For example, a nucleic acid encoding the Cas9 enzyme and a nucleic acid encoding for the appropriate guide RNA can be provided on separate vectors or together on a single vector and administered in vivo or in vitro to knockout or correct a genetic mutation. The approximately 20 nucleotides at the 5′ end of the guide RNA serves as the guide or spacer sequence that can be any sequence complementary to one strand of a genomic target location that has an adjacent protospacer adjacent motif (PAM). The PAM sequence is a short sequence adjacent to the Cas9 nuclease cut site that the Cas9 molecule requires for appropriate binding. The nucleotides 3′ of the guide or spacer sequence of the guide RNA serve as a scaffold sequence for interacting with Cas9. When a guide RNA and a Cas9 are expressed, the guide RNA will bind to Cas9 and direct it to the sequence complementary to the guide sequence, where it will then initiate a double-stranded break (DSB). To repair these breaks, cells typically use an error prone mechanism of non-homologous end joining (NHEJ) which can lead to disruption of function in the target gene through insertions or deletion of codons, shifts in the reading frame, or result in a premature stop codon triggering nonsense-mediated decay. See, e.g., Kumar et al. (2018) Front. Mol. Neurosci. Vol. 11, Article 413.

Adeno-associated virus (AAV) administration of the CRISPR-Cas components in vivo or in vitro is attractive due to the early and ongoing successes of AAV vector design, manufacturing, and clinical stage administration for gene therapy. See, e.g., Wang et al. (2019) Nature Reviews Drug Discovery 18:358-378; Ran et al. (2015a) Nature 520: 186-101. However, the commonly used Streptococcus pyogenes (spCas9) is very large, and when used in AAV-based CRISPR/Cas systems, requires two AAV vectors—one vector carrying the nucleic acid encoding the spCas9, and the other carrying the nucleic acid encoding the guide RNA. One possible way to overcome this technical hurdle is to take advantage of the smaller orthologs of Cas9 derived from different prokaryotic species. Smaller Cas9's may be able to be manufactured on a single AAV vector together with a nucleic acid encoding a guide RNA thereby reducing manufacturing costs and reducing complexity of administration routes and protocols.

Provided herein are compositions and methods for treating DM1 utilizing the smaller Cas9 from Staphylococcus lugdunensis (SluCas9). Compositions comprising i) a single AAV vector comprising a nucleic acid molecule encoding SluCas9, and one or more guide RNAs; and ii) an optional DNA-PK inhibitor are provided. In some embodiments, the single AAV vector comprises a nucleic acid molecule encoding SluCas9 and one or more copies of a single guide RNA (e.g., a guide RNA comprising the sequence of any one of SEQ ID Nos: 8, 63, 64 and 81). In some embodiments, the single AAV vector comprises a nucleic acid molecule encoding SluCas9 and one or more copies of a first guide RNA and one or more copies of a second guide RNA. Methods using disclosed compositions to treat DM1 are also provided. Compositions and methods disclosed herein may be used for excising a portion of the CTG repeat region to treat DM1, reduce RNA foci, and/or correct mis-splicing in DM1 patient cells. For example, disclosed herein are guide RNAs and combinations of guide RNAs particularly suitable for use with SluCas9 for use in methods of excising a CTG repeat in the 3′ UTR of DMPK, with or without a DNA-PK inhibitor.

Also provided herein are systems comprising more than one vector, whereby one or more guide RNAs are incorporated on a single vector together with a smaller SluCas9 and another vector comprises a nucleic acid encoding multiple copies of guide RNAs. Such systems allow extreme design flexibility in situations where more than one guide RNA is desired for optimal performance. For example, one vector may be utilized to express SluCas9 and optionally one or more guide RNAs targeting one or more genomic targets, and a second vector may be utilized to express multiple copies of the same or different guide RNAs targeting the same or different genomic targets. Compositions and methods utilizing these dual vector configurations are provided herein and have the benefit of reducing manufacturing costs, reducing complexity of administration routes and protocols, and allowing maximum flexibility with regard to using multiple copies of the same or different guide RNAs targeting the same or different genomic target sequences. In some instances, providing multiple copies of the same guide RNA improves the efficiency of the guide, improving an already successful system.

Accordingly, the following embodiments are provided:
[Embodiment 01] A composition comprising a single nucleic acid molecule encoding one or more guide RNAs and a Cas9, wherein the single nucleic acid molecule comprises:
   a. a first nucleic acid encoding one or more spacer sequence selected from any one of SEQ ID NOs: 1-65, 67-167, and 201-531 and a second nucleic acid encoding a Staphylococcus lugdunensis Cas9 (SluCas9);
   b. a first nucleic acid encoding one or more spacer sequence comprising at least 20, or 21 contiguous nucleotides of a spacer sequence selected from any one of SEQ ID NOs: 1-65, 67-167, and 201-531 and a second nucleic acid encoding a Staphylococcus lugdunensis Cas9 (SluCas9);
   c. a first nucleic acid encoding one or more spacer sequence that is at least 90% identical to any one of SEQ ID NOs: 1-65, 67-167, and 201-531 and a second nucleic acid encoding a Staphylococcus lugdunensis Cas9 (SluCas9);
   d. a first nucleic acid encoding 2 spacer sequences selected from any one of SEQ ID NOs: 63 and 100, and SEQ ID NOs: 64 and 100, and a second nucleic acid encoding a Staphylococcus lugdunensis Cas9 (SluCas9); or
   e. a first nucleic acid encoding one or more spacer sequence selected from any one of SEQ ID NOs: 8, 63, 64, and 81 and a second nucleic acid encoding a Staphylococcus lugdunensis Cas9 (SluCas9).
[Embodiment 02] The composition of embodiment 1, further comprising a DNA-PK inhibitor.
[Embodiment 03] The composition of embodiment 1 or 2, further comprising a DNA-PK inhibitor, wherein the DNA-PK inhibitor is Compound 6.
[Embodiment 04] The composition of embodiment 1 or 2, further comprising a DNA-PK inhibitor, wherein the DNA-PK inhibitor is Compound 1.
[Embodiment 05] The composition of embodiment 1 or 2, further comprising a DNA-PK inhibitor, wherein the DNA-PK inhibitor is Compound 2.
[Embodiment 06] The composition of any one of embodiments 1-5, wherein the guide RNA is an sgRNA.
[Embodiment 07] The composition of any one of embodiments 1-6, wherein the guide RNA is modified.
[Embodiment 08] The composition of embodiment 7, wherein the modification alters one or more 2′ positions and/or phosphodiester linkages.
[Embodiment 09] The composition of any one of embodiments 7-8, wherein the modification alters one or more, or all, of the first three nucleotides of the guide RNA.
[Embodiment 10] The composition of any one of embodiments 7-9, wherein the modification alters one or more, or all, of the last three nucleotides of the guide RNA.
[Embodiment 11] The composition of any one of embodiments 7-10, wherein the modification includes one or more of a phosphorothioate modification, a 2′-OMe modification, a 2′-O-MOE modification, a 2′-F modification, a 2′-O-methine-4′ bridge modification, a 3′-thiophosphonoacetate modification, or a 2′-deoxy modification.
[Embodiment 12] The composition of any one of the preceding embodiments, wherein the single nucleic acid molecule is associated with a lipid nanoparticle (LNP).
[Embodiment 13] The composition of any one of embodiments 1-12, wherein the single nucleic acid molecule is a viral vector.
[Embodiment 14] The composition of embodiment 13, wherein the viral vector is an adeno-associated virus vector, a lentiviral vector, an integrase-deficient lentiviral vector, an adenoviral vector, a vaccinia viral vector, an alphaviral vector, or a herpes simplex viral vector.
[Embodiment 15] The composition of embodiment 13, wherein the viral vector is an adeno-associated virus (AAV) vector.
[Embodiment 16] The composition of embodiment 15, wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.
[Embodiment 17] The composition of embodiment 16, wherein the AAV vector is an AAV serotype 9 vector.
[Embodiment 18] The composition of embodiment 16, wherein the AAV vector is an AAVrh10 vector.
[Embodiment 19] The composition of embodiment 16, wherein the AAV vector is an AAVrh74 vector.
[Embodiment 20] The composition of any one of embodiments 13-19, comprising a viral vector, wherein the viral vector comprises a tissue-specific promoter.
[Embodiment 21] The composition of any one of embodiments 13-19, comprising a viral vector, wherein the viral vector comprises a muscle-specific promoter, optionally wherein the muscle-specific promoter is a muscle creatine kinase promoter, a desmin promoter, an MHCK7 promoter, an SPc5-12 promoter, or a CK8e promoter.
[Embodiment 22] The composition of any one of embodiments 13-19, comprising a viral vector, wherein the viral vector comprises a U6, H1, or 7SK promoter.
[Embodiment 23] The composition of any one of embodiments 1-22, comprising a nucleic acid encoding SluCas9, wherein the SluCas9 comprises the amino acid sequence of SEQ ID NO: 712.
[Embodiment 24] The composition of any one of embodiments 1-22, comprising a nucleic acid encoding SluCas9, wherein the SluCas9 is a variant of the amino acid sequence of SEQ ID NO: 712.
[Embodiment 25] The composition of any one of embodiments 1-22, comprising a nucleic acid encoding SluCas9, wherein the SluCas9 comprises an amino acid sequence selected from any one of SEQ ID NOs: 718-720.
[Embodiment 26] The composition of any one of embodiments 1-25 and a pharmaceutically acceptable excipient.
[Embodiment 27] A composition comprising a guide RNA encoded by a sequence comprising any one of SEQ ID NOs: 1-65, 67-167, and 201-531 or complements thereof.
[Embodiment 28] The composition of any one of embodiments 1-27 for use in treating Myotonic Dystrophy Type 1 (DM1).
[Embodiment 29] The composition of any one of embodiments 1-27 for use in making a double strand break in the DMPK gene.
[Embodiment 30] The composition of any one of embodiments 1-27 for use in excising a CTG repeat in the 3′ UTR of the DMPK gene.
[Embodiment 31] A method of treating Myotonic Dystrophy Type 1 (DM1), the method comprising delivering to a cell the composition of any one of embodiments 1-27, and optionally a DNA-PK inhibitor.
[Embodiment 32] A method of treating Myotonic Dystrophy Type 1 (DM1), the method comprising delivering to a cell a single nucleic acid molecule comprising:
   a nucleic acid encoding a guide RNA, wherein the guide RNA comprises:
      a. one or more spacer sequence selected from any one of SEQ ID NOs: 1-65, 67-167, and 201-531;
      b. one or more spacer sequence comprising at least 20, or 21 contiguous nucleotides of a spacer sequence selected from any one of SEQ ID NOs: 1-65, 67-167, and 201-531; or
      c. one or more spacer sequence that is at least 90% identical to any one of SEQ ID NOs: 1-65, 67-167, and 201-531;
   a nucleic acid encoding a Staphylococcus lugdunensis Cas9 (SluCas9); and
   optionally a DNA-PK inhibitor.
[Embodiment 33] A method of treating Myotonic Dystrophy Type 1 (DM1), the method comprising delivering to a cell a single nucleic acid molecule comprising:
   a nucleic acid encoding a pair of guide RNAs comprising:
      a. a first spacer sequence selected from any one of SEQ ID NOs: 1-65 and a second spacer sequence selected from any one of SEQ ID NOs: 67-167;
      b. a first and second spacer sequence comprising at least 20, or 21 contiguous nucleotides of any of the first and second spacer sequences of i) a.;
      c. a first and second spacer sequence that is at least 90% identical to any of the first and second spacer sequences of i) a. or i) b.;
      d. a first and second spacer, or one or more vectors encoding the pair of guide RNAs, wherein the first and second spacer sequences comprise any one of the following pairs of SEQ ID NOs: 6 and 72; 6 and 81; 6 and 84; 6 and 98; 6 and 100; 6 and 114; 6 and 122; 6 and 134; 6 and 139; 6 and 149; 6 and 166; 8 and 72; 8 and 72; 8 and 81; 8 and 84; 8 and 98; 8 and 100; 8 and 114; 8 and 122; 8 and 134; 8 and 139; 8 and 149; 8 and 166; 10 and 72; 10 and 81; 10 and 84; 10 and 98; 10 and 100; 10 and 114; 10 and 122; 10 and 134; 10 and 139; 10 and 149; 10 and 166; 21 and 72; 21 and 81; 21 and 84; 21 and 98; 21 and 100; 21 and 114; 21 and 122; 21 and 134; 21 and 139; 21 and 149; 21 and 166; 58 and 72; 58 and 81; 58 and 84; 58 and 98; 58 and 100; 58 and 114; 58 and 122; 58 and 134; 58 and 139; 58 and 149; 58 and 166; 62 and 72; 62 and 81; 62 and 84; 62 and 98; 62 and 100; 62 and 114; 62 and 122; 62 and 134; 62 and 139; 62 and 149; 62 and 166; 63 and 72; 63 and 81; 63 and 84; 63 and 98; 63 and 100; 63 and 114; 63 and 122; 63 and 134; 63 and 139; 63 and 149; 63 and 166; 64 and 72; 64 and 81; 64 and 84; 64 and 98; 64 and 100; 64 and 114; 64 and 122; 64 and 134; 64 and 139; 64 and 149; and 64 and 166;
      e. a first and second spacer, or one or more vectors encoding the pair of guide RNAs, wherein the first and second spacer sequences comprise SEQ ID NOs: 63 and 100 or SEQ ID NOs: 64 and 100;
   a nucleic acid encoding a Staphylococcus lugdunensis Cas9 (SluCas9); and
   optionally a DNA-PK inhibitor.
[Embodiment 34] A method of excising a CTG repeat in the 3′ UTR of the DMPK gene, the method comprising delivering to a cell the composition of any one of embodiments 1-27.
[Embodiment 35] A method of excising a CTG repeat in the 3′ UTR of the DMPK gene, the method comprising delivering to a cell a single nucleic acid molecule comprising:
   a nucleic acid encoding a guide RNA, wherein the guide RNA comprises:
      a. one or more spacer sequence selected from any one of SEQ ID NOs: 1-65, 67-167, and 201-531;
      b. one or more spacer sequence selected from any one of SEQ ID NOs: 8, 63, 64, and 81;
      c. one or more spacer sequence comprising at least 20, or 21 contiguous nucleotides of a spacer sequence selected from any one of SEQ ID NOs: 1-65, 67-167, and 201-531; or
      d. one or more spacer sequence that is at least 90% identical to any one of SEQ ID NOs: 1-65, 67-167, and 201-531;
      e. two (2) spacer sequences selected from any one of SEQ ID NOs: 63 and 100, and 64 and 100, and a second nucleic acid encoding a Staphylococcus lugdunensis Cas9 (SluCas9); or
   a nucleic acid encoding a Staphylococcus lugdunensis Cas9 (SluCas9); and
   optionally a DNA-PK inhibitor.
[Embodiment 36] A method of excising a CTG repeat in the 3′ UTR of the DMPK gene, the method comprising delivering to a cell a single nucleic acid molecule comprising:
   a nucleic acid encoding a pair of guide RNAs comprising:
      a. a first spacer sequence selected from any one of SEQ ID NOs: 1-65 and a second spacer sequence selected from any one of SEQ ID NOs: 67-167;
      b. a first and second spacer sequence comprising at least 20, or 21 contiguous nucleotides of any of the first and second spacer sequences of i) and;
      c. a first and second spacer sequence that is at least 90% identical to any of the first and second spacer sequences of i) a. or i) b.;
   a nucleic acid encoding a Staphylococcus lugdunensis Cas9 (SluCas9); and
   optionally DNA-PK inhibitor.
[Embodiment 37] The method of any one of embodiments 32-36, wherein the single nucleic acid molecule is delivered to the cell on a single vector.
[Embodiment 38] The method of any one of embodiments 32-37, comprising administering a DNA-PK inhibitor.
[Embodiment 39] The method of embodiment 38, wherein the DNA-PK inhibitor is Compound 6.
[Embodiment 40] The method of embodiment 38, wherein the DNA-PK inhibitor is Compound 1.
[Embodiment 41] The method of embodiment 38, wherein the DNA-PK inhibitor is Compound 2.
[Embodiment 42] The method of any one of embodiments 32-39, wherein the SluCas9 comprises the amino acid sequence of SEQ ID NO: 712.
[Embodiment 43] The method of any one of embodiments 32-40, wherein the SluCas9 is a variant of the amino acid sequence of SEQ ID NO: 712.
[Embodiment 44] The method of any one of embodiments 32-41, wherein the SluCas9 comprises an amino acid sequence selected from any one of SEQ ID NOs: 718-720.
[Embodiment 45] The composition or method of any one of embodiments 1-26 or 28-44, wherein the nucleic acid encoding the gRNA or the nucleic acid encoding the pair of gRNAs comprises a sequence selected from any one of SEQ ID NOs: 600-601, or 900-917.
[Embodiment 46] The composition or method of any one of embodiments 1-26 or 28-44, wherein the nucleic acid encoding the gRNA or the nucleic acid encoding the pair of gRNAs comprises a sequence selected from any one of SEQ ID NOs: 901-917.
[Embodiment 47] The composition of any one of the preceding embodiments, wherein the nucleic acid molecule encodes at least a first guide RNA and a second guide RNA.
[Embodiment 48] The composition of embodiment 47, wherein the nucleic acid molecule encodes a spacer sequence for the first guide RNA, a scaffold sequence for the first guide RNA, a spacer sequence for the second RNA, and a scaffold sequence for the second guide RNA.
[Embodiment 49] The composition of embodiment 48, wherein the spacer sequence for the first guide RNA and the spacer sequence for the second guide RNA are the same.
[Embodiment 50] The composition of embodiment 48, wherein the spacer sequence for the first guide RNA and the spacer sequence for the second guide RNA are different.
[Embodiment 51] The composition of embodiment 49 or 50, wherein the scaffold sequence for the first guide RNA and the scaffold sequence for the second guide RNA are the same.
[Embodiment 52] The composition of embodiment 49 or 50, wherein the scaffold sequence for the first guide RNA and the scaffold sequence for the second guide RNA are different.
[Embodiment 53] The composition of embodiment 52, wherein the scaffold sequence for the first guide RNA comprises a sequence selected from the group consisting of SEQ ID NOs: 901-916, and wherein the scaffold sequence for the second guide RNA comprises a different sequence selected from the group consisting of SEQ ID NOs: 901-916.
[Embodiment 54] A method of reducing the number of foci-positive cells, the method comprising delivering to a cell one or more nucleic acid molecules comprising:
   a nucleic acid encoding a guide RNA, wherein the guide RNA comprises:
      a. one or more spacer sequence selected from any one of SEQ ID NOs: 1-65, 67-167, and 201-531;
      b. one or more spacer sequence comprising at least 20, or 21 contiguous nucleotides of a spacer sequence selected from any one of SEQ ID NOs: 1-65, 67-167, and 201-531; or
      c. one or more spacer sequence that is at least 90% identical to any one of SEQ ID NOs: 1-65, 67-167, and 201-531;
   a nucleic acid encoding a Staphylococcus lugdunensis Cas9 (SluCas9); and
   optionally a DNA-PK inhibitor.
[Embodiment 55] A method of reducing the number of foci-positive cells, the method comprising delivering to a cell one or more nucleic acid molecules comprising:
   a nucleic acid encoding a pair of guide RNAs comprising:
      a. a first spacer sequence selected from any one of SEQ ID NOs: 1-65 and a second spacer sequence selected from any one of SEQ ID NOs: 67-167;
      b. a first and second spacer sequence comprising at least 20, or 21 contiguous nucleotides of any of the first and second spacer sequences of i) a.;
      c. a first and second spacer sequence that is at least 90% identical to any of the first and second spacer sequences of i) a. or i) b.;
      d. a first and second spacer, or one or more vectors encoding the pair of guide RNAs, wherein the first and second spacer sequences comprise any one of the following pairs of SEQ ID NOs: 6 and 72; 6 and 81; 6 and 84; 6 and 98; 6 and 100; 6 and 114; 6 and 122; 6 and 134; 6 and 139; 6 and 149; 6 and 166; 8 and 72; 8 and 72; 8 and 81; 8 and 84; 8 and 98; 8 and 100; 8 and 114; 8 and 122; 8 and 134; 8 and 139; 8 and 149; 8 and 166; 10 and 72; 10 and 81; 10 and 84; 10 and 98; 10 and 100; 10 and 114; 10 and 122; 10 and 134; 10 and 139; 10 and 149; 10 and 166; 21 and 72; 21 and 81; 21 and 84; 21 and 98; 21 and 100; 21 and 114; 21 and 122; 21 and 134; 21 and 139; 21 and 149; 21 and 166; 58 and 72; 58 and 81; 58 and 84; 58 and 98; 58 and 100; 58 and 114; 58 and 122; 58 and 134; 58 and 139; 58 and 149; 58 and 166; 62 and 72; 62 and 81; 62 and 84; 62 and 98; 62 and 100; 62 and 114; 62 and 122; 62 and 134; 62 and 139; 62 and 149; 62 and 166; 63 and 72; 63 and 81; 63 and 84; 63 and 98; 63 and 100; 63 and 114; 63 and 122; 63 and 134; 63 and 139; 63 and 149; 63 and 166; 64 and 72; 64 and 81; 64 and 84; 64 and 98; 64 and 100; 64 and 114; 64 and 122; 64 and 134; 64 and 139; 64 and 149; and 64 and 166;
      e. a first and second spacer, or one or more vectors encoding the pair of guide RNAs, wherein the first and second spacer sequences comprise SEQ ID NOs: 63 and 100 or SEQ ID NOs: 64 and 100;
   a nucleic acid encoding a Staphylococcus lugdunensis Cas9 (SluCas9); and
   optionally a DNA-PK inhibitor.
[Embodiment 56] The composition or method of any one of the preceding embodiments, comprising a pair of guide RNAs, wherein the pair of guide RNAs function to excise and also function as single guide cutters.
[Embodiment 57] The method of embodiment 54 or 55, wherein the first nucleic acid and the second nucleic acid are in the same nucleic acid molecule.
[Embodiment 58] The method of embodiment 54 or 55, wherein the first nucleic acid and the second nucleic acid are in separate nucleic acid molecules.
[Embodiment 59] The method of embodiment 58, wherein the separate nucleic acid molecules are each in separate vectors.
[Embodiment 60] The method of any one of embodiments 54-59, wherein the nucleic acid encoding the SluCas9 does not encode a guide RNA.
[Embodiment 61] The method of any one of embodiments 54-60, wherein the nucleic acid encoding the SluCas9 encodes one or more guide RNAs comprising:
   a. one or more spacer sequence selected from any one of SEQ ID NOs: 1-65, 67-167, and 201-531;
   b. one or more spacer sequence comprising at least 20, or 21 contiguous nucleotides of a spacer sequence selected from any one of SEQ ID NOs: 1-65, 67-167, and 201-531; or
   c. c. one or more spacer sequence that is at least 90% identical to any one of SEQ ID NOs: 1-65, 67-167, and 201-531.
[Embodiment 62] A composition comprising a first nucleic acid molecule and a second nucleic acid molecule, wherein the nucleic acid molecule encodes a Staphylococcus lugdunensis Cas9 (SluCas9) and the second nucleic acid molecule encodes: one or more guide RNAs comprising:
   a. one or more spacer sequence selected from any one of SEQ ID NOs: 1-65, 67-167, and 201-531;
   b. one or more spacer sequence comprising at least 20, or 21 contiguous nucleotides of a spacer sequence selected from any one of SEQ ID NOs: 1-65, 67-167, and 201-531; or
   c. c. one or more spacer sequence that is at least 90% identical to any one of SEQ ID NOs: 1-65, 67-167, and 201-531.
[Embodiment 63] The composition of embodiment 62, wherein the first nucleic acid molecule does not encode a guide RNA.
[Embodiment 64] The composition of embodiment 62, wherein the first nucleic acid molecule encodes:
   a. one or more spacer sequence selected from any one of SEQ ID NOs: 1-65, 67-167, and 201-531;
   b. one or more spacer sequence comprising at least 20, or 21 contiguous nucleotides of a spacer sequence selected from any one of SEQ ID NOs: 1-65, 67-167, and 201-531; or
   c. one or more spacer sequence that is at least 90% identical to any one of SEQ ID NOs: 1-65, 67-167, and 201-531.
[Embodiment 65] The composition of any one of embodiments 62-64, wherein the first nucleic acid molecule is in a first vector, and the second nucleic acid molecule is in a separate second vector.
[Embodiment 66] The composition of embodiment 65, wherein the first and second vectors are AAV vectors.
[Embodiment 67] The composition of embodiment 66, wherein the AAV vectors are AAV9 vectors.
[Embodiment 68] A composition comprising an AAV vector, wherein the vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence, a hU6c promoter for expression of a second sgRNA, a second sgRNA guide sequence, and a second sgRNA scaffold sequence, a promoter for expression of a nucleic acid encoding a Cas9 (e.g., CK8e), a nucleic acid encoding a SluCas9, and a polyadenylation sequence.
[Embodiment 69] A composition comprising an AAV vector, wherein the vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence, a 7SK2 promoter for expression of a second sgRNA, a second sgRNA guide sequence, and a second sgRNA scaffold sequence, a promoter for expression of a nucleic acid encoding a Cas9 (e.g., CK8e), a nucleic acid encoding a SluCas9, and a polyadenylation sequence.
[Embodiment 70] A composition comprising an AAV vector, wherein the vector comprises from 5′ to 3′ with respect to the plus strand: a promoter for expression of a nucleic acid encoding a Cas9 (e.g., CK8e), a nucleic acid encoding a SluCas9, a polyadenylation sequence, an hU6c promoter for expression of a nucleic acid encoding a first sgRNA, a nucleic acid encoding the first sgRNA guide sequence, a first sgRNA scaffold sequence, a hU6c promoter for expression of a second sgRNA, a second sgRNA guide sequence, and a second sgRNA scaffold sequence.
[Embodiment 71] A composition comprising an AAV vector, wherein the vector comprises from 5′ to 3′ with respect to the plus strand: a promoter for expression of a nucleic acid encoding a Cas9 (e.g., CK8e), a nucleic acid encoding a SluCas9, a polyadenylation sequence, an hU6c promoter for expression of a nucleic acid encoding a first sgRNA, a nucleic acid encoding the first sgRNA guide sequence, a first sgRNA scaffold sequence, a 7SK2 promoter for expression of a second sgRNA, a second sgRNA guide sequence, and a second sgRNA scaffold sequence.
[Embodiment 72] A composition comprising an AAV vector, wherein the vector comprises from 5′ to 3′ with respect to the plus strand: the reverse complement of a sequence encoding a first sgRNA scaffold sequence, the reverse complement of a sequence encoding a first sgRNA, the reverse complement of an 7SK2 or hU6c promoter for expression of a nucleic acid encoding a first sgRNA, a promoter for expression of a nucleic acid encoding a Cas9 (e.g., CK8e), a nucleic acid encoding a SluCas9, a polyadenylation sequence, a hU6c promoter for expression of a second sgRNA, a second sgRNA guide sequence, and a second sgRNA scaffold sequence.
[Embodiment 73] The composition of any one of embodiments 68-72, wherein the first sgRNA guide sequence comprises SEQ ID NO: 63, and the second sgRNA guide sequence comprises SEQ ID NO: 100.
[Embodiment 74] The composition of any one of embodiments 68-72, wherein the first sgRNA guide sequence comprises SEQ ID NO: 64, and the second sgRNA guide sequence comprises SEQ ID NO: 100.
[Embodiment 75] A composition comprising a nucleic acid molecule comprising nucleic acid encoding two different sgRNA guide sequences, wherein the first sgRNA guide sequence comprises SEQ ID NO: 63, and the second sgRNA guide sequence comprises SEQ ID NO: 100.
[Embodiment 76] A composition comprising a nucleic acid molecule comprising nucleic acid encoding two different sgRNA guide sequences, wherein the first sgRNA guide sequence comprises SEQ ID NO: 64, and the second sgRNA guide sequence comprises SEQ ID NO: 100.
[Embodiment 77] A composition comprising a first nucleic acid encoding one or more spacer sequence selected from any one of SEQ ID NOs: 8, 63, 64, and 81; and a second nucleic acid encoding a Staphylococcus lugdunensis Cas9 (SluCas9).
[Embodiment 78] A method of treating Myotonic Dystrophy Type 1 (DM1), the method comprising delivering to a cell the composition of any one of embodiments 68-77, and optionally a DNA-PK inhibitor.
[Embodiment 79] A method of excising a CTG repeat in the 3′ UTR of the DMPK gene, the method comprising delivering to a cell the composition of any one of embodiments 68-77.
[Embodiment 80] A method of treating Myotonic Dystrophy Type 1 (DM1), the method comprising delivering to a cell a single nucleic acid molecule comprising:
   i) a nucleic acid encoding a pair of guide RNAs comprising:
      a. a first and second spacer sequence, wherein the first spacer sequence comprises SEQ ID NO: 63, and the second spacer sequence comprises SEQ ID NO: 100; or
      b. a first and second spacer sequence, wherein the first spacer sequence comprises SEQ ID NO: 64, and the second spacer sequence comprises SEQ ID NO: 100;
   ii) a nucleic acid encoding a Staphylococcus lugdunensis Cas9 (SluCas9); and
   iii) optionally a DNA-PK inhibitor.
[Embodiment 81] A method of excising a CTG repeat in the 3′ UTR of the DMPK gene, the method comprising delivering to a cell a single nucleic acid molecule comprising:
   i) a nucleic acid encoding a pair of guide RNAs comprising:
      a. a first and second spacer sequence, wherein the first spacer sequence comprises SEQ ID NO: 63, and the second spacer sequence comprises SEQ ID NO: 100; or
      b. a first and second spacer sequence, wherein the first spacer sequence comprises SEQ ID NO: 64, and the second spacer sequence comprises SEQ ID NO: 100;
   ii) a nucleic acid encoding a Staphylococcus lugdunensis Cas9 (SluCas9); and
   iii) optionally a DNA-PK inhibitor.
[Embodiment 82] The composition of embodiment 75 or 76, wherein the composition further comprises a Staphylococcus lugdunensis Cas9 (SluCas9) or a nucleic acid encoding an SluCas9.
[Embodiment 83] The composition of any one of embodiments 75, 76 or 82, wherein the composition is associated with a lipid nanoparticle.
[Embodiment 84] The composition or method of any one of embodiments 1-74 or 77-83, wherein an SV40 nuclear localization signal (NLS) is fused to the N-terminus of the Cas9 and a nucleoplasmin NLS is fused to the C-terminus of the Cas9 protein.
[Embodiment 85] The composition or method of any one of embodiments 1-74 or 77-83, wherein a c-myc nuclear localization signal (NLS) is fused to the N-terminus of the Cas9 and an SV40 NLS and/or nucleoplasmin NLS is fused to the C-terminus of the Cas9.
[Embodiment 86] The composition or method of any one of embodiments 1-74 or 77-83, wherein a c-myc NLS is fused to the N-terminus of the Cas9 (e.g., by means of a linker such as GSVD (SEQ ID NO: 940)), an SV40 NLS is fused to the C-terminus of the Cas9 (e.g., by means of a linker such as GSGS (SEQ ID NO: 941)), and a nucleoplasmin NLS is fused to the C-terminus of the SV-40 NLS (e.g., by means of a linker such as GSGS (SEQ ID NO: 941)).
[Embodiment 87] The composition or method of any one of embodiments 1-86, wherein the guide RNA(s) comprise the sequence of SEQ ID NO: 901.

### BRIEF DESCRIPTION OF THE DRAWINGS

FIG. 1 shows the location of the 166 selected SluCas9 sgRNAs.

FIG. 2 shows the editing efficiency of 166 SluCas9 sgRNAs in primary DM1 patient myoblasts.

FIGS. 3A-3B show the TapeStation analysis of the PCR products amplified from DM1 myoblasts nucleofected with SluCas9 protein and 65 SluCas9 upstream sgRNAs. FIG. 3A without DNA-PKi and FIG. 3B with DNA-PKi.

FIGS. 4A-4B show the TapeStation analysis of the PCR products amplified from DM1 myoblasts nucleofected with SluCas9 protein and 101 SluCas9 downstream sgRNAs. FIG. 4A without DNA-PKi and FIG. 4B with DNA-PKi.

FIGS. 5A-5B show RNA foci reduction by individual SluCas9 sgRNAs. FIG. 5A shows upstream guides and FIG. 5B shows downstream guides.

FIGS. 6A-6B shows RNA foci reduction by SluU63 and SluD14. FIG. 6A shows immunofluorescence images showing CUG foci staining (small dots in cells) in myoblast nuclei (darker shading in images). FIG. 6B shows the frequency distribution of myoblast nuclei with different numbers of CUG foci.

FIG. 7 shows the location of the 19 selected SluCas9 sgRNAs for Dual-cut screening.

FIGS. 8A-B show a schematic of a loss-of-signal ddPCR assay (FIG. 8A) and the editing efficiency (CTG repeat excision efficiency %) of 88 SluCas9 sgRNA pairs tested in primary DM1 patient myoblasts (FIG. 8B).

FIGS. 9A-B show a TapeStation analysis of the PCR products amplified from DM1 myoblasts nucleofected with SluCas9 protein and 88 SluCas9 sgRNA pairs. FIG. 9A shows vehicle (DMSO) without DNA-PKi, and FIG. 9B shows with DNA-PKi.

FIG. 10 shows the RNA foci reduction by individual SluCas9 sgRNA pairs.

FIGS. 11A-B show RNA foci reduction by SluCas9 sgRNA-U63+D34 and sgRNA-U64+D34. FIG. 11A shows immunofluorescence images showing CUG foci staining (small dots in cells) in myoblast nuclei (darker shading in images). FIG. 11B shows the frequency distribution of myoblast nuclei with different numbers of CUG foci.

FIG. 12 is a schematic showing the representative vector configurations referred to as Design 1, Design 2, Design 3, and Design 4.

### DETAILED DESCRIPTION

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a guide” includes a plurality of guides and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

“Polynucleotide,” “nucleic acid,” and “nucleic acid molecule,” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA”, “guide RNA”, and simply “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “guide RNA” refers to each type. The trRNA may be a naturally-occurring sequence, or a trRNA sequence with modifications or variations compared to naturally-occurring sequences.

As used herein, a “spacer sequence,” sometimes also referred to herein and in the literature as a “spacer,” “protospacer,” “guide sequence,” or “targeting sequence” refers to a sequence within a guide RNA that is complementary to a target sequence and functions to direct a guide RNA to a target sequence for cleavage by a Cas9. A guide sequence can be 24, 23, 22, 21, 20 or fewer base pairs in length, e.g., in the case of Staphylococcus lugdunensis (i.e., SluCas9) and related Cas9 homologs/orthologs. Shorter or longer sequences can also be used as guides, e.g., 15-, 16-, 17-, 18-, 19-, 20-, 21-, 22-, 23-, 24-, or 25-nucleotides in length. In preferred embodiments, a guide/spacer sequence in the case of SluCas9 is at least 20 base pairs in length, or more specifically, within 20-25 base pairs in length (see, e.g., Schmidt et al., 2021, Nature Communications, “Improved CRISPR genome editing using small highly active and specific engineered RNA-guided nucleases”). For example, in some embodiments, the guide sequence comprises at least 17, 18, 19, 20, 21, 22, 23, 24, or 25 contiguous nucleotides of a sequence selected from SEQ ID NOs: 1-65, 67-167, and 201-531. In some embodiments, the guide sequence comprises a sequence selected from SEQ ID NOs: 1-65, 67-167, and 201-531. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. For example, in some embodiments, the guide sequence comprises a sequence with about 75%, 80%, 85%, 90%, 95%, 96%, 97%, 98%, 99%, or 100% identity to at least 17, 18, 19, 20, 21, 22, 23, 24, or 25 contiguous nucleotides of a sequence selected from SEQ ID NOs: 1-65, 67-167, and 201-531. In some embodiments, the guide sequence comprises a sequence with about 75%, 80%, 85%, 90%, 95%, 96%, 97%, 98%, 99%, or 100% identity to a sequence selected from SEQ ID NOs: 1-65, 67-167, and 201-531. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides. In some embodiments, the guide sequence and the target region do not contain any mismatches.

In some embodiments, the guide sequence comprises a sequence selected from SEQ ID NOs: 1-65, 67-167, and 201-531, wherein if the 5′ terminal nucleotide is not guanine, one or more guanine (g) is added to the sequence at its 5′ end. The 5′ g or gg may be necessary in some instances for transcription, for example, for expression by the RNA polymerase III-dependent U6 promoter or the T7 promoter. In some embodiments, a 5′ guanine is added to any one of the guide sequences or pairs of guide sequences disclosed herein.

Target sequences for Cas9s include both the positive and negative strands of genomic DNA (i.e., the sequence given and the sequence's reverse compliment), as a nucleic acid substrate for a Cas9 is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, “ribonucleoprotein” (RNP) or “RNP complex” refers to a guide RNA together with a Cas9. In some embodiments, the guide RNA guides the Cas9 such as Cas9 to a target sequence, and the guide RNA hybridizes with and the agent binds to the target sequence, which can be followed by cleaving or nicking (in the context of a modified “nickase” Cas9).

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide that is not DNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

Guide sequences useful in the guide RNA compositions and methods described herein are shown in Table 1A, and Table 1B and throughout the application.

As used herein, a “target sequence” refers to a sequence of nucleic acid in a target gene that has complementarity to at least a portion of the guide sequence of the guide RNA. The interaction of the target sequence and the guide sequence directs a Cas9 to bind, and potentially nick or cleave (depending on the activity of the agent), within the target sequence.

As used herein, “treatment” refers to any administration or application of a therapeutic for disease or disorder in a subject, and includes inhibiting the disease or development of the disease (which may occur before or after the disease is formally diagnosed, e.g., in cases where a subject has a genotype that has the potential or is likely to result in development of the disease), arresting its development, relieving one or more symptoms of the disease, curing the disease, or preventing reoccurrence of one or more symptoms of the disease. For example, treatment of DM1 may comprise alleviating symptoms of DM1.

As used herein, “ameliorating” refers to any beneficial effect on a phenotype or symptom, such as reducing its severity, slowing or delaying its development, arresting its development, or partially or completely reversing or eliminating it. In the case of quantitative phenotypes such as expression levels, ameliorating encompasses changing the expression level so that it is closer to the expression level seen in healthy or unaffected cells or individuals.

A “pharmaceutically acceptable excipient” refers to an agent that is included in a pharmaceutical formulation that is not the active ingredient. Pharmaceutically acceptable excipients may e.g., aid in drug delivery or support or enhance stability or bioavailability.

The term “about” or “approximately” means an acceptable error for a particular value as determined by one of ordinary skill in the art, which depends in part on how the value is measured or determined.

As used herein, “Staphylococcus lugdunensis Cas9” may also be referred to as SluCas9, and includes wild type SluCas9 (e.g., SEQ ID NO: 712) and variants thereof. A variant of SluCas9 comprises one or more amino acid changes as compared to SEQ ID NO: 712, including insertion, deletion, or substitution of one or more amino acids, or a chemical modification to one or more amino acids.

### II. Compositions

Provided herein are compositions useful for treating Myotonic Dystrophy Type 1 (DM1), e.g., using a single nucleic acid molecule encoding 1) one or more guide RNAs comprising one or more guide sequences of Table 1A and Table 1B; and 2) SluCas9. Such compositions may be administered to subjects having or suspected of having DM1. Any of the guide sequences disclosed herein may be in any of the pair combinations disclosed herein, and may be in a composition comprising any of the Cas9 proteins disclosed herein or a nucleic acid encoding any of the Cas9 proteins disclosed herein. Such compositions may be in any of the vectors disclosed herein (e.g., any of the AAV vectors disclosed herein) or be associated with a lipid nanoparticle.

In some embodiments, the disclosure provides for specific nucleic acid sequences encoding one or more guide RNA components (e.g., any of the spacer and or scaffold sequences disclosed herein). The disclosure contemplates RNA equivalents of any of the DNA sequences provided herein (i.e., in which “T”s are replaced with “U”s), or DNA equivalents of any of the RNA sequences provided herein (e.g., in which “U”s are replaced with “T”s), as well as complements (including reverse complements) of any of the sequences disclosed herein.

In some embodiments, the one or more guide RNAs direct the Cas9 to a site in or near a CTG repeat in the 3′ UTR of the DM1 protein kinase (DMPK) gene. For example, the Cas9 may be directed to cut within 10, 20, 30, 40, or 50 nucleotides of a target sequence.

In some embodiments, a composition comprising a single nucleic acid molecule encoding one or more guide RNAs and a Cas9 is provided, wherein the single nucleic acid molecule comprises:
a. a first nucleic acid encoding one or more spacer sequence selected from any one of SEQ ID NOs: 1-65, 67-167, and 201-531 and a second nucleic acid encoding a Staphylococcus lugdunensis Cas9 (SluCas9);
b. a first nucleic acid encoding one or more spacer sequence comprising at least 17, 18, 19, 20, or 21 contiguous nucleotides of a spacer sequence selected from any one of SEQ ID NOs: 1-65, 67-167, and 201-531 and a second nucleic acid encoding a Staphylococcus lugdunensis Cas9 (SluCas9);
c. a first nucleic acid encoding one or more spacer sequence that is at least 90% identical to any one of SEQ ID NOs: 1-65, 67-167, and 201-531 and a second nucleic acid encoding a Staphylococcus lugdunensis Cas9 (SluCas9).

In some embodiments, the composition further comprises a DNA-PK inhibitor. In some embodiments, the DNA-PK inhibitor is Compound 1. In some embodiments, the DNA-PK inhibitor is Compound 2. In some embodiments, the DNA-PK inhibitor is Compound 6.

In some embodiments, a first nucleic acid encoding 2 spacer sequences selected from any one of SEQ ID NOs: 63 and 100, and 64 and 100, and a second nucleic acid encoding a Staphylococcus lugdunensis Cas9 (SluCas9) is provided. In some embodiments, a first nucleic acid encoding one or more spacer sequence selected from any one of SEQ ID NOs: 8, 63, 64, and 81 and a second nucleic acid encoding a Staphylococcus lugdunensis Cas9 (SluCas9) is provided.

In some embodiments, a composition comprising a single nucleic acid molecule encoding one or more guide RNAs and a Cas9 is provided, wherein the single nucleic acid molecule comprises:
a. a first nucleic acid encoding a pair of guide RNAs comprising a first and second spacer sequence selected from any one of SEQ ID NOs:
   1 and 67; 1 and 68; 1 and 69; 1 and 70; 1 and 71; 1 and 72; 1 and 73; 1 and 74; 1 and 75; 1 and 76; 1 and 77; 1 and 78; 1 and 79; 1 and 80; 1 and 81; 1 and 82; 1 and 83; 1 and 84; 1 and 85; 1 and 86; 1 and 87; 1 and 88; 1 and 89; 1 and 90; 1 and 91; 1 and 92; 1 and 93; 1 and 94; 1 and 95; 1 and 96; 1 and 97; 1 and 98; 1 and 99; 1 and 100; 1 and 101; 1 and 102; 1 and 103; 1 and 104; 1 and 105; 1 and 106; 1 and 107; 1 and 108; 1 and 109; 1 and 110; 1 and 111; 1 and 112; 1 and 113; 1 and 114; 1 and 115; 1 and 116; 1 and 117; 1 and 118; 1 and 119; 1 and 120; 1 and 121; 1 and 122; 1 and 123; 1 and 124; 1 and 125; 1 and 126; 1 and 127; 1 and 128; 1 and 129; 1 and 130; 1 and 131; 1 and 132; 1 and 133; 1 and 134; 1 and 135; 1 and 136; 1 and 137; 1 and 138; 1 and 139; 1 and 140; 1 and 141; 1 and 142; 1 and 143; 1 and 144; 1 and 145; 1 and 146; 1 and 147; 1 and 148; 1 and 149; 1 and 150; 1 and 151; 1 and 152; 1 and 153; 1 and 154; 1 and 155; 1 and 156; 1 and 157; 1 and 158; 1 and 159; 1 and 160; 1 and 161; 1 and 162; 1 and 163; 1 and 164; 1 and 165; 1 and 166; 1 and 167; 2 and 67; 2 and 68; 2 and 69; 2 and 70; 2 and 71; 2 and 72; 2 and 73; 2 and 74; 2 and 75; 2 and 76; 2 and 77; 2 and 78; 2 and 79; 2 and 80; 2 and 81; 2 and 82; 2 and 83; 2 and 84; 2 and 85; 2 and 86; 2 and 87; 2 and 88; 2 and 89; 2 and 90; 2 and 91; 2 and 92; 2 and 93; 2 and 94; 2 and 95; 2 and 96; 2 and 97; 2 and 98; 2 and 99; 2 and 100; 2 and 101; 2 and 102; 2 and 103; 2 and 104; 2 and 105; 2 and 106; 2 and 107; 2 and 108; 2 and 109; 2 and 110; 2 and 111; 2 and 112; 2 and 113; 2 and 114; 2 and 115; 2 and 116; 2 and 117; 2 and 118; 2 and 119; 2 and 120; 2 and 121; 2 and 122; 2 and 123; 2 and 124; 2 and 125; 2 and 126; 2 and 127; 2 and 128; 2 and 129; 2 and 130; 2 and 131; 2 and 132; 2 and 133; 2 and 134; 2 and 135; 2 and 136; 2 and 137; 2 and 138; 2 and 139; 2 and 140; 2 and 141; 2 and 142; 2 and 143; 2 and 144; 2 and 145; 2 and 146; 2 and 147; 2 and 148; 2 and 149; 2 and 150; 2 and 151; 2 and 152; 2 and 153; 2 and 154; 2 and 155; 2 and 156; 2 and 157; 2 and 158; 2 and 159; 2 and 160; 2 and 161; 2 and 162; 2 and 163; 2 and 164; 2 and 165; 2 and 166; 2 and 167; 3 and 67; 3 and 68; 3 and 69; 3 and 70; 3 and 71; 3 and 72; 3 and 73; 3 and 74; 3 and 75; 3 and 76; 3 and 77; 3 and 78; 3 and 79; 3 and 80; 3 and 81; 3 and 82; 3 and 83; 3 and 84; 3 and 85; 3 and 86; 3 and 87; 3 and 88; 3 and 89; 3 and 90; 3 and 91; 3 and 92; 3 and 93; 3 and 94; 3 and 95; 3 and 96; 3 and 97; 3 and 98; 3 and 99; 3 and 100; 3 and 101; 3 and 102; 3 and 103; 3 and 104; 3 and 105; 3 and 106; 3 and 107; 3 and 108; 3 and 109; 3 and 110; 3 and 111; 3 and 112; 3 and 113; 3 and 114; 3 and 115; 3 and 116; 3 and 117; 3 and 118; 3 and 119; 3 and 120; 3 and 121; 3 and 122; 3 and 123; 3 and 124; 3 and 125; 3 and 126; 3 and 127; 3 and 128; 3 and 129; 3 and 130; 3 and 131; 3 and 132; 3 and 133; 3 and 134; 3 and 135; 3 and 136; 3 and 137; 3 and 138; 3 and 139; 3 and 140; 3 and 141; 3 and 142; 3 and 143; 3 and 144; 3 and 145; 3 and 146; 3 and 147; 3 and 148; 3 and 149; 3 and 150; 3 and 151; 3 and 152; 3 and 153; 3 and 154; 3 and 155; 3 and 156; 3 and 157; 3 and 158; 3 and 159; 3 and 160; 3 and 161; 3 and 162; 3 and 163; 3 and 164; 3 and 165; 3 and 166; 3 and 167; 4 and 67; 4 and 68; 4 and 69; 4 and 70; 4 and 71; 4 and 72; 4 and 73; 4 and 74; 4 and 75; 4 and 76; 4 and 77; 4 and 78; 4 and 79; 4 and 80; 4 and 81; 4 and 82; 4 and 83; 4 and 84; 4 and 85; 4 and 86; 4 and 87; 4 and 88; 4 and 89; 4 and 90; 4 and 91; 4 and 92; 4 and 93; 4 and 94; 4 and 95; 4 and 96; 4 and 97; 4 and 98; 4 and 99; 4 and 100; 4 and 101; 4 and 102; 4 and 103; 4 and 104; 4 and 105; 4 and 106; 4 and 107; 4 and 108; 4 and 109; 4 and 110; 4 and 111; 4 and 112; 4 and 113; 4 and 114; 4 and 115; 4 and 116; 4 and 117; 4 and 118; 4 and 119; 4 and 120; 4 and 121; 4 and 122; 4 and 123; 4 and 124; 4 and 125; 4 and 126; 4 and 127; 4 and 128; 4 and 129; 4 and 130; 4 and 131; 4 and 132; 4 and 133; 4 and 134; 4 and 135; 4 and 136; 4 and 137; 4 and 138; 4 and 139; 4 and 140; 4 and 141; 4 and 142; 4 and 143; 4 and 144; 4 and 145; 4 and 146; 4 and 147; 4 and 148; 4 and 149; 4 and 150; 4 and 151; 4 and 152; 4 and 153; 4 and 154; 4 and 155; 4 and 156; 4 and 157; 4 and 158; 4 and 159; 4 and 160; 4 and 161; 4 and 162; 4 and 163; 4 and 164; 4 and 165; 4 and 166; 4 and 167; 5 and 67; 5 and 68; 5 and 69; 5 and 70; 5 and 71; 5 and 72; 5 and 73; 5 and 74; 5 and 75; 5 and 76; 5 and 77; 5 and 78; 5 and 79; 5 and 80; 5 and 81; 5 and 82; 5 and 83; 5 and 84; 5 and 85; 5 and 86; 5 and 87; 5 and 88; 5 and 89; 5 and 90; 5 and 91; 5 and 92; 5 and 93; 5 and 94; 5 and 95; 5 and 96; 5 and 97; 5 and 98; 5 and 99; 5 and 100; 5 and 101; 5 and 102; 5 and 103; 5 and 104; 5 and 105; 5 and 106; 5 and 107; 5 and 108; 5 and 109; 5 and 110; 5 and 111; 5 and 112; 5 and 113; 5 and 114; 5 and 115; 5 and 116; 5 and 117; 5 and 118; 5 and 119; 5 and 120; 5 and 121; 5 and 122; 5 and 123; 5 and 124; 5 and 125; 5 and 126; 5 and 127; 5 and 128; 5 and 129; 5 and 130; 5 and 131; 5 and 132; 5 and 133; 5 and 134; 5 and 135; 5 and 136; 5 and 137; 5 and 138; 5 and 139; 5 and 140; 5 and 141; 5 and 142; 5 and 143; 5 and 144; 5 and 145; 5 and 146; 5 and 147; 5 and 148; 5 and 149; 5 and 150; 5 and 151; 5 and 152; 5 and 153; 5 and 154; 5 and 155; 5 and 156; 5 and 157; 5 and 158; 5 and 159; 5 and 160; 5 and 161; 5 and 162; 5 and 163; 5 and 164; 5 and 165; 5 and 166; 5 and 167; 6 and 67; 6 and 68; 6 and 69; 6 and 70; 6 and 71; 6 and 72; 6 and 73; 6 and 74; 6 and 75; 6 and 76; 6 and 77; 6 and 78; 6 and 79; 6 and 80; 6 and 81; 6 and 82; 6 and 83; 6 and 84; 6 and 85; 6 and 86; 6 and 87; 6 and 88; 6 and 89; 6 and 90; 6 and 91; 6 and 92; 6 and 93; 6 and 94; 6 and 95; 6 and 96; 6 and 97; 6 and 98; 6 and 99; 6 and 100; 6 and 101; 6 and 102; 6 and 103; 6 and 104; 6 and 105; 6 and 106; 6 and 107; 6 and 108; 6 and 109; 6 and 110; 6 and 111; 6 and 112; 6 and 113; 6 and 114; 6 and 115; 6 and 116; 6 and 117; 6 and 118; 6 and 119; 6 and 120; 6 and 121; 6 and 122; 6 and 123; 6 and 124; 6 and 125; 6 and 126; 6 and 127; 6 and 128; 6 and 129; 6 and 130; 6 and 131; 6 and 132; 6 and 133; 6 and 134; 6 and 135; 6 and 136; 6 and 137; 6 and 138; 6 and 139; 6 and 140; 6 and 141; 6 and 142; 6 and 143; 6 and 144; 6 and 145; 6 and 146; 6 and 147; 6 and 148; 6 and 149; 6 and 150; 6 and 151; 6 and 152; 6 and 153; 6 and 154; 6 and 155; 6 and 156; 6 and 157; 6 and 158; 6 and 159; 6 and 160; 6 and 161; 6 and 162; 6 and 163; 6 and 164; 6 and 165; 6 and 166; 6 and 167; 7 and 67; 7 and 68; 7 and 69; 7 and 70; 7 and 71; 7 and 72; 7 and 73; 7 and 74; 7 and 75; 7 and 76; 7 and 77; 7 and 78; 7 and 79; 7 and 80; 7 and 81; 7 and 82; 7 and 83; 7 and 84; 7 and 85; 7 and 86; 7 and 87; 7 and 88; 7 and 89; 7 and 90; 7 and 91; 7 and 92; 7 and 93; 7 and 94; 7 and 95; 7 and 96; 7 and 97; 7 and 98; 7 and 99; 7 and 100; 7 and 101; 7 and 102; 7 and 103; 7 and 104; 7 and 105; 7 and 106; 7 and 107; 7 and 108; 7 and 109; 7 and 110; 7 and 111; 7 and 112; 7 and 113; 7 and 114; 7 and 115; 7 and 116; 7 and 117; 7 and 118; 7 and 119; 7 and 120; 7 and 121; 7 and 122; 7 and 123; 7 and 124; 7 and 125; 7 and 126; 7 and 127; 7 and 128; 7 and 129; 7 and 130; 7 and 131; 7 and 132; 7 and 133; 7 and 134; 7 and 135; 7 and 136; 7 and 137; 7 and 138; 7 and 139; 7 and 140; 7 and 141; 7 and 142; 7 and 143; 7 and 144; 7 and 145; 7 and 146; 7 and 147; 7 and 148; 7 and 149; 7 and 150; 7 and 151; 7 and 152; 7 and 153; 7 and 154; 7 and 155; 7 and 156; 7 and 157; 7 and 158; 7 and 159; 7 and 160; 7 and 161; 7 and 162; 7 and 163; 7 and 164; 7 and 165; 7 and 166; 7 and 167; 8 and 67; 8 and 68; 8 and 69; 8 and 70; 8 and 71; 8 and 72; 8 and 73; 8 and 74; 8 and 75; 8 and 76; 8 and 77; 8 and 78; 8 and 79; 8 and 80; 8 and 81; 8 and 82; 8 and 83; 8 and 84; 8 and 85; 8 and 86; 8 and 87; 8 and 88; 8 and 89; 8 and 90; 8 and 91; 8 and 92; 8 and 93; 8 and 94; 8 and 95; 8 and 96; 8 and 97; 8 and 98; 8 and 99; 8 and 100; 8 and 101; 8 and 102; 8 and 103; 8 and 104; 8 and 105; 8 and 106; 8 and 107; 8 and 108; 8 and 109; 8 and 110; 8 and 111; 8 and 112; 8 and 113; 8 and 114; 8 and 115; 8 and 116; 8 and 117; 8 and 118; 8 and 119; 8 and 120; 8 and 121; 8 and 122; 8 and 123; 8 and 124; 8 and 125; 8 and 126; 8 and 127; 8 and 128; 8 and 129; 8 and 130; 8 and 131; 8 and 132; 8 and 133; 8 and 134; 8 and 135; 8 and 136; 8 and 137; 8 and 138; 8 and 139; 8 and 140; 8 and 141; 8 and 142; 8 and 143; 8 and 144; 8 and 145; 8 and 146; 8 and 147; 8 and 148; 8 and 149; 8 and 150; 8 and 151; 8 and 152; 8 and 153; 8 and 154; 8 and 155; 8 and 156; 8 and 157; 8 and 158; 8 and 159; 8 and 160; 8 and 161; 8 and 162; 8 and 163; 8 and 164; 8 and 165; 8 and 166; 8 and 167; 9 and 67; 9 and 68; 9 and 69; 9 and 70; 9 and 71; 9 and 72; 9 and 73; 9 and 74; 9 and 75; 9 and 76; 9 and 77; 9 and 78; 9 and 79; 9 and 80; 9 and 81; 9 and 82; 9 and 83; 9 and 84; 9 and 85; 9 and 86; 9 and 87; 9 and 88; 9 and 89; 9 and 90; 9 and 91; 9 and 92; 9 and 93; 9 and 94; 9 and 95; 9 and 96; 9 and 97; 9 and 98; 9 and 99; 9 and 100; 9 and 101; 9 and 102; 9 and 103; 9 and 104; 9 and 105; 9 and 106; 9 and 107; 9 and 108; 9 and 109; 9 and 110; 9 and 111; 9 and 112; 9 and 113; 9 and 114; 9 and 115; 9 and 116; 9 and 117; 9 and 118; 9 and 119; 9 and 120; 9 and 121; 9 and 122; 9 and 123; 9 and 124; 9 and 125; 9 and 126; 9 and 127; 9 and 128; 9 and 129; 9 and 130; 9 and 131; 9 and 132; 9 and 133; 9 and 134; 9 and 135; 9 and 136; 9 and 137; 9 and 138; 9 and 139; 9 and 140; 9 and 141; 9 and 142; 9 and 143; 9 and 144; 9 and 145; 9 and 146; 9 and 147; 9 and 148; 9 and 149; 9 and 150; 9 and 151; 9 and 152; 9 and 153; 9 and 154; 9 and 155; 9 and 156; 9 and 157; 9 and 158; 9 and 159; 9 and 160; 9 and 161; 9 and 162; 9 and 163; 9 and 164; 9 and 165; 9 and 166; 9 and 167; 10 and 67; 10 and 68; 10 and 69; 10 and 70; 10 and 71; 10 and 72; 10 and 73; 10 and 74; 10 and 75; 10 and 76; 10 and 77; 10 and 78; 10 and 79; 10 and 80; 10 and 81; 10 and 82; 10 and 83; 10 and 84; 10 and 85; 10 and 86; 10 and 87; 10 and 88; 10 and 89; 10 and 90; 10 and 91; 10 and 92; 10 and 93; 10 and 94; 10 and 95; 10 and 96; 10 and 97; 10 and 98; 10 and 99; 10 and 100; 10 and 101; 10 and 102; 10 and 103; 10 and 104; 10 and 105; 10 and 106; 10 and 107; 10 and 108; 10 and 109; 10 and 110; 10 and 111; 10 and 112; 10 and 113; 10 and 114; 10 and 115; 10 and 116; 10 and 117; 10 and 118; 10 and 119; 10 and 120; 10 and 121; 10 and 122; 10 and 123; 10 and 124; 10 and 125; 10 and 126; 10 and 127; 10 and 128; 10 and 129; 10 and 130; 10 and 131; 10 and 132; 10 and 133; 10 and 134; 10 and 135; 10 and 136; 10 and 137; 10 and 138; 10 and 139; 10 and 140; 10 and 141; 10 and 142; 10 and 143; 10 and 144; 10 and 145; 10 and 146; 10 and 147; 10 and 148; 10 and 149; 10 and 150; 10 and 151; 10 and 152; 10 and 153; 10 and 154; 10 and 155; 10 and 156; 10 and 157; 10 and 158; 10 and 159; 10 and 160; 10 and 161; 10 and 162; 10 and 163; 10 and 164; 10 and 165; 10 and 166; 10 and 167; 11 and 67; 11 and 68; 11 and 69; 11 and 70; 11 and 71; 11 and 72; 11 and 73; 11 and 74; 11 and 75; 11 and 76; 11 and 77; 11 and 78; 11 and 79; 11 and 80; 11 and 81; 11 and 82; 11 and 83; 11 and 84; 11 and 85; 11 and 86; 11 and 87; 11 and 88; 11 and 89; 11 and 90; 11 and 91; 11 and 92; 11 and 93; 11 and 94; 11 and 95; 11 and 96; 11 and 97; 11 and 98; 11 and 99; 11 and 100; 11 and 101; 11 and 102; 11 and 103; 11 and 104; 11 and 105; 11 and 106; 11 and 107; 11 and 108; 11 and 109; 11 and 110; 11 and 111; 11 and 112; 11 and 113; 11 and 114; 11 and 115; 11 and 116; 11 and 117; 11 and 118; 11 and 119; 11 and 120; 11 and 121; 11 and 122; 11 and 123; 11 and 124; 11 and 125; 11 and 126; 11 and 127; 11 and 128; 11 and 129; 11 and 130; 11 and 131; 11 and 132; 11 and 133; 11 and 134; 11 and 135; 11 and 136; 11 and 137; 11 and 138; 11 and 139; 11 and 140; 11 and 141; 11 and 142; 11 and 143; 11 and 144; 11 and 145; 11 and 146; 11 and 147; 11 and 148; 11 and 149; 11 and 150; 11 and 151; 11 and 152; 11 and 153; 11 and 154; 11 and 155; 11 and 156; 11 and 157; 11 and 158; 11 and 159; 11 and 160; 11 and 161; 11 and 162; 11 and 163; 11 and 164; 11 and 165; 11 and 166; 11 and 167; 12 and 67; 12 and 68; 12 and 69; 12 and 70; 12 and 71; 12 and 72; 12 and 73; 12 and 74; 12 and 75; 12 and 76; 12 and 77; 12 and 78; 12 and 79; 12 and 80; 12 and 81; 12 and 82; 12 and 83; 12 and 84; 12 and 85; 12 and 86; 12 and 87; 12 and 88; 12 and 89; 12 and 90; 12 and 91; 12 and 92; 12 and 93; 12 and 94; 12 and 95; 12 and 96; 12 and 97; 12 and 98; 12 and 99; 12 and 100; 12 and 101; 12 and 102; 12 and 103; 12 and 104; 12 and 105; 12 and 106; 12 and 107; 12 and 108; 12 and 109; 12 and 110; 12 and 111; 12 and 112; 12 and 113; 12 and 114; 12 and 115; 12 and 116; 12 and 117; 12 and 118; 12 and 119; 12 and 120; 12 and 121; 12 and 122; 12 and 123; 12 and 124; 12 and 125; 12 and 126; 12 and 127; 12 and 128; 12 and 129; 12 and 130; 12 and 131; 12 and 132; 12 and 133; 12 and 134; 12 and 135; 12 and 136; 12 and 137; 12 and 138; 12 and 139; 12 and 140; 12 and 141; 12 and 142; 12 and 143; 12 and 144; 12 and 145; 12 and 146; 12 and 147; 12 and 148; 12 and 149; 12 and 150; 12 and 151; 12 and 152; 12 and 153; 12 and 154; 12 and 155; 12 and 156; 12 and 157; 12 and 158; 12 and 159; 12 and 160; 12 and 161; 12 and 162; 12 and 163; 12 and 164; 12 and 165; 12 and 166; 12 and 167; 13 and 67; 13 and 68; 13 and 69; 13 and 70; 13 and 71; 13 and 72; 13 and 73; 13 and 74; 13 and 75; 13 and 76; 13 and 77; 13 and 78; 13 and 79; 13 and 80; 13 and 81; 13 and 82; 13 and 83; 13 and 84; 13 and 85; 13 and 86; 13 and 87; 13 and 88; 13 and 89; 13 and 90; 13 and 91; 13 and 92; 13 and 93; 13 and 94; 13 and 95; 13 and 96; 13 and 97; 13 and 98; 13 and 99; 13 and 100; 13 and 101; 13 and 102; 13 and 103; 13 and 104; 13 and 105; 13 and 106; 13 and 107; 13 and 108; 13 and 109; 13 and 110; 13 and 111; 13 and 112; 13 and 113; 13 and 114; 13 and 115; 13 and 116; 13 and 117; 13 and 118; 13 and 119; 13 and 120; 13 and 121; 13 and 122; 13 and 123; 13 and 124; 13 and 125; 13 and 126; 13 and 127; 13 and 128; 13 and 129; 13 and 130; 13 and 131; 13 and 132; 13 and 133; 13 and 134; 13 and 135; 13 and 136; 13 and 137; 13 and 138; 13 and 139; 13 and 140; 13 and 141; 13 and 142; 13 and 143; 13 and 144; 13 and 145; 13 and 146; 13 and 147; 13 and 148; 13 and 149; 13 and 150; 13 and 151; 13 and 152; 13 and 153; 13 and 154; 13 and 155; 13 and 156; 13 and 157; 13 and 158; 13 and 159; 13 and 160; 13 and 161; 13 and 162; 13 and 163; 13 and 164; 13 and 165; 13 and 166; 13 and 167; 14 and 67; 14 and 68; 14 and 69; 14 and 70; 14 and 71; 14 and 72; 14 and 73; 14 and 74; 14 and 75; 14 and 76; 14 and 77; 14 and 78; 14 and 79; 14 and 80; 14 and 81; 14 and 82; 14 and 83; 14 and 84; 14 and 85; 14 and 86; 14 and 87; 14 and 88; 14 and 89; 14 and 90; 14 and 91; 14 and 92; 14 and 93; 14 and 94; 14 and 95; 14 and 96; 14 and 97; 14 and 98; 14 and 99; 14 and 100; 14 and 101; 14 and 102; 14 and 103; 14 and 104; 14 and 105; 14 and 106; 14 and 107; 14 and 108; 14 and 109; 14 and 110; 14 and 111; 14 and 112; 14 and 113; 14 and 114; 14 and 115; 14 and 116; 14 and 117; 14 and 118; 14 and 119; 14 and 120; 14 and 121; 14 and 122; 14 and 123; 14 and 124; 14 and 125; 14 and 126; 14 and 127; 14 and 128; 14 and 129; 14 and 130; 14 and 131; 14 and 132; 14 and 133; 14 and 134; 14 and 135; 14 and 136; 14 and 137; 14 and 138; 14 and 139; 14 and 140; 14 and 141; 14 and 142; 14 and 143; 14 and 144; 14 and 145; 14 and 146; 14 and 147; 14 and 148; 14 and 149; 14 and 150; 14 and 151; 14 and 152; 14 and 153; 14 and 154; 14 and 155; 14 and 156; 14 and 157; 14 and 158; 14 and 159; 14 and 160; 14 and 161; 14 and 162; 14 and 163; 14 and 164; 14 and 165; 14 and 166; 14 and 167; 15 and 67; 15 and 68; 15 and 69; 15 and 70; 15 and 71; 15 and 72; 15 and 73; 15 and 74; 15 and 75; 15 and 76; 15 and 77; 15 and 78; 15 and 79; 15 and 80; 15 and 81; 15 and 82; 15 and 83; 15 and 84; 15 and 85; 15 and 86; 15 and 87; 15 and 88; 15 and 89; 15 and 90; 15 and 91; 15 and 92; 15 and 93; 15 and 94; 15 and 95; 15 and 96; 15 and 97; 15 and 98; 15 and 99; 15 and 100; 15 and 101; 15 and 102; 15 and 103; 15 and 104; 15 and 105; 15 and 106; 15 and 107; 15 and 108; 15 and 109; 15 and 110; 15 and 111; 15 and 112; 15 and 113; 15 and 114; 15 and 115; 15 and 116; 15 and 117; 15 and 118; 15 and 119; 15 and 120; 15 and 121; 15 and 122; 15 and 123; 15 and 124; 15 and 125; 15 and 126; 15 and 127; 15 and 128; 15 and 129; 15 and 130; 15 and 131; 15 and 132; 15 and 133; 15 and 134; 15 and 135; 15 and 136; 15 and 137; 15 and 138; 15 and 139; 15 and 140; 15 and 141; 15 and 142; 15 and 143; 15 and 144; 15 and 145; 15 and 146; 15 and 147; 15 and 148; 15 and 149; 15 and 150; 15 and 151; 15 and 152; 15 and 153; 15 and 154; 15 and 155; 15 and 156; 15 and 157; 15 and 158; 15 and 159; 15 and 160; 15 and 161; 15 and 162; 15 and 163; 15 and 164; 15 and 165; 15 and 166; 15 and 167; 16 and 67; 16 and 68; 16 and 69; 16 and 70; 16 and 71; 16 and 72; 16 and 73; 16 and 74; 16 and 75; 16 and 76; 16 and 77; 16 and 78; 16 and 79; 16 and 80; 16 and 81; 16 and 82; 16 and 83; 16 and 84; 16 and 85; 16 and 86; 16 and 87; 16 and 88; 16 and 89; 16 and 90; 16 and 91; 16 and 92; 16 and 93; 16 and 94; 16 and 95; 16 and 96; 16 and 97; 16 and 98; 16 and 99; 16 and 100; 16 and 101; 16 and 102; 16 and 103; 16 and 104; 16 and 105; 16 and 106; 16 and 107; 16 and 108; 16 and 109; 16 and 110; 16 and 111; 16 and 112; 16 and 113; 16 and 114; 16 and 115; 16 and 116; 16 and 117; 16 and 118; 16 and 119; 16 and 120; 16 and 121; 16 and 122; 16 and 123; 16 and 124; 16 and 125; 16 and 126; 16 and 127; 16 and 128; 16 and 129; 16 and 130; 16 and 131; 16 and 132; 16 and 133; 16 and 134; 16 and 135; 16 and 136; 16 and 137; 16 and 138; 16 and 139; 16 and 140; 16 and 141; 16 and 142; 16 and 143; 16 and 144; 16 and 145; 16 and 146; 16 and 147; 16 and 148; 16 and 149; 16 and 150; 16 and 151; 16 and 152; 16 and 153; 16 and 154; 16 and 155; 16 and 156; 16 and 157; 16 and 158; 16 and 159; 16 and 160; 16 and 161; 16 and 162; 16 and 163; 16 and 164; 16 and 165; 16 and 166; 16 and 167; 17 and 67; 17 and 68; 17 and 69; 17 and 70; 17 and 71; 17 and 72; 17 and 73; 17 and 74; 17 and 75; 17 and 76; 17 and 77; 17 and 78; 17 and 79; 17 and 80; 17 and 81; 17 and 82; 17 and 83; 17 and 84; 17 and 85; 17 and 86; 17 and 87; 17 and 88; 17 and 89; 17 and 90; 17 and 91; 17 and 92; 17 and 93; 17 and 94; 17 and 95; 17 and 96; 17 and 97; 17 and 98; 17 and 99; 17 and 100; 17 and 101; 17 and 102; 17 and 103; 17 and 104; 17 and 105; 17 and 106; 17 and 107; 17 and 108; 17 and 109; 17 and 110; 17 and 111; 17 and 112; 17 and 113; 17 and 114; 17 and 115; 17 and 116; 17 and 117; 17 and 118; 17 and 119; 17 and 120; 17 and 121; 17 and 122; 17 and 123; 17 and 124; 17 and 125; 17 and 126; 17 and 127; 17 and 128; 17 and 129; 17 and 130; 17 and 131; 17 and 132; 17 and 133; 17 and 134; 17 and 135; 17 and 136; 17 and 137; 17 and 138; 17 and 139; 17 and 140; 17 and 141; 17 and 142; 17 and 143; 17 and 144; 17 and 145; 17 and 146; 17 and 147; 17 and 148; 17 and 149; 17 and 150; 17 and 151; 17 and 152; 17 and 153; 17 and 154; 17 and 155; 17 and 156; 17 and 157; 17 and 158; 17 and 159; 17 and 160; 17 and 161; 17 and 162; 17 and 163; 17 and 164; 17 and 165; 17 and 166; 17 and 167; 18 and 67; 18 and 68; 18 and 69; 18 and 70; 18 and 71; 18 and 72; 18 and 73; 18 and 74; 18 and 75; 18 and 76; 18 and 77; 18 and 78; 18 and 79; 18 and 80; 18 and 81; 18 and 82; 18 and 83; 18 and 84; 18 and 85; 18 and 86; 18 and 87; 18 and 88; 18 and 89; 18 and 90; 18 and 91; 18 and 92; 18 and 93; 18 and 94; 18 and 95; 18 and 96; 18 and 97; 18 and 98; 18 and 99; 18 and 100; 18 and 101; 18 and 102; 18 and 103; 18 and 104; 18 and 105; 18 and 106; 18 and 107; 18 and 108; 18 and 109; 18 and 110; 18 and 111; 18 and 112; 18 and 113; 18 and 114; 18 and 115; 18 and 116; 18 and 117; 18 and 118; 18 and 119; 18 and 120; 18 and 121; 18 and 122; 18 and 123; 18 and 124; 18 and 125; 18 and 126; 18 and 127; 18 and 128; 18 and 129; 18 and 130; 18 and 131; 18 and 132; 18 and 133; 18 and 134; 18 and 135; 18 and 136; 18 and 137; 18 and 138; 18 and 139; 18 and 140; 18 and 141; 18 and 142; 18 and 143; 18 and 144; 18 and 145; 18 and 146; 18 and 147; 18 and 148; 18 and 149; 18 and 150; 18 and 151; 18 and 152; 18 and 153; 18 and 154; 18 and 155; 18 and 156; 18 and 157; 18 and 158; 18 and 159; 18 and 160; 18 and 161; 18 and 162; 18 and 163; 18 and 164; 18 and 165; 18 and 166; 18 and 167; 19 and 67; 19 and 68; 19 and 69; 19 and 70; 19 and 71; 19 and 72; 19 and 73; 19 and 74; 19 and 75; 19 and 76; 19 and 77; 19 and 78; 19 and 79; 19 and 80; 19 and 81; 19 and 82; 19 and 83; 19 and 84; 19 and 85; 19 and 86; 19 and 87; 19 and 88; 19 and 89; 19 and 90; 19 and 91; 19 and 92; 19 and 93; 19 and 94; 19 and 95; 19 and 96; 19 and 97; 19 and 98; 19 and 99; 19 and 100; 19 and 101; 19 and 102; 19 and 103; 19 and 104; 19 and 105; 19 and 106; 19 and 107; 19 and 108; 19 and 109; 19 and 110; 19 and 111; 19 and 112; 19 and 113; 19 and 114; 19 and 115; 19 and 116; 19 and 117; 19 and 118; 19 and 119; 19 and 120; 19 and 121; 19 and 122; 19 and 123; 19 and 124; 19 and 125; 19 and 126; 19 and 127; 19 and 128; 19 and 129; 19 and 130; 19 and 131; 19 and 132; 19 and 133; 19 and 134; 19 and 135; 19 and 136; 19 and 137; 19 and 138; 19 and 139; 19 and 140; 19 and 141; 19 and 142; 19 and 143; 19 and 144; 19 and 145; 19 and 146; 19 and 147; 19 and 148; 19 and 149; 19 and 150; 19 and 151; 19 and 152; 19 and 153; 19 and 154; 19 and 155; 19 and 156; 19 and 157; 19 and 158; 19 and 159; 19 and 160; 19 and 161; 19 and 162; 19 and 163; 19 and 164; 19 and 165; 19 and 166; 19 and 167; 20 and 67; 20 and 68; 20 and 69; 20 and 70; 20 and 71; 20 and 72; 20 and 73; 20 and 74; 20 and 75; 20 and 76; 20 and 77; 20 and 78; 20 and 79; 20 and 80; 20 and 81; 20 and 82; 20 and 83; 20 and 84; 20 and 85; 20 and 86; 20 and 87; 20 and 88; 20 and 89; 20 and 90; 20 and 91; 20 and 92; 20 and 93; 20 and 94; 20 and 95; 20 and 96; 20 and 97; 20 and 98; 20 and 99; 20 and 100; 20 and 101; 20 and 102; 20 and 103; 20 and 104; 20 and 105; 20 and 106; 20 and 107; 20 and 108; 20 and 109; 20 and 110; 20 and 111; 20 and 112; 20 and 113; 20 and 114; 20 and 115; 20 and 116; 20 and 117; 20 and 118; 20 and 119; 20 and 120; 20 and 121; 20 and 122; 20 and 123; 20 and 124; 20 and 125; 20 and 126; 20 and 127; 20 and 128; 20 and 129; 20 and 130; 20 and 131; 20 and 132; 20 and 133; 20 and 134; 20 and 135; 20 and 136; 20 and 137; 20 and 138; 20 and 139; 20 and 140; 20 and 141; 20 and 142; 20 and 143; 20 and 144; 20 and 145; 20 and 146; 20 and 147; 20 and 148; 20 and 149; 20 and 150; 20 and 151; 20 and 152; 20 and 153; 20 and 154; 20 and 155; 20 and 156; 20 and 157; 20 and 158; 20 and 159; 20 and 160; 20 and 161; 20 and 162; 20 and 163; 20 and 164; 20 and 165; 20 and 166; 20 and 167; 21 and 67; 21 and 68; 21 and 69; 21 and 70; 21 and 71; 21 and 72; 21 and 73; 21 and 74; 21 and 75; 21 and 76; 21 and 77; 21 and 78; 21 and 79; 21 and 80; 21 and 81; 21 and 82; 21 and 83; 21 and 84; 21 and 85; 21 and 86; 21 and 87; 21 and 88; 21 and 89; 21 and 90; 21 and 91; 21 and 92; 21 and 93; 21 and 94; 21 and 95; 21 and 96; 21 and 97; 21 and 98; 21 and 99; 21 and 100; 21 and 101; 21 and 102; 21 and 103; 21 and 104; 21 and 105; 21 and 106; 21 and 107; 21 and 108; 21 and 109; 21 and 110; 21 and 111; 21 and 112; 21 and 113; 21 and 114; 21 and 115; 21 and 116; 21 and 117; 21 and 118; 21 and 119; 21 and 120; 21 and 121; 21 and 122; 21 and 123; 21 and 124; 21 and 125; 21 and 126; 21 and 127; 21 and 128; 21 and 129; 21 and 130; 21 and 131; 21 and 132; 21 and 133; 21 and 134; 21 and 135; 21 and 136; 21 and 137; 21 and 138; 21 and 139; 21 and 140; 21 and 141; 21 and 142; 21 and 143; 21 and 144; 21 and 145; 21 and 146; 21 and 147; 21 and 148; 21 and 149; 21 and 150; 21 and 151; 21 and 152; 21 and 153; 21 and 154; 21 and 155; 21 and 156; 21 and 157; 21 and 158; 21 and 159; 21 and 160; 21 and 161; 21 and 162; 21 and 163; 21 and 164; 21 and 165; 21 and 166; 21 and 167; 22 and 67; 22 and 68; 22 and 69; 22 and 70; 22 and 71; 22 and 72; 22 and 73; 22 and 74; 22 and 75; 22 and 76; 22 and 77; 22 and 78; 22 and 79; 22 and 80; 22 and 81; 22 and 82; 22 and 83; 22 and 84; 22 and 85; 22 and 86; 22 and 87; 22 and 88; 22 and 89; 22 and 90; 22 and 91; 22 and 92; 22 and 93; 22 and 94; 22 and 95; 22 and 96; 22 and 97; 22 and 98; 22 and 99; 22 and 100; 22 and 101; 22 and 102; 22 and 103; 22 and 104; 22 and 105; 22 and 106; 22 and 107; 22 and 108; 22 and 109; 22 and 110; 22 and 111; 22 and 112; 22 and 113; 22 and 114; 22 and 115; 22 and 116; 22 and 117; 22 and 118; 22 and 119; 22 and 120; 22 and 121; 22 and 122; 22 and 123; 22 and 124; 22 and 125; 22 and 126; 22 and 127; 22 and 128; 22 and 129; 22 and 130; 22 and 131; 22 and 132; 22 and 133; 22 and 134; 22 and 135; 22 and 136; 22 and 137; 22 and 138; 22 and 139; 22 and 140; 22 and 141; 22 and 142; 22 and 143; 22 and 144; 22 and 145; 22 and 146; 22 and 147; 22 and 148; 22 and 149; 22 and 150; 22 and 151; 22 and 152; 22 and 153; 22 and 154; 22 and 155; 22 and 156; 22 and 157; 22 and 158; 22 and 159; 22 and 160; 22 and 161; 22 and 162; 22 and 163; 22 and 164; 22 and 165; 22 and 166; 22 and 167; 23 and 67; 23 and 68; 23 and 69; 23 and 70; 23 and 71; 23 and 72; 23 and 73; 23 and 74; 23 and 75; 23 and 76; 23 and 77; 23 and 78; 23 and 79; 23 and 80; 23 and 81; 23 and 82; 23 and 83; 23 and 84; 23 and 85; 23 and 86; 23 and 87; 23 and 88; 23 and 89; 23 and 90; 23 and 91; 23 and 92; 23 and 93; 23 and 94; 23 and 95; 23 and 96; 23 and 97; 23 and 98; 23 and 99; 23 and 100; 23 and 101; 23 and 102; 23 and 103; 23 and 104; 23 and 105; 23 and 106; 23 and 107; 23 and 108; 23 and 109; 23 and 110; 23 and 111; 23 and 112; 23 and 113; 23 and 114; 23 and 115; 23 and 116; 23 and 117; 23 and 118; 23 and 119; 23 and 120; 23 and 121; 23 and 122; 23 and 123; 23 and 124; 23 and 125; 23 and 126; 23 and 127; 23 and 128; 23 and 129; 23 and 130; 23 and 131; 23 and 132; 23 and 133; 23 and 134; 23 and 135; 23 and 136; 23 and 137; 23 and 138; 23 and 139; 23 and 140; 23 and 141; 23 and 142; 23 and 143; 23 and 144; 23 and 145; 23 and 146; 23 and 147; 23 and 148; 23 and 149; 23 and 150; 23 and 151; 23 and 152; 23 and 153; 23 and 154; 23 and 155; 23 and 156; 23 and 157; 23 and 158; 23 and 159; 23 and 160; 23 and 161; 23 and 162; 23 and 163; 23 and 164; 23 and 165; 23 and 166; 23 and 167; 24 and 67; 24 and 68; 24 and 69; 24 and 70; 24 and 71; 24 and 72; 24 and 73; 24 and 74; 24 and 75; 24 and 76; 24 and 77; 24 and 78; 24 and 79; 24 and 80; 24 and 81; 24 and 82; 24 and 83; 24 and 84; 24 and 85; 24 and 86; 24 and 87; 24 and 88; 24 and 89; 24 and 90; 24 and 91; 24 and 92; 24 and 93; 24 and 94; 24 and 95; 24 and 96; 24 and 97; 24 and 98; 24 and 99; 24 and 100; 24 and 101; 24 and 102; 24 and 103; 24 and 104; 24 and 105; 24 and 106; 24 and 107; 24 and 108; 24 and 109; 24 and 110; 24 and 111; 24 and 112; 24 and 113; 24 and 114; 24 and 115; 24 and 116; 24 and 117; 24 and 118; 24 and 119; 24 and 120; 24 and 121; 24 and 122; 24 and 123; 24 and 124; 24 and 125; 24 and 126; 24 and 127; 24 and 128; 24 and 129; 24 and 130; 24 and 131; 24 and 132; 24 and 133; 24 and 134; 24 and 135; 24 and 136; 24 and 137; 24 and 138; 24 and 139; 24 and 140; 24 and 141; 24 and 142; 24 and 143; 24 and 144; 24 and 145; 24 and 146; 24 and 147; 24 and 148; 24 and 149; 24 and 150; 24 and 151; 24 and 152; 24 and 153; 24 and 154; 24 and 155; 24 and 156; 24 and 157; 24 and 158; 24 and 159; 24 and 160; 24 and 161; 24 and 162; 24 and 163; 24 and 164; 24 and 165; 24 and 166; 24 and 167; 25 and 67; 25 and 68; 25 and 69; 25 and 70; 25 and 71; 25 and 72; 25 and 73; 25 and 74; 25 and 75; 25 and 76; 25 and 77; 25 and 78; 25 and 79; 25 and 80; 25 and 81; 25 and 82; 25 and 83; 25 and 84; 25 and 85; 25 and 86; 25 and 87; 25 and 88; 25 and 89; 25 and 90; 25 and 91; 25 and 92; 25 and 93; 25 and 94; 25 and 95; 25 and 96; 25 and 97; 25 and 98; 25 and 99; 25 and 100; 25 and 101; 25 and 102; 25 and 103; 25 and 104; 25 and 105; 25 and 106; 25 and 107; 25 and 108; 25 and 109; 25 and 110; 25 and 111; 25 and 112; 25 and 113; 25 and 114; 25 and 115; 25 and 116; 25 and 117; 25 and 118; 25 and 119; 25 and 120; 25 and 121; 25 and 122; 25 and 123; 25 and 124; 25 and 125; 25 and 126; 25 and 127; 25 and 128; 25 and 129; 25 and 130; 25 and 131; 25 and 132; 25 and 133; 25 and 134; 25 and 135; 25 and 136; 25 and 137; 25 and 138; 25 and 139; 25 and 140; 25 and 141; 25 and 142; 25 and 143; 25 and 144; 25 and 145; 25 and 146; 25 and 147; 25 and 148; 25 and 149; 25 and 150; 25 and 151; 25 and 152; 25 and 153; 25 and 154; 25 and 155; 25 and 156; 25 and 157; 25 and 158; 25 and 159; 25 and 160; 25 and 161; 25 and 162; 25 and 163; 25 and 164; 25 and 165; 25 and 166; 25 and 167; 26 and 67; 26 and 68; 26 and 69; 26 and 70; 26 and 71; 26 and 72; 26 and 73; 26 and 74; 26 and 75; 26 and 76; 26 and 77; 26 and 78; 26 and 79; 26 and 80; 26 and 81; 26 and 82; 26 and 83; 26 and 84; 26 and 85; 26 and 86; 26 and 87; 26 and 88; 26 and 89; 26 and 90; 26 and 91; 26 and 92; 26 and 93; 26 and 94; 26 and 95; 26 and 96; 26 and 97; 26 and 98; 26 and 99; 26 and 100; 26 and 101; 26 and 102; 26 and 103; 26 and 104; 26 and 105; 26 and 106; 26 and 107; 26 and 108; 26 and 109; 26 and 110; 26 and 111; 26 and 112; 26 and 113; 26 and 114; 26 and 115; 26 and 116; 26 and 117; 26 and 118; 26 and 119; 26 and 120; 26 and 121; 26 and 122; 26 and 123; 26 and 124; 26 and 125; 26 and 126; 26 and 127; 26 and 128; 26 and 129; 26 and 130; 26 and 131; 26 and 132; 26 and 133; 26 and 134; 26 and 135; 26 and 136; 26 and 137; 26 and 138; 26 and 139; 26 and 140; 26 and 141; 26 and 142; 26 and 143; 26 and 144; 26 and 145; 26 and 146; 26 and 147; 26 and 148; 26 and 149; 26 and 150; 26 and 151; 26 and 152; 26 and 153; 26 and 154; 26 and 155; 26 and 156; 26 and 157; 26 and 158; 26 and 159; 26 and 160; 26 and 161; 26 and 162; 26 and 163; 26 and 164; 26 and 165; 26 and 166; 26 and 167; 27 and 67; 27 and 68; 27 and 69; 27 and 70; 27 and 71; 27 and 72; 27 and 73; 27 and 74; 27 and 75; 27 and 76; 27 and 77; 27 and 78; 27 and 79; 27 and 80; 27 and 81; 27 and 82; 27 and 83; 27 and 84; 27 and 85; 27 and 86; 27 and 87; 27 and 88; 27 and 89; 27 and 90; 27 and 91; 27 and 92; 27 and 93; 27 and 94; 27 and 95; 27 and 96; 27 and 97; 27 and 98; 27 and 99; 27 and 100; 27 and 101; 27 and 102; 27 and 103; 27 and 104; 27 and 105; 27 and 106; 27 and 107; 27 and 108; 27 and 109; 27 and 110; 27 and 111; 27 and 112; 27 and 113; 27 and 114; 27 and 115; 27 and 116; 27 and 117; 27 and 118; 27 and 119; 27 and 120; 27 and 121; 27 and 122; 27 and 123; 27 and 124; 27 and 125; 27 and 126; 27 and 127; 27 and 128; 27 and 129; 27 and 130; 27 and 131; 27 and 132; 27 and 133; 27 and 134; 27 and 135; 27 and 136; 27 and 137; 27 and 138; 27 and 139; 27 and 140; 27 and 141; 27 and 142; 27 and 143; 27 and 144; 27 and 145; 27 and 146; 27 and 147; 27 and 148; 27 and 149; 27 and 150; 27 and 151; 27 and 152; 27 and 153; 27 and 154; 27 and 155; 27 and 156; 27 and 157; 27 and 158; 27 and 159; 27 and 160; 27 and 161; 27 and 162; 27 and 163; 27 and 164; 27 and 165; 27 and 166; 27 and 167; 28 and 67; 28 and 68; 28 and 69; 28 and 70; 28 and 71; 28 and 72; 28 and 73; 28 and 74; 28 and 75; 28 and 76; 28 and 77; 28 and 78; 28 and 79; 28 and 80; 28 and 81; 28 and 82; 28 and 83; 28 and 84; 28 and 85; 28 and 86; 28 and 87; 28 and 88; 28 and 89; 28 and 90; 28 and 91; 28 and 92; 28 and 93; 28 and 94; 28 and 95; 28 and 96; 28 and 97; 28 and 98; 28 and 99; 28 and 100; 28 and 101; 28 and 102; 28 and 103; 28 and 104; 28 and 105; 28 and 106; 28 and 107; 28 and 108; 28 and 109; 28 and 110; 28 and 111; 28 and 112; 28 and 113; 28 and 114; 28 and 115; 28 and 116; 28 and 117; 28 and 118; 28 and 119; 28 and 120; 28 and 121; 28 and 122; 28 and 123; 28 and 124; 28 and 125; 28 and 126; 28 and 127; 28 and 128; 28 and 129; 28 and 130; 28 and 131; 28 and 132; 28 and 133; 28 and 134; 28 and 135; 28 and 136; 28 and 137; 28 and 138; 28 and 139; 28 and 140; 28 and 141; 28 and 142; 28 and 143; 28 and 144; 28 and 145; 28 and 146; 28 and 147; 28 and 148; 28 and 149; 28 and 150; 28 and 151; 28 and 152; 28 and 153; 28 and 154; 28 and 155; 28 and 156; 28 and 157; 28 and 158; 28 and 159; 28 and 160; 28 and 161; 28 and 162; 28 and 163; 28 and 164; 28 and 165; 28 and 166; 28 and 167; 29 and 67; 29 and 68; 29 and 69; 29 and 70; 29 and 71; 29 and 72; 29 and 73; 29 and 74; 29 and 75; 29 and 76; 29 and 77; 29 and 78; 29 and 79; 29 and 80; 29 and 81; 29 and 82; 29 and 83; 29 and 84; 29 and 85; 29 and 86; 29 and 87; 29 and 88; 29 and 89; 29 and 90; 29 and 91; 29 and 92; 29 and 93; 29 and 94; 29 and 95; 29 and 96; 29 and 97; 29 and 98; 29 and 99; 29 and 100; 29 and 101; 29 and 102; 29 and 103; 29 and 104; 29 and 105; 29 and 106; 29 and 107; 29 and 108; 29 and 109; 29 and 110; 29 and 111; 29 and 112; 29 and 113; 29 and 114; 29 and 115; 29 and 116; 29 and 117; 29 and 118; 29 and 119; 29 and 120; 29 and 121; 29 and 122; 29 and 123; 29 and 124; 29 and 125; 29 and 126; 29 and 127; 29 and 128; 29 and 129; 29 and 130; 29 and 131; 29 and 132; 29 and 133; 29 and 134; 29 and 135; 29 and 136; 29 and 137; 29 and 138; 29 and 139; 29 and 140; 29 and 141; 29 and 142; 29 and 143; 29 and 144; 29 and 145; 29 and 146; 29 and 147; 29 and 148; 29 and 149; 29 and 150; 29 and 151; 29 and 152; 29 and 153; 29 and 154; 29 and 155; 29 and 156; 29 and 157; 29 and 158; 29 and 159; 29 and 160; 29 and 161; 29 and 162; 29 and 163; 29 and 164; 29 and 165; 29 and 166; 29 and 167; 30 and 67; 30 and 68; 30 and 69; 30 and 70; 30 and 71; 30 and 72; 30 and 73; 30 and 74; 30 and 75; 30 and 76; 30 and 77; 30 and 78; 30 and 79; 30 and 80; 30 and 81; 30 and 82; 30 and 83; 30 and 84; 30 and 85; 30 and 86; 30 and 87; 30 and 88; 30 and 89; 30 and 90; 30 and 91; 30 and 92; 30 and 93; 30 and 94; 30 and 95; 30 and 96; 30 and 97; 30 and 98; 30 and 99; 30 and 100; 30 and 101; 30 and 102; 30 and 103; 30 and 104; 30 and 105; 30 and 106; 30 and 107; 30 and 108; 30 and 109; 30 and 110; 30 and 111; 30 and 112; 30 and 113; 30 and 114; 30 and 115; 30 and 116; 30 and 117; 30 and 118; 30 and 119; 30 and 120; 30 and 121; 30 and 122; 30 and 123; 30 and 124; 30 and 125; 30 and 126; 30 and 127; 30 and 128; 30 and 129; 30 and 130; 30 and 131; 30 and 132; 30 and 133; 30 and 134; 30 and 135; 30 and 136; 30 and 137; 30 and 138; 30 and 139; 30 and 140; 30 and 141; 30 and 142; 30 and 143; 30 and 144; 30 and 145; 30 and 146; 30 and 147; 30 and 148; 30 and 149; 30 and 150; 30 and 151; 30 and 152; 30 and 153; 30 and 154; 30 and 155; 30 and 156; 30 and 157; 30 and 158; 30 and 159; 30 and 160; 30 and 161; 30 and 162; 30 and 163; 30 and 164; 30 and 165; 30 and 166; 30 and 167; 31 and 67; 31 and 68; 31 and 69; 31 and 70; 31 and 71; 31 and 72; 31 and 73; 31 and 74; 31 and 75; 31 and 76; 31 and 77; 31 and 78; 31 and 79; 31 and 80; 31 and 81; 31 and 82; 31 and 83; 31 and 84; 31 and 85; 31 and 86; 31 and 87; 31 and 88; 31 and 89; 31 and 90; 31 and 91; 31 and 92; 31 and 93; 31 and 94; 31 and 95; 31 and 96; 31 and 97; 31 and 98; 31 and 99; 31 and 100; 31 and 101; 31 and 102; 31 and 103; 31 and 104; 31 and 105; 31 and 106; 31 and 107; 31 and 108; 31 and 109; 31 and 110; 31 and 111; 31 and 112; 31 and 113; 31 and 114; 31 and 115; 31 and 116; 31 and 117; 31 and 118; 31 and 119; 31 and 120; 31 and 121; 31 and 122; 31 and 123; 31 and 124; 31 and 125; 31 and 126; 31 and 127; 31 and 128; 31 and 129; 31 and 130; 31 and 131; 31 and 132; 31 and 133; 31 and 134; 31 and 135; 31 and 136; 31 and 137; 31 and 138; 31 and 139; 31 and 140; 31 and 141; 31 and 142; 31 and 143; 31 and 144; 31 and 145; 31 and 146; 31 and 147; 31 and 148; 31 and 149; 31 and 150; 31 and 151; 31 and 152; 31 and 153; 31 and 154; 31 and 155; 31 and 156; 31 and 157; 31 and 158; 31 and 159; 31 and 160; 31 and 161; 31 and 162; 31 and 163; 31 and 164; 31 and 165; 31 and 166; 31 and 167; 32 and 67; 32 and 68; 32 and 69; 32 and 70; 32 and 71; 32 and 72; 32 and 73; 32 and 74; 32 and 75; 32 and 76; 32 and 77; 32 and 78; 32 and 79; 32 and 80; 32 and 81; 32 and 82; 32 and 83; 32 and 84; 32 and 85; 32 and 86; 32 and 87; 32 and 88; 32 and 89; 32 and 90; 32 and 91; 32 and 92; 32 and 93; 32 and 94; 32 and 95; 32 and 96; 32 and 97; 32 and 98; 32 and 99; 32 and 100; 32 and 101; 32 and 102; 32 and 103; 32 and 104; 32 and 105; 32 and 106; 32 and 107; 32 and 108; 32 and 109; 32 and 110; 32 and 111; 32 and 112; 32 and 113; 32 and 114; 32 and 115; 32 and 116; 32 and 117; 32 and 118; 32 and 119; 32 and 120; 32 and 121; 32 and 122; 32 and 123; 32 and 124; 32 and 125; 32 and 126; 32 and 127; 32 and 128; 32 and 129; 32 and 130; 32 and 131; 32 and 132; 32 and 133; 32 and 134; 32 and 135; 32 and 136; 32 and 137; 32 and 138; 32 and 139; 32 and 140; 32 and 141; 32 and 142; 32 and 143; 32 and 144; 32 and 145; 32 and 146; 32 and 147; 32 and 148; 32 and 149; 32 and 150; 32 and 151; 32 and 152; 32 and 153; 32 and 154; 32 and 155; 32 and 156; 32 and 157; 32 and 158; 32 and 159; 32 and 160; 32 and 161; 32 and 162; 32 and 163; 32 and 164; 32 and 165; 32 and 166; 32 and 167; 33 and 67; 33 and 68; 33 and 69; 33 and 70; 33 and 71; 33 and 72; 33 and 73; 33 and 74; 33 and 75; 33 and 76; 33 and 77; 33 and 78; 33 and 79; 33 and 80; 33 and 81; 33 and 82; 33 and 83; 33 and 84; 33 and 85; 33 and 86; 33 and 87; 33 and 88; 33 and 89; 33 and 90; 33 and 91; 33 and 92; 33 and 93; 33 and 94; 33 and 95; 33 and 96; 33 and 97; 33 and 98; 33 and 99; 33 and 100; 33 and 101; 33 and 102; 33 and 103; 33 and 104; 33 and 105; 33 and 106; 33 and 107; 33 and 108; 33 and 109; 33 and 110; 33 and 111; 33 and 112; 33 and 113; 33 and 114; 33 and 115; 33 and 116; 33 and 117; 33 and 118; 33 and 119; 33 and 120; 33 and 121; 33 and 122; 33 and 123; 33 and 124; 33 and 125; 33 and 126; 33 and 127; 33 and 128; 33 and 129; 33 and 130; 33 and 131; 33 and 132; 33 and 133; 33 and 134; 33 and 135; 33 and 136; 33 and 137; 33 and 138; 33 and 139; 33 and 140; 33 and 141; 33 and 142; 33 and 143; 33 and 144; 33 and 145; 33 and 146; 33 and 147; 33 and 148; 33 and 149; 33 and 150; 33 and 151; 33 and 152; 33 and 153; 33 and 154; 33 and 155; 33 and 156; 33 and 157; 33 and 158; 33 and 159; 33 and 160; 33 and 161; 33 and 162; 33 and 163; 33 and 164; 33 and 165; 33 and 166; 33 and 167; 34 and 67; 34 and 68; 34 and 69; 34 and 70; 34 and 71; 34 and 72; 34 and 73; 34 and 74; 34 and 75; 34 and 76; 34 and 77; 34 and 78; 34 and 79; 34 and 80; 34 and 81; 34 and 82; 34 and 83; 34 and 84; 34 and 85; 34 and 86; 34 and 87; 34 and 88; 34 and 89; 34 and 90; 34 and 91; 34 and 92; 34 and 93; 34 and 94; 34 and 95; 34 and 96; 34 and 97; 34 and 98; 34 and 99; 34 and 100; 34 and 101; 34 and 102; 34 and 103; 34 and 104; 34 and 105; 34 and 106; 34 and 107; 34 and 108; 34 and 109; 34 and 110; 34 and 111; 34 and 112; 34 and 113; 34 and 114; 34 and 115; 34 and 116; 34 and 117; 34 and 118; 34 and 119; 34 and 120; 34 and 121; 34 and 122; 34 and 123; 34 and 124; 34 and 125; 34 and 126; 34 and 127; 34 and 128; 34 and 129; 34 and 130; 34 and 131; 34 and 132; 34 and 133; 34 and 134; 34 and 135; 34 and 136; 34 and 137; 34 and 138; 34 and 139; 34 and 140; 34 and 141; 34 and 142; 34 and 143; 34 and 144; 34 and 145; 34 and 146; 34 and 147; 34 and 148; 34 and 149; 34 and 150; 34 and 151; 34 and 152; 34 and 153; 34 and 154; 34 and 155; 34 and 156; 34 and 157; 34 and 158; 34 and 159; 34 and 160; 34 and 161; 34 and 162; 34 and 163; 34 and 164; 34 and 165; 34 and 166; 34 and 167; 35 and 67; 35 and 68; 35 and 69; 35 and 70; 35 and 71; 35 and 72; 35 and 73; 35 and 74; 35 and 75; 35 and 76; 35 and 77; 35 and 78; 35 and 79; 35 and 80; 35 and 81; 35 and 82; 35 and 83; 35 and 84; 35 and 85; 35 and 86; 35 and 87; 35 and 88; 35 and 89; 35 and 90; 35 and 91; 35 and 92; 35 and 93; 35 and 94; 35 and 95; 35 and 96; 35 and 97; 35 and 98; 35 and 99; 35 and 100; 35 and 101; 35 and 102; 35 and 103; 35 and 104; 35 and 105; 35 and 106; 35 and 107; 35 and 108; 35 and 109; 35 and 110; 35 and 111; 35 and 112; 35 and 113; 35 and 114; 35 and 115; 35 and 116; 35 and 117; 35 and 118; 35 and 119; 35 and 120; 35 and 121; 35 and 122; 35 and 123; 35 and 124; 35 and 125; 35 and 126; 35 and 127; 35 and 128; 35 and 129; 35 and 130; 35 and 131; 35 and 132; 35 and 133; 35 and 134; 35 and 135; 35 and 136; 35 and 137; 35 and 138; 35 and 139; 35 and 140; 35 and 141; 35 and 142; 35 and 143; 35 and 144; 35 and 145; 35 and 146; 35 and 147; 35 and 148; 35 and 149; 35 and 150; 35 and 151; 35 and 152; 35 and 153; 35 and 154; 35 and 155; 35 and 156; 35 and 157; 35 and 158; 35 and 159; 35 and 160; 35 and 161; 35 and 162; 35 and 163; 35 and 164; 35 and 165; 35 and 166; 35 and 167; 36 and 67; 36 and 68; 36 and 69; 36 and 70; 36 and 71; 36 and 72; 36 and 73; 36 and 74; 36 and 75; 36 and 76; 36 and 77; 36 and 78; 36 and 79; 36 and 80; 36 and 81; 36 and 82; 36 and 83; 36 and 84; 36 and 85; 36 and 86; 36 and 87; 36 and 88; 36 and 89; 36 and 90; 36 and 91; 36 and 92; 36 and 93; 36 and 94; 36 and 95; 36 and 96; 36 and 97; 36 and 98; 36 and 99; 36 and 100; 36 and 101; 36 and 102; 36 and 103; 36 and 104; 36 and 105; 36 and 106; 36 and 107; 36 and 108; 36 and 109; 36 and 110; 36 and 111; 36 and 112; 36 and 113; 36 and 114; 36 and 115; 36 and 116; 36 and 117; 36 and 118; 36 and 119; 36 and 120; 36 and 121; 36 and 122; 36 and 123; 36 and 124; 36 and 125; 36 and 126; 36 and 127; 36 and 128; 36 and 129; 36 and 130; 36 and 131; 36 and 132; 36 and 133; 36 and 134; 36 and 135; 36 and 136; 36 and 137; 36 and 138; 36 and 139; 36 and 140; 36 and 141; 36 and 142; 36 and 143; 36 and 144; 36 and 145; 36 and 146; 36 and 147; 36 and 148; 36 and 149; 36 and 150; 36 and 151; 36 and 152; 36 and 153; 36 and 154; 36 and 155; 36 and 156; 36 and 157; 36 and 158; 36 and 159; 36 and 160; 36 and 161; 36 and 162; 36 and 163; 36 and 164; 36 and 165; 36 and 166; 36 and 167; 37 and 67; 37 and 68; 37 and 69; 37 and 70; 37 and 71; 37 and 72; 37 and 73; 37 and 74; 37 and 75; 37 and 76; 37 and 77; 37 and 78; 37 and 79; 37 and 80; 37 and 81; 37 and 82; 37 and 83; 37 and 84; 37 and 85; 37 and 86; 37 and 87; 37 and 88; 37 and 89; 37 and 90; 37 and 91; 37 and 92; 37 and 93; 37 and 94; 37 and 95; 37 and 96; 37 and 97; 37 and 98; 37 and 99; 37 and 100; 37 and 101; 37 and 102; 37 and 103; 37 and 104; 37 and 105; 37 and 106; 37 and 107; 37 and 108; 37 and 109; 37 and 110; 37 and 111; 37 and 112; 37 and 113; 37 and 114; 37 and 115; 37 and 116; 37 and 117; 37 and 118; 37 and 119; 37 and 120; 37 and 121; 37 and 122; 37 and 123; 37 and 124; 37 and 125; 37 and 126; 37 and 127; 37 and 128; 37 and 129; 37 and 130; 37 and 131; 37 and 132; 37 and 133; 37 and 134; 37 and 135; 37 and 136; 37 and 137; 37 and 138; 37 and 139; 37 and 140; 37 and 141; 37 and 142; 37 and 143; 37 and 144; 37 and 145; 37 and 146; 37 and 147; 37 and 148; 37 and 149; 37 and 150; 37 and 151; 37 and 152; 37 and 153; 37 and 154; 37 and 155; 37 and 156; 37 and 157; 37 and 158; 37 and 159; 37 and 160; 37 and 161; 37 and 162; 37 and 163; 37 and 164; 37 and 165; 37 and 166; 37 and 167; 38 and 67; 38 and 68; 38 and 69; 38 and 70; 38 and 71; 38 and 72; 38 and 73; 38 and 74; 38 and 75; 38 and 76; 38 and 77; 38 and 78; 38 and 79; 38 and 80; 38 and 81; 38 and 82; 38 and 83; 38 and 84; 38 and 85; 38 and 86; 38 and 87; 38 and 88; 38 and 89; 38 and 90; 38 and 91; 38 and 92; 38 and 93; 38 and 94; 38 and 95; 38 and 96; 38 and 97; 38 and 98; 38 and 99; 38 and 100; 38 and 101; 38 and 102; 38 and 103; 38 and 104; 38 and 105; 38 and 106; 38 and 107; 38 and 108; 38 and 109; 38 and 110; 38 and 111; 38 and 112; 38 and 113; 38 and 114; 38 and 115; 38 and 116; 38 and 117; 38 and 118; 38 and 119; 38 and 120; 38 and 121; 38 and 122; 38 and 123; 38 and 124; 38 and 125; 38 and 126; 38 and 127; 38 and 128; 38 and 129; 38 and 130; 38 and 131; 38 and 132; 38 and 133; 38 and 134; 38 and 135; 38 and 136; 38 and 137; 38 and 138; 38 and 139; 38 and 140; 38 and 141; 38 and 142; 38 and 143; 38 and 144; 38 and 145; 38 and 146; 38 and 147; 38 and 148; 38 and 149; 38 and 150; 38 and 151; 38 and 152; 38 and 153; 38 and 154; 38 and 155; 38 and 156; 38 and 157; 38 and 158; 38 and 159; 38 and 160; 38 and 161; 38 and 162; 38 and 163; 38 and 164; 38 and 165; 38 and 166; 38 and 167; 39 and 67; 39 and 68; 39 and 69; 39 and 70; 39 and 71; 39 and 72; 39 and 73; 39 and 74; 39 and 75; 39 and 76; 39 and 77; 39 and 78; 39 and 79; 39 and 80; 39 and 81; 39 and 82; 39 and 83; 39 and 84; 39 and 85; 39 and 86; 39 and 87; 39 and 88; 39 and 89; 39 and 90; 39 and 91; 39 and 92; 39 and 93; 39 and 94; 39 and 95; 39 and 96; 39 and 97; 39 and 98; 39 and 99; 39 and 100; 39 and 101; 39 and 102; 39 and 103; 39 and 104; 39 and 105; 39 and 106; 39 and 107; 39 and 108; 39 and 109; 39 and 110; 39 and 111; 39 and 112; 39 and 113; 39 and 114; 39 and 115; 39 and 116; 39 and 117; 39 and 118; 39 and 119; 39 and 120; 39 and 121; 39 and 122; 39 and 123; 39 and 124; 39 and 125; 39 and 126; 39 and 127; 39 and 128; 39 and 129; 39 and 130; 39 and 131; 39 and 132; 39 and 133; 39 and 134; 39 and 135; 39 and 136; 39 and 137; 39 and 138; 39 and 139; 39 and 140; 39 and 141; 39 and 142; 39 and 143; 39 and 144; 39 and 145; 39 and 146; 39 and 147; 39 and 148; 39 and 149; 39 and 150; 39 and 151; 39 and 152; 39 and 153; 39 and 154; 39 and 155; 39 and 156; 39 and 157; 39 and 158; 39 and 159; 39 and 160; 39 and 161; 39 and 162; 39 and 163; 39 and 164; 39 and 165; 39 and 166; 39 and 167; 40 and 67; 40 and 68; 40 and 69; 40 and 70; 40 and 71; 40 and 72; 40 and 73; 40 and 74; 40 and 75; 40 and 76; 40 and 77; 40 and 78; 40 and 79; 40 and 80; 40 and 81; 40 and 82; 40 and 83; 40 and 84; 40 and 85; 40 and 86; 40 and 87; 40 and 88; 40 and 89; 40 and 90; 40 and 91; 40 and 92; 40 and 93; 40 and 94; 40 and 95; 40 and 96; 40 and 97; 40 and 98; 40 and 99; 40 and 100; 40 and 101; 40 and 102; 40 and 103; 40 and 104; 40 and 105; 40 and 106; 40 and 107; 40 and 108; 40 and 109; 40 and 110; 40 and 111; 40 and 112; 40 and 113; 40 and 114; 40 and 115; 40 and 116; 40 and 117; 40 and 118; 40 and 119; 40 and 120; 40 and 121; 40 and 122; 40 and 123; 40 and 124; 40 and 125; 40 and 126; 40 and 127; 40 and 128; 40 and 129; 40 and 130; 40 and 131; 40 and 132; 40 and 133; 40 and 134; 40 and 135; 40 and 136; 40 and 137; 40 and 138; 40 and 139; 40 and 140; 40 and 141; 40 and 142; 40 and 143; 40 and 144; 40 and 145; 40 and 146; 40 and 147; 40 and 148; 40 and 149; 40 and 150; 40 and 151; 40 and 152; 40 and 153; 40 and 154; 40 and 155; 40 and 156; 40 and 157; 40 and 158; 40 and 159; 40 and 160; 40 and 161; 40 and 162; 40 and 163; 40 and 164; 40 and 165; 40 and 166; 40 and 167; 41 and 67; 41 and 68; 41 and 69; 41 and 70; 41 and 71; 41 and 72; 41 and 73; 41 and 74; 41 and 75; 41 and 76; 41 and 77; 41 and 78; 41 and 79; 41 and 80; 41 and 81; 41 and 82; 41 and 83; 41 and 84; 41 and 85; 41 and 86; 41 and 87; 41 and 88; 41 and 89; 41 and 90; 41 and 91; 41 and 92; 41 and 93; 41 and 94; 41 and 95; 41 and 96; 41 and 97; 41 and 98; 41 and 99; 41 and 100; 41 and 101; 41 and 102; 41 and 103; 41 and 104; 41 and 105; 41 and 106; 41 and 107; 41 and 108; 41 and 109; 41 and 110; 41 and 111; 41 and 112; 41 and 113; 41 and 114; 41 and 115; 41 and 116; 41 and 117; 41 and 118; 41 and 119; 41 and 120; 41 and 121; 41 and 122; 41 and 123; 41 and 124; 41 and 125; 41 and 126; 41 and 127; 41 and 128; 41 and 129; 41 and 130; 41 and 131; 41 and 132; 41 and 133; 41 and 134; 41 and 135; 41 and 136; 41 and 137; 41 and 138; 41 and 139; 41 and 140; 41 and 141; 41 and 142; 41 and 143; 41 and 144; 41 and 145; 41 and 146; 41 and 147; 41 and 148; 41 and 149; 41 and 150; 41 and 151; 41 and 152; 41 and 153; 41 and 154; 41 and 155; 41 and 156; 41 and 157; 41 and 158; 41 and 159; 41 and 160; 41 and 161; 41 and 162; 41 and 163; 41 and 164; 41 and 165; 41 and 166; 41 and 167; 42 and 67; 42 and 68; 42 and 69; 42 and 70; 42 and 71; 42 and 72; 42 and 73; 42 and 74; 42 and 75; 42 and 76; 42 and 77; 42 and 78; 42 and 79; 42 and 80; 42 and 81; 42 and 82; 42 and 83; 42 and 84; 42 and 85; 42 and 86; 42 and 87; 42 and 88; 42 and 89; 42 and 90; 42 and 91; 42 and 92; 42 and 93; 42 and 94; 42 and 95; 42 and 96; 42 and 97; 42 and 98; 42 and 99; 42 and 100; 42 and 101; 42 and 102; 42 and 103; 42 and 104; 42 and 105; 42 and 106; 42 and 107; 42 and 108; 42 and 109; 42 and 110; 42 and 111; 42 and 112; 42 and 113; 42 and 114; 42 and 115; 42 and 116; 42 and 117; 42 and 118; 42 and 119; 42 and 120; 42 and 121; 42 and 122; 42 and 123; 42 and 124; 42 and 125; 42 and 126; 42 and 127; 42 and 128; 42 and 129; 42 and 130; 42 and 131; 42 and 132; 42 and 133; 42 and 134; 42 and 135; 42 and 136; 42 and 137; 42 and 138; 42 and 139; 42 and 140; 42 and 141; 42 and 142; 42 and 143; 42 and 144; 42 and 145; 42 and 146; 42 and 147; 42 and 148; 42 and 149; 42 and 150; 42 and 151; 42 and 152; 42 and 153; 42 and 154; 42 and 155; 42 and 156; 42 and 157; 42 and 158; 42 and 159; 42 and 160; 42 and 161; 42 and 162; 42 and 163; 42 and 164; 42 and 165; 42 and 166; 42 and 167; 43 and 67; 43 and 68; 43 and 69; 43 and 70; 43 and 71; 43 and 72; 43 and 73; 43 and 74; 43 and 75; 43 and 76; 43 and 77; 43 and 78; 43 and 79; 43 and 80; 43 and 81; 43 and 82; 43 and 83; 43 and 84; 43 and 85; 43 and 86; 43 and 87; 43 and 88; 43 and 89; 43 and 90; 43 and 91; 43 and 92; 43 and 93; 43 and 94; 43 and 95; 43 and 96; 43 and 97; 43 and 98; 43 and 99; 43 and 100; 43 and 101; 43 and 102; 43 and 103; 43 and 104; 43 and 105; 43 and 106; 43 and 107; 43 and 108; 43 and 109; 43 and 110; 43 and 111; 43 and 112; 43 and 113; 43 and 114; 43 and 115; 43 and 116; 43 and 117; 43 and 118; 43 and 119; 43 and 120; 43 and 121; 43 and 122; 43 and 123; 43 and 124; 43 and 125; 43 and 126; 43 and 127; 43 and 128; 43 and 129; 43 and 130; 43 and 131; 43 and 132; 43 and 133; 43 and 134; 43 and 135; 43 and 136; 43 and 137; 43 and 138; 43 and 139; 43 and 140; 43 and 141; 43 and 142; 43 and 143; 43 and 144; 43 and 145; 43 and 146; 43 and 147; 43 and 148; 43 and 149; 43 and 150; 43 and 151; 43 and 152; 43 and 153; 43 and 154; 43 and 155; 43 and 156; 43 and 157; 43 and 158; 43 and 159; 43 and 160; 43 and 161; 43 and 162; 43 and 163; 43 and 164; 43 and 165; 43 and 166; 43 and 167; 44 and 67; 44 and 68; 44 and 69; 44 and 70; 44 and 71; 44 and 72; 44 and 73; 44 and 74; 44 and 75; 44 and 76; 44 and 77; 44 and 78; 44 and 79; 44 and 80; 44 and 81; 44 and 82; 44 and 83; 44 and 84; 44 and 85; 44 and 86; 44 and 87; 44 and 88; 44 and 89; 44 and 90; 44 and 91; 44 and 92; 44 and 93; 44 and 94; 44 and 95; 44 and 96; 44 and 97; 44 and 98; 44 and 99; 44 and 100; 44 and 101; 44 and 102; 44 and 103; 44 and 104; 44 and 105; 44 and 106; 44 and 107; 44 and 108; 44 and 109; 44 and 110; 44 and 111; 44 and 112; 44 and 113; 44 and 114; 44 and 115; 44 and 116; 44 and 117; 44 and 118; 44 and 119; 44 and 120; 44 and 121; 44 and 122; 44 and 123; 44 and 124; 44 and 125; 44 and 126; 44 and 127; 44 and 128; 44 and 129; 44 and 130; 44 and 131; 44 and 132; 44 and 133; 44 and 134; 44 and 135; 44 and 136; 44 and 137; 44 and 138; 44 and 139; 44 and 140; 44 and 141; 44 and 142; 44 and 143; 44 and 144; 44 and 145; 44 and 146; 44 and 147; 44 and 148; 44 and 149; 44 and 150; 44 and 151; 44 and 152; 44 and 153; 44 and 154; 44 and 155; 44 and 156; 44 and 157; 44 and 158; 44 and 159; 44 and 160; 44 and 161; 44 and 162; 44 and 163; 44 and 164; 44 and 165; 44 and 166; 44 and 167; 45 and 67; 45 and 68; 45 and 69; 45 and 70; 45 and 71; 45 and 72; 45 and 73; 45 and 74; 45 and 75; 45 and 76; 45 and 77; 45 and 78; 45 and 79; 45 and 80; 45 and 81; 45 and 82; 45 and 83; 45 and 84; 45 and 85; 45 and 86; 45 and 87; 45 and 88; 45 and 89; 45 and 90; 45 and 91; 45 and 92; 45 and 93; 45 and 94; 45 and 95; 45 and 96; 45 and 97; 45 and 98; 45 and 99; 45 and 100; 45 and 101; 45 and 102; 45 and 103; 45 and 104; 45 and 105; 45 and 106; 45 and 107; 45 and 108; 45 and 109; 45 and 110; 45 and 111; 45 and 112; 45 and 113; 45 and 114; 45 and 115; 45 and 116; 45 and 117; 45 and 118; 45 and 119; 45 and 120; 45 and 121; 45 and 122; 45 and 123; 45 and 124; 45 and 125; 45 and 126; 45 and 127; 45 and 128; 45 and 129; 45 and 130; 45 and 131; 45 and 132; 45 and 133; 45 and 134; 45 and 135; 45 and 136; 45 and 137; 45 and 138; 45 and 139; 45 and 140; 45 and 141; 45 and 142; 45 and 143; 45 and 144; 45 and 145; 45 and 146; 45 and 147; 45 and 148; 45 and 149; 45 and 150; 45 and 151; 45 and 152; 45 and 153; 45 and 154; 45 and 155; 45 and 156; 45 and 157; 45 and 158; 45 and 159; 45 and 160; 45 and 161; 45 and 162; 45 and 163; 45 and 164; 45 and 165; 45 and 166; 45 and 167; 46 and 67; 46 and 68; 46 and 69; 46 and 70; 46 and 71; 46 and 72; 46 and 73; 46 and 74; 46 and 75; 46 and 76; 46 and 77; 46 and 78; 46 and 79; 46 and 80; 46 and 81; 46 and 82; 46 and 83; 46 and 84; 46 and 85; 46 and 86; 46 and 87; 46 and 88; 46 and 89; 46 and 90; 46 and 91; 46 and 92; 46 and 93; 46 and 94; 46 and 95; 46 and 96; 46 and 97; 46 and 98; 46 and 99; 46 and 100; 46 and 101; 46 and 102; 46 and 103; 46 and 104; 46 and 105; 46 and 106; 46 and 107; 46 and 108; 46 and 109; 46 and 110; 46 and 111; 46 and 112; 46 and 113; 46 and 114; 46 and 115; 46 and 116; 46 and 117; 46 and 118; 46 and 119; 46 and 120; 46 and 121; 46 and 122; 46 and 123; 46 and 124; 46 and 125; 46 and 126; 46 and 127; 46 and 128; 46 and 129; 46 and 130; 46 and 131; 46 and 132; 46 and 133; 46 and 134; 46 and 135; 46 and 136; 46 and 137; 46 and 138; 46 and 139; 46 and 140; 46 and 141; 46 and 142; 46 and 143; 46 and 144; 46 and 145; 46 and 146; 46 and 147; 46 and 148; 46 and 149; 46 and 150; 46 and 151; 46 and 152; 46 and 153; 46 and 154; 46 and 155; 46 and 156; 46 and 157; 46 and 158; 46 and 159; 46 and 160; 46 and 161; 46 and 162; 46 and 163; 46 and 164; 46 and 165; 46 and 166; 46 and 167; 47 and 67; 47 and 68; 47 and 69; 47 and 70; 47 and 71; 47 and 72; 47 and 73; 47 and 74; 47 and 75; 47 and 76; 47 and 77; 47 and 78; 47 and 79; 47 and 80; 47 and 81; 47 and 82; 47 and 83; 47 and 84; 47 and 85; 47 and 86; 47 and 87; 47 and 88; 47 and 89; 47 and 90; 47 and 91; 47 and 92; 47 and 93; 47 and 94; 47 and 95; 47 and 96; 47 and 97; 47 and 98; 47 and 99; 47 and 100; 47 and 101; 47 and 102; 47 and 103; 47 and 104; 47 and 105; 47 and 106; 47 and 107; 47 and 108; 47 and 109; 47 and 110; 47 and 111; 47 and 112; 47 and 113; 47 and 114; 47 and 115; 47 and 116; 47 and 117; 47 and 118; 47 and 119; 47 and 120; 47 and 121; 47 and 122; 47 and 123; 47 and 124; 47 and 125; 47 and 126; 47 and 127; 47 and 128; 47 and 129; 47 and 130; 47 and 131; 47 and 132; 47 and 133; 47 and 134; 47 and 135; 47 and 136; 47 and 137; 47 and 138; 47 and 139; 47 and 140; 47 and 141; 47 and 142; 47 and 143; 47 and 144; 47 and 145; 47 and 146; 47 and 147; 47 and 148; 47 and 149; 47 and 150; 47 and 151; 47 and 152; 47 and 153; 47 and 154; 47 and 155; 47 and 156; 47 and 157; 47 and 158; 47 and 159; 47 and 160; 47 and 161; 47 and 162; 47 and 163; 47 and 164; 47 and 165; 47 and 166; 47 and 167; 48 and 67; 48 and 68; 48 and 69; 48 and 70; 48 and 71; 48 and 72; 48 and 73; 48 and 74; 48 and 75; 48 and 76; 48 and 77; 48 and 78; 48 and 79; 48 and 80; 48 and 81; 48 and 82; 48 and 83; 48 and 84; 48 and 85; 48 and 86; 48 and 87; 48 and 88; 48 and 89; 48 and 90; 48 and 91; 48 and 92; 48 and 93; 48 and 94; 48 and 95; 48 and 96; 48 and 97; 48 and 98; 48 and 99; 48 and 100; 48 and 101; 48 and 102; 48 and 103; 48 and 104; 48 and 105; 48 and 106; 48 and 107; 48 and 108; 48 and 109; 48 and 110; 48 and 111; 48 and 112; 48 and 113; 48 and 114; 48 and 115; 48 and 116; 48 and 117; 48 and 118; 48 and 119; 48 and 120; 48 and 121; 48 and 122; 48 and 123; 48 and 124; 48 and 125; 48 and 126; 48 and 127; 48 and 128; 48 and 129; 48 and 130; 48 and 131; 48 and 132; 48 and 133; 48 and 134; 48 and 135; 48 and 136; 48 and 137; 48 and 138; 48 and 139; 48 and 140; 48 and 141; 48 and 142; 48 and 143; 48 and 144; 48 and 145; 48 and 146; 48 and 147; 48 and 148; 48 and 149; 48 and 150; 48 and 151; 48 and 152; 48 and 153; 48 and 154; 48 and 155; 48 and 156; 48 and 157; 48 and 158; 48 and 159; 48 and 160; 48 and 161; 48 and 162; 48 and 163; 48 and 164; 48 and 165; 48 and 166; 48 and 167; 49 and 67; 49 and 68; 49 and 69; 49 and 70; 49 and 71; 49 and 72; 49 and 73; 49 and 74; 49 and 75; 49 and 76; 49 and 77; 49 and 78; 49 and 79; 49 and 80; 49 and 81; 49 and 82; 49 and 83; 49 and 84; 49 and 85; 49 and 86; 49 and 87; 49 and 88; 49 and 89; 49 and 90; 49 and 91; 49 and 92; 49 and 93; 49 and 94; 49 and 95; 49 and 96; 49 and 97; 49 and 98; 49 and 99; 49 and 100; 49 and 101; 49 and 102; 49 and 103; 49 and 104; 49 and 105; 49 and 106; 49 and 107; 49 and 108; 49 and 109; 49 and 110; 49 and 111; 49 and 112; 49 and 113; 49 and 114; 49 and 115; 49 and 116; 49 and 117; 49 and 118; 49 and 119; 49 and 120; 49 and 121; 49 and 122; 49 and 123; 49 and 124; 49 and 125; 49 and 126; 49 and 127; 49 and 128; 49 and 129; 49 and 130; 49 and 131; 49 and 132; 49 and 133; 49 and 134; 49 and 135; 49 and 136; 49 and 137; 49 and 138; 49 and 139; 49 and 140; 49 and 141; 49 and 142; 49 and 143; 49 and 144; 49 and 145; 49 and 146; 49 and 147; 49 and 148; 49 and 149; 49 and 150; 49 and 151; 49 and 152; 49 and 153; 49 and 154; 49 and 155; 49 and 156; 49 and 157; 49 and 158; 49 and 159; 49 and 160; 49 and 161; 49 and 162; 49 and 163; 49 and 164; 49 and 165; 49 and 166; 49 and 167; 50 and 67; 50 and 68; 50 and 69; 50 and 70; 50 and 71; 50 and 72; 50 and 73; 50 and 74; 50 and 75; 50 and 76; 50 and 77; 50 and 78; 50 and 79; 50 and 80; 50 and 81; 50 and 82; 50 and 83; 50 and 84; 50 and 85; 50 and 86; 50 and 87; 50 and 88; 50 and 89; 50 and 90; 50 and 91; 50 and 92; 50 and 93; 50 and 94; 50 and 95; 50 and 96; 50 and 97; 50 and 98; 50 and 99; 50 and 100; 50 and 101; 50 and 102; 50 and 103; 50 and 104; 50 and 105; 50 and 106; 50 and 107; 50 and 108; 50 and 109; 50 and 110; 50 and 111; 50 and 112; 50 and 113; 50 and 114; 50 and 115; 50 and 116; 50 and 117; 50 and 118; 50 and 119; 50 and 120; 50 and 121; 50 and 122; 50 and 123; 50 and 124; 50 and 125; 50 and 126; 50 and 127; 50 and 128; 50 and 129; 50 and 130; 50 and 131; 50 and 132; 50 and 133; 50 and 134; 50 and 135; 50 and 136; 50 and 137; 50 and 138; 50 and 139; 50 and 140; 50 and 141; 50 and 142; 50 and 143; 50 and 144; 50 and 145; 50 and 146; 50 and 147; 50 and 148; 50 and 149; 50 and 150; 50 and 151; 50 and 152; 50 and 153; 50 and 154; 50 and 155; 50 and 156; 50 and 157; 50 and 158; 50 and 159; 50 and 160; 50 and 161; 50 and 162; 50 and 163; 50 and 164; 50 and 165; 50 and 166; 50 and 167; 51 and 67; 51 and 68; 51 and 69; 51 and 70; 51 and 71; 51 and 72; 51 and 73; 51 and 74; 51 and 75; 51 and 76; 51 and 77; 51 and 78; 51 and 79; 51 and 80; 51 and 81; 51 and 82; 51 and 83; 51 and 84; 51 and 85; 51 and 86; 51 and 87; 51 and 88; 51 and 89; 51 and 90; 51 and 91; 51 and 92; 51 and 93; 51 and 94; 51 and 95; 51 and 96; 51 and 97; 51 and 98; 51 and 99; 51 and 100; 51 and 101; 51 and 102; 51 and 103; 51 and 104; 51 and 105; 51 and 106; 51 and 107; 51 and 108; 51 and 109; 51 and 110; 51 and 111; 51 and 112; 51 and 113; 51 and 114; 51 and 115; 51 and 116; 51 and 117; 51 and 118; 51 and 119; 51 and 120; 51 and 121; 51 and 122; 51 and 123; 51 and 124; 51 and 125; 51 and 126; 51 and 127; 51 and 128; 51 and 129; 51 and 130; 51 and 131; 51 and 132; 51 and 133; 51 and 134; 51 and 135; 51 and 136; 51 and 137; 51 and 138; 51 and 139; 51 and 140; 51 and 141; 51 and 142; 51 and 143; 51 and 144; 51 and 145; 51 and 146; 51 and 147; 51 and 148; 51 and 149; 51 and 150; 51 and 151; 51 and 152; 51 and 153; 51 and 154; 51 and 155; 51 and 156; 51 and 157; 51 and 158; 51 and 159; 51 and 160; 51 and 161; 51 and 162; 51 and 163; 51 and 164; 51 and 165; 51 and 166; 51 and 167; 52 and 67; 52 and 68; 52 and 69; 52 and 70; 52 and 71; 52 and 72; 52 and 73; 52 and 74; 52 and 75; 52 and 76; 52 and 77; 52 and 78; 52 and 79; 52 and 80; 52 and 81; 52 and 82; 52 and 83; 52 and 84; 52 and 85; 52 and 86; 52 and 87; 52 and 88; 52 and 89; 52 and 90; 52 and 91; 52 and 92; 52 and 93; 52 and 94; 52 and 95; 52 and 96; 52 and 97; 52 and 98; 52 and 99; 52 and 100; 52 and 101; 52 and 102; 52 and 103; 52 and 104; 52 and 105; 52 and 106; 52 and 107; 52 and 108; 52 and 109; 52 and 110; 52 and 111; 52 and 112; 52 and 113; 52 and 114; 52 and 115; 52 and 116; 52 and 117; 52 and 118; 52 and 119; 52 and 120; 52 and 121; 52 and 122; 52 and 123; 52 and 124; 52 and 125; 52 and 126; 52 and 127; 52 and 128; 52 and 129; 52 and 130; 52 and 131; 52 and 132; 52 and 133; 52 and 134; 52 and 135; 52 and 136; 52 and 137; 52 and 138; 52 and 139; 52 and 140; 52 and 141; 52 and 142; 52 and 143; 52 and 144; 52 and 145; 52 and 146; 52 and 147; 52 and 148; 52 and 149; 52 and 150; 52 and 151; 52 and 152; 52 and 153; 52 and 154; 52 and 155; 52 and 156; 52 and 157; 52 and 158; 52 and 159; 52 and 160; 52 and 161; 52 and 162; 52 and 163; 52 and 164; 52 and 165; 52 and 166; 52 and 167; 53 and 67; 53 and 68; 53 and 69; 53 and 70; 53 and 71; 53 and 72; 53 and 73; 53 and 74; 53 and 75; 53 and 76; 53 and 77; 53 and 78; 53 and 79; 53 and 80; 53 and 81; 53 and 82; 53 and 83; 53 and 84; 53 and 85; 53 and 86; 53 and 87; 53 and 88; 53 and 89; 53 and 90; 53 and 91; 53 and 92; 53 and 93; 53 and 94; 53 and 95; 53 and 96; 53 and 97; 53 and 98; 53 and 99; 53 and 100; 53 and 101; 53 and 102; 53 and 103; 53 and 104; 53 and 105; 53 and 106; 53 and 107; 53 and 108; 53 and 109; 53 and 110; 53 and 111; 53 and 112; 53 and 113; 53 and 114; 53 and 115; 53 and 116; 53 and 117; 53 and 118; 53 and 119; 53 and 120; 53 and 121; 53 and 122; 53 and 123; 53 and 124; 53 and 125; 53 and 126; 53 and 127; 53 and 128; 53 and 129; 53 and 130; 53 and 131; 53 and 132; 53 and 133; 53 and 134; 53 and 135; 53 and 136; 53 and 137; 53 and 138; 53 and 139; 53 and 140; 53 and 141; 53 and 142; 53 and 143; 53 and 144; 53 and 145; 53 and 146; 53 and 147; 53 and 148; 53 and 149; 53 and 150; 53 and 151; 53 and 152; 53 and 153; 53 and 154; 53 and 155; 53 and 156; 53 and 157; 53 and 158; 53 and 159; 53 and 160; 53 and 161; 53 and 162; 53 and 163; 53 and 164; 53 and 165; 53 and 166; 53 and 167; 54 and 67; 54 and 68; 54 and 69; 54 and 70; 54 and 71; 54 and 72; 54 and 73; 54 and 74; 54 and 75; 54 and 76; 54 and 77; 54 and 78; 54 and 79; 54 and 80; 54 and 81; 54 and 82; 54 and 83; 54 and 84; 54 and 85; 54 and 86; 54 and 87; 54 and 88; 54 and 89; 54 and 90; 54 and 91; 54 and 92; 54 and 93; 54 and 94; 54 and 95; 54 and 96; 54 and 97; 54 and 98; 54 and 99; 54 and 100; 54 and 101; 54 and 102; 54 and 103; 54 and 104; 54 and 105; 54 and 106; 54 and 107; 54 and 108; 54 and 109; 54 and 110; 54 and 111; 54 and 112; 54 and 113; 54 and 114; 54 and 115; 54 and 116; 54 and 117; 54 and 118; 54 and 119; 54 and 120; 54 and 121; 54 and 122; 54 and 123; 54 and 124; 54 and 125; 54 and 126; 54 and 127; 54 and 128; 54 and 129; 54 and 130; 54 and 131; 54 and 132; 54 and 133; 54 and 134; 54 and 135; 54 and 136; 54 and 137; 54 and 138; 54 and 139; 54 and 140; 54 and 141; 54 and 142; 54 and 143; 54 and 144; 54 and 145; 54 and 146; 54 and 147; 54 and 148; 54 and 149; 54 and 150; 54 and 151; 54 and 152; 54 and 153; 54 and 154; 54 and 155; 54 and 156; 54 and 157; 54 and 158; 54 and 159; 54 and 160; 54 and 161; 54 and 162; 54 and 163; 54 and 164; 54 and 165; 54 and 166; 54 and 167; 55 and 67; 55 and 68; 55 and 69; 55 and 70; 55 and 71; 55 and 72; 55 and 73; 55 and 74; 55 and 75; 55 and 76; 55 and 77; 55 and 78; 55 and 79; 55 and 80; 55 and 81; 55 and 82; 55 and 83; 55 and 84; 55 and 85; 55 and 86; 55 and 87; 55 and 88; 55 and 89; 55 and 90; 55 and 91; 55 and 92; 55 and 93; 55 and 94; 55 and 95; 55 and 96; 55 and 97; 55 and 98; 55 and 99; 55 and 100; 55 and 101; 55 and 102; 55 and 103; 55 and 104; 55 and 105; 55 and 106; 55 and 107; 55 and 108; 55 and 109; 55 and 110; 55 and 111; 55 and 112; 55 and 113; 55 and 114; 55 and 115; 55 and 116; 55 and 117; 55 and 118; 55 and 119; 55 and 120; 55 and 121; 55 and 122; 55 and 123; 55 and 124; 55 and 125; 55 and 126; 55 and 127; 55 and 128; 55 and 129; 55 and 130; 55 and 131; 55 and 132; 55 and 133; 55 and 134; 55 and 135; 55 and 136; 55 and 137; 55 and 138; 55 and 139; 55 and 140; 55 and 141; 55 and 142; 55 and 143; 55 and 144; 55 and 145; 55 and 146; 55 and 147; 55 and 148; 55 and 149; 55 and 150; 55 and 151; 55 and 152; 55 and 153; 55 and 154; 55 and 155; 55 and 156; 55 and 157; 55 and 158; 55 and 159; 55 and 160; 55 and 161; 55 and 162; 55 and 163; 55 and 164; 55 and 165; 55 and 166; 55 and 167; 56 and 67; 56 and 68; 56 and 69; 56 and 70; 56 and 71; 56 and 72; 56 and 73; 56 and 74; 56 and 75; 56 and 76; 56 and 77; 56 and 78; 56 and 79; 56 and 80; 56 and 81; 56 and 82; 56 and 83; 56 and 84; 56 and 85; 56 and 86; 56 and 87; 56 and 88; 56 and 89; 56 and 90; 56 and 91; 56 and 92; 56 and 93; 56 and 94; 56 and 95; 56 and 96; 56 and 97; 56 and 98; 56 and 99; 56 and 100; 56 and 101; 56 and 102; 56 and 103; 56 and 104; 56 and 105; 56 and 106; 56 and 107; 56 and 108; 56 and 109; 56 and 110; 56 and 111; 56 and 112; 56 and 113; 56 and 114; 56 and 115; 56 and 116; 56 and 117; 56 and 118; 56 and 119; 56 and 120; 56 and 121; 56 and 122; 56 and 123; 56 and 124; 56 and 125; 56 and 126; 56 and 127; 56 and 128; 56 and 129; 56 and 130; 56 and 131; 56 and 132; 56 and 133; 56 and 134; 56 and 135; 56 and 136; 56 and 137; 56 and 138; 56 and 139; 56 and 140; 56 and 141; 56 and 142; 56 and 143; 56 and 144; 56 and 145; 56 and 146; 56 and 147; 56 and 148; 56 and 149; 56 and 150; 56 and 151; 56 and 152; 56 and 153; 56 and 154; 56 and 155; 56 and 156; 56 and 157; 56 and 158; 56 and 159; 56 and 160; 56 and 161; 56 and 162; 56 and 163; 56 and 164; 56 and 165; 56 and 166; 56 and 167; 57 and 67; 57 and 68; 57 and 69; 57 and 70; 57 and 71; 57 and 72; 57 and 73; 57 and 74; 57 and 75; 57 and 76; 57 and 77; 57 and 78; 57 and 79; 57 and 80; 57 and 81; 57 and 82; 57 and 83; 57 and 84; 57 and 85; 57 and 86; 57 and 87; 57 and 88; 57 and 89; 57 and 90; 57 and 91; 57 and 92; 57 and 93; 57 and 94; 57 and 95; 57 and 96; 57 and 97; 57 and 98; 57 and 99; 57 and 100; 57 and 101; 57 and 102; 57 and 103; 57 and 104; 57 and 105; 57 and 106; 57 and 107; 57 and 108; 57 and 109; 57 and 110; 57 and 111; 57 and 112; 57 and 113; 57 and 114; 57 and 115; 57 and 116; 57 and 117; 57 and 118; 57 and 119; 57 and 120; 57 and 121; 57 and 122; 57 and 123; 57 and 124; 57 and 125; 57 and 126; 57 and 127; 57 and 128; 57 and 129; 57 and 130; 57 and 131; 57 and 132; 57 and 133; 57 and 134; 57 and 135; 57 and 136; 57 and 137; 57 and 138; 57 and 139; 57 and 140; 57 and 141; 57 and 142; 57 and 143; 57 and 144; 57 and 145; 57 and 146; 57 and 147; 57 and 148; 57 and 149; 57 and 150; 57 and 151; 57 and 152; 57 and 153; 57 and 154; 57 and 155; 57 and 156; 57 and 157; 57 and 158; 57 and 159; 57 and 160; 57 and 161; 57 and 162; 57 and 163; 57 and 164; 57 and 165; 57 and 166; 57 and 167; 58 and 67; 58 and 68; 58 and 69; 58 and 70; 58 and 71; 58 and 72; 58 and 73; 58 and 74; 58 and 75; 58 and 76; 58 and 77; 58 and 78; 58 and 79; 58 and 80; 58 and 81; 58 and 82; 58 and 83; 58 and 84; 58 and 85; 58 and 86; 58 and 87; 58 and 88; 58 and 89; 58 and 90; 58 and 91; 58 and 92; 58 and 93; 58 and 94; 58 and 95; 58 and 96; 58 and 97; 58 and 98; 58 and 99; 58 and 100; 58 and 101; 58 and 102; 58 and 103; 58 and 104; 58 and 105; 58 and 106; 58 and 107; 58 and 108; 58 and 109; 58 and 110; 58 and 111; 58 and 112; 58 and 113; 58 and 114; 58 and 115; 58 and 116; 58 and 117; 58 and 118; 58 and 119; 58 and 120; 58 and 121; 58 and 122; 58 and 123; 58 and 124; 58 and 125; 58 and 126; 58 and 127; 58 and 128; 58 and 129; 58 and 130; 58 and 131; 58 and 132; 58 and 133; 58 and 134; 58 and 135; 58 and 136; 58 and 137; 58 and 138; 58 and 139; 58 and 140; 58 and 141; 58 and 142; 58 and 143; 58 and 144; 58 and 145; 58 and 146; 58 and 147; 58 and 148; 58 and 149; 58 and 150; 58 and 151; 58 and 152; 58 and 153; 58 and 154; 58 and 155; 58 and 156; 58 and 157; 58 and 158; 58 and 159; 58 and 160; 58 and 161; 58 and 162; 58 and 163; 58 and 164; 58 and 165; 58 and 166; 58 and 167; 59 and 67; 59 and 68; 59 and 69; 59 and 70; 59 and 71; 59 and 72; 59 and 73; 59 and 74; 59 and 75; 59 and 76; 59 and 77; 59 and 78; 59 and 79; 59 and 80; 59 and 81; 59 and 82; 59 and 83; 59 and 84; 59 and 85; 59 and 86; 59 and 87; 59 and 88; 59 and 89; 59 and 90; 59 and 91; 59 and 92; 59 and 93; 59 and 94; 59 and 95; 59 and 96; 59 and 97; 59 and 98; 59 and 99; 59 and 100; 59 and 101; 59 and 102; 59 and 103; 59 and 104; 59 and 105; 59 and 106; 59 and 107; 59 and 108; 59 and 109; 59 and 110; 59 and 111; 59 and 112; 59 and 113; 59 and 114; 59 and 115; 59 and 116; 59 and 117; 59 and 118; 59 and 119; 59 and 120; 59 and 121; 59 and 122; 59 and 123; 59 and 124; 59 and 125; 59 and 126; 59 and 127; 59 and 128; 59 and 129; 59 and 130; 59 and 131; 59 and 132; 59 and 133; 59 and 134; 59 and 135; 59 and 136; 59 and 137; 59 and 138; 59 and 139; 59 and 140; 59 and 141; 59 and 142; 59 and 143; 59 and 144; 59 and 145; 59 and 146; 59 and 147; 59 and 148; 59 and 149; 59 and 150; 59 and 151; 59 and 152; 59 and 153; 59 and 154; 59 and 155; 59 and 156; 59 and 157; 59 and 158; 59 and 159; 59 and 160; 59 and 161; 59 and 162; 59 and 163; 59 and 164; 59 and 165; 59 and 166; 59 and 167; 60 and 67; 60 and 68; 60 and 69; 60 and 70; 60 and 71; 60 and 72; 60 and 73; 60 and 74; 60 and 75; 60 and 76; 60 and 77; 60 and 78; 60 and 79; 60 and 80; 60 and 81; 60 and 82; 60 and 83; 60 and 84; 60 and 85; 60 and 86; 60 and 87; 60 and 88; 60 and 89; 60 and 90; 60 and 91; 60 and 92; 60 and 93; 60 and 94; 60 and 95; 60 and 96; 60 and 97; 60 and 98; 60 and 99; 60 and 100; 60 and 101; 60 and 102; 60 and 103; 60 and 104; 60 and 105; 60 and 106; 60 and 107; 60 and 108; 60 and 109; 60 and 110; 60 and 111; 60 and 112; 60 and 113; 60 and 114; 60 and 115; 60 and 116; 60 and 117; 60 and 118; 60 and 119; 60 and 120; 60 and 121; 60 and 122; 60 and 123; 60 and 124; 60 and 125; 60 and 126; 60 and 127; 60 and 128; 60 and 129; 60 and 130; 60 and 131; 60 and 132; 60 and 133; 60 and 134; 60 and 135; 60 and 136; 60 and 137; 60 and 138; 60 and 139; 60 and 140; 60 and 141; 60 and 142; 60 and 143; 60 and 144; 60 and 145; 60 and 146; 60 and 147; 60 and 148; 60 and 149; 60 and 150; 60 and 151; 60 and 152; 60 and 153; 60 and 154; 60 and 155; 60 and 156; 60 and 157; 60 and 158; 60 and 159; 60 and 160; 60 and 161; 60 and 162; 60 and 163; 60 and 164; 60 and 165; 60 and 166; 60 and 167; 61 and 67; 61 and 68; 61 and 69; 61 and 70; 61 and 71; 61 and 72; 61 and 73; 61 and 74; 61 and 75; 61 and 76; 61 and 77; 61 and 78; 61 and 79; 61 and 80; 61 and 81; 61 and 82; 61 and 83; 61 and 84; 61 and 85; 61 and 86; 61 and 87; 61 and 88; 61 and 89; 61 and 90; 61 and 91; 61 and 92; 61 and 93; 61 and 94; 61 and 95; 61 and 96; 61 and 97; 61 and 98; 61 and 99; 61 and 100; 61 and 101; 61 and 102; 61 and 103; 61 and 104; 61 and 105; 61 and 106; 61 and 107; 61 and 108; 61 and 109; 61 and 110; 61 and 111; 61 and 112; 61 and 113; 61 and 114; 61 and 115; 61 and 116; 61 and 117; 61 and 118; 61 and 119; 61 and 120; 61 and 121; 61 and 122; 61 and 123; 61 and 124; 61 and 125; 61 and 126; 61 and 127; 61 and 128; 61 and 129; 61 and 130; 61 and 131; 61 and 132; 61 and 133; 61 and 134; 61 and 135; 61 and 136; 61 and 137; 61 and 138; 61 and 139; 61 and 140; 61 and 141; 61 and 142; 61 and 143; 61 and 144; 61 and 145; 61 and 146; 61 and 147; 61 and 148; 61 and 149; 61 and 150; 61 and 151; 61 and 152; 61 and 153; 61 and 154; 61 and 155; 61 and 156; 61 and 157; 61 and 158; 61 and 159; 61 and 160; 61 and 161; 61 and 162; 61 and 163; 61 and 164; 61 and 165; 61 and 166; 61 and 167; 62 and 67; 62 and 68; 62 and 69; 62 and 70; 62 and 71; 62 and 72; 62 and 73; 62 and 74; 62 and 75; 62 and 76; 62 and 77; 62 and 78; 62 and 79; 62 and 80; 62 and 81; 62 and 82; 62 and 83; 62 and 84; 62 and 85; 62 and 86; 62 and 87; 62 and 88; 62 and 89; 62 and 90; 62 and 91; 62 and 92; 62 and 93; 62 and 94; 62 and 95; 62 and 96; 62 and 97; 62 and 98; 62 and 99; 62 and 100; 62 and 101; 62 and 102; 62 and 103; 62 and 104; 62 and 105; 62 and 106; 62 and 107; 62 and 108; 62 and 109; 62 and 110; 62 and 111; 62 and 112; 62 and 113; 62 and 114; 62 and 115; 62 and 116; 62 and 117; 62 and 118; 62 and 119; 62 and 120; 62 and 121; 62 and 122; 62 and 123; 62 and 124; 62 and 125; 62 and 126; 62 and 127; 62 and 128; 62 and 129; 62 and 130; 62 and 131; 62 and 132; 62 and 133; 62 and 134; 62 and 135; 62 and 136; 62 and 137; 62 and 138; 62 and 139; 62 and 140; 62 and 141; 62 and 142; 62 and 143; 62 and 144; 62 and 145; 62 and 146; 62 and 147; 62 and 148; 62 and 149; 62 and 150; 62 and 151; 62 and 152; 62 and 153; 62 and 154; 62 and 155; 62 and 156; 62 and 157; 62 and 158; 62 and 159; 62 and 160; 62 and 161; 62 and 162; 62 and 163; 62 and 164; 62 and 165; 62 and 166; 62 and 167; 63 and 67; 63 and 68; 63 and 69; 63 and 70; 63 and 71; 63 and 72; 63 and 73; 63 and 74; 63 and 75; 63 and 76; 63 and 77; 63 and 78; 63 and 79; 63 and 80; 63 and 81; 63 and 82; 63 and 83; 63 and 84; 63 and 85; 63 and 86; 63 and 87; 63 and 88; 63 and 89; 63 and 90; 63 and 91; 63 and 92; 63 and 93; 63 and 94; 63 and 95; 63 and 96; 63 and 97; 63 and 98; 63 and 99; 63 and 100; 63 and 101; 63 and 102; 63 and 103; 63 and 104; 63 and 105; 63 and 106; 63 and 107; 63 and 108; 63 and 109; 63 and 110; 63 and 111; 63 and 112; 63 and 113; 63 and 114; 63 and 115; 63 and 116; 63 and 117; 63 and 118; 63 and 119; 63 and 120; 63 and 121; 63 and 122; 63 and 123; 63 and 124; 63 and 125; 63 and 126; 63 and 127; 63 and 128; 63 and 129; 63 and 130; 63 and 131; 63 and 132; 63 and 133; 63 and 134; 63 and 135; 63 and 136; 63 and 137; 63 and 138; 63 and 139; 63 and 140; 63 and 141; 63 and 142; 63 and 143; 63 and 144; 63 and 145; 63 and 146; 63 and 147; 63 and 148; 63 and 149; 63 and 150; 63 and 151; 63 and 152; 63 and 153; 63 and 154; 63 and 155; 63 and 156; 63 and 157; 63 and 158; 63 and 159; 63 and 160; 63 and 161; 63 and 162; 63 and 163; 63 and 164; 63 and 165; 63 and 166; 63 and 167; 64 and 67; 64 and 68; 64 and 69; 64 and 70; 64 and 71; 64 and 72; 64 and 73; 64 and 74; 64 and 75; 64 and 76; 64 and 77; 64 and 78; 64 and 79; 64 and 80; 64 and 81; 64 and 82; 64 and 83; 64 and 84; 64 and 85; 64 and 86; 64 and 87; 64 and 88; 64 and 89; 64 and 90; 64 and 91; 64 and 92; 64 and 93; 64 and 94; 64 and 95; 64 and 96; 64 and 97; 64 and 98; 64 and 99; 64 and 100; 64 and 101; 64 and 102; 64 and 103; 64 and 104; 64 and 105; 64 and 106; 64 and 107; 64 and 108; 64 and 109; 64 and 110; 64 and 111; 64 and 112; 64 and 113; 64 and 114; 64 and 115; 64 and 116; 64 and 117; 64 and 118; 64 and 119; 64 and 120; 64 and 121; 64 and 122; 64 and 123; 64 and 124; 64 and 125; 64 and 126; 64 and 127; 64 and 128; 64 and 129; 64 and 130; 64 and 131; 64 and 132; 64 and 133; 64 and 134; 64 and 135; 64 and 136; 64 and 137; 64 and 138; 64 and 139; 64 and 140; 64 and 141; 64 and 142; 64 and 143; 64 and 144; 64 and 145; 64 and 146; 64 and 147; 64 and 148; 64 and 149; 64 and 150; 64 and 151; 64 and 152; 64 and 153; 64 and 154; 64 and 155; 64 and 156; 64 and 157; 64 and 158; 64 and 159; 64 and 160; 64 and 161; 64 and 162; 64 and 163; 64 and 164; 64 and 165; 64 and 166; 64 and 167; 65 and 67; 65 and 68; 65 and 69; 65 and 70; 65 and 71; 65 and 72; 65 and 73; 65 and 74; 65 and 75; 65 and 76; 65 and 77; 65 and 78; 65 and 79; 65 and 80; 65 and 81; 65 and 82; 65 and 83; 65 and 84; 65 and 85; 65 and 86; 65 and 87; 65 and 88; 65 and 89; 65 and 90; 65 and 91; 65 and 92; 65 and 93; 65 and 94; 65 and 95; 65 and 96; 65 and 97; 65 and 98; 65 and 99; 65 and 100; 65 and 101; 65 and 102; 65 and 103; 65 and 104; 65 and 105; 65 and 106; 65 and 107; 65 and 108; 65 and 109; 65 and 110; 65 and 111; 65 and 112; 65 and 113; 65 and 114; 65 and 115; 65 and 116; 65 and 117; 65 and 118; 65 and 119; 65 and 120; 65 and 121; 65 and 122; 65 and 123; 65 and 124; 65 and 125; 65 and 126; 65 and 127; 65 and 128; 65 and 129; 65 and 130; 65 and 131; 65 and 132; 65 and 133; 65 and 134; 65 and 135; 65 and 136; 65 and 137; 65 and 138; 65 and 139; 65 and 140; 65 and 141; 65 and 142; 65 and 143; 65 and 144; 65 and 145; 65 and 146; 65 and 147; 65 and 148; 65 and 149; 65 and 150; 65 and 151; 65 and 152; 65 and 153; 65 and 154; 65 and 155; 65 and 156; 65 and 157; 65 and 158; 65 and 159; 65 and 160; 65 and 161; 65 and 162; 65 and 163; 65 and 164; 65 and 165; 65 and 166; and 65 and 167 and a second nucleic acid encoding a Staphylococcus lugdunensis Cas9 (SluCas9);
b. a first nucleic acid encoding a pair of guide RNAs comprising at least 17, 18, 19, 20, or 21 contiguous nucleotides of a first and second spacer sequence selected from any one of a. and a second nucleic acid encoding a Staphylococcus lugdunensis Cas9 (SluCas9); or
c. a first nucleic acid encoding a pair of guide RNAs that is at least 90% identical to a first and second spacer sequence selected from any one of a. and a second nucleic acid encoding a Staphylococcus lugdunensis Cas9 (SluCas9).
   In some embodiments, the composition further comprises a DNA-PK inhibitor. In some embodiments, the DNA-PK inhibitor is Compound 1. In some embodiments, the DNA-PK inhibitor is Compound 2. In some embodiments, the DNA-PK inhibitor is Compound 6.

In some embodiments, a nucleic acid encoding a guide RNA and a nucleic acid encoding a Cas9 are provided on a single nucleic acid molecule. In some embodiments, the single nucleic acid molecule comprises a nucleic acid encoding one or more guide RNAs and a nucleic acid encoding a SluCas9. In some embodiments, nucleotide sequences encoding a Cas9 (e.g., SluCas9) and one or more copies of a single guide RNA (e.g., a guide RNA comprising the sequence of any one of SEQ ID Nos: 8, 63, 64, or 81) are provided on a single nucleic acid molecule. In some embodiments, nucleotide sequences encoding two guide RNAs and a Cas9 are provided on a single nucleic acid molecule. In some embodiments, the nucleic acid encoding three guide RNAs and a nucleic acid encoding a SluCas9 are provided on a single nucleic acid molecule. In some embodiments, single nucleic acid molecule comprises a nucleic acid encoding a Cas9, and a nucleic acid encoding two guide RNAs, wherein the nucleic acid molecule encodes no more than two guide RNAs. In some embodiments, the single nucleic acid molecule comprises a nucleic acid encoding a first guide RNA, a nucleic acid encoding a second guide RNA, and a nucleic acid encoding a SluCas9, where the first and second guide RNA can be the same or different. In some embodiments, the first guide RNA comprises a sequence selected from any one of SEQ ID Nos: 6, 8, 10, 21, 58, 62, 63, or 64, and the second guide RNA comprises a sequence selected from any one of SEQ ID Nos: 72, 81, 84, 98, 100, 114, 122, 134, 139, 149 or 166. In some embodiments, the single nucleic acid molecule comprises a nucleic acid encoding a first guide RNA, a nucleic acid encoding a second guide RNA, a nucleic acid encoding a third guide RNA, and a nucleic acid encoding a SluCas9, where the first, second, and third guide RNA can be the same or different. In some embodiments, the spacer sequences of the first and second guide RNAs are identical. In some embodiments, the spacer sequences of the first and second guide RNAs are non-identical (e.g., a pair of guide RNAs). In some embodiments, a system is provided comprising two vectors, wherein the first vector comprises one or more (e.g., 1, 2, 3, 4, 5, or 6) guide RNAs, which can be the same or different, and a second vector comprises one or more guide RNAs (e.g., 1, 2, or 3), which can be the same or different as compared to the other guide RNAs in the second vector or as compared to the other guide RNAs in the first vector, and a nucleic acid encoding a SluCas9.

In some embodiments, the disclosure provides for a composition comprising two nucleic acid molecules, wherein the first nucleic acid molecule comprises a sequence encoding a SluCas9 protein, and wherein the second nucleic acid molecule encodes for a first guide RNA. In some embodiments, the first nucleic acid molecule also encodes for the first guide RNA. In other embodiments, the first nucleic acid molecule does not encode for any guide RNA. In some embodiments, the second nucleic acid molecule encodes for a second guide RNA. In some embodiments, the first nucleic acid molecule also encodes for the second guide RNA. In particular embodiments, the first guide RNA and the second guide RNA are not identical. In some embodiments, the second nucleic acid molecule encodes for two copies of the first guide RNA. In some embodiments, the second nucleic acid molecule encodes for two copies of the second guide RNA. In some embodiments, the second nucleic acid molecule encodes for three copies of the first guide RNA. In some embodiments, the second nucleic acid molecule encodes for three copies of the second guide RNA. In some embodiments, the second nucleic acid molecule encodes for two copies of the first guide RNA and two copies of the second guide RNA. In some embodiments, the second nucleic acid molecule encodes for two copies of the first guide RNA and one copy of the second guide RNA. In some embodiments, the second nucleic acid molecule encodes for one copy of the first guide RNA and two copies of the second guide RNA. In some embodiments, the second nucleic acid molecule encodes for three copies of the first guide RNA and three copies of the second guide RNA. In particular embodiments, the first guide RNA and the second guide RNA are not identical. In some embodiments, the first nucleic acid is in a first viral vector and the second nucleic acid is in a separate second viral vector. In some embodiments, the first guide RNA comprises a sequence selected from any one of SEQ ID Nos: 6, 8, 10, 21, 58, 62, 63, or 64, and the second guide RNA comprises a sequence selected from any one of SEQ ID Nos: 72, 81, 84, 98, 100, 114, 122, 134, 139, 149 or 166. In some embodiments, the second nucleic acid encodes for one or more copies of a first guide RNA (e.g., a guide RNA comprising a sequence from any one of SEQ ID Nos: 6, 8, 10, 21, 58, 62, 63, 64, 72, 81, 84, 98, 100, 114, 122, 134, 139, 149 or 166), and does not encode for any additional different guide RNAs. In some embodiments, the second nucleic acid encodes for one or more copies of a first guide RNA comprising the nucleotide sequence of SEQ ID NO: 8, 63, 64, or 81, and does not encode for any additional different guide RNAs. In some embodiments, the first nucleic acid molecule encodes for a Cas9 molecule and also encodes for one or more copies of a first guide RNA and one or more copies of a second guide RNA. In some embodiments, the first nucleic acid molecule encodes for a Cas9 molecule, but does not encode for any guide RNAs. In some embodiments, the second nucleic acid molecule encodes for one or more copies of a first guide RNA and one or more copies of a second guide RNA, wherein the second nucleic acid molecule does not encode for a Cas9 molecule.

In some embodiments, the single nucleic acid molecule is a single vector. In some embodiments, the single vector expresses the one or two or three guide RNAs and Cas9. In some embodiments, one or more guide RNAs and a Cas9 are encoded by a nucleic acid provided on a single vector. In some embodiments, the single vector comprises a nucleic acid encoding a guide RNA and a nucleic acid encoding a SluCas9. In some embodiments, two guide RNAs and a Cas9 are encoded by a nucleic acid provided on a single vector. In some embodiments, three guide RNAs and a Cas9 are provided on a single vector. In some embodiments, the single vector comprises a nucleic acid encoding a first guide RNA, a nucleic acid encoding a second guide RNA, and a nucleic acid encoding a SluCas9. In some embodiments, the single vector comprises a nucleic acid encoding a first guide RNA, a nucleic acid encoding a second guide RNA, a nucleic acid encoding a third guide RNA, and a nucleic acid encoding a SluCas9. In some embodiments, the spacer sequences of the first, second, and third guide RNAs, if present, are identical. In some embodiments, the spacer sequences of the first, second, and third guide RNAs, if present, are non-identical (e.g., a pair of guide RNAs).

Each of the guide sequences shown in Table 1A and Table 1B may further comprise additional nucleotides to form or encode a crRNA, e.g., using any known sequence appropriate for the Cas9 being used. In some embodiments, the crRNA comprises (5′ to 3′) at least a spacer sequence and a first complementarity domain. The first complementary domain is sufficiently complementary to a second complementarity domain, which may be part of the same molecule in the case of an sgRNA or in a tracrRNA in the case of a dual or modular gRNA, to form a duplex. See, e.g., US 2017/0007679 for detailed discussion of crRNA and gRNA domains, including first and second complementarity domains.

A single-molecule guide RNA (sgRNA) can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and/or an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins.

Two exemplary scaffold sequences suitable for use with SluCas9 to follow the guide sequence at its 3′ end is:

### GTTTTAGTACTCTGGAAACAGAATCTACTGAAACAAGACAATATGTCGTGTTTATCCCAT CAATTTATTGGTGGGA (SEQ ID NO: 600), and

GTTTAAGTACTCTGTGCTGGAAACAGCACAGAATCTACTGAAACAAGACAATATGTCGT GTTTATCCCATCAATTTATTGGTGGGA (SEQ ID NO: 601) in 5′ to 3′ orientation. In some embodiments, an exemplary sequence for use with SluCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 600 or SEQ ID NO: 601, or a sequence that differs from SEQ ID NO: 600 or SEQ ID NO: 601 by no more than 1, 2, 3, 4, 5, 10, 15, 20, or 25 nucleotides.

Exemplary scaffold sequences suitable for use with SluCas9 to follow the guide sequence at its 3′ end are also shown below in the 5′ to 3′ orientation:

| | | | | Streak of |
|---|---|---|---|---|
| | | | | Homology to |
| Scaffold | SEQ | | Homology | Slu v5 |
| ID | ID NO | Scaffold Sequence (5′ to 3′) | to Slu v5 | (# nucleotides) |
| Wildtype | 900 | GTTTTAGTACTCTGGAAACAGAATCTACTGAA | N/A | N/A |
| | | ACAAGACAATATGTCGTGTTTATCCCATCAAT | | |
| | | TTATTGGTGGGAT | | |
| Slu- | 601 | GTTTAAGTACTCTGTGCTGGAAACAGCACAG | N/A | N/A |
| VCGT- | | AATCTACTGAAACAAGACAATATGTCGTGTTT | | |
| 4.5 | | ATCCCATCAATTTATTGGTGGGA | | |
| Slu_v5 | 901 | GTTTCAGTACTCTGGAAACAGAATCTACTGAA | 100.00% | 77 |
| | | ACAAGACAATATGTCGTGTTTATCCCATCAAT | | |
| | | TTATTGGTGGGAT | | |
| Slu_v5-1 | 902 | GTTTggTaACcTaGGAAACTagATCTTaccAAACA | 87.50% | 47 |
| | | AGACAATATGTCGTGTTTATCCCATCAATTTA | | |
| | | TTGGTGGGAT | | |
| Slu_v5-2 | 903 | GTTTCAGTACTCTGGAAACAGAATCTACTGAA | 96.10% | 37 |
| | | ACAAGgCAAaATGcCGTGTTTATCCCATCAATT | | |
| | | TATTGGTGGGAT | | |
| Slu_v5-3 | 904 | GTTTCAGTACTCTGGAAACAGAATCTACTGAA | 94.81% | 48 |
| | | ACAAGACAATATGTCGcgcccaTCCCATCAATTT | | |
| | | ATTGGTGGGAT | | |
| Slu_v5-4 | 905 | GTTTCAGTACTCTGGAAACAGAATCTACTGAA | 91.55% | 55 |
| | | ACAAGACAATATGTCGTGTTTATgggTTgAATT | | |
| | | TATTcGacccAT | | |
| Slu_v5-5 | 906 | GTTTggTaACcTaGGAAACTagATCTTaccAAACA | 83.75% | 31 |
| | | AGgCAAaATGcCGTGTTTATCCCATCAATTTAT | | |
| | | TGGTGGGAT | | |
| Slu_v5-6 | 907 | GTTTggTaACcTaGGAAACTagATCTTaccAAACA | 82.50% | 23 |
| | | AGACAATATGTCGcgcccaTCCCATCAATTTATT | | |
| | | GGTGGGAT | | |
| Slu_v5-7 | 908 | GTTTggTaACcTaGGAAACTagATCTTaccAAACA | 78.38% | 25 |
| | | AGACAATATGTCGTGTTTATgggTTgAATTTAT | | |
| | | TcGacccAT | | |
| Slu_v5-8 | 909 | GTTTCAGTACTCTGGAAACAGAATCTACTGAA | 90.91% | 37 |
| | | ACAAGgCAAaATGcCGcgcccaTCCCATCAATTTA | | |
| | | TTGGTGGGAT | | |
| Slu_v5-9 | 910 | GTTTCAGTACTCTGGAAACAGAATCTACTGAA | 87.32% | 37 |
| | | ACAAGgCAAaATGcCGTGTTTATgggTTgAATTT | | |
| | | ATTcGacccAT | | |
| Slu_v5- | 911 | GTTTCAGTACTCTGGAAACAGAATCTACTGAA | 82.89% | 48 |
| 10 | | ACAAGACAATATGTCGcgcccaTgggTTgAATTTA | | |
| | | TTcGacccAT | | |
| Slu_v5- | 912 | GTTTggTaACcTaGGAAACTagATCTTaccAAACA | 78.75% | 23 |
| 11 | | AGgCAAaATGcCGcgcccaTCCCATCAATTTATTG | | |
| | | GTGGGAT | | |
| Slu_v5- | 913 | GTTTggTaACcTaGGAAACTagATCTTaccAAACA | 74.32% | 9 |
| 12 | | AGgCAAaATGcCGTGTTTATgggTTgAATTTATTc | | |
| | | GacccAT | | |
| Slu_v5- | 914 | GTTTggTaACcTaGGAAACTagATCTTaccAAACA | 70.89% | 18 |
| 13 | | AGACAATATGTCGcgcccaTgggTTgAATTTATTc | | |
| | | GacccAT | | |
| Slu_v5- | 915 | GTTTCAGTACTCTGGAAACAGAATCTACTGAA | 78.95% | 37 |
| 14 | | ACAAGgCAAaATGcCGcgcccaTgggTTgAATTTAT | | |
| | | TcGacccAT | | |
| Slu_v5- | 916 | GTTTggTaACcTaGGAAACTagATCTTaccAAACA | 67.09% | 8 |
| 15 | | AGgCAAaATGcCGcgcccaTgggTTgAATTTATTcGa | | |
| | | cccAT | | |
| Slu_v4 | 917 | GTTTCAGTACTCTGTGCTGGAAACAGCACAGA | N/A | N/A |
| | | ATCTACTGAAACAAGACAATATGTCGTGTTTA | | |
| | | TCCCATCAATTTATTGGTGGGAT | | |

In some embodiments, the scaffold sequence suitable for use with SluCas9 to follow the guide sequence at its 3′ end is selected from any one of SEQ ID NOs: 600-601, or 900-917 in 5′ to 3 orientation (see below). In some embodiments, an exemplary sequence for use with SluCas9 to follow the 3′ end of the guide sequence is a sequence that is at least 65%, 70%, 75%, 80%, 85%, 90%, 91%, 92%, 93%, 94%, 95%, 96%, 97%, 98%, 99% or 100% identical to any one off SEQ ID NOs: 600-601, or 900-917, or a sequence that differs from any one of SEQ ID NOs: 600-601, or 900-917 by no more than 1, 2, 3, 4, 5, 10, 15, 20, or 25 nucleotides.

In some embodiments, the scaffold sequence suitable for use with SluCas9 to follow the guide sequence at its 3′ end is selected from any one of SEQ ID NOs: 901-917 in 5′ to 3 orientation (see below). In some embodiments, an exemplary sequence for use with SluCas9 to follow the 3′ end of the guide sequence is a sequence that is at least 65%, 70%, 75%, 80%, 85%, 90%, 91%, 92%, 93%, 94%, 95%, 96%, 97%, 98%, 99% or 100% identical to any one off SEQ ID NOs: 901-917, or a sequence that differs from any one of SEQ ID NOs: 901-917 by no more than 1, 2, 3, 4, 5, 10, 15, 20, or 25 nucleotides.

In some embodiments, the nucleic acid encoding the gRNA or the nucleic acid encoding the pair of gRNAs comprises a sequence comprising SEQ ID NO: 600. In some embodiments, the nucleic acid encoding the gRLNA or the nucleic acid encoding the pair of gRLNAs comprises a sequence comprising SEQ ID NO: 601. In some embodiments, the nucleic acid encoding the gRNA or the nucleic acid encoding the pair of gRNAs comprises a sequence comprising SEQ ID NO: 900. In some embodiments, the nucleic acid encoding the gRNA or the nucleic acid encoding the pair of gRNAs comprises a sequence comprising SEQ ID NO: 901. In some embodiments, the nucleic acid encoding the gRNA or the nucleic acid encoding the pair of gRNAs comprises a sequence comprising SEQ ID NO: 902. In some embodiments, the nucleic acid encoding the gRNA or the nucleic acid encoding the pair of gRNAs comprises a sequence comprising SEQ ID NO: 903. In some embodiments, the nucleic acid encoding the gRNA or the nucleic acid encoding the pair of gRNAs comprises a sequence comprising SEQ ID NO: 904. In some embodiments, the nucleic acid encoding the gRNA or the nucleic acid encoding the pair of gRNAs comprises a sequence comprising SEQ ID NO: 905. In some embodiments, the nucleic acid encoding the gRNA or the nucleic acid encoding the pair of gRNAs comprises a sequence comprising SEQ ID NO: 906. In some embodiments, the nucleic acid encoding the gRNA or the nucleic acid encoding the pair of gRNAs comprises a sequence comprising SEQ ID NO: 907. In some embodiments, the nucleic acid encoding the gRNA or the nucleic acid encoding the pair of gRNAs comprises a sequence comprising SEQ ID NO: 908. In some embodiments, the nucleic acid encoding the gRNA or the nucleic acid encoding the pair of gRNAs comprises a sequence comprising SEQ ID NO: 909. In some embodiments, the nucleic acid encoding the gRNA or the nucleic acid encoding the pair of gRNAs comprises a sequence comprising SEQ ID NO: 910. In some embodiments, the nucleic acid encoding the gRNA or the nucleic acid encoding the pair of gRNAs comprises a sequence comprising SEQ ID NO: 911. In some embodiments, the nucleic acid encoding the gRNA or the nucleic acid encoding the pair of gRNAs comprises a sequence comprising SEQ ID NO: 912. In some embodiments, the nucleic acid encoding the gRNA or the nucleic acid encoding the pair of gRNAs comprises a sequence comprising SEQ ID NO: 913. In some embodiments, the nucleic acid encoding the gRNA or the nucleic acid encoding the pair of gRNAs comprises a sequence comprising SEQ ID NO: 914. In some embodiments, the nucleic acid encoding the gRNA or the nucleic acid encoding the pair of gRNAs comprises a sequence comprising SEQ ID NO: 915. In some embodiments, the nucleic acid encoding the gRNA or the nucleic acid encoding the pair of gRNAs comprises a sequence comprising SEQ ID NO: 916. In some embodiments, the nucleic acid encoding the gRNA or the nucleic acid encoding the pair of gRNAs comprises a sequence comprising SEQ ID NO: 917. In some embodiments, in a nucleic acid molecule comprising a pair of gRNAs, one of the gRNAs comprises a sequence selected from any one of SEQ ID NOs: 600-601, or 900-917. In some embodiments, in a nucleic acid molecule comprising a pair of gRNAs, both of the gRNAs comprise a sequence selected from any one of SEQ ID NOs: 600-601, or 900-917. In some embodiments, in a nucleic acid molecule comprising a pair of gRNAs, the first gRNA in the pair comprises a sequence selected from any one of SEQ ID Nos: 600-601 or 900-917, and the second gRNA in the pair comprises a different sequence selected from any one of SEQ ID Nos: 600-601 or 900-917. In some embodiments, in a nucleic acid molecule comprising a pair of gRNAs, the nucleotides 3′ of the guide sequence of the gRNAs are the same sequence. In some embodiments, in a nucleic acid molecule comprising a pair of gRNAs, the nucleotides 3′ of the guide sequence of the gRNAs are different sequences.

In some embodiments, the scaffold sequence comprises one or more alterations in the stem loop 1 as compared to the stem loop 1 of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the stem loop 2 as compared to the stem loop 2 of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the tetraloop as compared to the tetraloop of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the repeat region as compared to the repeat region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the anti-repeat region as compared to the anti-repeat region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the linker region as compared to the linker region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). See, e.g., Nishimasu et al., 2015, Cell, 162:1113-1126 for description of regions of a scaffold.

Where a tracrRNA is used, in some embodiments, it comprises (5′ to 3′) a second complementary domain and a proximal domain. In the case of a sgRNA, guide sequences together with additional nucleotides (e.g., SEQ ID NOs: 600-601, or 900-917) form or encode a sgRNA. In some embodiments, an sgRNA comprises (5′ to 3′) at least a spacer sequence, a first complementary domain, a linking domain, a second complementary domain, and a proximal domain. A sgRNA or tracrRNA may further comprise a tail domain. The linking domain may be hairpin-forming. See, e.g., US 2017/0007679 for detailed discussion and examples of crRNA and gRNA domains, including second complementarity domains, linking domains, proximal domains, and tail domains.

In general, in the case of a DNA nucleic acid construct encoding a guide RNA, the U residues in any of the RNA sequences described herein may be replaced with T residues, and in the case of a guide RNA construct encoded by a DNA, the T residues may be replaced with U residues.

Provided herein are compositions comprising one or more guide RNAs or one or more nucleic acids encoding one or more guide RNAs comprising a guide sequence disclosed herein in Table 1A and Table 1B and throughout the specification.

In some embodiments, a composition is provided comprising a guide RNA, or nucleic acid encoding a guide RNA, wherein the guide RNA comprises 17, 18, 19, 20, or 21 contiguous nucleotides of any one of the guide sequences disclosed herein in Table 1A and Table 1B and throughout the specification.

In some embodiments, a composition is provided comprising a guide RNA, or nucleic acid encoding a guide RNA, wherein the guide RNA comprises a sequence with about 75%, 80%, 85%, 90%, 95%, 96%, 97%, 98%, 99%, or 100% identity to at least 17, 18, 19, 20, or 21 contiguous nucleotides of a guide sequence shown in Table 1A and Table 1B and throughout the specification.

In some embodiments, a composition is provided comprising a guide RNA, or nucleic acid encoding a guide RNA, wherein the guide RNA comprises a sequence with about 75%, 80%, 85%, 90%, 95%, 96%, 97%, 98%, 99%, or 100% identity to a guide sequence shown in Table 1A and Table 1B and throughout the specification.

In some embodiments, a composition is provided comprising at least one guide RNA, or nucleic acid encoding at least one guide RNA, wherein at least one of the guide RNA comprises a spacer sequence selected from any one of SEQ ID NOs: 1-65, 67-167, and 201-531. In some embodiments, the spacer sequence is SEQ ID NO: 1. In some embodiments, the spacer sequence is SEQ ID NO: 2. In some embodiments, the spacer sequence is SEQ ID NO: 3. In some embodiments, the spacer sequence is SEQ ID NO: 4. In some embodiments, the spacer sequence is SEQ ID NO: 5. In some embodiments, the spacer sequence is SEQ ID NO: 6. In some embodiments, the spacer sequence is SEQ ID NO: 7. In some embodiments, the spacer sequence is SEQ ID NO: 8. In some embodiments, the spacer sequence is SEQ ID NO: 9. In some embodiments, the spacer sequence is SEQ ID NO: 10. In some embodiments, the spacer sequence is SEQ ID NO: 11. In some embodiments, the spacer sequence is SEQ ID NO: 12. In some embodiments, the spacer sequence is SEQ ID NO: 13. In some embodiments, the spacer sequence is SEQ ID NO: 14. In some embodiments, the spacer sequence is SEQ ID NO: 15. In some embodiments, the spacer sequence is SEQ ID NO: 16. In some embodiments, the spacer sequence is SEQ ID NO: 17. In some embodiments, the spacer sequence is SEQ ID NO: 18. In some embodiments, the spacer sequence is SEQ ID NO: 19. In some embodiments, the spacer sequence is SEQ ID NO: 20. In some embodiments, the spacer sequence is SEQ ID NO: 21. In some embodiments, the spacer sequence is SEQ ID NO: 22. In some embodiments, the spacer sequence is SEQ ID NO: 23. In some embodiments, the spacer sequence is SEQ ID NO: 24. In some embodiments, the spacer sequence is SEQ ID NO: 25. In some embodiments, the spacer sequence is SEQ ID NO: 26. In some embodiments, the spacer sequence is SEQ ID NO: 27. In some embodiments, the spacer sequence is SEQ ID NO: 28. In some embodiments, the spacer sequence is SEQ ID NO: 29. In some embodiments, the spacer sequence is SEQ ID NO: 30. In some embodiments, the spacer sequence is SEQ ID NO: 31. In some embodiments, the spacer sequence is SEQ ID NO: 32. In some embodiments, the spacer sequence is SEQ ID NO: 33. In some embodiments, the spacer sequence is SEQ ID NO: 34. In some embodiments, the spacer sequence is SEQ ID NO: 35. In some embodiments, the spacer sequence is SEQ ID NO: 36. In some embodiments, the spacer sequence is SEQ ID NO: 37. In some embodiments, the spacer sequence is SEQ ID NO: 38. In some embodiments, the spacer sequence is SEQ ID NO: 39. In some embodiments, the spacer sequence is SEQ ID NO: 40. In some embodiments, the spacer sequence is SEQ ID NO: 41. In some embodiments, the spacer sequence is SEQ ID NO: 42. In some embodiments, the spacer sequence is SEQ ID NO: 43. In some embodiments, the spacer sequence is SEQ ID NO: 44. In some embodiments, the spacer sequence is SEQ ID NO: 45. In some embodiments, the spacer sequence is SEQ ID NO: 46. In some embodiments, the spacer sequence is SEQ ID NO: 47. In some embodiments, the spacer sequence is SEQ ID NO: 48. In some embodiments, the spacer sequence is SEQ ID NO: 49. In some embodiments, the spacer sequence is SEQ ID NO: 50. In some embodiments, the spacer sequence is SEQ ID NO: 51. In some embodiments, the spacer sequence is SEQ ID NO: 51. In some embodiments, the spacer sequence is SEQ ID NO: 52. In some embodiments, the spacer sequence is SEQ ID NO: 53. In some embodiments, the spacer sequence is SEQ ID NO: 54. In some embodiments, the spacer sequence is SEQ ID NO: 55. In some embodiments, the spacer sequence is SEQ ID NO: 56. In some embodiments, the spacer sequence is SEQ ID NO: 57. In some embodiments, the spacer sequence is SEQ ID NO: 58. In some embodiments, the spacer sequence is SEQ ID NO: 59. In some embodiments, the spacer sequence is SEQ ID NO: 60. In some embodiments, the spacer sequence is SEQ ID NO: 61. In some embodiments, the spacer sequence is SEQ ID NO: 62. In some embodiments, the spacer sequence is SEQ ID NO: 63. In some embodiments, the spacer sequence is SEQ ID NO: 64. In some embodiments, the spacer sequence is SEQ ID NO: 65. In some embodiments, the spacer sequence is SEQ ID NO: 66. In some embodiments, the spacer sequence is SEQ ID NO: 67. In some embodiments, the spacer sequence is SEQ ID NO: 68. In some embodiments, the spacer sequence is SEQ ID NO: 69. In some embodiments, the spacer sequence is SEQ ID NO: 70. In some embodiments, the spacer sequence is SEQ ID NO: 71. In some embodiments, the spacer sequence is SEQ ID NO: 72. In some embodiments, the spacer sequence is SEQ ID NO: 73. In some embodiments, the spacer sequence is SEQ ID NO: 74. In some embodiments, the spacer sequence is SEQ ID NO: 75. In some embodiments, the spacer sequence is SEQ ID NO: 76. In some embodiments, the spacer sequence is SEQ ID NO: 77. In some embodiments, the spacer sequence is SEQ ID NO: 78. In some embodiments, the spacer sequence is SEQ ID NO: 79. In some embodiments, the spacer sequence is SEQ ID NO: 80. In some embodiments, the spacer sequence is SEQ ID NO: 81. In some embodiments, the spacer sequence is SEQ ID NO: 82. In some embodiments, the spacer sequence is SEQ ID NO: 83. In some embodiments, the spacer sequence is SEQ ID NO: 84. In some embodiments, the spacer sequence is SEQ ID NO: 85. In some embodiments, the spacer sequence is SEQ ID NO: 86. In some embodiments, the spacer sequence is SEQ ID NO: 87. In some embodiments, the spacer sequence is SEQ ID NO: 88. In some embodiments, the spacer sequence is SEQ ID NO: 89. In some embodiments, the spacer sequence is SEQ ID NO: 90. In some embodiments, the spacer sequence is SEQ ID NO: 91. In some embodiments, the spacer sequence is SEQ ID NO: 92. In some embodiments, the spacer sequence is SEQ ID NO: 93. In some embodiments, the spacer sequence is SEQ ID NO: 94. In some embodiments, the spacer sequence is SEQ ID NO: 95. In some embodiments, the spacer sequence is SEQ ID NO: 96. In some embodiments, the spacer sequence is SEQ ID NO: 97. In some embodiments, the spacer sequence is SEQ ID NO: 98. In some embodiments, the spacer sequence is SEQ ID NO: 99. In some embodiments, the spacer sequence is SEQ ID NO: 100. In some embodiments, the spacer sequence is SEQ ID NO: 101. In some embodiments, the spacer sequence is SEQ ID NO: 102. In some embodiments, the spacer sequence is SEQ ID NO: 103. In some embodiments, the spacer sequence is SEQ ID NO: 104. In some embodiments, the spacer sequence is SEQ ID NO: 105. In some embodiments, the spacer sequence is SEQ ID NO: 106. In some embodiments, the spacer sequence is SEQ ID NO: 107. In some embodiments, the spacer sequence is SEQ ID NO: 108. In some embodiments, the spacer sequence is SEQ ID NO: 109. In some embodiments, the spacer sequence is SEQ ID NO: 110. In some embodiments, the spacer sequence is SEQ ID NO: 111. In some embodiments, the spacer sequence is SEQ ID NO: 112. In some embodiments, the spacer sequence is SEQ ID NO: 113. In some embodiments, the spacer sequence is SEQ ID NO: 114. In some embodiments, the spacer sequence is SEQ ID NO: 115. In some embodiments, the spacer sequence is SEQ ID NO: 116. In some embodiments, the spacer sequence is SEQ ID NO: 117. In some embodiments, the spacer sequence is SEQ ID NO: 118. In some embodiments, the spacer sequence is SEQ ID NO: 119. In some embodiments, the spacer sequence is SEQ ID NO: 120. In some embodiments, the spacer sequence is SEQ ID NO: 121. In some embodiments, the spacer sequence is SEQ ID NO: 122. In some embodiments, the spacer sequence is SEQ ID NO: 123. In some embodiments, the spacer sequence is SEQ ID NO: 124. In some embodiments, the spacer sequence is SEQ ID NO: 125. In some embodiments, the spacer sequence is SEQ ID NO: 126. In some embodiments, the spacer sequence is SEQ ID NO: 127. In some embodiments, the spacer sequence is SEQ ID NO: 128. In some embodiments, the spacer sequence is SEQ ID NO: 129. In some embodiments, the spacer sequence is SEQ ID NO: 130. In some embodiments, the spacer sequence is SEQ ID NO: 131. In some embodiments, the spacer sequence is SEQ ID NO: 132. In some embodiments, the spacer sequence is SEQ ID NO: 133. In some embodiments, the spacer sequence is SEQ ID NO: 134. In some embodiments, the spacer sequence is SEQ ID NO: 135. In some embodiments, the spacer sequence is SEQ ID NO: 136. In some embodiments, the spacer sequence is SEQ ID NO: 137. In some embodiments, the spacer sequence is SEQ ID NO: 138. In some embodiments, the spacer sequence is SEQ ID NO: 139. In some embodiments, the spacer sequence is SEQ ID NO: 140. In some embodiments, the spacer sequence is SEQ ID NO: 141. In some embodiments, the spacer sequence is SEQ ID NO: 142. In some embodiments, the spacer sequence is SEQ ID NO: 143. In some embodiments, the spacer sequence is SEQ ID NO: 144. In some embodiments, the spacer sequence is SEQ ID NO: 145. In some embodiments, the spacer sequence is SEQ ID NO: 146. In some embodiments, the spacer sequence is SEQ ID NO: 147. In some embodiments, the spacer sequence is SEQ ID NO: 148. In some embodiments, the spacer sequence is SEQ ID NO: 149. In some embodiments, the spacer sequence is SEQ ID NO: 150. In some embodiments, the spacer sequence is SEQ ID NO: 151. In some embodiments, the spacer sequence is SEQ ID NO: 152. In some embodiments, the spacer sequence is SEQ ID NO: 153. In some embodiments, the spacer sequence is SEQ ID NO: 154. In some embodiments, the spacer sequence is SEQ ID NO: 155. In some embodiments, the spacer sequence is SEQ ID NO: 156. In some embodiments, the spacer sequence is SEQ ID NO: 157. In some embodiments, the spacer sequence is SEQ ID NO: 158. In some embodiments, the spacer sequence is SEQ ID NO: 159. In some embodiments, the spacer sequence is SEQ ID NO: 160. In some embodiments, the spacer sequence is SEQ ID NO: 161. In some embodiments, the spacer sequence is SEQ ID NO: 161. In some embodiments, the spacer sequence is SEQ ID NO: 162. In some embodiments, the spacer sequence is SEQ ID NO: 163. In some embodiments, the spacer sequence is SEQ ID NO: 164. In some embodiments, the spacer sequence is SEQ ID NO: 165. In some embodiments, the spacer sequence is SEQ ID NO: 166. In some embodiments, the spacer sequence is SEQ ID NO: 167. In some embodiments, the spacer sequence is SEQ ID NO: 28. In some embodiments, the composition further comprises a DNA-PK inhibitor. In some embodiments, the DNA-PK inhibitor is Compound 1. In some embodiments, the DNA-PK inhibitor is Compound 2. In some embodiments, the DNA-PK inhibitor is Compound 6.

In some embodiments, a composition is provided comprising at least one guide RNA, or nucleic acid encoding at least one guide RNA, wherein at least one of the guide RNA comprises a spacer sequence selected from any one of SEQ ID NOs: 201-531.

In some embodiments, a composition is provided comprising a guide RNA, or nucleic acid encoding a guide RNA, wherein the guide RNA further comprises a trRNA. In each composition and method embodiment described herein, the crRNA (comprising the spacer sequence) and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond. In some embodiments, the composition further comprises a DNA-PK inhibitor.

In one aspect, a composition is provided comprising a single nucleic acid molecule encoding 1) one or more guide RNA that comprises a guide sequence selected from any one of SEQ ID NOs: 1-65, 67-167, and 201-531; and 2) a SluCas9. In some embodiments, the composition further comprises a DNA-PK inhibitor.

In one aspect, a composition is provided comprising a single nucleic acid molecule encoding 1) one or more guide RNA that comprises a guide sequence that is at least 99%, 98%, 97%, 96%, 95%, 94%, 93%, 92%, 91%, or 90% identical to any one of SEQ ID NOs: 1-65, 67-167, and 201-531; and 2) a SluCas9. In some embodiments, the composition further comprises a DNA-PK inhibitor.

In another aspect, a composition is provided comprising a single nucleic acid molecule encoding 1) one or more guide RNA that comprises a guide sequence comprising at least 17, 18, 19, 20, or 21 contiguous nucleotides of a spacer sequence selected from any one of SEQ ID NOs: 1-65, 67-167, and 201-531; and 2) a SluCas9. In some embodiments, the composition further comprises a DNA-PK inhibitor.

In one aspect, a composition is provided comprising a single nucleic acid molecule encoding 1) one or more guide RNA that comprises a guide sequence selected from any one of SEQ ID NOs: 1-172, and 201-531; and 2) a SluCas9. In some embodiments, the composition further comprises a DNA-PK inhibitor.

In one aspect, a composition is provided comprising a single nucleic acid molecule encoding 1) one or more guide RNA that comprises a guide sequence that is at least 99%, 98%, 97%, 96%, 95%, 94%, 93%, 92%, 91%, or 90% identical to any one of SEQ ID NOs: 1-172, and 201-531; and 2) a SluCas9. In some embodiments, the composition further comprises a DNA-PK inhibitor.

In another aspect, a composition is provided comprising a single nucleic acid molecule encoding 1) one or more guide RNA that comprises a guide sequence comprising at least 17, 18, 19, 20, or 21 contiguous nucleotides of a spacer sequence selected from any one of SEQ ID NOs: 1-172, and 201-531; and 2) a SluCas9. In some embodiments, the composition further comprises a DNA-PK inhibitor.

In one aspect, a composition is provided comprising a single nucleic acid molecule encoding 1) a pair of guide RNAs that comprise a first spacer sequence selected from any one of SEQ ID NOs: 1-65 and a second spacer sequence selected from any one of SEQ ID NOs: 67-167; or a pair of guide RNAs that comprise a first and second spacer sequence that is at least 99%, 98%, 97%, 96%, 95%, 94%, 93%, 92%, 91%, or 90% identical to any one of 1); or a pair of guide RNAs that comprise a first and second spacer sequence comprising at least 17, 18, 19, 20, or 21 contiguous nucleotides of a spacer sequence selected from any one of 1); and 2) a SluCas9. In some embodiments, the composition further comprises a DNA-PK inhibitor.

In any embodiment comprising a nucleic acid molecule encoding a guide RNA and/or a Cas9, the nucleic acid molecule may be a vector. In some embodiments, a composition is provided comprising a single nucleic acid molecule encoding a guide RNA and Cas9, wherein the nucleic acid molecule is a vector.

Any type of vector, such as any of those described herein, may be used. In some embodiments, the vector is a viral vector. In some embodiments, the viral vector is a non-integrating viral vector (i.e., that does not insert sequence from the vector into a host chromosome). In some embodiments, the viral vector is an adeno-associated virus vector (AAV), a lentiviral vector, an integrase-deficient lentiviral vector, an adenoviral vector, a vaccinia viral vector, an alphaviral vector, or a herpes simplex viral vector. In some embodiments, the vector comprises a muscle-specific promoter. Exemplary muscle-specific promoters include a muscle creatine kinase promoter, a desmin promoter, an MHCK7 promoter, or an SPc5-12 promoter. See US 2004/0175727 A1; Wang et al., Expert Opin Drug Deliv. (2014) 11, 345-364; Wang et al., Gene Therapy (2008) 15, 1489-1499. In some embodiments, the muscle-specific promoter is a CK8 promoter. In some embodiments, the muscle-specific promoter is a CK8e promoter. In any of the foregoing embodiments, the vector may be an adeno-associated virus vector (AAV). In some embodiments, the vector is an AAV9 vector.

In some embodiments, the muscle specific promoter is the CK8 promoter. The CK8 promoter has the following sequence (SEQ ID NO. 700):

| | |
|---|---|
| 1 | CTAGACTAGC ATGCTGCCCA TGTAAGGAGG CAAGGCCTGG GGACACCCGA GATGCCTGGT |
| 61 | TATAATTAAC CCAGACATGT GGCTGCCCCC CCCCCCCCAA CACCTGCTGC CTCTAAAAAT |
| 121 | AACCCTGCAT GCCATGTTCC CGGCGAAGGG CCAGCTGTCC CCCGCCAGCT AGACTCAGCA |
| 181 | CTTAGTTTAG GAACCAGTGA GCAAGTCAGC CCTTGGGGCA GCCCATACAA GGCCATGGGG |
| 241 | CTGGGCAAGC TGCACGCCTG GGTCCGGGGT GGGCACGGTG CCCGGGCAAC GAGCTGAAAG |
| 301 | CTCATCTGCT CTCAGGGGCC CCTCCCTGGG GACAGCCCCT CCTGGCTAGT CACACCCTGT |
| 361 | AGGCTCCTCT ATATAACCCA GGGGCACAGG GGCTGCCCTC ATTCTACCAC CACCTCCACA |
| 421 | GCACAGACAG ACACTCAGGA GCCAGCCAGC |

In some embodiments, the muscle-cell cell specific promoter is a variant of the CK8 promoter, called CK8e. The CK8e promoter has the following sequence (SEQ ID NO. 701):

| | |
|---|---|
| 1 | TGCCCATGTA AGGAGGCAAG GCCTGGGGAC ACCCGAGATG CCTGGTTATA ATTAACCCAG |
| 61 | ACATGTGGCT GCCCCCCCCC CCCCAACACC TGCTGCCTCT AAAAATAACC CTGCATGCCA |
| 121 | TGTTCCCGGC GAAGGGCCAG CTGTCCCCCG CCAGCTAGAC TCAGCACTTA GTTTAGGAAC |
| 181 | CAGTGAGCAA GTCAGCCCTT GGGGCAGCCC ATACAAGGCC ATGGGGCTGG GCAAGCTGCA |
| 241 | CGCCTGGGTC CGGGGTGGGC ACGGTGCCCG GGCAACGAGC TGAAAGCTCA TCTGCTCTCA |
| 301 | GGGGCCCCTC CCTGGGGACA GCCCCTCCTG GCTAGTCACA CCCTGTAGGC TCCTCTATAT |
| 361 | AACCCAGGGG CACAGGGGCT GCCCTCATTC TACCACCACC TCCACAGCAC AGACAGACAC |
| 421 | TCAGGAGCCA GCCAGC |

In some embodiments, the vector comprises one or more of a U6, H1, or 7SK promoter. In some embodiments, the U6 promoter is the human U6 promoter (e.g., the U6L promoter or U6S promoter). In some embodiments, the promoter is the murine U6 promoter. In some embodiments, the 7SK promoter is a human 7SK promoter. In some embodiments, the 7SK promoter is the 7SK1 promoter. In some embodiments, the 7SK promoter is the 7SK2 promoter. In some embodiments, the H1 promoter is a human H1 promoter (e.g., the H1L promoter or the HIS promoter). In some embodiments, the vector comprises multiple guide sequences, wherein each guide sequence is under the control of a separate promoter. In some embodiments, each of the multiple guide sequences comprises a different sequence. In some embodiments, each of the multiple guide sequences comprise the same sequence (e.g., each of the multiple guide sequences comprise the same spacer sequence). In some embodiments, each of the multiple guide sequences comprises the same spacer sequence and the same scaffold sequence. In some embodiments, each of the multiple guide sequences comprises different spacer sequences and different scaffold sequences. In some embodiments, each of the multiple guide sequences comprises the same spacer sequence, but comprises a different scaffold sequence. In some embodiments, each of the multiple guide sequences comprises different spacer sequences and different scaffold sequences. In some embodiments, each of the separate promoters comprises the same nucleotide sequence (e.g., the U6 promoter sequence). In some embodiments, each of the separate promoters comprises a different nucleotide sequence (e.g., the U6, H1, and/or 7SK promoter sequence).

In some embodiments, the U6 promoter comprises a nucleotide sequence that is at least 90%, 91%, 92%, 93%, 94%, 95%, 96%, 97%, 98%, 99% or 100% identical to the sequence of SEQ ID NO: 702:

cgagtccaac acccgtggga atcccatggg caccatggcc cctcgctcca aaaatgcttt 60
cgcgtcgcgc agacactgct cggtagtttc ggggatcagc gtttgagtaa gagcccgcgt 120
ctgaaccctc cgcgccgccc cggccccagt ggaaagacgc gcaggcaaaa cgcaccacgt 180
gacggagcgt gaccgcgcgc cgagcgcgcg ccaaggtcgg gcaggaagag ggcctatttc 240
ccatgattcc ttcatatttg catatacgat acaaggctgt tagagagata attagaatta 300
atttgactgt aaacacaaag atattagtac aaaatacgtg acgtagaaag taataatttc 360
ttgggtagtt tgcagtttta aaattatgtt ttaaaatgga ctatcatatg cttaccgtaa 420
cttgaaagta tttcgatttc ttggctttat atatcttgtg gaaaggacga aa 472

In some embodiments, the H1 promoter comprises a nucleotide sequence that is at least 90%, 91%, 92%, 93%, 94%, 95%, 96%, 97%, 98%, 99% or 100% identical to the sequence of SEQ ID NO: 703:

gctcggcgcg cccatatttg catgtcgcta tgtgttctgg gaaatcacca taaacgtgaa 60
atgtctttgg atttgggaat cttataagtt ctgtatgaga ccacggta 108

In some embodiments, the 7SK promoter comprises a nucleotide sequence that is at least 90%, 91%, 92%, 93%, 94%, 95%, 96%, 97%, 98%, 99% or 100% identical to the sequence of SEQ ID NO: 704:

tgacggcgcg ccctgcagta tttagcatgc cccacccatc tgcaaggcat tctggatagt 60
gtcaaaacag ccggaaatca agtccgttta tctcaaactt tagcattttg ggaataaatg 120
atatttgcta tgctggttaa attagatttt agttaaattt cctgctgaag ctctagtacg 180
ataagtaact tgacctaagt gtaaagttga gatttccttc aggtttatat agcttgtgcg 240
ccgcctgggt a 251

In some embodiments, the U6 promoter is a hU6c promoter and comprises a nucleotide sequence that is at least 90%, 91%, 92%, 93%, 94%, 95%, 96%, 97%, 98%, 99% or 100% identical to the sequence of SEQ ID NO: 705:

GAGGGCCTATTTCCCATGATTCCTTCATATTTGCATATACGATAC
AAGGCTGTTAGAGAGATAATTGGAATTAATTTGACTGTAAACACA
AAGATATTAGTACAAAATACGTGACGTAGAAAGTAATAATTTCTT
GGGTAGTTTGCAGTTTTAAAATTATGTTTTAAAATGGACTATCAT
ATGCTTACCGTAACTTGAAAGTATTTCGATTTCTTGGCTTTATAT
ATCTTGTGGAAAGGACGAAACACC.

In some embodiments, the 7SK promoter is a 7SK2 promoter and comprises a nucleotide sequence that is at least 90%, 91%, 92%, 93%, 94%, 95%, 96%, 97%, 98%, 99% or 100% identical to the sequence of SEQ ID NO: 706:

CTGCAGTATTTAGCATGCCCCACCCATCTGCAAGGCATTCTGGAT
AGTGTCAAAACAGCCGGAAATCAAGTCCGTTTATCTCAAACTTTA
GCATTTTGGGAATAAATGATATTTGCTATGCTGGTTAAATTAGAT
TTTAGTTAAATTTCCTGCTGAAGCTCTAGTACGATAAGCAACTTG
ACCTAAGTGTAAAGTTGAGACTTCCTTCAGGTTTATATAGCTTGT
GCGCCGCTTGGGTACCTC.

In some embodiments, the H1 promoter is a H1m or mH1 promoter and comprises a nucleotide sequence that is at least 90%, 91%, 92%, 93%, 94%, 95%, 96%, 97%, 98%, 99% or 100% identical to the sequence of SEQ ID NO: 707:

AATATTTGCATGTCGCTATGTGTTCTGGGAAATCACCATAAACGT
GAAATGTCTTTGGATTTGGGAATCTTATAAGTTCTGTATGAGACC
ACTCTTTCCC.

In some embodiments, the Ck8e promoter comprises a nucleotide sequence that is at least 90%, 91%, 92%, 93%, 94%, 95%, 96%, 97%, 98%, 99% or 100% identical to the sequence of SEQ ID NO: 701

TGCCCATGTAAGGAGGCAAGGCCTGGGGACACCCGAGATGCCTGG
TTATAATTAACCCAGACATGTGGCTGCCCCCCCCCCCCCAACACC
TGCTGCCTCTAAAAATAACCCTGCATGCCATGTTCCCGGCGAAGG
GCCAGCTGTCCCCCGCCAGCTAGACTCAGCACTTAGTTTAGGAAC
CAGTGAGCAAGTCAGCCCTTGGGGCAGCCCATACAAGGCCATGGG
GCTGGGCAAGCTGCACGCCTGGGTCCGGGGTGGGCACGGTGCCCG
GGCAACGAGCTGAAAGCTCATCTGCTCTCAGGGGCCCCTCCCTGG
GGACAGCCCCTCCTGGCTAGTCACACCCTGTAGGCTCCTCTATAT
AACCCAGGGGCACAGGGGCTGCCCTCATTCTACCACCACCTCCAC
AGCACAGACAGACACTCAGGAGCCAGCCAGC.

In some embodiments, the vector comprises multiple inverted terminal repeats (ITRs). These ITRs may be of an AAV1, AAV2, AAV3, AAV4, AAV5, AAV6, AAV7, AAV8, or AAV9 serotype. In some embodiments, the ITRs are of an AAV2 serotype. In some embodiments, the 5′ ITR comprises the sequence of SEQ ID NO: 709:

GGCCACTCCCTCTCTGCGCGCTCGCTCGCTCACTGAGGCCGGGCG
ACCAAAGGTCGCCCGACGCCCGGGCTTTGCCCGGGCGGCCTCAGT
GAGCGAGCGAGCGCGCAGAGAGGGAGTGGCCAACTCCATCACTAG
GGGTTCCT.

In some embodiments, the 3′ITR comprises the sequence of SEQ ID NO: 710:

AGGAACCCCTAGTGATGGAGTTGGCCACTCCCTCTCTGCGCGCTC
GCTCGCTCACTGAGGCCGGGCGACCAAAGGTCGCCCGACGCCCGG
GCTTTGCCCGGGCGGCCTCAGTGAGCGAGCGAGCGCGCAGAGAGG
GA.

In some embodiments, a vector comprising a single nucleic acid molecule encoding 1) one or more guide RNA comprising any one or more of the spacer sequences of SEQ ID NOs: 1-65, 67-167, and 201-531; and 2) a SluCas9 is provided. In some embodiments, the vector is an AAV vector. In some embodiments, the vector is an AAV9 vector. In some embodiments, the AAV vector is administered to a subject to treat DM1. In some embodiments, only one vector is needed due to the use of a particular guide sequence that is useful in the context of SluCas9. In some embodiments, the composition further comprises a DNA-PK inhibitor. In some embodiments, a composition or system comprising more than one vector is provided wherein the first vector comprises a single nucleic acid molecule encoding 1) one or more guide RNA comprising any one or more of the spacer sequences of SEQ ID NOs: 1-65, 67-167, and 201-531; and 2) a SluCas9, and a second vector comprises a nucleic acid encoding multiple copies of a guide RNA (e.g., any one or more of the spacer sequences of SEQ ID NOs: 1-65, 67-167, and 201-531). In some embodiments, a composition or system comprising a first vector and a second vector is provided wherein the first vector comprises a single nucleic acid molecule encoding a SluCas9 and not any guide RNAs, and a second vector comprises a nucleic acid encoding multiple copies of a guide RNA (e.g., any one or more of the spacer sequences of SEQ ID NOs: 1-65, 67-167, and 201-531). In such composition or system encoding multiple guide RNAs, the guide RNAs can be the same or different.

In some embodiments, a vector comprising a single nucleic acid molecule encoding 1) a pair of guide RNAs that comprise a first spacer sequence selected from any one of SEQ ID NOs: 1-65 and a second spacer sequence selected from any one of SEQ ID NOs: 67-167; and 2) a SluCas9 is provided. In some embodiments, the vector is an AAV vector. In some embodiments, the AAV vector is administered to a subject to treat DM1. In some embodiments, only one vector is needed due to the use of a particular guide sequence that is useful in the context of SluCas9. In some embodiments, the composition further comprises a DNA-PK inhibitor.

In some embodiments, the vector comprises a nucleic acid encoding a Cas9 protein (e.g., a SluCas9 protein) and further comprises a nucleic acid encoding one or more single guide RNA(s). In some embodiments, the nucleic acid encoding the Cas9 protein is under the control of a CK8e promoter. In some embodiments, the nucleic acid encoding the guide RNA sequence is under the control of a hU6c promoter. In some embodiments, the vector is AAV9. In preferred embodiments, the AAV9 vector is less than 5 kb from ITR to ITR in size, inclusive of both ITRs. In particular embodiments, the AAV9 vector is less than 4.9 kb from ITR to ITR in size, inclusive of both ITRs. In further embodiments, the AAV9 vector is less than 4.85 kb from ITR to ITR in size, inclusive of both ITRs. In further embodiments, the AAV9 vector is less than 4.8 kb from ITR to ITR in size, inclusive of both ITRs. In further embodiments, the AAV9 vector is less than 4.75 kb from ITR to ITR in size, inclusive of both ITRs. In further embodiments, the AAV9 vector is less than 4.7 kb from ITR to ITR in size, inclusive of both ITRs. In some embodiments, the AAV9 vector is between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb from ITR to ITR in size, inclusive of both ITRs. In some embodiments, the AAV9 vector is between 4.4-4.85 kb from ITR to ITR in size, inclusive of both ITRs.

In some embodiments, the vector comprises multiple nucleic acids encoding more than one guide RNA. In some embodiments, the vector comprises two nucleic acids encoding two guide RNA sequences.

In some embodiments, the vector comprises a nucleic acid encoding a Cas9 protein (e.g., a SluCas9 protein), a nucleic acid encoding a first guide RNA, and a nucleic acid encoding a second guide RNA. In some embodiments, the vector does not comprise a nucleic acid encoding more than two guide RNAs. In some embodiments, the nucleic acid encoding the first guide RNA is the same as the nucleic acid encoding the second guide RNA. In some embodiments, the nucleic acid encoding the first guide RNA is different from the nucleic acid encoding the second guide RNA. In some embodiments, the vector comprises a single nucleic acid molecule, wherein the single nucleic acid molecule comprises a nucleic acid encoding a Cas9 protein, a nucleic acid encoding a first guide RNA, and a nucleic acid that is the reverse complement to the coding sequence for the second guide RNA. In some embodiments, the vector comprises a single nucleic acid molecule, wherein the single nucleic acid molecule comprises a nucleic acid encoding a Cas9 protein, a nucleic acid that is the reverse complement to the coding sequence for the first guide RNA, and a nucleic acid that is the reverse complement to the coding sequence for the second guide RNA. In some embodiments, the nucleic acid encoding a Cas9 protein (e.g., a SluCas9 protein) is under the control of the CK8e promoter. In some embodiments, the first guide is under the control of the 7SK2 promoter, and the second guide is under the control of the H1m promoter. In some embodiments, the first guide is under the control of the H1m promoter, and the second guide is under the control of the 7SK2 promoter. In some embodiments, the first guide is under the control of the hU6c promoter, and the second guide is under the control of the H1m promoter. In some embodiments, the first guide is under the control of the H1m promoter, and the second guide is under the control of the hU6c promoter. In some embodiments, the nucleic acid encoding the Cas9 protein is: a) between the nucleic acids encoding the guide RNAs, b) between the nucleic acids that are the reverse complement to the coding sequences for the guide RNAs, c) between the nucleic acid encoding the first guide RNA and the nucleic acid that is the reverse complement to the coding sequence for the second guide RNA, d) between the nucleic acid encoding the second guide RNA and the nucleic acid that is the reverse complement to the coding sequence for the first guide RNA, e) 5′ to the nucleic acids encoding the guide RNAs, f) 5′ to the nucleic acids that are the reverse complements to the coding sequences for the guide RNAs, g) 5′ to a nucleic acid encoding one of the guide RNAs and 5′ to a nucleic acid that is the reverse complement to the coding sequence for the other guide RNA, h) 3′ to the nucleic acids encoding the guide RNAs, i) 3′ to the nucleic acids that are the reverse complements to the coding sequences for the guide RNAs, or j) 3′ to a nucleic acid encoding one of the guide RNAs and 3′ to a nucleic acid that is the reverse complement to the coding sequence for the other guide RNA. In some embodiments, the AAV vector size is measured in length of nucleotides from ITR to ITR, inclusive of both ITRs. In some embodiments, the AAV vector is less than 5 kb in size from ITR to ITR, inclusive of both ITRs. In particular embodiments, the AAV vector is less than 4.9 kb from ITR to ITR in size, inclusive of both ITRs. In further embodiments, the AAV vector is less than 4.85 kb in size from ITR to ITR, inclusive of both ITRs. In further embodiments, the AAV vector is less than 4.8 kb in size from ITR to ITR, inclusive of both ITRs. In further embodiments, the AAV vector is less than 4.75 kb in size from ITR to ITR, inclusive of both ITRs. In further embodiments, the AAV vector is less than 4.7 kb in size from ITR to ITR, inclusive of both ITRs. In some embodiments, the vector is between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb from ITR to ITR in size, inclusive of both ITRs. In some embodiments, the vector is between 4.4-4.85 kb in size from ITR to ITR, inclusive of both ITRs. In some embodiments, the vector is AAV9.

In some embodiments, the disclosure provides for a nucleic acid comprising from 5′ to 3′ with respect to the plus strand: the reverse complement of a first guide RNA scaffold sequence (a scaffold comprising the nucleotide sequence of SEQ ID NO: 901), the reverse complement of a nucleotide sequence encoding the first guide RNA sequence, the reverse complement of a promoter for expression of the nucleotide sequence encoding the first guide RNA sequence (e.g., hU6c), a promoter for expression of the second guide RNA in the same direction as the promoter for the endonuclease (e.g., 7SK2), the second guide RNA sequence, and a second guide RNA scaffold sequence (a scaffold comprising the nucleotide sequence of SEQ ID NO: 901), a promoter for expression of a nucleotide sequence encoding the endonuclease (e.g., CK8e), a nucleotide sequence encoding an endonuclease (e.g., any of the SluCas9 proteins disclosed herein), a polyadenylation sequence.

The disclosure provides for novel AAV vector configurations. Some examples of these novel AAV vector configurations are provided herein, and the order of elements in these exemplary vectors are referenced in a 5′ to 3′ manner with respect to the plus strand. For these configurations, it should be understood that the recited elements may not be directly contiguous, and that one or more nucleotides or one or more additional elements may be present between the recited elements. However, in some embodiments, it is possible that no nucleotides or no additional elements are present between the recited elements. Also, unless otherwise stated, “a promoter for expression of element X” means that the promoter is oriented in a manner to facilitate expression of the recited element X. In some embodiments, the disclosure provides for a nucleic acid encoding an SluCas9.

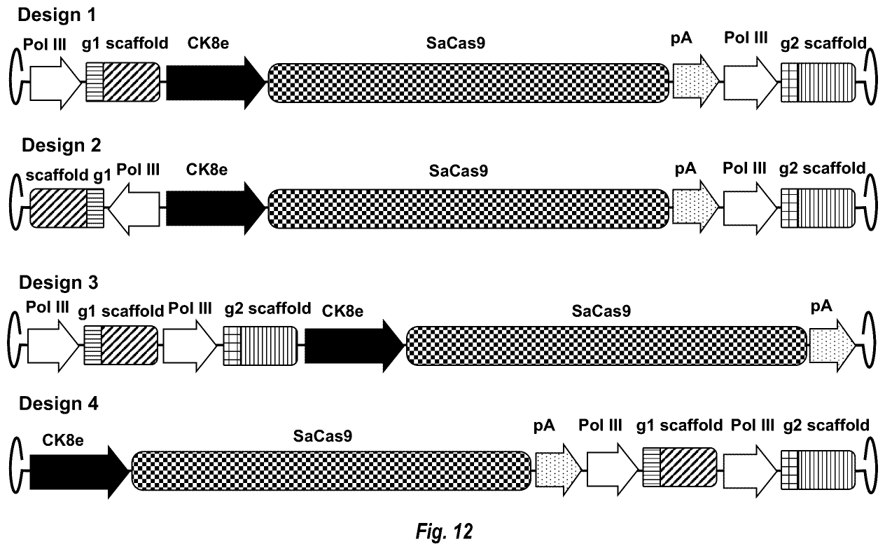

In some embodiments, the AAV vector comprises from 5′ to 3′ with respect to the plus strand: a promoter for expression of a nucleic acid encoding a first sgRNA, a nucleic acid encoding the first sgRNA guide sequence, the first sgRNA scaffold sequence, a promoter for expression of SluCas9 (e.g., CK8e), a nucleic acid encoding SluCas9, a polyadenylation sequence, a promoter for expression of a second sgRNA, the second sgRNA guide sequence, and a second sgRNA scaffold sequence. See FIG. 12 at “Design 1” below. In some embodiments the promoter for expression of the nucleic acid encoding the first sgRNA is any of the hU6c promoters disclosed herein. In some embodiments the promoter for expression of the nucleic acid encoding the first sgRNA comprises SEQ ID NO: 705. In some embodiments the promoter for expression of the nucleic acid encoding the first sgRNA is any of the 7SK2 promoters disclosed herein. In some embodiments the promoter for expression of the nucleic acid encoding the first sgRNA comprises SEQ ID NO: 706. In some embodiments the promoter for expression of the nucleic acid encoding the second sgRNA is any of the hU6c promoters disclosed herein. In some embodiments the promoter for expression of the nucleic acid encoding the second sgRNA comprises SEQ ID NO: 705. In some embodiments the promoter for expression of the nucleic acid encoding the second sgRNA is any of the 7SK2 promoters disclosed herein. In some embodiments the promoter for expression of the nucleic acid encoding the second sgRNA comprises SEQ ID NO: 706. In some embodiments, the sgRNA scaffold is SEQ ID NO: 900. In some embodiments, the sgRNA scaffold is SEQ ID NO: 901. In some embodiments, the first sgRNA targets a nucleic acid region upstream of a trinucleotide repeat expansion, and the second sgRNA targets a nucleic acid region downstream of a trinucleotide repeat expansion. In some embodiments, the first sgRNA targets a nucleic acid region downstream of a trinucleotide repeat expansion, and the second sgRNA targets a nucleic acid region upstream of a trinucleotide repeat expansion.

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, the first sgRNA scaffold sequence, a promoter for expression of a nucleic acid encoding SluCas9 (e.g., CK8e), a nucleic acid encoding SluCas9, a polyadenylation sequence, an hU6c promoter for expression of a second sgRNA, a second sgRNA guide sequence, and a second sgRNA scaffold sequence.

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence, a promoter for expression of a nucleic acid encoding SluCas9 (e.g., CK8e), a nucleic acid encoding SluCas9, a polyadenylation sequence, a 7SK2 promoter for expression of a second sgRNA, the second sgRNA guide sequence, and a second sgRNA scaffold sequence.

In some embodiments, the AAV vector comprises from 5′ to 3′ with respect to the plus strand: an hU6c promoter for expression of the nucleic acid encoding a first sgRNA, a nucleic acid encoding the first sgRNA guide sequence, a first sgRNA scaffold sequence, a promoter for expression of a nucleic acid encoding SluCas9 (e.g., CK8e), a nucleic acid encoding SluCas9, a polyadenylation sequence, an Him promoter for expression of a second sgRNA, a second sgRNA guide sequence, and a second sgRNA scaffold sequence.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of a promoter for expression of the nucleic acid encoding the first sgRNA, a promoter for expression of a nucleic acid encoding SluCas9 (e.g., CK8e), a nucleic acid encoding SluCas9, a polyadenylation sequence, a promoter for expression of a second sgRNA, a second sgRNA guide sequence, and a second sgRNA scaffold sequence. See FIG. 12 at “Design 2”. In some embodiments the promoter for expression of the nucleic acid encoding the first sgRNA is any of the hU6c promoters disclosed herein. In some embodiments the promoter for expression of the nucleic acid encoding the first sgRNA comprises SEQ ID NO: 705. In some embodiments the promoter for expression of the nucleic acid encoding the first sgRNA is any of the 7SK2 promoters disclosed herein. In some embodiments the promoter for expression of the nucleic acid encoding the first sgRNA comprises SEQ ID NO: 706. In some embodiments the promoter for expression of the nucleic acid encoding the second sgRNA is any of the hU6c promoters disclosed herein. In some embodiments the promoter for expression of the nucleic acid encoding the second sgRNA comprises SEQ ID NO: 705. In some embodiments the promoter for expression of the nucleic acid encoding the second sgRNA is any of the 7SK2 promoters disclosed herein. In some embodiments the promoter for expression of the nucleic acid encoding the second sgRNA comprises SEQ ID NO: 706. In some embodiments, the sgRNA scaffold is SEQ ID NO: 900. In some embodiments, the sgRNA scaffold is SEQ ID NO: 901. In some embodiments, the first sgRNA targets a nucleic acid region upstream of a trinucleotide repeat expansion, and the second sgRNA targets a nucleic acid region downstream of a trinucleotide repeat expansion. In some embodiments, the first sgRNA targets a nucleic acid region downstream of a trinucleotide repeat expansion, and the second sgRNA targets a nucleic acid region upstream of a trinucleotide repeat expansion.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding SluCas9 (e.g., CK8e), a nucleic acid encoding SluCas9, a polyadenylation sequence, an hU6c promoter for expression of a second sgRNA, a second sgRNA guide sequence, and a second sgRNA scaffold sequence.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an 7SK2 promoter for expression of the nucleic acid encoding the first sgRNA, a promoter for expression of a nucleic acid encoding SluCas9 (e.g., CK8e), a nucleic acid encoding SluCas9, a polyadenylation sequence, an hU6c promoter for expression of a second sgRNA, a second sgRNA guide sequence, and a second sgRNA scaffold sequence.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding SluCas9 (e.g., CK8e), a nucleic acid encoding SluCas9, a polyadenylation sequence, an 7SK2 promoter for expression of a second sgRNA, a second sgRNA guide sequence, and a second sgRNA scaffold sequence.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding SluCas9 (e.g., CK8e), a nucleic acid encoding SluCas9, a polyadenylation sequence, an H1m promoter for expression of a second sgRNA, a second sgRNA guide sequence, and a second sgRNA scaffold sequence.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of a 7SK2 promoter for expression of the nucleic acid encoding the first sgRNA, a promoter for expression of a nucleic acid encoding SluCas9 (e.g., CK8e), a nucleic acid encoding SluCas9, a polyadenylation sequence, an hU6 promoter for expression of a second sgRNA, a second sgRNA guide sequence, and a second sgRNA scaffold sequence.

In some embodiments, the AAV vector comprises from 5′ to 3′ with respect to the plus strand: a promoter for expression of a nucleic acid encoding a first sgRNA, a nucleic acid encoding the first sgRNA guide sequence, a first sgRNA scaffold sequence, a promoter for expression of a second sgRNA, a second sgRNA guide sequence, and a second sgRNA scaffold sequence, a promoter for SluCas9 (e.g., CK8e), a nucleic acid encoding SluCas9, and a polyadenylation sequence. See FIG. 12 at “Design 3”. In some embodiments the promoter for expression of the nucleic acid encoding the first sgRNA is any of the hU6c promoters disclosed herein. In some embodiments the promoter for expression of the nucleic acid encoding the first sgRNA comprises SEQ ID NO: 705. In some embodiments the promoter for expression of the nucleic acid encoding the first sgRNA is any of the 7SK2 promoters disclosed herein. In some embodiments the promoter for expression of the nucleic acid encoding the first sgRNA comprises SEQ ID NO: 706. In some embodiments the promoter for expression of the nucleic acid encoding the second sgRNA is any of the hU6c promoters disclosed herein. In some embodiments the promoter for expression of the nucleic acid encoding the second sgRNA comprises SEQ ID NO: 705. In some embodiments the promoter for expression of the nucleic acid encoding the second sgRNA is any of the 7SK2 promoters disclosed herein. In some embodiments the promoter for expression of the nucleic acid encoding the second sgRNA comprises SEQ ID NO: 706. In some embodiments, the sgRNA scaffold is SEQ ID NO: 900. In some embodiments, the sgRNA scaffold is SEQ ID NO: 901. In some embodiments, the first sgRNA targets a nucleic acid region upstream of a trinucleotide repeat expansion, and the second sgRNA targets a nucleic acid region downstream of a trinucleotide repeat expansion. In some embodiments, the first sgRNA targets a nucleic acid region downstream of a trinucleotide repeat expansion, and the second sgRNA targets a nucleic acid region upstream of a trinucleotide repeat expansion.

In some embodiments, the AAV vector comprises from 5′ to 3′ with respect to the plus strand: an hU6c promoter for expression of a nucleic acid encoding a first sgRNAa nucleic acid encoding a first sgRNA guide sequence, a first sgRNA scaffold sequence, an hU6c promoter for expression of a second sgRNA, a second sgRNA guide sequence, and a second sgRNA scaffold sequence, a promoter for expression of a nucleic acid encoding SluCas9 (e.g., CK8e), a nucleic acid encoding SluCas9, and a polyadenylation sequence.

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence, an 7SK2 promoter for expression of a second sgRNA, a second sgRNA guide sequence, and a second sgRNA scaffold sequence, a promoter for expression of a nucleic acid encoding SluCas9 (e.g., CK8e), a nucleic acid encoding SluCas9, and a polyadenylation sequence.

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence, an 7SK2 promoter for expression of a second sgRNA, a second sgRNA guide sequence, and a second sgRNA scaffold sequence, a promoter for expression of a nucleic acid encoding SluCas9 (e.g., CK8e), a nucleic acid encoding SluCas9, and a polyadenylation sequence.

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence, an 7SK2 promoter for expression of a second sgRNA, a second sgRNA guide sequence, and a second sgRNA scaffold sequence, a promoter for expression of a nucleic acid encoding SluCas9 (e.g., CK8e), a nucleic acid encoding SluCas9, and a polyadenylation sequence.

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 901, an 7SK2 promoter for expression of a second sgRNA, a second sgRNA guide sequence, and a second sgRNA scaffold sequence comprising SEQ ID NO: 901, a promoter for expression of a nucleic acid encoding SluCas9 (e.g., CK8e), a nucleic acid encoding SluCas9, and a polyadenylation sequence.

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 901, an 7SK2 promoter for expression of a second sgRNA, a second sgRNA guide sequence, and a second sgRNA scaffold sequence comprising SEQ ID NO: 901, a promoter for expression of a nucleic acid encoding SluCas9 (e.g., CK8e), an SV40 nuclear localization sequence (NLS), a nucleic acid encoding SluCas9, and a polyadenylation sequence.

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 901, an 7SK2 promoter for expression of a second sgRNA, a second sgRNA guide sequence, and a second sgRNA scaffold sequence comprising SEQ ID NO: 901, a promoter for expression of a nucleic acid encoding SluCas9 (e.g., CK8e), a nucleic acid encoding SluCas9, an SV40 nuclear localization sequence (NLS), and a polyadenylation sequence.

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence, an H1m promoter for expression of a second sgRNA, a second sgRNA guide sequence, and a second sgRNA scaffold sequence, a promoter for expression of a nucleic acid encoding SluCas9 (e.g., CK8e), a nucleic acid encoding SluCas9, and a polyadenylation sequence.

In some embodiments, the AAV vector comprises from 5′ to 3′ with respect to the plus strand: an hU6c promoter for expression of a nucleic acid encoding a first sgRNA, a nucleic acid encoding the first sgRNA guide sequence, a first sgRNA scaffold sequence comprising SEQ ID NO: 901, an 7SK2 promoter for expression of a second sgRNA, a second sgRNA guide sequence, and a second sgRNA scaffold sequence comprising SEQ ID NO: 901, a promoter for expression of a nucleic acid encoding SluCas9 (e.g., CK8e), a nucleic acid encoding SluCas9, and a polyadenylation sequence.

In some embodiments, the AAV vector comprises from 5′ to 3′ with respect to the plus strand: a promoter for expression of a nucleic acid encoding SluCas9 (e.g., CK8e), a nucleic acid encoding SluCas9, a polyadenylation sequence, a promoter for expression of the nucleic acid encoding a first guide RNA, a nucleic acid encoding the first sgRNA guide sequence, a first sgRNA scaffold sequence, a promoter for expression of the second sgRNA, a second sgRNA guide sequence, and a second sgRNA scaffold sequence. See FIG. 12 at “Design 4”. In some embodiments the promoter for expression of the nucleic acid encoding the first sgRNA is any of the hU6c promoters disclosed herein. In some embodiments the promoter for expression of the nucleic acid encoding the first sgRNA comprises SEQ ID NO: 705. In some embodiments the promoter for expression of the nucleic acid encoding the first sgRNA is any of the 7SK2 promoters disclosed herein. In some embodiments the promoter for expression of the nucleic acid encoding the first sgRNA comprises SEQ ID NO: 706. In some embodiments the promoter for expression of the nucleic acid encoding the second sgRNA is any of the hU6c promoters disclosed herein. In some embodiments the promoter for expression of the nucleic acid encoding the second sgRNA comprises SEQ ID NO: 705. In some embodiments the promoter for expression of the nucleic acid encoding the second sgRNA is any of the 7SK2 promoters disclosed herein. In some embodiments the promoter for expression of the nucleic acid encoding the second sgRNA comprises SEQ ID NO: 706. In some embodiments, the sgRNA scaffold is SEQ ID NO: 900. In some embodiments, the sgRNA scaffold is SEQ ID NO: 901. In some embodiments, the first sgRNA targets a nucleic acid region upstream of a trinucleotide repeat expansion, and the second sgRNA targets a nucleic acid region downstream of a trinucleotide repeat expansion. In some embodiments, the first sgRNA targets a nucleic acid region downstream of a trinucleotide repeat expansion, and the second sgRNA targets a nucleic acid region upstream of a trinucleotide repeat expansion.

In some embodiments, the AAV vector comprises from 5′ to 3′ with respect to the plus strand: a promoter for expression of a nucleic acid encoding SluCas9 (e.g., CK8e), a nucleic acid encoding SluCas9, a polyadenylation sequence, an hU6c promoter for expression of a nucleic acid encoding a first guide RNA, a nucleic acid encoding the first sgRNA guide sequence, a first sgRNA scaffold sequence, an hU6c promoter for expression of a second sgRNA, a second sgRNA guide sequence, and a second sgRNA scaffold sequence.

In some embodiments, the AAV vector comprises from 5′ to 3′ with respect to the plus strand: a promoter for expression of a nucleic acid encoding SluCas9 (e.g., CK8e), a nucleic acid encoding SluCas9, a polyadenylation sequence, an hU6c promoter for expression of a nucleic acid encoding a first guide RNA, a nucleic acid encoding the first sgRNA guide sequence, a first sgRNA scaffold sequence, a 7SK2 promoter for expression of a second sgRNA, a second sgRNA guide sequence, and a second sgRNA scaffold sequence.

In some embodiments, the AAV vector comprises from 5′ to 3′ with respect to the plus strand: a promoter for expression of a nucleic acid encoding SluCas9 (e.g., CK8e), a nucleic acid encoding SluCas9, a polyadenylation sequence, an hU6c promoter for expression of a nucleic acid encoding a first guide RNA, a nucleic acid encoding a first sgRNA guide sequence, a first sgRNA scaffold sequence, a H1m promoter for expression of a second sgRNA, a second sgRNA guide sequence, and a second sgRNA scaffold sequence.

In some embodiments, the AAV vector comprises any of the configurations outlined in Table 6.

| Promoter arrangement | Guide promoter combinations |
|---|---|
| Cas9 in Middle | hU6c-guide1-Cas9-hU6c-guide2 |
| (in line) | hU6c-guide1-Cas9-7SK2-guide2 |
| (“Design 1” geometries | hU6c-guide1-Cas9-H1m-guide2 |
| from FIG. 12) | |
| Cas9 in Middle | hU6c-guide1-Cas9-hU6c-guide2 |
| (divergent) | hU6c-guide1-Cas9-7SK2-guide2 |
| (“Design 2” geometries | hU6c-guide1-Cas9-H1m-guide2 |
| from FIG. 12) | 7SK2-guide1-Cas9-hU6c-guide2 |
| Cas9 on Right | hU6c-guide1-hU6c-guide2-Cas9 |
| (in line) | hU6c-guide1-7SK2-guide2-Cas9 |
| (“Design 3” geometries | hU6c-guide1-H1m-guide2-Cas9 |
| from FIG. 12) | hU6c-guide1(v5)-7SK2-guide2(v5)-Cas9 |
| | hU6c-guide1(v2)-7SK2-guide2(v2)-Cas9 |
| Cas9 on Left | Cas9-hU6c-guide1-hU6c-guide2 |
| (in line) | Cas9-hU6c-guide1-7SK2-guide2 |
| (“Design 4” geometries | Cas9-hU6c-guide1-H1m-guide2 |
| from FIG. 12) | Cas9-hU6m-guide1-hU6c-guide2 |
| | Cas9-hU6m-guide1-7SK2-guide2 |
| | Cas9-hU6m-guide1-H1m-guide2 |
| | Cas9-7SK2-guide1-hU6c-guide2 |

In particular embodiments, the AAV vector comprises from 5′ to 3′ with respect to the plus strand: an hU6c promoter for expression of the nucleic acid encoding a first sgRNA, a nucleic acid encoding the first sgRNA guide sequence, a first sgRNA scaffold sequence, the hU6c promoter for expression of a second sgRNA, a second sgRNA guide sequence, and a second sgRNA scaffold sequence, a promoter for expression of a nucleic acid encoding SluCas9 (e.g., CK8e), a nucleic acid encoding SluCas9, and a polyadenylation sequence.

In particular embodiments, the AAV vector comprises from 5′ to 3′ with respect to the plus strand: an hU6c promoter for expression of the nucleic acid encoding a first sgRNA, a nucleic acid encoding the first sgRNA guide sequence, a first sgRNA scaffold sequence, a 7SK2 promoter for expression of a second sgRNA, a second sgRNA guide sequence, and a second sgRNA scaffold sequence, a promoter for expression of a nucleic acid encoding SluCas9 (e.g., CK8e), a nucleic acid encoding SluCas9, and a polyadenylation sequence.

In particular embodiments, the AAV vector comprises from 5′ to 3′ with respect to the plus strand: a promoter for expression of a nucleic acid encoding SluCas9 (e.g., CK8e), a nucleic acid encoding SluCas9, a polyadenylation sequence, an hU6c promoter for expression of a nucleic acid encoding a first guide RNA, a nucleic acid encoding a first sgRNA guide sequence, a first sgRNA scaffold sequence, a hU6c promoter for expression of a second sgRNA, a second sgRNA guide sequence, and a second sgRNA scaffold sequence.

In particular embodiments, the AAV vector comprises from 5′ to 3′ with respect to the plus strand: a promoter for expression of a nucleic acid encoding SluCas9 (e.g., CK8e), a nucleic acid encoding SluCas9, a polyadenylation sequence, an hU6c promoter for expression of a nucleic acid encoding a first guide RNA, a nucleic acid encoding a first sgRNA guide sequence, a first sgRNA scaffold sequence, a 7SK2 promoter for expression of a second sgRNA, a second sgRNA guide sequence, and a second sgRNA scaffold sequence.

In some embodiments, any of the vectors disclosed herein comprises a nucleic acid encoding at least a first guide RNA and a second guide RNA. In some embodiments, the nucleic acid comprises a spacer-encoding sequence for the first guide RNA, a scaffold-encoding sequence for the first guide RNA, a spacer-encoding sequence for the second guide RNA, and a scaffold-encoding sequence of the second guide RNA. In some embodiments, the spacer-encoding sequence (e.g., encoding any of the spacer sequences disclosed herein) for the first guide RNA is identical to the spacer-encoding sequence for the second guide RNA. In some embodiments, the spacer-encoding sequence (e.g., encoding any of the spacer sequences disclosed herein) for the first guide RNA is different from the spacer-encoding sequence for the second guide RNA. In some embodiments, the scaffold-encoding sequence for the first guide RNA is identical to the scaffold-encoding sequence for the nucleic acid encoding the second guide RNA. In some embodiments, the scaffold-encoding sequence for the first guide RNA is different from the scaffold-encoding sequence for the second guide RNA. In some embodiments, the scaffold-encoding sequence for the first guide RNA comprises a sequence selected from the group consisting of SEQ ID Nos: 901-916, and the scaffold-encoding sequence for the second guide RNA comprises a different sequence selected from the group consisting of SEQ ID Nos: 901-916. In some embodiments, the scaffold-encoding sequence for the first guide RNA comprises the sequence of SEQ ID NO: 901, and the scaffold-encoding sequence for the second guide RNA comprises the sequence of SEQ ID NO: 901. In some embodiments, the scaffold-encoding sequence for the first guide RNA comprises the sequence of SEQ ID NO: 901, and the scaffold-encoding sequence for the second guide RNA comprises the sequence of SEQ ID NO: 902. In some embodiments, the scaffold-encoding sequence for the first guide RNA comprises the sequence of SEQ ID NO: 901, and the scaffold-encoding sequence for the second guide RNA comprises the sequence of SEQ ID NO: 903. In some embodiments, the scaffold-encoding sequence for the first guide RNA comprises the sequence of SEQ ID NO: 901, and the scaffold-encoding sequence for the second guide RNA comprises the sequence of SEQ ID NO: 904. In some embodiments, the scaffold-encoding sequence for the first guide RNA comprises the sequence of SEQ ID NO: 901, and the scaffold-encoding sequence for the second guide RNA comprises the sequence of SEQ ID NO: 905. In some embodiments, the scaffold-encoding sequence for the first guide RNA comprises the sequence of SEQ ID NO: 901, and the scaffold-encoding sequence for the second guide RNA comprises the sequence of SEQ ID NO: 906. In some embodiments, the scaffold-encoding sequence for the first guide RNA comprises the sequence of SEQ ID NO: 901, and the scaffold-encoding sequence for the second guide RNA comprises the sequence of SEQ ID NO: 907. In some embodiments, the scaffold-encoding sequence for the first guide RNA comprises the sequence of SEQ ID NO: 901, and the scaffold-encoding sequence for the second guide RNA comprises the sequence of SEQ ID NO: 908. In some embodiments, the scaffold-encoding sequence for the first guide RNA comprises the sequence of SEQ ID NO: 901, and the scaffold-encoding sequence for the second guide RNA comprises the sequence of SEQ ID NO: 909. In some embodiments, the scaffold-encoding sequence for the first guide RNA comprises the sequence of SEQ ID NO: 901, and the scaffold-encoding sequence for the second guide RNA comprises the sequence of SEQ ID NO: 910. In some embodiments, the scaffold-encoding sequence for the first guide RNA comprises the sequence of SEQ ID NO: 901, and the scaffold-encoding sequence for the second guide RNA comprises the sequence of SEQ ID NO: 911. In some embodiments, the scaffold-encoding sequence for the first guide RNA comprises the sequence of SEQ ID NO: 901, and the scaffold-encoding sequence for the second guide RNA comprises the sequence of SEQ ID NO: 912. In some embodiments, the scaffold-encoding sequence for the first guide RNA comprises the sequence of SEQ ID NO: 901, and the scaffold-encoding sequence for the second guide RNA comprises the sequence of SEQ ID NO: 913. In some embodiments, the scaffold-encoding sequence for the first guide RNA comprises the sequence of SEQ ID NO: 901, and the scaffold-encoding sequence for the second guide RNA comprises the sequence of SEQ ID NO: 914. In some embodiments, the scaffold-encoding sequence for the first guide RNA comprises the sequence of SEQ ID NO: 901, and the scaffold-encoding sequence for the second guide RNA comprises the sequence of SEQ ID NO: 915. In some embodiments, the scaffold-encoding sequence for the first guide RNA comprises the sequence of SEQ ID NO: 901, and the scaffold-encoding sequence for the second guide RNA comprises the sequence of SEQ ID NO: 916. In some embodiments, the spacer encoding sequence for the first guide RNA is the same as the spacer-encoding sequence in the second guide RNA, and the scaffold-encoding sequence for the first guide RNA is different from the scaffold-encoding sequence in the nucleic acid encoding the second guide RNA.

In some embodiments, the nucleic acid encoding SluCas9 encodes a SluCas9 comprising an amino acid sequence that is at least 80%, 85%, 90%, 91%, 92%, 93%, 94%, 95%, 96%, 97%, 98%, 99% or 100% identical to the sequence of SEQ ID NO: 712:

NQKFILGLDIGITSVGYGLIDYETKNIIDAGVRLFPEANVENNEG
RRSKRGSRRLKRRRIHRLERVKKLLEDYNLLDQSQIPQSTNPYAI
RVKGLSEALSKDELVIALLHIAKRRGIHKIDVIDSNDDVGNELST
KEQLNKNSKLLKDKFVCQIQLERMNEGQVRGEKNRFKTADIIKEI
IQLLNVQKNFHQLDENFINKYIELVEMRREYFEGPGKGSPYGWEG
DPKAWYETLMGHCTYFPDELRSVKYAYSADLFNALNDLNNLVIQR
DGLSKLEYHEKYHIIENVFKQKKKPTLKQIANEINVNPEDIKGYR
ITKSGKPQFTEFKLYHDLKSVLFDQSILENEDVLDQIAEILTIYQ
DKDSIKSKLTELDILLNEEDKENIAQLTGYTGTHRLSLKCIRLVL
EEQWYSSRNQMEIFTHLNIKPKKINLTAANKIPKAMIDEFILSPV
VKRTFGQAINLINKIIEKYGVPEDIIIELARENNSKDKQKFINEM
QKKNENTRKRINEIIGKYGNQNAKRLVEKIRLHDEQEGKCLYSLE
SIPLEDLLNNPNHYEVDHIIPRSVSFDNSYHNKVLVKQSENSKKS
NLTPYQYFNSGKSKLSYNQFKQHILNLSKSQDRISKKKKEYLLEE
RDINKFEVQKEFINRNLVDTRYATRELTNYLKAYFSANNMNVKVK
TINGSFTDYLRKVWKFKKERNHGYKHHAEDALIIANADFLFKENK
KLKAVNSVLEKPEIETKQLDIQVDSEDNYSEMFIIPKQVQDIKDF
RNFKYSHRVDKKPNRQLINDTLYSTRKKDNSTYIVQTIKDIYAKD
NTTLKKQFDKSPEKFLMYQHDPRTFEKLEVIMKQYANEKNPLAKY
HEETGEYLTKYSKKNNGPIVKSLKYIGNKLGSHLDVTHQFKSSTK
KLVKLSIKPYRFDVYLTDKGYKFITISYLDVLKKDNYYYIPEQKY
DKLKLGKAIDKNAKFIASFYKNDLIKLDGEIYKIIGVNSDTRNMI
ELDLPDIRYKEYCELNNIKGEPRIKKTIGKKVNSIEKLTTDVLGN
VFTNTQYTKPQLLFKRGN.

In some embodiments, the SluCas9 is a variant of the amino acid sequence of SEQ ID NO: 712. A variant of SluCas9 comprises one or more amino acid changes as compared to SEQ ID NO: 712, including insertion, deletion, or substitution of one or more amino acids, or a chemical modification to one or more amino acids. In some embodiments, the SluCas9 comprises an amino acid other than an Q at the position corresponding to position 781 of SEQ ID NO: 712. In some embodiments, the SluCas9 comprises an amino acid other than an R at the position corresponding to position 1013 of SEQ ID NO: 712. In some embodiments, the SluCas9 comprises a K at the position corresponding to position 781 of SEQ ID NO: 712. In some embodiments, the SluCas9 comprises a K at the position corresponding to position 966 of SEQ ID NO: 712. In some embodiments, the SluCas9 comprises an H at the position corresponding to position 1013 of SEQ ID NO: 712. In some embodiments, the SluCas9 comprises an amino acid other than an Q at the position corresponding to position 781 of SEQ ID NO: 712; and an amino acid other than an R at the position corresponding to position 1013 of SEQ ID NO: 712. In some embodiments, the SluCas9 comprises a K at the position corresponding to position 781 of SEQ ID NO: 712; a K at the position corresponding to position 966 of SEQ ID NO: 712; and an H at the position corresponding to position 1013 of SEQ ID NO: 712.

In some embodiments, the SluCas9 comprises an amino acid other than an R at the position corresponding to position 246 of SEQ ID NO: 712. In some embodiments, the SluCas9 comprises an amino acid other than an N at the position corresponding to position 414 of SEQ ID NO: 712. In some embodiments, the SluCas9 comprises an amino acid other than a T at the position corresponding to position 420 of SEQ ID NO: 712. In some embodiments, the SluCas9 comprises an amino acid other than an R at the position corresponding to position 655 of SEQ ID NO: 712. In some embodiments, the SluCas9 comprises an amino acid other than an R at the position corresponding to position 246 of SEQ ID NO: 712; an amino acid other than an N at the position corresponding to position 414 of SEQ ID NO: 712; an amino acid other than a T at the position corresponding to position 420 of SEQ ID NO: 712; and an amino acid other than an R at the position corresponding to position 655 of SEQ ID NO: 712. In some embodiments, the SluCas9 comprises an A at the position corresponding to position 246 of SEQ ID NO: 712. In some embodiments, the SluCas9 comprises an A at the position corresponding to position 414 of SEQ ID NO: 712. In some embodiments, the SluCas9 comprises an A at the position corresponding to position 420 of SEQ ID NO: 712. In some embodiments, the SluCas9 comprises an A at the position corresponding to position 655 of SEQ ID NO: 712. In some embodiments, the SluCas9 comprises an A at the position corresponding to position 246 of SEQ ID NO: 712; an A at the position corresponding to position 414 of SEQ ID NO: 712; an A at the position corresponding to position 420 of SEQ ID NO: 712; and an A at the position corresponding to position 655 of SEQ ID NO: 712.

In some embodiments, the SluCas9 comprises an amino acid other than an R at the position corresponding to position 246 of SEQ ID NO: 712; an amino acid other than an N at the position corresponding to position 414 of SEQ ID NO: 712; an amino acid other than a T at the position corresponding to position 420 of SEQ ID NO: 712; an amino acid other than an R at the position corresponding to position 655 of SEQ ID NO: 712; an amino acid other than an Q at the position corresponding to position 781 of SEQ ID NO: 712; a K at the position corresponding to position 966 of SEQ ID NO: 712; and an amino acid other than an R at the position corresponding to position 1013 of SEQ ID NO: 712. In some embodiments, the SluCas9 comprises an A at the position corresponding to position 246 of SEQ ID NO: 712; an A at the position corresponding to position 414 of SEQ ID NO: 712; an A at the position corresponding to position 420 of SEQ ID NO: 712; an A at the position corresponding to position 655 of SEQ ID NO: 712; a K at the position corresponding to position 781 of SEQ ID NO: 712; a K at the position corresponding to position 966 of SEQ ID NO: 712; and an H at the position corresponding to position 1013 of SEQ ID NO: 712.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 718 (designated herein as SluCas9-KH or SLUCAS9KH):

NQKFILGLDIGITSVGYGLIDYETKNIIDAGVRLFPEANVENNEG
RRSKRGSRRLKRRRIHRLERVKKLLEDYNLLDQSQIPQSTNPYAI
RVKGLSEALSKDELVIALLHIAKRRGIHKIDVIDSNDDVGNELST
KEQLNKNSKLLKDKFVCQIQLERMNEGQVRGEKNRFKTADIIKEI
IQLLNVQKNFHQLDENFINKYIELVEMRREYFEGPGKGSPYGWEG
DPKAWYETLMGHCTYFPDELRSVKYAYSADLFNALNDLNNLVIQR
DGLSKLEYHEKYHIIENVFKQKKKPTLKQIANEINVNPEDIKGYR
ITKSGKPQFTEFKLYHDLKSVLFDQSILENEDVLDQIAEILTIYQ
DKDSIKSKLTELDILLNEEDKENIAQLTGYTGTHRLSLKCIRLVL
EEQWYSSRNQMEIFTHLNIKPKKINLTAANKIPKAMIDEFILSPV
VKRTFGQAINLINKIIEKYGVPEDIIIELARENNSKDKQKFINEM
QKKNENTRKRINEIIGKYGNQNAKRLVEKIRLHDEQEGKCLYSLE
SIPLEDLLNNPNHYEVDHIIPRSVSFDNSYHNKVLVKQSENSKKS
NLTPYQYFNSGKSKLSYNQFKQHILNLSKSQDRISKKKKEYLLEE
RDINKFEVQKEFINRNLVDTRYATRELTNYLKAYFSANNMNVKVK
TINGSFTDYLRKVWKFKKERNHGYKHHAEDALIIANADFLFKENK
KLKAVNSVLEKPEIETKQLDIQVDSEDNYSEMFIIPKQVQDIKDF
RNFKYSHRVDKKPNRKLINDTLYSTRKKDNSTYIVQTIKDIYAKD
NTTLKKQFDKSPEKFLMYQHDPRTFEKLEVIMKQYANEKNPLAKY
HEETGEYLTKYSKKNNGPIVKSLKYIGNKLGSHLDVTHQFKSSTK
KLVKLSIKPYRFDVYLTDKGYKFITISYLDVLKKDNYYYIPEQKY
DKLKLGKAIDKNAKFIASFYKNDLIKLDGEIYKIIGVNSDTRNMI
ELDLPDIRYKEYCELNNIKGEPHIKKTIGKKVNSIEKLTTDVLGN
VFTNTQYTKPQLLFKRGN.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 719 (designated herein as SluCas9-HF):

NQKFILGLDIGITSVGYGLIDYETKNIIDAGVRLFPEANVENNEG
RRSKRGSRRLKRRRIHRLERVKKLLEDYNLLDQSQIPQSTNPYAI
RVKGLSEALSKDELVIALLHIAKRRGIHKIDVIDSNDDVGNELST
KEQLNKNSKLLKDKFVCQIQLERMNEGQVRGEKNRFKTADIIKEI
IQLLNVQKNFHQLDENFINKYIELVEMRREYFEGPGKGSPYGWEG
DPKAWYETLMGHCTYFPDELASVKYAYSADLFNALNDLNNLVIQR
DGLSKLEYHEKYHIIENVFKQKKKPTLKQIANEINVNPEDIKGYR
ITKSGKPQFTEFKLYHDLKSVLFDQSILENEDVLDQIAEILTIYQ
DKDSIKSKLTELDILLNEEDKENIAQLTGYTGTHRLSLKCIRLVL
EEQWYSSRAQMEIFAHLNIKPKKINLTAANKIPKAMIDEFILSPV
VKRTFGQAINLINKIIEKYGVPEDIIIELARENNSKDKQKFINEM
QKKNENTRKRINEIIGKYGNQNAKRLVEKIRLHDEQEGKCLYSLE
SIPLEDLLNNPNHYEVDHIIPRSVSFDNSYHNKVLVKQSENSKKS
NLTPYQYFNSGKSKLSYNQFKQHILNLSKSQDRISKKKKEYLLEE
RDINKFEVQKEFINRNLVDTRYATAELTNYLKAYFSANNMNVKVK
TINGSFTDYLRKVWKFKKERNHGYKHHAEDALIIANADFLFKENK
KLKAVNSVLEKPEIETKQLDIQVDSEDNYSEMFIIPKQVQDIKDF
RNFKYSHRVDKKPNRQLINDTLYSTRKKDNSTYIVQTIKDIYAKD
NTTLKKQFDKSPEKFLMYQHDPRTFEKLEVIMKQYANEKNPLAKY
HEETGEYLTKYSKKNNGPIVKSLKYIGNKLGSHLDVTHQFKSSTK
KLVKLSIKPYRFDVYLTDKGYKFITISYLDVLKKDNYYYIPEQKY
DKLKLGKAIDKNAKFIASFYKNDLIKLDGEIYKIIGVNSDTRNMI
ELDLPDIRYKEYCELNNIKGEPRIKKTIGKKVNSIEKLTTDVLGN
VFTNTQYTKPQLLFKRGN.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 720 (designated herein as SluCas9-HF-KH):

NQKFILGLDIGITSVGYGLIDYETKNIIDAGVRLFPEANVENNEG
RRSKRGSRRLKRRRIHRLERVKKLLEDYNLLDQSQIPQSTNPYAI
RVKGLSEALSKDELVIALLHIAKRRGIHKIDVIDSNDDVGNELST
KEQLNKNSKLLKDKFVCQIQLERMNEGQVRGEKNRFKTADIIKEI
IQLLNVQKNFHQLDENFINKYIELVEMRREYFEGPGKGSPYGWEG
DPKAWYETLMGHCTYFPDELASVKYAYSADLFNALNDLNNLVIQR
DGLSKLEYHEKYHIIENVFKQKKKPTLKQIANEINVNPEDIKGYR
ITKSGKPQFTEFKLYHDLKSVLFDQSILENEDVLDQIAEILTIYQ
DKDSIKSKLTELDILLNEEDKENIAQLTGYTGTHRLSLKCIRLVL
EEQWYSSRAQMEIFAHLNIKPKKINLTAANKIPKAMIDEFILSPV
VKRTFGQAINLINKIIEKYGVPEDIIIELARENNSKDKQKFINEM
QKKNENTRKRINEIIGKYGNQNAKRLVEKIRLHDEQEGKCLYSLE
SIPLEDLLNNPNHYEVDHIIPRSVSFDNSYHNKVLVKQSENSKKS
NLTPYQYFNSGKSKLSYNQFKQHILNLSKSQDRISKKKKEYLLEE
RDINKFEVQKEFINRNLVDTRYATAELTNYLKAYFSANNMNVKVK
TINGSFTDYLRKVWKFKKERNHGYKHHAEDALIIANADFLFKENK
KLKAVNSVLEKPEIETKQLDIQVDSEDNYSEMFIIPKQVQDIKDF
RNFKYSHRVDKKPNRKLINDTLYSTRKKDNSTYIVQTIKDIYAKD
NTTLKKQFDKSPEKFLMYQHDPRTFEKLEVIMKQYANEKNPLAKY
HEETGEYLTKYSKKNNGPIVKSLKYIGNKLGSHLDVTHQFKSSTK
KLVKLSIKPYRFDVYLTDKGYKFITISYLDVLKKDNYYYIPEQKY
DKLKLGKAIDKNAKFIASFYKNDLIKLDGEIYKIIGVNSDTRNMI
ELDLPDIRYKEYCELNNIKGEPHIKKTIGKKVNSIEKLTTDVLGN
VFTNTQYTKPQLLFKRGN.

In some embodiments, the Cas protein is any of the engineered Cas proteins disclosed in Schmidt et al., 2021, Nature Communications, “Improved CRISPR genome editing using small highly active and specific engineered RNA-guided nucleases.”

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 716 (designated herein as sRGN1):

MNQKFILGLDIGITSVGYGLIDYETKNIIDAGVRLFPEANVENNE
GRRSKRGSRRLKRRRIHRLDRVKHLLAEYDLLDLTNIPKSTNPYQ
TRVKGLNEKLSKDELVIALLHIAKRRGIHNVDVAADKEETASDSL
STKDQINKNAKFLESRYVCELQKERLENEGHVRGVENRFLTKDIV
REAKKIIDTQMQYYPEIDETFKEKYISLVETRREYFEGPGKGSPF
GWEGNIKKWFEQMMGHCTYFPEELRSVKYSYSAELFNALNDLNNL
VITRDEDAKLNYGEKFQIIENVFKQKKTPNLKQIAIEIGVHETEI
KGYRVNKSGTPEFTEFKLYHDLKSIVFDKSILENEAILDQIAEIL
TIYQDEQSIKEELNKLPEILNEQDKAEIAKLIGYNGTHRLSLKCI
HLINEELWQTSRNQMEIFNYLNIKPNKVDLSEQNKIPKDMVNDFI
LSPVVKRTFIQSINVINKVIEKYGIPEDIIIELARENNSDDRKKF
INNLQKKNEATRKRINEIIGQTGNQNAKRIVEKIRLHDQQEGKCL
YSLKDIPLEDLLRNPNNYDIDHIIPRSVSFDDSMHNKVLVRREQN
AKKNNQTPYQYLTSGYADIKYSVFKQHVLNLAENKDRMTKKKREY
LLEERDINKFEVQKEFINRNLVDTRYATRELTNYLKAYFSANNMN
VKVKTINGSFTDYLRKVWKFKKERNHGYKHHAEDALIIANADFLF
KENKKLKAVNSVLEKPEIETKQLDIQVDSEDNYSEMFIIPKQVQD
IKDFRNFKYSHRVDKKPNRQLINDTLYSTRKKDNSTYIVQTIKDI
YAKDNTTLKKQFDKSPEKFLMYQHDPRTFEKLEVIMKQYANEKNP
LAKYHEETGEYLTKYSKKNNGPIVKSLKYIGNKLGSHLDVTHQFK
SSTKKLVKLSIKPYRFDVYLTDKGYKFITISYLDVLKKDNYYYIP
EQKYDKLKLGKAIDKNAKFIASFYKNDLIKLDGEIYKIIGVNSDT
RNMIELDLPDIRYKEYCELNNIKGEPRIKKTIGKKVNSIEKLTTD
VLGNVFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 717 (designated herein as sRGN2):

MNQKFILGLDIGITSVGYGLIDYETKNIIDAGVRLFPEANVENNE
GRRSKRGSRRLKRRRIHRLERVKSLLSEYKIISGLAPTNNQPYNI
RVKGLTEQLTKDELAVALLHIAKRRGIHKIDVIDSNDDVGNELST
KEQLNKNSKLLKDKFVCQIQLERMNEGQVRGEKNRFKTADIIKEI
IQLLNVQKNFHQLDENFINKYIELVEMRREYFEGPGQGSPFGWNG
DLKKWYEMLMGHCTYFPQELRSVKYAYSADLFNALNDLNNLIIQR
DNSEKLEYHEKYHIIENVFKQKKKPTLKQIAKEIGVNPEDIKGYR
ITKSGTPEFTEFKLYHDLKSVLFDQSILENEDVLDQIAEILTIYQ
DKDSIKSKLTELDILLNEEDKENIAQLTGYNGTHRLSLKCIRLVL
EEQWYSSRNQMEIFTHLNIKPKKINLTAANKIPKAMIDEFILSPV
VKRTFIQSINVINKVIEKYGIPEDIIIELARENNSDDRKKFINNL
QKKNEATRKRINEIIGQTGNQNAKRIVEKIRLHDQQEGKCLYSLE
SIALMDLLNNPQNYEVDHIIPRSVAFDNSIHNKVLVKQIENSKKG
NRTPYQYLNSSDAKLSYNQFKQHILNLSKSKDRISKKKKDYLLEE
RDINKFEVQKEFINRNLVDTRYATRELTSYLKAYFSANNMDVKVK
TINGSFTNHLRKVWRFDKYRNHGYKHHAEDALIIANADFLFKENK
KLKAVNSVLEKPEIETKQLDIQVDSEDNYSEMFIIPKQVQDIKDF
RNFKYSHRVDKKPNRQLINDTLYSTRKKDNSTYIVQTIKDIYAKD
NTTLKKQFDKSPEKFLMYQHDPRTFEKLEVIMKQYANEKNPLAKY
HEETGEYLTKYSKKNNGPIVKSLKYIGNKLGSHLDVTHQFKSSTK
KLVKLSIKPYRFDVYLTDKGYKFITISYLDVLKKDNYYYIPEQKY
DKLKLGKAIDKNAKFIASFYKNDLIKLDGEIYKIIGVNSDTRNMI
ELDLPDIRYKEYCELNNIKGEPRIKKTIGKKVNSIEKLTTDVLGN
VFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 723 (designated herein as sRGN3):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKYSHRVDKKPNRQLINDTLYSTRKKDNSTYIVQTIKDIYAKDNTTLKKQF
DKSPEKFLMYQHDPRTFEKLEVIMKQYANEKNPLAKYHEETGEYLTKYSKKNNGPIVKSLK
YIGNKLGSHLDVTHQFKSSTKKLVKLSIKPYRFDVYLTDKGYKFITISYLDVLKKDNYYYIPE
QKYDKLKLGKAIDKNAKFIASFYKNDLIKLDGEIYKIIGVNSDTRNMIELDLPDIRYKEYCELN
NIKGEPRIKKTIGKKVNSIEKLTTDVLGNVFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 724 (designated herein as sRGN3.1):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKYSHRVDKKPNRQLINDTLYSTRKKDNSTYIVQTIKDIYAKDNTTLKKQF
DKSPEKFLMYQHDPRTFEKLEVIMKQYANEKNPLAKYHEETGEYLTKYSKKNNGPIVKSLK
YIGNKLGSHLDVTHQFKSSTKKLVKLSIKNYRFDVYLTEKGYKFVTIAYLNVFKKDNYYYIP
KDKYQELKEKKKIKDTDQFIASFYKNDLIKLNGDLYKIIGVNSDDRNIIELDYYDIKYKDYCEI
NNIKGEPRIKKTIGKKTESIEKFTTDVLGNLYLHSTEKAPQLIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 725 (designated herein as sRGN3.2):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKFSHRVDKKPNRQLINDTLYSTRMKDEHDYIVQTITDIYGKDNTNLKKQ
FNKNPEKFLMYQNDPKTFEKLSIIMKQYSDEKNPLAKYYEETGEYLTKYSKKNNGPIVKKIK
LLGNKVGNHLDVTNKYENSTKKLVKLSIKNYRFDVYLTEKGYKFVTIAYLNVFKKDNYYYI
PKDKYQELKEKKKIKDTDQFIASFYKNDLIKLNGDLYKIIGVNSDDRNIIELDYYDIKYKDYC
EINNIKGEPRIKKTIGKKTESIEKFTTDVLGNLYLHSTEKAPQLIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 721 (designated herein as sRGN3.3):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTSYLKAYFSANNMDVKVKTINGSFTNHLRKVWRFD
KYRNHGYKHHAEDALIIANADFLFKENKKLQNTNKILEKPTIENNTKKVTVEKEEDYNNVFE
TPKLVEDIKQYRDYKFSHRVDKKPNRQLINDTLYSTRMKDEHDYIVQTITDIYGKDNTNLKK
QFNKNPEKFLMYQNDPKTFEKLSIIMKQYSDEKNPLAKYYEETGEYLTKYSKKNNGPIVKKI
KLLGNKVGNHLDVTNKYENSTKKLVKLSIKNYRFDVYLTEKGYKFVTIAYLNVFKKDNYYY
IPKDKYQELKEKKKIKDTDQFIASFYKNDLIKLNGDLYKIIGVNSDDRNIIELDYYDIKYKDYC
EINNIKGEPRIKKTIGKKTESIEKFTTDVLGNLYLHSTEKAPQLIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 722 (designated herein as sRGN4):

MNQKFILGLDIGITSVGYGLIDYETKNIIDAGVRLFPEANVENNEGRRSKRGSRRLKRRRIHRL
ERVKKLLEDYNLLDQSQIPQSTNPYAIRVKGLSEALSKDELVIALLHIAKRRGIHNINVSSEDE
DASNELSTKEQINRNNKLLKDKYVCEVQLQRLKEGQIRGEKNRFKTTDILKEIDQLLKVQKD
YHNLDIDFINQYKEIVETRREYFEGPGKGSPYGWEGDPKAWYETLMGHCTYFPDELRSVKY
AYSADLFNALNDLNNLVIQRDGLSKLEYHEKYHIIENVFKQKKKPTLKQIANEINVNPEDIKG
YRITKSGKPEFTSFKLFHDLKKVVKDHAILDDIDLLNQIAEILTIYQDKDSIVAELGQLEYLMS
EADKQSISELTGYTGTHSLSLKCMNMIIDELWHSSMNQMEVFTYLNMRPKKYELKGYQRIPT
DMIDDAILSPVVKRTFIQSINVINKVIEKYGIPEDIIIELARENNSDDRKKFINNLQKKNEATRKR
INEIIGQTGNQNAKRIVEKIRLHDQQEGKCLYSLESIPLEDLLNNPNHYEVDHIIPRSVSFDNSY
HNKVLVKQSENSKKSNLTPYQYFNSGKSKLSYNQFKQHILNLSKSQDRISKKKKEYLLEERDI
NKFEVQKEFINRNLVDTRYATRELTNYLKAYFSANNMNVKVKTINGSFTDYLRKVWKFKKE
RNHGYKHHAEDALIIANADFLFKENKKLKAVNSVLEKPEIETKQLDIQVDSEDNYSEMFIIPK
QVQDIKDFRNFKYSHRVDKKPNRQLINDTLYSTRKKDNSTYIVQTIKDIYAKDNTTLKKQFD
KSPEKFLMYQHDPRTFEKLEVIMKQYANEKNPLAKYHEETGEYLTKYSKKNNGPIVKSLKYI
GNKLGSHLDVTHQFKSSTKKLVKLSIKPYRFDVYLTDKGYKFITISYLDVLKKDNYYYIPEQK
YDKLKLGKAIDKNAKFIASFYKNDLIKLDGEIYKIIGVNSDTRNMIELDLPDIRYKEYCELNNI
KGEPRIKKTIGKKVNSIEKLTTDVLGNVFTNTQYTKPQLLFKRGN.

Modified Guide RNAs

In some embodiments, the guide RNA is chemically modified. A guide RNA comprising one or more modified nucleosides or nucleotides is called a “modified” guide RNA or “chemically modified” guide RNA, to describe the presence of one or more non-naturally and/or naturally occurring components or configurations that are used instead of or in addition to the canonical A, G, C, and U residues. In some embodiments, a modified guide RNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified guide RNAs comprising nucleosides and nucleotides (collectively “residues”) that can have two, three, four, or more modifications. For example, a modified residue can have a modified sugar and a modified nucleobase, or a modified sugar and a modified phosphodiester. In some embodiments, every base of a guide RNA is modified, e.g., all bases have a modified phosphate group, such as a phosphorothioate group. In certain embodiments, all, or substantially all, of the phosphate groups of an guide RNA molecule are replaced with phosphorothioate groups. In some embodiments, modified guide RNAs comprise at least one modified residue at or near the 5′ end of the RNA. In some embodiments, modified guide RNAs comprise at least one modified residue at or near the 3′ end of the RNA.

In some embodiments, the guide RNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified guide RNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the guide RNAs described herein can contain one or more modified nucleosides or nucleotides, e.g., to introduce stability toward intracellular or serum-based nucleases. In some embodiments, the modified guide RNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH2CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising sgRNA, one or more residues at one or both ends of the sgRNA may be chemically modified, and/or internal nucleosides may be modified, and/or the entire sgRNA may be chemically modified. Certain embodiments comprise a 5′ end modification. Certain embodiments comprise a 3′ end modification.

Modifications of 2′-O-methyl are encompassed.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Modifications of 2′-fluoro (2′-F) are encompassed.

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

Abasic nucleotides refer to those which lack nitrogenous bases.

Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability and/or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide.

### Ribonucleoprotein Complex

In some embodiments, a composition is encompassed comprising: a) one or more guide RNAs comprising one or more guide sequences from Table 1A and Table 1B and b) SluCas9, or any of the variant Cas9 proteins disclosed herein. In some embodiments, the guide RNA together with a Cas9 is called a ribonucleoprotein complex (RNP).

In some embodiments, the disclosure provides for an RNP complex, wherein the guide RNA (e.g., any of the guide RNAs disclosed herein) binds to or is capable of binding to a target sequence in the DMPK gene, or a target sequence bound by any of the sequences disclosed in Table 1A and Table 1B, wherein the DMPK gene comprises a PAM recognition sequence position upstream of the target sequence, and wherein the RNP cuts at a position that is 3 nucleotides upstream (−3) of the PAM in the DMPK gene. In some embodiments, the RNP also cuts at a position that is 2 nucleotides upstream (−2), 4 nucleotides upstream (−4), 5 nucleotides upstream (−5), or 6 nucleotides upstream (−6) of the PAM in the DMPK gene. In some embodiments, the RNP cuts at a position that is 3 nucleotides upstream (−3) and 4 nucleotides upstream (−4) of the PAM in the DMPK gene.

In some embodiments, chimeric Cas9 (SluCas9) nucleases are used, where one domain or region of the protein is replaced by a portion of a different protein. In some embodiments, a Cas9 nuclease domain may be replaced with a domain from a different nuclease such as Fok1. In some embodiments, a Cas9 nuclease may be a modified nuclease.

In some embodiments, the Cas9 is modified to contain only one functional nuclease domain. For example, the agent protein may be modified such that one of the nuclease domains is mutated or fully or partially deleted to reduce its nucleic acid cleavage activity.

In some embodiments, a conserved amino acid within a Cas9 protein nuclease domain is substituted to reduce or alter nuclease activity. In some embodiments, a Cas9 nuclease may comprise an amino acid substitution in the RuvC or RuvC-like nuclease domain. Exemplary amino acid substitutions in the RuvC or RuvC-like nuclease domain include DOA (based on the S. pyogenes Cas9 protein). See, e.g., Zetsche et al. (2015) Cell October 22:163(3): 759-771. In some embodiments, the Cas9 nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB—A0Q7Q2 (CPF1 FRATN)). Further exemplary amino acid substitutions include D10A and N580A (based on the S. aureus Cas9 protein). See, e.g., Friedland et al., 2015, Genome Biol., 16:257.

In some embodiments, the Cas9 lacks cleavase activity. In some embodiments, the Cas9 comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the Cas9 lacking cleavase activity or the dCas DNA-binding polypeptide is a version of a Cas nuclease (e.g., a Cas9 nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the Cas9 comprises one or more heterologous functional domains (e.g., is or comprises a fusion polypeptide).

In some embodiments, the heterologous functional domain may facilitate transport of the Cas9 into the nucleus of a cell. For example, the heterologous functional domain may be a nuclear localization signal (NLS). In some embodiments, the Cas9 may be fused with 1-10 NLS(s). In some embodiments, the Cas9 may be fused with 1-5 NLS(s). In some embodiments, the Cas9 may be fused with one NLS. Where one NLS is used, the NLS may be attached at the N-terminus or the C-terminus of the Cas9 sequence, and may be directly attached. In some embodiments, where more than one NLS is used, one or more NLS may be attached at the N-terminus and/or one or more NLS may be attached at the C-terminus. In some embodiments, one or more NLSs are directly attached to the Cas9. In some embodiments, one or more NLSs are attached to the Cas9 by means of a linker. In some embodiments, the linker is between 3-25 amino acids in length. In some embodiments, the linker is between 3-6 amino acids in length. In some embodiments, the linker comprises glycine and serine. In some embodiments, the linker comprises the sequence of GSVD (SEQ ID NO: 940) or GSGS (SEQ ID NO: 941). It may also be inserted within the Cas9 sequence. In other embodiments, the Cas9 may be fused with more than one NLS. In some embodiments, the Cas9 may be fused with 2, 3, 4, or 5 NLSs. In some embodiments, the Cas9 may be fused with two NLSs. In certain circumstances, the two NLSs may be the same (e.g., two SV40 NLSs) or different. In some embodiments, the Cas9 protein is fused with an SV40 NLS. In some embodiments, the SV40 NLS comprises the amino acid sequence of SEQ ID NO: 713 (PKKKRKV). In some embodiments, the Cas9 protein (e.g., the SluCas9 protein) is fused to a nucleoplasmin NLS. In some embodiments, the nucleoplasmin NLS comprises the amino acid sequence of SEQ ID NO: 714 (KRPAATKKAGQAKKKK). In some embodiments, the Cas9 protein is fused with a c-Myc NLS. In some embodiments, the c-Myc NLS is SEQ ID NO: 942 (PAAKKKKLD) and/or is encoded by the nucleic acid sequence of SEQ ID NO: 943 (CCGGCAGCTAAGAAAAAGAAACTGGAT). In some embodiments, the Cas9 is fused to two SV40 NLS sequences linked at the carboxy terminus. In some embodiments, the Cas9 may be fused with two NLSs, one linked at the N-terminus and one at the C-terminus. In some embodiments, the Cas9 may be fused with 3 NLSs. In some embodiments, the Cas9 may be fused with no NLS. In some embodiments, the Cas9 protein is fused to an SV40 NLS and to a nucleoplasmin NLS. In some embodiments, the SV40 NLS is fused to the C-terminus of the Cas9, while the nucleoplasmin NLS is fused to the N-terminus of the Cas9 protein. In some embodiments, the SV40 NLS is fused to the N-terminus of the Cas9, while the nucleoplasmin NLS is fused to the C-terminus of the Cas9 protein. In some embodiments, a c-myc NLS is fused to the N-terminus of the Cas9 and an SV40 NLS and/or nucleoplasmin NLS is fused to the C-terminus of the Cas9. In some embodiments, a c-myc NLS is fused to the N-terminus of the Cas9 (e.g., by means of a linker such as GSVD (SEQ ID NO: 940)), an SV40 NLS is fused to the C-terminus of the Cas9 (e.g., by means of a linker such as GSGS (SEQ ID NO: 941)), and a nucleoplasmin NLS is fused to the C-terminus of the SV-40 NLS (e.g., by means of a linker such as GSGS (SEQ ID NO: 941)). In some embodiments, the SV40 NLS is fused to the Cas9 protein by means of a linker. In some embodiments, the nucleoplasmin NLS is fused to the Cas9 protein by means of a linker.

In some embodiments, the heterologous functional domain may be capable of modifying the intracellular half-life of the Cas9. In some embodiments, the half-life of the Cas9 may be increased. In some embodiments, the half-life of the Cas9 may be reduced. In some embodiments, the heterologous functional domain may be capable of increasing the stability of the Cas9. In some embodiments, the heterologous functional domain may be capable of reducing the stability of the Cas9. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the Cas9 may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub1 in S. cerevisiae), human leukocyte antigen F-associated (FAT10), autophagy-8 (ATG8) and −12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellowl), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl caterprotein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the Cas9 to a specific organelle, cell type, tissue, or organ. In some embodiments, the heterologous functional domain may target the Cas9 to muscle.

In further embodiments, the heterologous functional domain may be an effector domain. When the Cas9 is directed to its target sequence, e.g., when a Cas9 is directed to a target sequence by a guide RNA, the effector domain may modify or affect the target sequence. In some embodiments, the effector domain may be chosen from a nucleic acid binding domain or a nuclease domain (e.g., a non-Cas nuclease domain). In some embodiments, the heterologous functional domain is a nuclease, such as a FokI nuclease. See, e.g., U.S. Pat. No. 9,023,649.

### Determination of Efficacy of Guide RNAs

In some embodiments, the efficacy of a guide RNA is determined when delivered or expressed together with other components forming an RNP. In some embodiments, the guide RNA is expressed together with a SluCas9. In some embodiments, the guide RNA is delivered to or expressed in a cell line that already stably expresses a SluCas9. In some embodiments the guide RNA is delivered to a cell as part of an RNP. In some embodiments, the guide RNA is delivered to a cell along with a nucleic acid (e.g., mRNA) encoding SluCas9.

In some embodiments, the efficacy of particular guide RNAs is determined based on in vitro models. In some embodiments, the in vitro model is a cell line.

In some embodiments, the efficacy of particular guide RNAs is determined across multiple in vitro cell models for a guide RNA selection process. In some embodiments, a cell line comparison of data with selected guide RNAs is performed. In some embodiments, cross screening in multiple cell models is performed.

In some embodiments, the efficacy of particular guide RNAs is determined based on in vivo models. In some embodiments, the in vivo model is a rodent model. In some embodiments, the rodent model is a mouse which expresses a gene comprising an expanded trinucleotide repeat or a self-complementary region. The gene may be the human version or a rodent (e.g., murine) homolog of any of the genes listed in Table 1. In some embodiments, the gene is human DMPK. In some embodiments, the gene is a rodent (e.g., murine) homolog of DMPK. In some embodiments, the in vivo model is a non-human primate, for example cynomolgus monkey. See, e.g., the mouse model described in Huguet et al., 2012, PLoS Genet, 8(11):e1003043. In some embodiments, the in vivo model is a non-human primate, for example cynomolgus monkey.

### III. Methods of Gene Editing, CTG Repeat Excision, and Treating DM1

This disclosure provides methods and uses for treating Myotonic Dystrophy Type 1 (DM1). In some embodiments, any of the compositions or systems described herein may be administered to a subject in need thereof for use in making a double strand break in the DMPK gene. In some embodiments, any of the compositions or systems described herein may be administered to a subject in need thereof for use in excising a CTG repeat in the 3′ untranslated region (UTR) of the DMPK gene. In some embodiments, any of the compositions or systems described herein may be administered to a subject in need thereof for use in treating DM1. In some embodiments, a nucleic acid molecule comprising a first nucleic acid encoding one or more guide RNAs of Table 1A and Table 1B and a second nucleic acid encoding SluCas9 is administered to a subject to treat DM1. In some embodiments, a single nucleic acid molecule (which may be a vector, including an AAV vector) comprising a first nucleic acid encoding one or more guide RNAs of Table 1A and Table 1B and a second nucleic acid encoding SluCas9 is administered to a subject to treat DM1.

In some embodiments, any of the compositions described herein is administered to a subject in need thereof to treat Myotonic Dystrophy Type 1 (DM1).

For treatment of a subject (e.g., a human), any of the compositions disclosed herein may be administered in a manner compatible with the dosage formulation and in such amount as is therapeutically effective. The compositions may be readily administered in a variety of dosage forms, such as injectable solutions. For parenteral administration in an aqueous solution, for example, the solution will generally be suitably buffered and the liquid diluent first rendered isotonic with, for example, sufficient saline or glucose. Such aqueous solutions may be used, for example, for intravenous, intramuscular, subcutaneous, and/or intraperitoneal administration.

In some embodiments, any of the compositions described herein is administered to a subject in need thereof to induce a double strand break in the DMPK gene.

In some embodiments, any of the compositions described herein is administered to a subject in need thereof to excise a CTG repeat in the 3′ UTR of the DMPK gene.

In some embodiments, any of the compositions described herein is administered to a subject in need thereof to treat DM1, e.g., in a subject having a CTG repeat in the 3′ UTR of the DMPK gene.

In some embodiments, a method of treating Myotonic Dystrophy Type 1 (DM1) is provided, the method comprising delivering to a cell any one of the compositions described herein. In some embodiments, the method further comprises administering a DNA-PK inhibitor. In some embodiments, the DNA-PK inhibitor is Compound 1. In some embodiments, the DNA-PK inhibitor is Compound 2. In some embodiments, the DNA-PK inhibitor is Compound 6.

In particular, in some embodiments, a method of treating Myotonic Dystrophy Type 1 (DM1) is provided, the method comprising delivering to a cell: 1) a nucleic acid molecule comprising: a nucleic acid encoding a spacer sequence selected from SEQ ID NOs: 1-65, 67-167, and 201-531; a nucleic acid encoding a spacer sequence comprising at least 17, 18, 19, 20, or 21 contiguous nucleotides of a spacer sequence selected from SEQ ID NOs: 1-65, 67-167, and 201-531; or a nucleic acid encoding a spacer sequence that is at least 90% identical to any one of SEQ ID NOs: 1-65, 67-167, and 201-531; and 2) a Staphylococcus lugdunensis Cas9 (SluCas9) or a nucleic acid encoding SluCas9. In some embodiments, the spacer sequence is SEQ ID NO: 1. In some embodiments, the spacer sequence is SEQ ID NO: 2. In some embodiments, the spacer sequence is SEQ ID NO: 3. In some embodiments, the spacer sequence is SEQ ID NO: 4. In some embodiments, the spacer sequence is SEQ ID NO: 5. In some embodiments, the spacer sequence is SEQ ID NO: 6. In some embodiments, the spacer sequence is SEQ ID NO: 7. In some embodiments, the spacer sequence is SEQ ID NO: 8. In some embodiments, the spacer sequence is SEQ ID NO: 9. In some embodiments, the spacer sequence is SEQ ID NO: 10. In some embodiments, the spacer sequence is SEQ ID NO: 11. In some embodiments, the spacer sequence is SEQ ID NO: 12. In some embodiments, the spacer sequence is SEQ ID NO: 13. In some embodiments, the spacer sequence is SEQ ID NO: 14. In some embodiments, the spacer sequence is SEQ ID NO: 15. In some embodiments, the spacer sequence is SEQ ID NO: 16. In some embodiments, the spacer sequence is SEQ ID NO: 17. In some embodiments, the spacer sequence is SEQ ID NO: 18. In some embodiments, the spacer sequence is SEQ ID NO: 19. In some embodiments, the spacer sequence is SEQ ID NO: 20. In some embodiments, the spacer sequence is SEQ ID NO: 21. In some embodiments, the spacer sequence is SEQ ID NO: 22. In some embodiments, the spacer sequence is SEQ ID NO: 23. In some embodiments, the spacer sequence is SEQ ID NO: 24. In some embodiments, the spacer sequence is SEQ ID NO: 25. In some embodiments, the spacer sequence is SEQ ID NO: 26. In some embodiments, the spacer sequence is SEQ ID NO: 27. In some embodiments, the spacer sequence is SEQ ID NO: 28. In some embodiments, the spacer sequence is SEQ ID NO: 29. In some embodiments, the spacer sequence is SEQ ID NO: 30. In some embodiments, the spacer sequence is SEQ ID NO: 31. In some embodiments, the spacer sequence is SEQ ID NO: 32. In some embodiments, the spacer sequence is SEQ ID NO: 33. In some embodiments, the spacer sequence is SEQ ID NO: 34. In some embodiments, the spacer sequence is SEQ ID NO: 35. In some embodiments, the spacer sequence is SEQ ID NO: 36. In some embodiments, the spacer sequence is SEQ ID NO: 37. In some embodiments, the spacer sequence is SEQ ID NO: 38. In some embodiments, the spacer sequence is SEQ ID NO: 39. In some embodiments, the spacer sequence is SEQ ID NO: 40. In some embodiments, the spacer sequence is SEQ ID NO: 41. In some embodiments, the spacer sequence is SEQ ID NO: 42. In some embodiments, the spacer sequence is SEQ ID NO: 43. In some embodiments, the spacer sequence is SEQ ID NO: 44. In some embodiments, the spacer sequence is SEQ ID NO: 45. In some embodiments, the spacer sequence is SEQ ID NO: 46. In some embodiments, the spacer sequence is SEQ ID NO: 47. In some embodiments, the spacer sequence is SEQ ID NO: 48. In some embodiments, the spacer sequence is SEQ ID NO: 49. In some embodiments, the spacer sequence is SEQ ID NO: 50. In some embodiments, the spacer sequence is SEQ ID NO: 51. In some embodiments, the spacer sequence is SEQ ID NO: 51. In some embodiments, the spacer sequence is SEQ ID NO: 52. In some embodiments, the spacer sequence is SEQ ID NO: 53. In some embodiments, the spacer sequence is SEQ ID NO: 54. In some embodiments, the spacer sequence is SEQ ID NO: 55. In some embodiments, the spacer sequence is SEQ ID NO: 56. In some embodiments, the spacer sequence is SEQ ID NO: 57. In some embodiments, the spacer sequence is SEQ ID NO: 58. In some embodiments, the spacer sequence is SEQ ID NO: 59. In some embodiments, the spacer sequence is SEQ ID NO: 60. In some embodiments, the spacer sequence is SEQ ID NO: 61. In some embodiments, the spacer sequence is SEQ ID NO: 62. In some embodiments, the spacer sequence is SEQ ID NO: 63. In some embodiments, the spacer sequence is SEQ ID NO: 64. In some embodiments, the spacer sequence is SEQ ID NO: 65. In some embodiments, the spacer sequence is SEQ ID NO: 66. In some embodiments, the spacer sequence is SEQ ID NO: 67. In some embodiments, the spacer sequence is SEQ ID NO: 68. In some embodiments, the spacer sequence is SEQ ID NO: 69. In some embodiments, the spacer sequence is SEQ ID NO: 70. In some embodiments, the spacer sequence is SEQ ID NO: 71. In some embodiments, the spacer sequence is SEQ ID NO: 72. In some embodiments, the spacer sequence is SEQ ID NO: 73. In some embodiments, the spacer sequence is SEQ ID NO: 74. In some embodiments, the spacer sequence is SEQ ID NO: 75. In some embodiments, the spacer sequence is SEQ ID NO: 76. In some embodiments, the spacer sequence is SEQ ID NO: 77. In some embodiments, the spacer sequence is SEQ ID NO: 78. In some embodiments, the spacer sequence is SEQ ID NO: 79. In some embodiments, the spacer sequence is SEQ ID NO: 80. In some embodiments, the spacer sequence is SEQ ID NO: 81. In some embodiments, the spacer sequence is SEQ ID NO: 82. In some embodiments, the spacer sequence is SEQ ID NO: 83. In some embodiments, the spacer sequence is SEQ ID NO: 84. In some embodiments, the spacer sequence is SEQ ID NO: 85. In some embodiments, the spacer sequence is SEQ ID NO: 86. In some embodiments, the spacer sequence is SEQ ID NO: 87. In some embodiments, the spacer sequence is SEQ ID NO: 88. In some embodiments, the spacer sequence is SEQ ID NO: 89. In some embodiments, the spacer sequence is SEQ ID NO: 90. In some embodiments, the spacer sequence is SEQ ID NO: 91. In some embodiments, the spacer sequence is SEQ ID NO: 92. In some embodiments, the spacer sequence is SEQ ID NO: 93. In some embodiments, the spacer sequence is SEQ ID NO: 94. In some embodiments, the spacer sequence is SEQ ID NO: 95. In some embodiments, the spacer sequence is SEQ ID NO: 96. In some embodiments, the spacer sequence is SEQ ID NO: 97. In some embodiments, the spacer sequence is SEQ ID NO: 98. In some embodiments, the spacer sequence is SEQ ID NO: 99. In some embodiments, the spacer sequence is SEQ ID NO: 100. In some embodiments, the spacer sequence is SEQ ID NO: 101. In some embodiments, the spacer sequence is SEQ ID NO: 102. In some embodiments, the spacer sequence is SEQ ID NO: 103. In some embodiments, the spacer sequence is SEQ ID NO: 104. In some embodiments, the spacer sequence is SEQ ID NO: 105. In some embodiments, the spacer sequence is SEQ ID NO: 106. In some embodiments, the spacer sequence is SEQ ID NO: 107. In some embodiments, the spacer sequence is SEQ ID NO: 108. In some embodiments, the spacer sequence is SEQ ID NO: 109. In some embodiments, the spacer sequence is SEQ ID NO: 110. In some embodiments, the spacer sequence is SEQ ID NO: 111. In some embodiments, the spacer sequence is SEQ ID NO: 112. In some embodiments, the spacer sequence is SEQ ID NO: 113. In some embodiments, the spacer sequence is SEQ ID NO: 114. In some embodiments, the spacer sequence is SEQ ID NO: 115. In some embodiments, the spacer sequence is SEQ ID NO: 116. In some embodiments, the spacer sequence is SEQ ID NO: 117. In some embodiments, the spacer sequence is SEQ ID NO: 118. In some embodiments, the spacer sequence is SEQ ID NO: 119. In some embodiments, the spacer sequence is SEQ ID NO: 120. In some embodiments, the spacer sequence is SEQ ID NO: 121. In some embodiments, the spacer sequence is SEQ ID NO: 122. In some embodiments, the spacer sequence is SEQ ID NO: 123. In some embodiments, the spacer sequence is SEQ ID NO: 124. In some embodiments, the spacer sequence is SEQ ID NO: 125. In some embodiments, the spacer sequence is SEQ ID NO: 126. In some embodiments, the spacer sequence is SEQ ID NO: 127. In some embodiments, the spacer sequence is SEQ ID NO: 128. In some embodiments, the spacer sequence is SEQ ID NO: 129. In some embodiments, the spacer sequence is SEQ ID NO: 130. In some embodiments, the spacer sequence is SEQ ID NO: 131. In some embodiments, the spacer sequence is SEQ ID NO: 132. In some embodiments, the spacer sequence is SEQ ID NO: 133. In some embodiments, the spacer sequence is SEQ ID NO: 134. In some embodiments, the spacer sequence is SEQ ID NO: 135. In some embodiments, the spacer sequence is SEQ ID NO: 136. In some embodiments, the spacer sequence is SEQ ID NO: 137. In some embodiments, the spacer sequence is SEQ ID NO: 138. In some embodiments, the spacer sequence is SEQ ID NO: 139. In some embodiments, the spacer sequence is SEQ ID NO: 140. In some embodiments, the spacer sequence is SEQ ID NO: 141. In some embodiments, the spacer sequence is SEQ ID NO: 142. In some embodiments, the spacer sequence is SEQ ID NO: 143. In some embodiments, the spacer sequence is SEQ ID NO: 144. In some embodiments, the spacer sequence is SEQ ID NO: 145. In some embodiments, the spacer sequence is SEQ ID NO: 146. In some embodiments, the spacer sequence is SEQ ID NO: 147. In some embodiments, the spacer sequence is SEQ ID NO: 148. In some embodiments, the spacer sequence is SEQ ID NO: 149. In some embodiments, the spacer sequence is SEQ ID NO: 150. In some embodiments, the spacer sequence is SEQ ID NO: 151. In some embodiments, the spacer sequence is SEQ ID NO: 152. In some embodiments, the spacer sequence is SEQ ID NO: 153. In some embodiments, the spacer sequence is SEQ ID NO: 154. In some embodiments, the spacer sequence is SEQ ID NO: 155. In some embodiments, the spacer sequence is SEQ ID NO: 156. In some embodiments, the spacer sequence is SEQ ID NO: 157. In some embodiments, the spacer sequence is SEQ ID NO: 158. In some embodiments, the spacer sequence is SEQ ID NO: 159. In some embodiments, the spacer sequence is SEQ ID NO: 160. In some embodiments, the spacer sequence is SEQ ID NO: 161. In some embodiments, the spacer sequence is SEQ ID NO: 161. In some embodiments, the spacer sequence is SEQ ID NO: 162. In some embodiments, the spacer sequence is SEQ ID NO: 163. In some embodiments, the spacer sequence is SEQ ID NO: 164. In some embodiments, the spacer sequence is SEQ ID NO: 165. In some embodiments, the spacer sequence is SEQ ID NO: 166. In some embodiments, the spacer is selected from SEQ ID NOs: 8, 63, 64, and 81. In some embodiments, the spacer sequence is SEQ ID NO: 167. In some embodiments, the cell comprises a CTG repeat in the 3′ UTR of the DMPK gene. In some embodiments, the method further comprises administering a DNA-PK inhibitor.

In particular, in some embodiments, a method of treating Myotonic Dystrophy Type 1 (DM1) is provided, the method comprising delivering to a cell: 1) a nucleic acid molecule comprising: a nucleic acid encoding a spacer sequence selected from SEQ ID NOs: 1-172, and 201-531; a nucleic acid encoding a spacer sequence comprising at least 17, 18, 19, 20, or 21 contiguous nucleotides of a spacer sequence selected from SEQ ID NOs: 1-172, and 201-531; or a nucleic acid encoding a spacer sequence that is at least 90% identical to any one of SEQ ID NOs: 1-172, and 201-531; and 2) a Staphylococcus lugdunensis Cas9 (SluCas9) or a nucleic acid encoding SluCas9. In some embodiments, the nucleic acid encoding SluCas9 also encodes a spacer sequence selected from SEQ ID NOs: 1-172, and 201-531; a nucleic acid encoding a spacer sequence comprising at least 17, 18, 19, 20, or 21 contiguous nucleotides of a spacer sequence selected from SEQ ID NOs: 1-172, and 201-531; or a nucleic acid encoding a spacer sequence that is at least 90% identical to any one of SEQ ID NOs: 1-172, and 201-531. In some embodiments, the nucleic acid encoding SluCas9 does not encode for any guide RNA.

In certain preferred embodiments, the spacer sequence comprises at least 20 contiguous nucleotides of a spacer sequence selected from SEQ ID NOs: 1-172, and 201-531; or a nucleic acid encoding a spacer sequence that is at least 90% identical to any one of SEQ ID NOs: 1-172, and 201-531.

In some embodiments, a method of treating Myotonic Dystrophy Type 1 (DM1) is provided, the method comprising delivering to a cell a single nucleic acid molecule comprising: i) a nucleic acid encoding a pair of guide RNAs comprising: a) a first spacer sequence selected from any one of SEQ ID NOs: 1-65 and a second spacer sequence selected from any one of SEQ ID NOs: 67-167; b) a first and second spacer sequence comprising at least 17, 18, 19, 20, or 21 contiguous nucleotides of any of the first and second spacer sequences of i) a); or c) a first and second spacer sequence that is at least 90% identical to any of the first and second spacer sequences of i) a) or i) b); and ii) a nucleic acid encoding a Staphylococcus lugdunensis Cas9 (SluCas9). In some embodiments, the nucleic acid encoding SluCas9 also encodes a pair of guide RNAs comprising: a) a first spacer sequence selected from any one of SEQ ID NOs: 1-65 and a second spacer sequence selected from any one of SEQ ID NOs: 67-167; b) a first and second spacer sequence comprising at least 17, 18, 19, 20, or 21 contiguous nucleotides of any of the first and second spacer sequences of a); or c) a first and second spacer sequence that is at least 90% identical to any of the first and second spacer sequences of a) or b. In some embodiments, the nucleic acid encoding SluCas9 does not encode for any guide RNA. In some embodiments, the method further comprises administering a DNA-PK inhibitor.

In some embodiments, a method of excising a CTG repeat in the 3′ UTR of the DMPK gene is provided, the method comprising delivering to a cell a single nucleic acid molecule comprising: 1) a nucleic acid molecule comprising: a nucleic acid encoding a spacer sequence selected from SEQ ID NOs: 1-65, 67-167, and 201-531; a nucleic acid encoding a spacer sequence comprising at least 17, 18, 19, 20, or 21 contiguous nucleotides of a spacer sequence selected from SEQ ID NOs: 1-65, 67-167, and 201-531; or a nucleic acid encoding a spacer sequence that is at least 90% identical to any one of SEQ ID NOs: 1-65, 67-167, and 201-531; and 2) a Staphylococcus lugdunensis Cas9 (SluCas9) or a nucleic acid encoding SluCas9. In some embodiments, the nucleic acid encoding SluCas9 also encodes a spacer sequence selected from SEQ ID NOs: 1-172, and 201-531; a nucleic acid encoding a spacer sequence comprising at least 17, 18, 19, 20, or 21 contiguous nucleotides of a spacer sequence selected from SEQ ID NOs: 1-172, and 201-531; or a nucleic acid encoding a spacer sequence that is at least 90% identical to any one of SEQ ID NOs: 1-172, and 201-531. In some embodiments, the nucleic acid encoding SluCas9 does not encode for any guide RNA. In some embodiments, the method further comprises administering a DNA-PK inhibitor.

In some embodiments, only one guide RNA is administered and a CTG repeat in the 3′ UTR is excised. In some embodiments, a pair of guide RNAs is administered and a CTG repeat in the 3′ UTR is excised.

In some embodiments, a method of excising a CTG repeat in the 3′ UTR of the DMPK gene is provided, the method comprising delivering to a cell a single nucleic acid molecule comprising: 1) a nucleic acid molecule encoding a pair of guide RNAs comprising: a) a pair of guide RNAs that comprise a first spacer sequence selected from any one of SEQ ID NOs: 1-65 and a second spacer sequence selected from any one of SEQ ID NOs: 67-167; b) a first and second spacer sequence comprising at least 17, 18, 19, 20, or 21 contiguous nucleotides of a spacer sequence selected from any one of 1) a); or c) a first and second spacer sequence that is at least 90% identical to any one of 1) a) or 1) b); and 2) a Staphylococcus lugdunensis Cas9 (SluCas9) or a nucleic acid encoding SluCas9. In some embodiments, the nucleic acid encoding SluCas9 also encodes a pair of guide RNAs comprising: a) a first spacer sequence selected from any one of SEQ ID NOs: 1-65 and a second spacer sequence selected from any one of SEQ ID NOs: 67-167; b) a first and second spacer sequence comprising at least 17, 18, 19, 20, or 21 contiguous nucleotides of any of the first and second spacer sequences of a); or c) a first and second spacer sequence that is at least 90% identical to any of the first and second spacer sequences of a) or b. In some embodiments, the nucleic acid encoding SluCas9 does not encode for any guide RNA. In some embodiments, the method further comprises administering a DNA-PK inhibitor.

In some embodiments, the methods provided herein comprise a first and second spacer sequence selected from any one of SEQ ID NOs:

1 and 67; 1 and 68; 1 and 69; 1 and 70; 1 and 71; 1 and 72; 1 and 73; 1 and 74; 1 and 75; 1 and 76; 1 and 77; 1 and 78; 1 and 79; 1 and 80; 1 and 81; 1 and 82; 1 and 83; 1 and 84; 1 and 85; 1 and 86; 1 and 87; 1 and 88; 1 and 89; 1 and 90; 1 and 91; 1 and 92; 1 and 93; 1 and 94; 1 and 95; 1 and 96; 1 and 97; 1 and 98; 1 and 99; 1 and 100; 1 and 101; 1 and 102; 1 and 103; 1 and 104; 1 and 105; 1 and 106; 1 and 107; 1 and 108; 1 and 109; 1 and 110; 1 and 111; 1 and 112; 1 and 113; 1 and 114; 1 and 115; 1 and 116; 1 and 117; 1 and 118; 1 and 119; 1 and 120; 1 and 121; 1 and 122; 1 and 123; 1 and 124; 1 and 125; 1 and 126; 1 and 127; 1 and 128; 1 and 129; 1 and 130; 1 and 131; 1 and 132; 1 and 133; 1 and 134; 1 and 135; 1 and 136; 1 and 137; 1 and 138; 1 and 139; 1 and 140; 1 and 141; 1 and 142; 1 and 143; 1 and 144; 1 and 145; 1 and 146; 1 and 147; 1 and 148; 1 and 149; 1 and 150; 1 and 151; 1 and 152; 1 and 153; 1 and 154; 1 and 155; 1 and 156; 1 and 157; 1 and 158; 1 and 159; 1 and 160; 1 and 161; 1 and 162; 1 and 163; 1 and 164; 1 and 165; 1 and 166; 1 and 167; 2 and 67; 2 and 68; 2 and 69; 2 and 70; 2 and 71; 2 and 72; 2 and 73; 2 and 74; 2 and 75; 2 and 76; 2 and 77; 2 and 78; 2 and 79; 2 and 80; 2 and 81; 2 and 82; 2 and 83; 2 and 84; 2 and 85; 2 and 86; 2 and 87; 2 and 88; 2 and 89; 2 and 90; 2 and 91; 2 and 92; 2 and 93; 2 and 94; 2 and 95; 2 and 96; 2 and 97; 2 and 98; 2 and 99; 2 and 100; 2 and 101; 2 and 102; 2 and 103; 2 and 104; 2 and 105; 2 and 106; 2 and 107; 2 and 108; 2 and 109; 2 and 110; 2 and 111; 2 and 112; 2 and 113; 2 and 114; 2 and 115; 2 and 116; 2 and 117; 2 and 118; 2 and 119; 2 and 120; 2 and 121; 2 and 122; 2 and 123; 2 and 124; 2 and 125; 2 and 126; 2 and 127; 2 and 128; 2 and 129; 2 and 130; 2 and 131; 2 and 132; 2 and 133; 2 and 134; 2 and 135; 2 and 136; 2 and 137; 2 and 138; 2 and 139; 2 and 140; 2 and 141; 2 and 142; 2 and 143; 2 and 144; 2 and 145; 2 and 146; 2 and 147; 2 and 148; 2 and 149; 2 and 150; 2 and 151; 2 and 152; 2 and 153; 2 and 154; 2 and 155; 2 and 156; 2 and 157; 2 and 158; 2 and 159; 2 and 160; 2 and 161; 2 and 162; 2 and 163; 2 and 164; 2 and 165; 2 and 166; 2 and 167; 3 and 67; 3 and 68; 3 and 69; 3 and 70; 3 and 71; 3 and 72; 3 and 73; 3 and 74; 3 and 75; 3 and 76; 3 and 77; 3 and 78; 3 and 79; 3 and 80; 3 and 81; 3 and 82; 3 and 83; 3 and 84; 3 and 85; 3 and 86; 3 and 87; 3 and 88; 3 and 89; 3 and 90; 3 and 91; 3 and 92; 3 and 93; 3 and 94; 3 and 95; 3 and 96; 3 and 97; 3 and 98; 3 and 99; 3 and 100; 3 and 101; 3 and 102; 3 and 103; 3 and 104; 3 and 105; 3 and 106; 3 and 107; 3 and 108; 3 and 109; 3 and 110; 3 and 111; 3 and 112; 3 and 113; 3 and 114; 3 and 115; 3 and 116; 3 and 117; 3 and 118; 3 and 119; 3 and 120; 3 and 121; 3 and 122; 3 and 123; 3 and 124; 3 and 125; 3 and 126; 3 and 127; 3 and 128; 3 and 129; 3 and 130; 3 and 131; 3 and 132; 3 and 133; 3 and 134; 3 and 135; 3 and 136; 3 and 137; 3 and 138; 3 and 139; 3 and 140; 3 and 141; 3 and 142; 3 and 143; 3 and 144; 3 and 145; 3 and 146; 3 and 147; 3 and 148; 3 and 149; 3 and 150; 3 and 151; 3 and 152; 3 and 153; 3 and 154; 3 and 155; 3 and 156; 3 and 157; 3 and 158; 3 and 159; 3 and 160; 3 and 161; 3 and 162; 3 and 163; 3 and 164; 3 and 165; 3 and 166; 3 and 167; 4 and 67; 4 and 68; 4 and 69; 4 and 70; 4 and 71; 4 and 72; 4 and 73; 4 and 74; 4 and 75; 4 and 76; 4 and 77; 4 and 78; 4 and 79; 4 and 80; 4 and 81; 4 and 82; 4 and 83; 4 and 84; 4 and 85; 4 and 86; 4 and 87; 4 and 88; 4 and 89; 4 and 90; 4 and 91; 4 and 92; 4 and 93; 4 and 94; 4 and 95; 4 and 96; 4 and 97; 4 and 98; 4 and 99; 4 and 100; 4 and 101; 4 and 102; 4 and 103; 4 and 104; 4 and 105; 4 and 106; 4 and 107; 4 and 108; 4 and 109; 4 and 110; 4 and 111; 4 and 112; 4 and 113; 4 and 114; 4 and 115; 4 and 116; 4 and 117; 4 and 118; 4 and 119; 4 and 120; 4 and 121; 4 and 122; 4 and 123; 4 and 124; 4 and 125; 4 and 126; 4 and 127; 4 and 128; 4 and 129; 4 and 130; 4 and 131; 4 and 132; 4 and 133; 4 and 134; 4 and 135; 4 and 136; 4 and 137; 4 and 138; 4 and 139; 4 and 140; 4 and 141; 4 and 142; 4 and 143; 4 and 144; 4 and 145; 4 and 146; 4 and 147; 4 and 148; 4 and 149; 4 and 150; 4 and 151; 4 and 152; 4 and 153; 4 and 154; 4 and 155; 4 and 156; 4 and 157; 4 and 158; 4 and 159; 4 and 160; 4 and 161; 4 and 162; 4 and 163; 4 and 164; 4 and 165; 4 and 166; 4 and 167; 5 and 67; 5 and 68; 5 and 69; 5 and 70; 5 and 71; 5 and 72; 5 and 73; 5 and 74; 5 and 75; 5 and 76; 5 and 77; 5 and 78; 5 and 79; 5 and 80; 5 and 81; 5 and 82; 5 and 83; 5 and 84; 5 and 85; 5 and 86; 5 and 87; 5 and 88; 5 and 89; 5 and 90; 5 and 91; 5 and 92; 5 and 93; 5 and 94; 5 and 95; 5 and 96; 5 and 97; 5 and 98; 5 and 99; 5 and 100; 5 and 101; 5 and 102; 5 and 103; 5 and 104; 5 and 105; 5 and 106; 5 and 107; 5 and 108; 5 and 109; 5 and 110; 5 and 111; 5 and 112; 5 and 113; 5 and 114; 5 and 115; 5 and 116; 5 and 117; 5 and 118; 5 and 119; 5 and 120; 5 and 121; 5 and 122; 5 and 123; 5 and 124; 5 and 125; 5 and 126; 5 and 127; 5 and 128; 5 and 129; 5 and 130; 5 and 131; 5 and 132; 5 and 133; 5 and 134; 5 and 135; 5 and 136; 5 and 137; 5 and 138; 5 and 139; 5 and 140; 5 and 141; 5 and 142; 5 and 143; 5 and 144; 5 and 145; 5 and 146; 5 and 147; 5 and 148; 5 and 149; 5 and 150; 5 and 151; 5 and 152; 5 and 153; 5 and 154; 5 and 155; 5 and 156; 5 and 157; 5 and 158; 5 and 159; 5 and 160; 5 and 161; 5 and 162; 5 and 163; 5 and 164; 5 and 165; 5 and 166; 5 and 167; 6 and 67; 6 and 68; 6 and 69; 6 and 70; 6 and 71; 6 and 72; 6 and 73; 6 and 74; 6 and 75; 6 and 76; 6 and 77; 6 and 78; 6 and 79; 6 and 80; 6 and 81; 6 and 82; 6 and 83; 6 and 84; 6 and 85; 6 and 86; 6 and 87; 6 and 88; 6 and 89; 6 and 90; 6 and 91; 6 and 92; 6 and 93; 6 and 94; 6 and 95; 6 and 96; 6 and 97; 6 and 98; 6 and 99; 6 and 100; 6 and 101; 6 and 102; 6 and 103; 6 and 104; 6 and 105; 6 and 106; 6 and 107; 6 and 108; 6 and 109; 6 and 110; 6 and 111; 6 and 112; 6 and 113; 6 and 114; 6 and 115; 6 and 116; 6 and 117; 6 and 118; 6 and 119; 6 and 120; 6 and 121; 6 and 122; 6 and 123; 6 and 124; 6 and 125; 6 and 126; 6 and 127; 6 and 128; 6 and 129; 6 and 130; 6 and 131; 6 and 132; 6 and 133; 6 and 134; 6 and 135; 6 and 136; 6 and 137; 6 and 138; 6 and 139; 6 and 140; 6 and 141; 6 and 142; 6 and 143; 6 and 144; 6 and 145; 6 and 146; 6 and 147; 6 and 148; 6 and 149; 6 and 150; 6 and 151; 6 and 152; 6 and 153; 6 and 154; 6 and 155; 6 and 156; 6 and 157; 6 and 158; 6 and 159; 6 and 160; 6 and 161; 6 and 162; 6 and 163; 6 and 164; 6 and 165; 6 and 166; 6 and 167; 7 and 67; 7 and 68; 7 and 69; 7 and 70; 7 and 71; 7 and 72; 7 and 73; 7 and 74; 7 and 75; 7 and 76; 7 and 77; 7 and 78; 7 and 79; 7 and 80; 7 and 81; 7 and 82; 7 and 83; 7 and 84; 7 and 85; 7 and 86; 7 and 87; 7 and 88; 7 and 89; 7 and 90; 7 and 91; 7 and 92; 7 and 93; 7 and 94; 7 and 95; 7 and 96; 7 and 97; 7 and 98; 7 and 99; 7 and 100; 7 and 101; 7 and 102; 7 and 103; 7 and 104; 7 and 105; 7 and 106; 7 and 107; 7 and 108; 7 and 109; 7 and 110; 7 and 111; 7 and 112; 7 and 113; 7 and 114; 7 and 115; 7 and 116; 7 and 117; 7 and 118; 7 and 119; 7 and 120; 7 and 121; 7 and 122; 7 and 123; 7 and 124; 7 and 125; 7 and 126; 7 and 127; 7 and 128; 7 and 129; 7 and 130; 7 and 131; 7 and 132; 7 and 133; 7 and 134; 7 and 135; 7 and 136; 7 and 137; 7 and 138; 7 and 139; 7 and 140; 7 and 141; 7 and 142; 7 and 143; 7 and 144; 7 and 145; 7 and 146; 7 and 147; 7 and 148; 7 and 149; 7 and 150; 7 and 151; 7 and 152; 7 and 153; 7 and 154; 7 and 155; 7 and 156; 7 and 157; 7 and 158; 7 and 159; 7 and 160; 7 and 161; 7 and 162; 7 and 163; 7 and 164; 7 and 165; 7 and 166; 7 and 167; 8 and 67; 8 and 68; 8 and 69; 8 and 70; 8 and 71; 8 and 72; 8 and 73; 8 and 74; 8 and 75; 8 and 76; 8 and 77; 8 and 78; 8 and 79; 8 and 80; 8 and 81; 8 and 82; 8 and 83; 8 and 84; 8 and 85; 8 and 86; 8 and 87; 8 and 88; 8 and 89; 8 and 90; 8 and 91; 8 and 92; 8 and 93; 8 and 94; 8 and 95; 8 and 96; 8 and 97; 8 and 98; 8 and 99; 8 and 100; 8 and 101; 8 and 102; 8 and 103; 8 and 104; 8 and 105; 8 and 106; 8 and 107; 8 and 108; 8 and 109; 8 and 110; 8 and 111; 8 and 112; 8 and 113; 8 and 114; 8 and 115; 8 and 116; 8 and 117; 8 and 118; 8 and 119; 8 and 120; 8 and 121; 8 and 122; 8 and 123; 8 and 124; 8 and 125; 8 and 126; 8 and 127; 8 and 128; 8 and 129; 8 and 130; 8 and 131; 8 and 132; 8 and 133; 8 and 134; 8 and 135; 8 and 136; 8 and 137; 8 and 138; 8 and 139; 8 and 140; 8 and 141; 8 and 142; 8 and 143; 8 and 144; 8 and 145; 8 and 146; 8 and 147; 8 and 148; 8 and 149; 8 and 150; 8 and 151; 8 and 152; 8 and 153; 8 and 154; 8 and 155; 8 and 156; 8 and 157; 8 and 158; 8 and 159; 8 and 160; 8 and 161; 8 and 162; 8 and 163; 8 and 164; 8 and 165; 8 and 166; 8 and 167; 9 and 67; 9 and 68; 9 and 69; 9 and 70; 9 and 71; 9 and 72; 9 and 73; 9 and 74; 9 and 75; 9 and 76; 9 and 77; 9 and 78; 9 and 79; 9 and 80; 9 and 81; 9 and 82; 9 and 83; 9 and 84; 9 and 85; 9 and 86; 9 and 87; 9 and 88; 9 and 89; 9 and 90; 9 and 91; 9 and 92; 9 and 93; 9 and 94; 9 and 95; 9 and 96; 9 and 97; 9 and 98; 9 and 99; 9 and 100; 9 and 101; 9 and 102; 9 and 103; 9 and 104; 9 and 105; 9 and 106; 9 and 107; 9 and 108; 9 and 109; 9 and 110; 9 and 111; 9 and 112; 9 and 113; 9 and 114; 9 and 115; 9 and 116; 9 and 117; 9 and 118; 9 and 119; 9 and 120; 9 and 121; 9 and 122; 9 and 123; 9 and 124; 9 and 125; 9 and 126; 9 and 127; 9 and 128; 9 and 129; 9 and 130; 9 and 131; 9 and 132; 9 and 133; 9 and 134; 9 and 135; 9 and 136; 9 and 137; 9 and 138; 9 and 139; 9 and 140; 9 and 141; 9 and 142; 9 and 143; 9 and 144; 9 and 145; 9 and 146; 9 and 147; 9 and 148; 9 and 149; 9 and 150; 9 and 151; 9 and 152; 9 and 153; 9 and 154; 9 and 155; 9 and 156; 9 and 157; 9 and 158; 9 and 159; 9 and 160; 9 and 161; 9 and 162; 9 and 163; 9 and 164; 9 and 165; 9 and 166; 9 and 167; 10 and 67; 10 and 68; 10 and 69; 10 and 70; 10 and 71; 10 and 72; 10 and 73; 10 and 74; 10 and 75; 10 and 76; 10 and 77; 10 and 78; 10 and 79; 10 and 80; 10 and 81; 10 and 82; 10 and 83; 10 and 84; 10 and 85; 10 and 86; 10 and 87; 10 and 88; 10 and 89; 10 and 90; 10 and 91; 10 and 92; 10 and 93; 10 and 94; 10 and 95; 10 and 96; 10 and 97; 10 and 98; 10 and 99; 10 and 100; 10 and 101; 10 and 102; 10 and 103; 10 and 104; 10 and 105; 10 and 106; 10 and 107; 10 and 108; 10 and 109; 10 and 110; 10 and 111; 10 and 112; 10 and 113; 10 and 114; 10 and 115; 10 and 116; 10 and 117; 10 and 118; 10 and 119; 10 and 120; 10 and 121; 10 and 122; 10 and 123; 10 and 124; 10 and 125; 10 and 126; 10 and 127; 10 and 128; 10 and 129; 10 and 130; 10 and 131; 10 and 132; 10 and 133; 10 and 134; 10 and 135; 10 and 136; 10 and 137; 10 and 138; 10 and 139; 10 and 140; 10 and 141; 10 and 142; 10 and 143; 10 and 144; 10 and 145; 10 and 146; 10 and 147; 10 and 148; 10 and 149; 10 and 150; 10 and 151; 10 and 152; 10 and 153; 10 and 154; 10 and 155; 10 and 156; 10 and 157; 10 and 158; 10 and 159; 10 and 160; 10 and 161; 10 and 162; 10 and 163; 10 and 164; 10 and 165; 10 and 166; 10 and 167; 11 and 67; 11 and 68; 11 and 69; 11 and 70; 11 and 71; 11 and 72; 11 and 73; 11 and 74; 11 and 75; 11 and 76; 11 and 77; 11 and 78; 11 and 79; 11 and 80; 11 and 81; 11 and 82; 11 and 83; 11 and 84; 11 and 85; 11 and 86; 11 and 87; 11 and 88; 11 and 89; 11 and 90; 11 and 91; 11 and 92; 11 and 93; 11 and 94; 11 and 95; 11 and 96; 11 and 97; 11 and 98; 11 and 99; 11 and 100; 11 and 101; 11 and 102; 11 and 103; 11 and 104; 11 and 105; 11 and 106; 11 and 107; 11 and 108; 11 and 109; 11 and 110; 11 and 111; 11 and 112; 11 and 113; 11 and 114; 11 and 115; 11 and 116; 11 and 117; 11 and 118; 11 and 119; 11 and 120; 11 and 121; 11 and 122; 11 and 123; 11 and 124; 11 and 125; 11 and 126; 11 and 127; 11 and 128; 11 and 129; 11 and 130; 11 and 131; 11 and 132; 11 and 133; 11 and 134; 11 and 135; 11 and 136; 11 and 137; 11 and 138; 11 and 139; 11 and 140; 11 and 141; 11 and 142; 11 and 143; 11 and 144; 11 and 145; 11 and 146; 11 and 147; 11 and 148; 11 and 149; 11 and 150; 11 and 151; 11 and 152; 11 and 153; 11 and 154; 11 and 155; 11 and 156; 11 and 157; 11 and 158; 11 and 159; 11 and 160; 11 and 161; 11 and 162; 11 and 163; 11 and 164; 11 and 165; 11 and 166; 11 and 167; 12 and 67; 12 and 68; 12 and 69; 12 and 70; 12 and 71; 12 and 72; 12 and 73; 12 and 74; 12 and 75; 12 and 76; 12 and 77; 12 and 78; 12 and 79; 12 and 80; 12 and 81; 12 and 82; 12 and 83; 12 and 84; 12 and 85; 12 and 86; 12 and 87; 12 and 88; 12 and 89; 12 and 90; 12 and 91; 12 and 92; 12 and 93; 12 and 94; 12 and 95; 12 and 96; 12 and 97; 12 and 98; 12 and 99; 12 and 100; 12 and 101; 12 and 102; 12 and 103; 12 and 104; 12 and 105; 12 and 106; 12 and 107; 12 and 108; 12 and 109; 12 and 110; 12 and 111; 12 and 112; 12 and 113; 12 and 114; 12 and 115; 12 and 116; 12 and 117; 12 and 118; 12 and 119; 12 and 120; 12 and 121; 12 and 122; 12 and 123; 12 and 124; 12 and 125; 12 and 126; 12 and 127; 12 and 128; 12 and 129; 12 and 130; 12 and 131; 12 and 132; 12 and 133; 12 and 134; 12 and 135; 12 and 136; 12 and 137; 12 and 138; 12 and 139; 12 and 140; 12 and 141; 12 and 142; 12 and 143; 12 and 144; 12 and 145; 12 and 146; 12 and 147; 12 and 148; 12 and 149; 12 and 150; 12 and 151; 12 and 152; 12 and 153; 12 and 154; 12 and 155; 12 and 156; 12 and 157; 12 and 158; 12 and 159; 12 and 160; 12 and 161; 12 and 162; 12 and 163; 12 and 164; 12 and 165; 12 and 166; 12 and 167; 13 and 67; 13 and 68; 13 and 69; 13 and 70; 13 and 71; 13 and 72; 13 and 73; 13 and 74; 13 and 75; 13 and 76; 13 and 77; 13 and 78; 13 and 79; 13 and 80; 13 and 81; 13 and 82; 13 and 83; 13 and 84; 13 and 85; 13 and 86; 13 and 87; 13 and 88; 13 and 89; 13 and 90; 13 and 91; 13 and 92; 13 and 93; 13 and 94; 13 and 95; 13 and 96; 13 and 97; 13 and 98; 13 and 99; 13 and 100; 13 and 101; 13 and 102; 13 and 103; 13 and 104; 13 and 105; 13 and 106; 13 and 107; 13 and 108; 13 and 109; 13 and 110; 13 and 111; 13 and 112; 13 and 113; 13 and 114; 13 and 115; 13 and 116; 13 and 117; 13 and 118; 13 and 119; 13 and 120; 13 and 121; 13 and 122; 13 and 123; 13 and 124; 13 and 125; 13 and 126; 13 and 127; 13 and 128; 13 and 129; 13 and 130; 13 and 131; 13 and 132; 13 and 133; 13 and 134; 13 and 135; 13 and 136; 13 and 137; 13 and 138; 13 and 139; 13 and 140; 13 and 141; 13 and 142; 13 and 143; 13 and 144; 13 and 145; 13 and 146; 13 and 147; 13 and 148; 13 and 149; 13 and 150; 13 and 151; 13 and 152; 13 and 153; 13 and 154; 13 and 155; 13 and 156; 13 and 157; 13 and 158; 13 and 159; 13 and 160; 13 and 161; 13 and 162; 13 and 163; 13 and 164; 13 and 165; 13 and 166; 13 and 167; 14 and 67; 14 and 68; 14 and 69; 14 and 70; 14 and 71; 14 and 72; 14 and 73; 14 and 74; 14 and 75; 14 and 76; 14 and 77; 14 and 78; 14 and 79; 14 and 80; 14 and 81; 14 and 82; 14 and 83; 14 and 84; 14 and 85; 14 and 86; 14 and 87; 14 and 88; 14 and 89; 14 and 90; 14 and 91; 14 and 92; 14 and 93; 14 and 94; 14 and 95; 14 and 96; 14 and 97; 14 and 98; 14 and 99; 14 and 100; 14 and 101; 14 and 102; 14 and 103; 14 and 104; 14 and 105; 14 and 106; 14 and 107; 14 and 108; 14 and 109; 14 and 110; 14 and 111; 14 and 112; 14 and 113; 14 and 114; 14 and 115; 14 and 116; 14 and 117; 14 and 118; 14 and 119; 14 and 120; 14 and 121; 14 and 122; 14 and 123; 14 and 124; 14 and 125; 14 and 126; 14 and 127; 14 and 128; 14 and 129; 14 and 130; 14 and 131; 14 and 132; 14 and 133; 14 and 134; 14 and 135; 14 and 136; 14 and 137; 14 and 138; 14 and 139; 14 and 140; 14 and 141; 14 and 142; 14 and 143; 14 and 144; 14 and 145; 14 and 146; 14 and 147; 14 and 148; 14 and 149; 14 and 150; 14 and 151; 14 and 152; 14 and 153; 14 and 154; 14 and 155; 14 and 156; 14 and 157; 14 and 158; 14 and 159; 14 and 160; 14 and 161; 14 and 162; 14 and 163; 14 and 164; 14 and 165; 14 and 166; 14 and 167; 15 and 67; 15 and 68; 15 and 69; 15 and 70; 15 and 71; 15 and 72; 15 and 73; 15 and 74; 15 and 75; 15 and 76; 15 and 77; 15 and 78; 15 and 79; 15 and 80; 15 and 81; 15 and 82; 15 and 83; 15 and 84; 15 and 85; 15 and 86; 15 and 87; 15 and 88; 15 and 89; 15 and 90; 15 and 91; 15 and 92; 15 and 93; 15 and 94; 15 and 95; 15 and 96; 15 and 97; 15 and 98; 15 and 99; 15 and 100; 15 and 101; 15 and 102; 15 and 103; 15 and 104; 15 and 105; 15 and 106; 15 and 107; 15 and 108; 15 and 109; 15 and 110; 15 and 111; 15 and 112; 15 and 113; 15 and 114; 15 and 115; 15 and 116; 15 and 117; 15 and 118; 15 and 119; 15 and 120; 15 and 121; 15 and 122; 15 and 123; 15 and 124; 15 and 125; 15 and 126; 15 and 127; 15 and 128; 15 and 129; 15 and 130; 15 and 131; 15 and 132; 15 and 133; 15 and 134; 15 and 135; 15 and 136; 15 and 137; 15 and 138; 15 and 139; 15 and 140; 15 and 141; 15 and 142; 15 and 143; 15 and 144; 15 and 145; 15 and 146; 15 and 147; 15 and 148; 15 and 149; 15 and 150; 15 and 151; 15 and 152; 15 and 153; 15 and 154; 15 and 155; 15 and 156; 15 and 157; 15 and 158; 15 and 159; 15 and 160; 15 and 161; 15 and 162; 15 and 163; 15 and 164; 15 and 165; 15 and 166; 15 and 167; 16 and 67; 16 and 68; 16 and 69; 16 and 70; 16 and 71; 16 and 72; 16 and 73; 16 and 74; 16 and 75; 16 and 76; 16 and 77; 16 and 78; 16 and 79; 16 and 80; 16 and 81; 16 and 82; 16 and 83; 16 and 84; 16 and 85; 16 and 86; 16 and 87; 16 and 88; 16 and 89; 16 and 90; 16 and 91; 16 and 92; 16 and 93; 16 and 94; 16 and 95; 16 and 96; 16 and 97; 16 and 98; 16 and 99; 16 and 100; 16 and 101; 16 and 102; 16 and 103; 16 and 104; 16 and 105; 16 and 106; 16 and 107; 16 and 108; 16 and 109; 16 and 110; 16 and 111; 16 and 112; 16 and 113; 16 and 114; 16 and 115; 16 and 116; 16 and 117; 16 and 118; 16 and 119; 16 and 120; 16 and 121; 16 and 122; 16 and 123; 16 and 124; 16 and 125; 16 and 126; 16 and 127; 16 and 128; 16 and 129; 16 and 130; 16 and 131; 16 and 132; 16 and 133; 16 and 134; 16 and 135; 16 and 136; 16 and 137; 16 and 138; 16 and 139; 16 and 140; 16 and 141; 16 and 142; 16 and 143; 16 and 144; 16 and 145; 16 and 146; 16 and 147; 16 and 148; 16 and 149; 16 and 150; 16 and 151; 16 and 152; 16 and 153; 16 and 154; 16 and 155; 16 and 156; 16 and 157; 16 and 158; 16 and 159; 16 and 160; 16 and 161; 16 and 162; 16 and 163; 16 and 164; 16 and 165; 16 and 166; 16 and 167; 17 and 67; 17 and 68; 17 and 69; 17 and 70; 17 and 71; 17 and 72; 17 and 73; 17 and 74; 17 and 75; 17 and 76; 17 and 77; 17 and 78; 17 and 79; 17 and 80; 17 and 81; 17 and 82; 17 and 83; 17 and 84; 17 and 85; 17 and 86; 17 and 87; 17 and 88; 17 and 89; 17 and 90; 17 and 91; 17 and 92; 17 and 93; 17 and 94; 17 and 95; 17 and 96; 17 and 97; 17 and 98; 17 and 99; 17 and 100; 17 and 101; 17 and 102; 17 and 103; 17 and 104; 17 and 105; 17 and 106; 17 and 107; 17 and 108; 17 and 109; 17 and 110; 17 and 111; 17 and 112; 17 and 113; 17 and 114; 17 and 115; 17 and 116; 17 and 117; 17 and 118; 17 and 119; 17 and 120; 17 and 121; 17 and 122; 17 and 123; 17 and 124; 17 and 125; 17 and 126; 17 and 127; 17 and 128; 17 and 129; 17 and 130; 17 and 131; 17 and 132; 17 and 133; 17 and 134; 17 and 135; 17 and 136; 17 and 137; 17 and 138; 17 and 139; 17 and 140; 17 and 141; 17 and 142; 17 and 143; 17 and 144; 17 and 145; 17 and 146; 17 and 147; 17 and 148; 17 and 149; 17 and 150; 17 and 151; 17 and 152; 17 and 153; 17 and 154; 17 and 155; 17 and 156; 17 and 157; 17 and 158; 17 and 159; 17 and 160; 17 and 161; 17 and 162; 17 and 163; 17 and 164; 17 and 165; 17 and 166; 17 and 167; 18 and 67; 18 and 68; 18 and 69; 18 and 70; 18 and 71; 18 and 72; 18 and 73; 18 and 74; 18 and 75; 18 and 76; 18 and 77; 18 and 78; 18 and 79; 18 and 80; 18 and 81; 18 and 82; 18 and 83; 18 and 84; 18 and 85; 18 and 86; 18 and 87; 18 and 88; 18 and 89; 18 and 90; 18 and 91; 18 and 92; 18 and 93; 18 and 94; 18 and 95; 18 and 96; 18 and 97; 18 and 98; 18 and 99; 18 and 100; 18 and 101; 18 and 102; 18 and 103; 18 and 104; 18 and 105; 18 and 106; 18 and 107; 18 and 108; 18 and 109; 18 and 110; 18 and 111; 18 and 112; 18 and 113; 18 and 114; 18 and 115; 18 and 116; 18 and 117; 18 and 118; 18 and 119; 18 and 120; 18 and 121; 18 and 122; 18 and 123; 18 and 124; 18 and 125; 18 and 126; 18 and 127; 18 and 128; 18 and 129; 18 and 130; 18 and 131; 18 and 132; 18 and 133; 18 and 134; 18 and 135; 18 and 136; 18 and 137; 18 and 138; 18 and 139; 18 and 140; 18 and 141; 18 and 142; 18 and 143; 18 and 144; 18 and 145; 18 and 146; 18 and 147; 18 and 148; 18 and 149; 18 and 150; 18 and 151; 18 and 152; 18 and 153; 18 and 154; 18 and 155; 18 and 156; 18 and 157; 18 and 158; 18 and 159; 18 and 160; 18 and 161; 18 and 162; 18 and 163; 18 and 164; 18 and 165; 18 and 166; 18 and 167; 19 and 67; 19 and 68; 19 and 69; 19 and 70; 19 and 71; 19 and 72; 19 and 73; 19 and 74; 19 and 75; 19 and 76; 19 and 77; 19 and 78; 19 and 79; 19 and 80; 19 and 81; 19 and 82; 19 and 83; 19 and 84; 19 and 85; 19 and 86; 19 and 87; 19 and 88; 19 and 89; 19 and 90; 19 and 91; 19 and 92; 19 and 93; 19 and 94; 19 and 95; 19 and 96; 19 and 97; 19 and 98; 19 and 99; 19 and 100; 19 and 101; 19 and 102; 19 and 103; 19 and 104; 19 and 105; 19 and 106; 19 and 107; 19 and 108; 19 and 109; 19 and 110; 19 and 111; 19 and 112; 19 and 113; 19 and 114; 19 and 115; 19 and 116; 19 and 117; 19 and 118; 19 and 119; 19 and 120; 19 and 121; 19 and 122; 19 and 123; 19 and 124; 19 and 125; 19 and 126; 19 and 127; 19 and 128; 19 and 129; 19 and 130; 19 and 131; 19 and 132; 19 and 133; 19 and 134; 19 and 135; 19 and 136; 19 and 137; 19 and 138; 19 and 139; 19 and 140; 19 and 141; 19 and 142; 19 and 143; 19 and 144; 19 and 145; 19 and 146; 19 and 147; 19 and 148; 19 and 149; 19 and 150; 19 and 151; 19 and 152; 19 and 153; 19 and 154; 19 and 155; 19 and 156; 19 and 157; 19 and 158; 19 and 159; 19 and 160; 19 and 161; 19 and 162; 19 and 163; 19 and 164; 19 and 165; 19 and 166; 19 and 167; 20 and 67; 20 and 68; 20 and 69; 20 and 70; 20 and 71; 20 and 72; 20 and 73; 20 and 74; 20 and 75; 20 and 76; 20 and 77; 20 and 78; 20 and 79; 20 and 80; 20 and 81; 20 and 82; 20 and 83; 20 and 84; 20 and 85; 20 and 86; 20 and 87; 20 and 88; 20 and 89; 20 and 90; 20 and 91; 20 and 92; 20 and 93; 20 and 94; 20 and 95; 20 and 96; 20 and 97; 20 and 98; 20 and 99; 20 and 100; 20 and 101; 20 and 102; 20 and 103; 20 and 104; 20 and 105; 20 and 106; 20 and 107; 20 and 108; 20 and 109; 20 and 110; 20 and 111; 20 and 112; 20 and 113; 20 and 114; 20 and 115; 20 and 116; 20 and 117; 20 and 118; 20 and 119; 20 and 120; 20 and 121; 20 and 122; 20 and 123; 20 and 124; 20 and 125; 20 and 126; 20 and 127; 20 and 128; 20 and 129; 20 and 130; 20 and 131; 20 and 132; 20 and 133; 20 and 134; 20 and 135; 20 and 136; 20 and 137; 20 and 138; 20 and 139; 20 and 140; 20 and 141; 20 and 142; 20 and 143; 20 and 144; 20 and 145; 20 and 146; 20 and 147; 20 and 148; 20 and 149; 20 and 150; 20 and 151; 20 and 152; 20 and 153; 20 and 154; 20 and 155; 20 and 156; 20 and 157; 20 and 158; 20 and 159; 20 and 160; 20 and 161; 20 and 162; 20 and 163; 20 and 164; 20 and 165; 20 and 166; 20 and 167; 21 and 67; 21 and 68; 21 and 69; 21 and 70; 21 and 71; 21 and 72; 21 and 73; 21 and 74; 21 and 75; 21 and 76; 21 and 77; 21 and 78; 21 and 79; 21 and 80; 21 and 81; 21 and 82; 21 and 83; 21 and 84; 21 and 85; 21 and 86; 21 and 87; 21 and 88; 21 and 89; 21 and 90; 21 and 91; 21 and 92; 21 and 93; 21 and 94; 21 and 95; 21 and 96; 21 and 97; 21 and 98; 21 and 99; 21 and 100; 21 and 101; 21 and 102; 21 and 103; 21 and 104; 21 and 105; 21 and 106; 21 and 107; 21 and 108; 21 and 109; 21 and 110; 21 and 111; 21 and 112; 21 and 113; 21 and 114; 21 and 115; 21 and 116; 21 and 117; 21 and 118; 21 and 119; 21 and 120; 21 and 121; 21 and 122; 21 and 123; 21 and 124; 21 and 125; 21 and 126; 21 and 127; 21 and 128; 21 and 129; 21 and 130; 21 and 131; 21 and 132; 21 and 133; 21 and 134; 21 and 135; 21 and 136; 21 and 137; 21 and 138; 21 and 139; 21 and 140; 21 and 141; 21 and 142; 21 and 143; 21 and 144; 21 and 145; 21 and 146; 21 and 147; 21 and 148; 21 and 149; 21 and 150; 21 and 151; 21 and 152; 21 and 153; 21 and 154; 21 and 155; 21 and 156; 21 and 157; 21 and 158; 21 and 159; 21 and 160; 21 and 161; 21 and 162; 21 and 163; 21 and 164; 21 and 165; 21 and 166; 21 and 167; 22 and 67; 22 and 68; 22 and 69; 22 and 70; 22 and 71; 22 and 72; 22 and 73; 22 and 74; 22 and 75; 22 and 76; 22 and 77; 22 and 78; 22 and 79; 22 and 80; 22 and 81; 22 and 82; 22 and 83; 22 and 84; 22 and 85; 22 and 86; 22 and 87; 22 and 88; 22 and 89; 22 and 90; 22 and 91; 22 and 92; 22 and 93; 22 and 94; 22 and 95; 22 and 96; 22 and 97; 22 and 98; 22 and 99; 22 and 100; 22 and 101; 22 and 102; 22 and 103; 22 and 104; 22 and 105; 22 and 106; 22 and 107; 22 and 108; 22 and 109; 22 and 110; 22 and 111; 22 and 112; 22 and 113; 22 and 114; 22 and 115; 22 and 116; 22 and 117; 22 and 118; 22 and 119; 22 and 120; 22 and 121; 22 and 122; 22 and 123; 22 and 124; 22 and 125; 22 and 126; 22 and 127; 22 and 128; 22 and 129; 22 and 130; 22 and 131; 22 and 132; 22 and 133; 22 and 134; 22 and 135; 22 and 136; 22 and 137; 22 and 138; 22 and 139; 22 and 140; 22 and 141; 22 and 142; 22 and 143; 22 and 144; 22 and 145; 22 and 146; 22 and 147; 22 and 148; 22 and 149; 22 and 150; 22 and 151; 22 and 152; 22 and 153; 22 and 154; 22 and 155; 22 and 156; 22 and 157; 22 and 158; 22 and 159; 22 and 160; 22 and 161; 22 and 162; 22 and 163; 22 and 164; 22 and 165; 22 and 166; 22 and 167; 23 and 67; 23 and 68; 23 and 69; 23 and 70; 23 and 71; 23 and 72; 23 and 73; 23 and 74; 23 and 75; 23 and 76; 23 and 77; 23 and 78; 23 and 79; 23 and 80; 23 and 81; 23 and 82; 23 and 83; 23 and 84; 23 and 85; 23 and 86; 23 and 87; 23 and 88; 23 and 89; 23 and 90; 23 and 91; 23 and 92; 23 and 93; 23 and 94; 23 and 95; 23 and 96; 23 and 97; 23 and 98; 23 and 99; 23 and 100; 23 and 101; 23 and 102; 23 and 103; 23 and 104; 23 and 105; 23 and 106; 23 and 107; 23 and 108; 23 and 109; 23 and 110; 23 and 111; 23 and 112; 23 and 113; 23 and 114; 23 and 115; 23 and 116; 23 and 117; 23 and 118; 23 and 119; 23 and 120; 23 and 121; 23 and 122; 23 and 123; 23 and 124; 23 and 125; 23 and 126; 23 and 127; 23 and 128; 23 and 129; 23 and 130; 23 and 131; 23 and 132; 23 and 133; 23 and 134; 23 and 135; 23 and 136; 23 and 137; 23 and 138; 23 and 139; 23 and 140; 23 and 141; 23 and 142; 23 and 143; 23 and 144; 23 and 145; 23 and 146; 23 and 147; 23 and 148; 23 and 149; 23 and 150; 23 and 151; 23 and 152; 23 and 153; 23 and 154; 23 and 155; 23 and 156; 23 and 157; 23 and 158; 23 and 159; 23 and 160; 23 and 161; 23 and 162; 23 and 163; 23 and 164; 23 and 165; 23 and 166; 23 and 167; 24 and 67; 24 and 68; 24 and 69; 24 and 70; 24 and 71; 24 and 72; 24 and 73; 24 and 74; 24 and 75; 24 and 76; 24 and 77; 24 and 78; 24 and 79; 24 and 80; 24 and 81; 24 and 82; 24 and 83; 24 and 84; 24 and 85; 24 and 86; 24 and 87; 24 and 88; 24 and 89; 24 and 90; 24 and 91; 24 and 92; 24 and 93; 24 and 94; 24 and 95; 24 and 96; 24 and 97; 24 and 98; 24 and 99; 24 and 100; 24 and 101; 24 and 102; 24 and 103; 24 and 104; 24 and 105; 24 and 106; 24 and 107; 24 and 108; 24 and 109; 24 and 110; 24 and 111; 24 and 112; 24 and 113; 24 and 114; 24 and 115; 24 and 116; 24 and 117; 24 and 118; 24 and 119; 24 and 120; 24 and 121; 24 and 122; 24 and 123; 24 and 124; 24 and 125; 24 and 126; 24 and 127; 24 and 128; 24 and 129; 24 and 130; 24 and 131; 24 and 132; 24 and 133; 24 and 134; 24 and 135; 24 and 136; 24 and 137; 24 and 138; 24 and 139; 24 and 140; 24 and 141; 24 and 142; 24 and 143; 24 and 144; 24 and 145; 24 and 146; 24 and 147; 24 and 148; 24 and 149; 24 and 150; 24 and 151; 24 and 152; 24 and 153; 24 and 154; 24 and 155; 24 and 156; 24 and 157; 24 and 158; 24 and 159; 24 and 160; 24 and 161; 24 and 162; 24 and 163; 24 and 164; 24 and 165; 24 and 166; 24 and 167; 25 and 67; 25 and 68; 25 and 69; 25 and 70; 25 and 71; 25 and 72; 25 and 73; 25 and 74; 25 and 75; 25 and 76; 25 and 77; 25 and 78; 25 and 79; 25 and 80; 25 and 81; 25 and 82; 25 and 83; 25 and 84; 25 and 85; 25 and 86; 25 and 87; 25 and 88; 25 and 89; 25 and 90; 25 and 91; 25 and 92; 25 and 93; 25 and 94; 25 and 95; 25 and 96; 25 and 97; 25 and 98; 25 and 99; 25 and 100; 25 and 101; 25 and 102; 25 and 103; 25 and 104; 25 and 105; 25 and 106; 25 and 107; 25 and 108; 25 and 109; 25 and 110; 25 and 111; 25 and 112; 25 and 113; 25 and 114; 25 and 115; 25 and 116; 25 and 117; 25 and 118; 25 and 119; 25 and 120; 25 and 121; 25 and 122; 25 and 123; 25 and 124; 25 and 125; 25 and 126; 25 and 127; 25 and 128; 25 and 129; 25 and 130; 25 and 131; 25 and 132; 25 and 133; 25 and 134; 25 and 135; 25 and 136; 25 and 137; 25 and 138; 25 and 139; 25 and 140; 25 and 141; 25 and 142; 25 and 143; 25 and 144; 25 and 145; 25 and 146; 25 and 147; 25 and 148; 25 and 149; 25 and 150; 25 and 151; 25 and 152; 25 and 153; 25 and 154; 25 and 155; 25 and 156; 25 and 157; 25 and 158; 25 and 159; 25 and 160; 25 and 161; 25 and 162; 25 and 163; 25 and 164; 25 and 165; 25 and 166; 25 and 167; 26 and 67; 26 and 68; 26 and 69; 26 and 70; 26 and 71; 26 and 72; 26 and 73; 26 and 74; 26 and 75; 26 and 76; 26 and 77; 26 and 78; 26 and 79; 26 and 80; 26 and 81; 26 and 82; 26 and 83; 26 and 84; 26 and 85; 26 and 86; 26 and 87; 26 and 88; 26 and 89; 26 and 90; 26 and 91; 26 and 92; 26 and 93; 26 and 94; 26 and 95; 26 and 96; 26 and 97; 26 and 98; 26 and 99; 26 and 100; 26 and 101; 26 and 102; 26 and 103; 26 and 104; 26 and 105; 26 and 106; 26 and 107; 26 and 108; 26 and 109; 26 and 110; 26 and 111; 26 and 112; 26 and 113; 26 and 114; 26 and 115; 26 and 116; 26 and 117; 26 and 118; 26 and 119; 26 and 120; 26 and 121; 26 and 122; 26 and 123; 26 and 124; 26 and 125; 26 and 126; 26 and 127; 26 and 128; 26 and 129; 26 and 130; 26 and 131; 26 and 132; 26 and 133; 26 and 134; 26 and 135; 26 and 136; 26 and 137; 26 and 138; 26 and 139; 26 and 140; 26 and 141; 26 and 142; 26 and 143; 26 and 144; 26 and 145; 26 and 146; 26 and 147; 26 and 148; 26 and 149; 26 and 150; 26 and 151; 26 and 152; 26 and 153; 26 and 154; 26 and 155; 26 and 156; 26 and 157; 26 and 158; 26 and 159; 26 and 160; 26 and 161; 26 and 162; 26 and 163; 26 and 164; 26 and 165; 26 and 166; 26 and 167; 27 and 67; 27 and 68; 27 and 69; 27 and 70; 27 and 71; 27 and 72; 27 and 73; 27 and 74; 27 and 75; 27 and 76; 27 and 77; 27 and 78; 27 and 79; 27 and 80; 27 and 81; 27 and 82; 27 and 83; 27 and 84; 27 and 85; 27 and 86; 27 and 87; 27 and 88; 27 and 89; 27 and 90; 27 and 91; 27 and 92; 27 and 93; 27 and 94; 27 and 95; 27 and 96; 27 and 97; 27 and 98; 27 and 99; 27 and 100; 27 and 101; 27 and 102; 27 and 103; 27 and 104; 27 and 105; 27 and 106; 27 and 107; 27 and 108; 27 and 109; 27 and 110; 27 and 111; 27 and 112; 27 and 113; 27 and 114; 27 and 115; 27 and 116; 27 and 117; 27 and 118; 27 and 119; 27 and 120; 27 and 121; 27 and 122; 27 and 123; 27 and 124; 27 and 125; 27 and 126; 27 and 127; 27 and 128; 27 and 129; 27 and 130; 27 and 131; 27 and 132; 27 and 133; 27 and 134; 27 and 135; 27 and 136; 27 and 137; 27 and 138; 27 and 139; 27 and 140; 27 and 141; 27 and 142; 27 and 143; 27 and 144; 27 and 145; 27 and 146; 27 and 147; 27 and 148; 27 and 149; 27 and 150; 27 and 151; 27 and 152; 27 and 153; 27 and 154; 27 and 155; 27 and 156; 27 and 157; 27 and 158; 27 and 159; 27 and 160; 27 and 161; 27 and 162; 27 and 163; 27 and 164; 27 and 165; 27 and 166; 27 and 167; 28 and 67; 28 and 68; 28 and 69; 28 and 70; 28 and 71; 28 and 72; 28 and 73; 28 and 74; 28 and 75; 28 and 76; 28 and 77; 28 and 78; 28 and 79; 28 and 80; 28 and 81; 28 and 82; 28 and 83; 28 and 84; 28 and 85; 28 and 86; 28 and 87; 28 and 88; 28 and 89; 28 and 90; 28 and 91; 28 and 92; 28 and 93; 28 and 94; 28 and 95; 28 and 96; 28 and 97; 28 and 98; 28 and 99; 28 and 100; 28 and 101; 28 and 102; 28 and 103; 28 and 104; 28 and 105; 28 and 106; 28 and 107; 28 and 108; 28 and 109; 28 and 110; 28 and 111; 28 and 112; 28 and 113; 28 and 114; 28 and 115; 28 and 116; 28 and 117; 28 and 118; 28 and 119; 28 and 120; 28 and 121; 28 and 122; 28 and 123; 28 and 124; 28 and 125; 28 and 126; 28 and 127; 28 and 128; 28 and 129; 28 and 130; 28 and 131; 28 and 132; 28 and 133; 28 and 134; 28 and 135; 28 and 136; 28 and 137; 28 and 138; 28 and 139; 28 and 140; 28 and 141; 28 and 142; 28 and 143; 28 and 144; 28 and 145; 28 and 146; 28 and 147; 28 and 148; 28 and 149; 28 and 150; 28 and 151; 28 and 152; 28 and 153; 28 and 154; 28 and 155; 28 and 156; 28 and 157; 28 and 158; 28 and 159; 28 and 160; 28 and 161; 28 and 162; 28 and 163; 28 and 164; 28 and 165; 28 and 166; 28 and 167; 29 and 67; 29 and 68; 29 and 69; 29 and 70; 29 and 71; 29 and 72; 29 and 73; 29 and 74; 29 and 75; 29 and 76; 29 and 77; 29 and 78; 29 and 79; 29 and 80; 29 and 81; 29 and 82; 29 and 83; 29 and 84; 29 and 85; 29 and 86; 29 and 87; 29 and 88; 29 and 89; 29 and 90; 29 and 91; 29 and 92; 29 and 93; 29 and 94; 29 and 95; 29 and 96; 29 and 97; 29 and 98; 29 and 99; 29 and 100; 29 and 101; 29 and 102; 29 and 103; 29 and 104; 29 and 105; 29 and 106; 29 and 107; 29 and 108; 29 and 109; 29 and 110; 29 and 111; 29 and 112; 29 and 113; 29 and 114; 29 and 115; 29 and 116; 29 and 117; 29 and 118; 29 and 119; 29 and 120; 29 and 121; 29 and 122; 29 and 123; 29 and 124; 29 and 125; 29 and 126; 29 and 127; 29 and 128; 29 and 129; 29 and 130; 29 and 131; 29 and 132; 29 and 133; 29 and 134; 29 and 135; 29 and 136; 29 and 137; 29 and 138; 29 and 139; 29 and 140; 29 and 141; 29 and 142; 29 and 143; 29 and 144; 29 and 145; 29 and 146; 29 and 147; 29 and 148; 29 and 149; 29 and 150; 29 and 151; 29 and 152; 29 and 153; 29 and 154; 29 and 155; 29 and 156; 29 and 157; 29 and 158; 29 and 159; 29 and 160; 29 and 161; 29 and 162; 29 and 163; 29 and 164; 29 and 165; 29 and 166; 29 and 167; 30 and 67; 30 and 68; 30 and 69; 30 and 70; 30 and 71; 30 and 72; 30 and 73; 30 and 74; 30 and 75; 30 and 76; 30 and 77; 30 and 78; 30 and 79; 30 and 80; 30 and 81; 30 and 82; 30 and 83; 30 and 84; 30 and 85; 30 and 86; 30 and 87; 30 and 88; 30 and 89; 30 and 90; 30 and 91; 30 and 92; 30 and 93; 30 and 94; 30 and 95; 30 and 96; 30 and 97; 30 and 98; 30 and 99; 30 and 100; 30 and 101; 30 and 102; 30 and 103; 30 and 104; 30 and 105; 30 and 106; 30 and 107; 30 and 108; 30 and 109; 30 and 110; 30 and 111; 30 and 112; 30 and 113; 30 and 114; 30 and 115; 30 and 116; 30 and 117; 30 and 118; 30 and 119; 30 and 120; 30 and 121; 30 and 122; 30 and 123; 30 and 124; 30 and 125; 30 and 126; 30 and 127; 30 and 128; 30 and 129; 30 and 130; 30 and 131; 30 and 132; 30 and 133; 30 and 134; 30 and 135; 30 and 136; 30 and 137; 30 and 138; 30 and 139; 30 and 140; 30 and 141; 30 and 142; 30 and 143; 30 and 144; 30 and 145; 30 and 146; 30 and 147; 30 and 148; 30 and 149; 30 and 150; 30 and 151; 30 and 152; 30 and 153; 30 and 154; 30 and 155; 30 and 156; 30 and 157; 30 and 158; 30 and 159; 30 and 160; 30 and 161; 30 and 162; 30 and 163; 30 and 164; 30 and 165; 30 and 166; 30 and 167; 31 and 67; 31 and 68; 31 and 69; 31 and 70; 31 and 71; 31 and 72; 31 and 73; 31 and 74; 31 and 75; 31 and 76; 31 and 77; 31 and 78; 31 and 79; 31 and 80; 31 and 81; 31 and 82; 31 and 83; 31 and 84; 31 and 85; 31 and 86; 31 and 87; 31 and 88; 31 and 89; 31 and 90; 31 and 91; 31 and 92; 31 and 93; 31 and 94; 31 and 95; 31 and 96; 31 and 97; 31 and 98; 31 and 99; 31 and 100; 31 and 101; 31 and 102; 31 and 103; 31 and 104; 31 and 105; 31 and 106; 31 and 107; 31 and 108; 31 and 109; 31 and 110; 31 and 111; 31 and 112; 31 and 113; 31 and 114; 31 and 115; 31 and 116; 31 and 117; 31 and 118; 31 and 119; 31 and 120; 31 and 121; 31 and 122; 31 and 123; 31 and 124; 31 and 125; 31 and 126; 31 and 127; 31 and 128; 31 and 129; 31 and 130; 31 and 131; 31 and 132; 31 and 133; 31 and 134; 31 and 135; 31 and 136; 31 and 137; 31 and 138; 31 and 139; 31 and 140; 31 and 141; 31 and 142; 31 and 143; 31 and 144; 31 and 145; 31 and 146; 31 and 147; 31 and 148; 31 and 149; 31 and 150; 31 and 151; 31 and 152; 31 and 153; 31 and 154; 31 and 155; 31 and 156; 31 and 157; 31 and 158; 31 and 159; 31 and 160; 31 and 161; 31 and 162; 31 and 163; 31 and 164; 31 and 165; 31 and 166; 31 and 167; 32 and 67; 32 and 68; 32 and 69; 32 and 70; 32 and 71; 32 and 72; 32 and 73; 32 and 74; 32 and 75; 32 and 76; 32 and 77; 32 and 78; 32 and 79; 32 and 80; 32 and 81; 32 and 82; 32 and 83; 32 and 84; 32 and 85; 32 and 86; 32 and 87; 32 and 88; 32 and 89; 32 and 90; 32 and 91; 32 and 92; 32 and 93; 32 and 94; 32 and 95; 32 and 96; 32 and 97; 32 and 98; 32 and 99; 32 and 100; 32 and 101; 32 and 102; 32 and 103; 32 and 104; 32 and 105; 32 and 106; 32 and 107; 32 and 108; 32 and 109; 32 and 110; 32 and 111; 32 and 112; 32 and 113; 32 and 114; 32 and 115; 32 and 116; 32 and 117; 32 and 118; 32 and 119; 32 and 120; 32 and 121; 32 and 122; 32 and 123; 32 and 124; 32 and 125; 32 and 126; 32 and 127; 32 and 128; 32 and 129; 32 and 130; 32 and 131; 32 and 132; 32 and 133; 32 and 134; 32 and 135; 32 and 136; 32 and 137; 32 and 138; 32 and 139; 32 and 140; 32 and 141; 32 and 142; 32 and 143; 32 and 144; 32 and 145; 32 and 146; 32 and 147; 32 and 148; 32 and 149; 32 and 150; 32 and 151; 32 and 152; 32 and 153; 32 and 154; 32 and 155; 32 and 156; 32 and 157; 32 and 158; 32 and 159; 32 and 160; 32 and 161; 32 and 162; 32 and 163; 32 and 164; 32 and 165; 32 and 166; 32 and 167; 33 and 67; 33 and 68; 33 and 69; 33 and 70; 33 and 71; 33 and 72; 33 and 73; 33 and 74; 33 and 75; 33 and 76; 33 and 77; 33 and 78; 33 and 79; 33 and 80; 33 and 81; 33 and 82; 33 and 83; 33 and 84; 33 and 85; 33 and 86; 33 and 87; 33 and 88; 33 and 89; 33 and 90; 33 and 91; 33 and 92; 33 and 93; 33 and 94; 33 and 95; 33 and 96; 33 and 97; 33 and 98; 33 and 99; 33 and 100; 33 and 101; 33 and 102; 33 and 103; 33 and 104; 33 and 105; 33 and 106; 33 and 107; 33 and 108; 33 and 109; 33 and 110; 33 and 111; 33 and 112; 33 and 113; 33 and 114; 33 and 115; 33 and 116; 33 and 117; 33 and 118; 33 and 119; 33 and 120; 33 and 121; 33 and 122; 33 and 123; 33 and 124; 33 and 125; 33 and 126; 33 and 127; 33 and 128; 33 and 129; 33 and 130; 33 and 131; 33 and 132; 33 and 133; 33 and 134; 33 and 135; 33 and 136; 33 and 137; 33 and 138; 33 and 139; 33 and 140; 33 and 141; 33 and 142; 33 and 143; 33 and 144; 33 and 145; 33 and 146; 33 and 147; 33 and 148; 33 and 149; 33 and 150; 33 and 151; 33 and 152; 33 and 153; 33 and 154; 33 and 155; 33 and 156; 33 and 157; 33 and 158; 33 and 159; 33 and 160; 33 and 161; 33 and 162; 33 and 163; 33 and 164; 33 and 165; 33 and 166; 33 and 167; 34 and 67; 34 and 68; 34 and 69; 34 and 70; 34 and 71; 34 and 72; 34 and 73; 34 and 74; 34 and 75; 34 and 76; 34 and 77; 34 and 78; 34 and 79; 34 and 80; 34 and 81; 34 and 82; 34 and 83; 34 and 84; 34 and 85; 34 and 86; 34 and 87; 34 and 88; 34 and 89; 34 and 90; 34 and 91; 34 and 92; 34 and 93; 34 and 94; 34 and 95; 34 and 96; 34 and 97; 34 and 98; 34 and 99; 34 and 100; 34 and 101; 34 and 102; 34 and 103; 34 and 104; 34 and 105; 34 and 106; 34 and 107; 34 and 108; 34 and 109; 34 and 110; 34 and 111; 34 and 112; 34 and 113; 34 and 114; 34 and 115; 34 and 116; 34 and 117; 34 and 118; 34 and 119; 34 and 120; 34 and 121; 34 and 122; 34 and 123; 34 and 124; 34 and 125; 34 and 126; 34 and 127; 34 and 128; 34 and 129; 34 and 130; 34 and 131; 34 and 132; 34 and 133; 34 and 134; 34 and 135; 34 and 136; 34 and 137; 34 and 138; 34 and 139; 34 and 140; 34 and 141; 34 and 142; 34 and 143; 34 and 144; 34 and 145; 34 and 146; 34 and 147; 34 and 148; 34 and 149; 34 and 150; 34 and 151; 34 and 152; 34 and 153; 34 and 154; 34 and 155; 34 and 156; 34 and 157; 34 and 158; 34 and 159; 34 and 160; 34 and 161; 34 and 162; 34 and 163; 34 and 164; 34 and 165; 34 and 166; 34 and 167; 35 and 67; 35 and 68; 35 and 69; 35 and 70; 35 and 71; 35 and 72; 35 and 73; 35 and 74; 35 and 75; 35 and 76; 35 and 77; 35 and 78; 35 and 79; 35 and 80; 35 and 81; 35 and 82; 35 and 83; 35 and 84; 35 and 85; 35 and 86; 35 and 87; 35 and 88; 35 and 89; 35 and 90; 35 and 91; 35 and 92; 35 and 93; 35 and 94; 35 and 95; 35 and 96; 35 and 97; 35 and 98; 35 and 99; 35 and 100; 35 and 101; 35 and 102; 35 and 103; 35 and 104; 35 and 105; 35 and 106; 35 and 107; 35 and 108; 35 and 109; 35 and 110; 35 and 111; 35 and 112; 35 and 113; 35 and 114; 35 and 115; 35 and 116; 35 and 117; 35 and 118; 35 and 119; 35 and 120; 35 and 121; 35 and 122; 35 and 123; 35 and 124; 35 and 125; 35 and 126; 35 and 127; 35 and 128; 35 and 129; 35 and 130; 35 and 131; 35 and 132; 35 and 133; 35 and 134; 35 and 135; 35 and 136; 35 and 137; 35 and 138; 35 and 139; 35 and 140; 35 and 141; 35 and 142; 35 and 143; 35 and 144; 35 and 145; 35 and 146; 35 and 147; 35 and 148; 35 and 149; 35 and 150; 35 and 151; 35 and 152; 35 and 153; 35 and 154; 35 and 155; 35 and 156; 35 and 157; 35 and 158; 35 and 159; 35 and 160; 35 and 161; 35 and 162; 35 and 163; 35 and 164; 35 and 165; 35 and 166; 35 and 167; 36 and 67; 36 and 68; 36 and 69; 36 and 70; 36 and 71; 36 and 72; 36 and 73; 36 and 74; 36 and 75; 36 and 76; 36 and 77; 36 and 78; 36 and 79; 36 and 80; 36 and 81; 36 and 82; 36 and 83; 36 and 84; 36 and 85; 36 and 86; 36 and 87; 36 and 88; 36 and 89; 36 and 90; 36 and 91; 36 and 92; 36 and 93; 36 and 94; 36 and 95; 36 and 96; 36 and 97; 36 and 98; 36 and 99; 36 and 100; 36 and 101; 36 and 102; 36 and 103; 36 and 104; 36 and 105; 36 and 106; 36 and 107; 36 and 108; 36 and 109; 36 and 110; 36 and 111; 36 and 112; 36 and 113; 36 and 114; 36 and 115; 36 and 116; 36 and 117; 36 and 118; 36 and 119; 36 and 120; 36 and 121; 36 and 122; 36 and 123; 36 and 124; 36 and 125; 36 and 126; 36 and 127; 36 and 128; 36 and 129; 36 and 130; 36 and 131; 36 and 132; 36 and 133; 36 and 134; 36 and 135; 36 and 136; 36 and 137; 36 and 138; 36 and 139; 36 and 140; 36 and 141; 36 and 142; 36 and 143; 36 and 144; 36 and 145; 36 and 146; 36 and 147; 36 and 148; 36 and 149; 36 and 150; 36 and 151; 36 and 152; 36 and 153; 36 and 154; 36 and 155; 36 and 156; 36 and 157; 36 and 158; 36 and 159; 36 and 160; 36 and 161; 36 and 162; 36 and 163; 36 and 164; 36 and 165; 36 and 166; 36 and 167; 37 and 67; 37 and 68; 37 and 69; 37 and 70; 37 and 71; 37 and 72; 37 and 73; 37 and 74; 37 and 75; 37 and 76; 37 and 77; 37 and 78; 37 and 79; 37 and 80; 37 and 81; 37 and 82; 37 and 83; 37 and 84; 37 and 85; 37 and 86; 37 and 87; 37 and 88; 37 and 89; 37 and 90; 37 and 91; 37 and 92; 37 and 93; 37 and 94; 37 and 95; 37 and 96; 37 and 97; 37 and 98; 37 and 99; 37 and 100; 37 and 101; 37 and 102; 37 and 103; 37 and 104; 37 and 105; 37 and 106; 37 and 107; 37 and 108; 37 and 109; 37 and 110; 37 and 111; 37 and 112; 37 and 113; 37 and 114; 37 and 115; 37 and 116; 37 and 117; 37 and 118; 37 and 119; 37 and 120; 37 and 121; 37 and 122; 37 and 123; 37 and 124; 37 and 125; 37 and 126; 37 and 127; 37 and 128; 37 and 129; 37 and 130; 37 and 131; 37 and 132; 37 and 133; 37 and 134; 37 and 135; 37 and 136; 37 and 137; 37 and 138; 37 and 139; 37 and 140; 37 and 141; 37 and 142; 37 and 143; 37 and 144; 37 and 145; 37 and 146; 37 and 147; 37 and 148; 37 and 149; 37 and 150; 37 and 151; 37 and 152; 37 and 153; 37 and 154; 37 and 155; 37 and 156; 37 and 157; 37 and 158; 37 and 159; 37 and 160; 37 and 161; 37 and 162; 37 and 163; 37 and 164; 37 and 165; 37 and 166; 37 and 167; 38 and 67; 38 and 68; 38 and 69; 38 and 70; 38 and 71; 38 and 72; 38 and 73; 38 and 74; 38 and 75; 38 and 76; 38 and 77; 38 and 78; 38 and 79; 38 and 80; 38 and 81; 38 and 82; 38 and 83; 38 and 84; 38 and 85; 38 and 86; 38 and 87; 38 and 88; 38 and 89; 38 and 90; 38 and 91; 38 and 92; 38 and 93; 38 and 94; 38 and 95; 38 and 96; 38 and 97; 38 and 98; 38 and 99; 38 and 100; 38 and 101; 38 and 102; 38 and 103; 38 and 104; 38 and 105; 38 and 106; 38 and 107; 38 and 108; 38 and 109; 38 and 110; 38 and 111; 38 and 112; 38 and 113; 38 and 114; 38 and 115; 38 and 116; 38 and 117; 38 and 118; 38 and 119; 38 and 120; 38 and 121; 38 and 122; 38 and 123; 38 and 124; 38 and 125; 38 and 126; 38 and 127; 38 and 128; 38 and 129; 38 and 130; 38 and 131; 38 and 132; 38 and 133; 38 and 134; 38 and 135; 38 and 136; 38 and 137; 38 and 138; 38 and 139; 38 and 140; 38 and 141; 38 and 142; 38 and 143; 38 and 144; 38 and 145; 38 and 146; 38 and 147; 38 and 148; 38 and 149; 38 and 150; 38 and 151; 38 and 152; 38 and 153; 38 and 154; 38 and 155; 38 and 156; 38 and 157; 38 and 158; 38 and 159; 38 and 160; 38 and 161; 38 and 162; 38 and 163; 38 and 164; 38 and 165; 38 and 166; 38 and 167; 39 and 67; 39 and 68; 39 and 69; 39 and 70; 39 and 71; 39 and 72; 39 and 73; 39 and 74; 39 and 75; 39 and 76; 39 and 77; 39 and 78; 39 and 79; 39 and 80; 39 and 81; 39 and 82; 39 and 83; 39 and 84; 39 and 85; 39 and 86; 39 and 87; 39 and 88; 39 and 89; 39 and 90; 39 and 91; 39 and 92; 39 and 93; 39 and 94; 39 and 95; 39 and 96; 39 and 97; 39 and 98; 39 and 99; 39 and 100; 39 and 101; 39 and 102; 39 and 103; 39 and 104; 39 and 105; 39 and 106; 39 and 107; 39 and 108; 39 and 109; 39 and 110; 39 and 111; 39 and 112; 39 and 113; 39 and 114; 39 and 115; 39 and 116; 39 and 117; 39 and 118; 39 and 119; 39 and 120; 39 and 121; 39 and 122; 39 and 123; 39 and 124; 39 and 125; 39 and 126; 39 and 127; 39 and 128; 39 and 129; 39 and 130; 39 and 131; 39 and 132; 39 and 133; 39 and 134; 39 and 135; 39 and 136; 39 and 137; 39 and 138; 39 and 139; 39 and 140; 39 and 141; 39 and 142; 39 and 143; 39 and 144; 39 and 145; 39 and 146; 39 and 147; 39 and 148; 39 and 149; 39 and 150; 39 and 151; 39 and 152; 39 and 153; 39 and 154; 39 and 155; 39 and 156; 39 and 157; 39 and 158; 39 and 159; 39 and 160; 39 and 161; 39 and 162; 39 and 163; 39 and 164; 39 and 165; 39 and 166; 39 and 167; 40 and 67; 40 and 68; 40 and 69; 40 and 70; 40 and 71; 40 and 72; 40 and 73; 40 and 74; 40 and 75; 40 and 76; 40 and 77; 40 and 78; 40 and 79; 40 and 80; 40 and 81; 40 and 82; 40 and 83; 40 and 84; 40 and 85; 40 and 86; 40 and 87; 40 and 88; 40 and 89; 40 and 90; 40 and 91; 40 and 92; 40 and 93; 40 and 94; 40 and 95; 40 and 96; 40 and 97; 40 and 98; 40 and 99; 40 and 100; 40 and 101; 40 and 102; 40 and 103; 40 and 104; 40 and 105; 40 and 106; 40 and 107; 40 and 108; 40 and 109; 40 and 110; 40 and 111; 40 and 112; 40 and 113; 40 and 114; 40 and 115; 40 and 116; 40 and 117; 40 and 118; 40 and 119; 40 and 120; 40 and 121; 40 and 122; 40 and 123; 40 and 124; 40 and 125; 40 and 126; 40 and 127; 40 and 128; 40 and 129; 40 and 130; 40 and 131; 40 and 132; 40 and 133; 40 and 134; 40 and 135; 40 and 136; 40 and 137; 40 and 138; 40 and 139; 40 and 140; 40 and 141; 40 and 142; 40 and 143; 40 and 144; 40 and 145; 40 and 146; 40 and 147; 40 and 148; 40 and 149; 40 and 150; 40 and 151; 40 and 152; 40 and 153; 40 and 154; 40 and 155; 40 and 156; 40 and 157; 40 and 158; 40 and 159; 40 and 160; 40 and 161; 40 and 162; 40 and 163; 40 and 164; 40 and 165; 40 and 166; 40 and 167; 41 and 67; 41 and 68; 41 and 69; 41 and 70; 41 and 71; 41 and 72; 41 and 73; 41 and 74; 41 and 75; 41 and 76; 41 and 77; 41 and 78; 41 and 79; 41 and 80; 41 and 81; 41 and 82; 41 and 83; 41 and 84; 41 and 85; 41 and 86; 41 and 87; 41 and 88; 41 and 89; 41 and 90; 41 and 91; 41 and 92; 41 and 93; 41 and 94; 41 and 95; 41 and 96; 41 and 97; 41 and 98; 41 and 99; 41 and 100; 41 and 101; 41 and 102; 41 and 103; 41 and 104; 41 and 105; 41 and 106; 41 and 107; 41 and 108; 41 and 109; 41 and 110; 41 and 111; 41 and 112; 41 and 113; 41 and 114; 41 and 115; 41 and 116; 41 and 117; 41 and 118; 41 and 119; 41 and 120; 41 and 121; 41 and 122; 41 and 123; 41 and 124; 41 and 125; 41 and 126; 41 and 127; 41 and 128; 41 and 129; 41 and 130; 41 and 131; 41 and 132; 41 and 133; 41 and 134; 41 and 135; 41 and 136; 41 and 137; 41 and 138; 41 and 139; 41 and 140; 41 and 141; 41 and 142; 41 and 143; 41 and 144; 41 and 145; 41 and 146; 41 and 147; 41 and 148; 41 and 149; 41 and 150; 41 and 151; 41 and 152; 41 and 153; 41 and 154; 41 and 155; 41 and 156; 41 and 157; 41 and 158; 41 and 159; 41 and 160; 41 and 161; 41 and 162; 41 and 163; 41 and 164; 41 and 165; 41 and 166; 41 and 167; 42 and 67; 42 and 68; 42 and 69; 42 and 70; 42 and 71; 42 and 72; 42 and 73; 42 and 74; 42 and 75; 42 and 76; 42 and 77; 42 and 78; 42 and 79; 42 and 80; 42 and 81; 42 and 82; 42 and 83; 42 and 84; 42 and 85; 42 and 86; 42 and 87; 42 and 88; 42 and 89; 42 and 90; 42 and 91; 42 and 92; 42 and 93; 42 and 94; 42 and 95; 42 and 96; 42 and 97; 42 and 98; 42 and 99; 42 and 100; 42 and 101; 42 and 102; 42 and 103; 42 and 104; 42 and 105; 42 and 106; 42 and 107; 42 and 108; 42 and 109; 42 and 110; 42 and 111; 42 and 112; 42 and 113; 42 and 114; 42 and 115; 42 and 116; 42 and 117; 42 and 118; 42 and 119; 42 and 120; 42 and 121; 42 and 122; 42 and 123; 42 and 124; 42 and 125; 42 and 126; 42 and 127; 42 and 128; 42 and 129; 42 and 130; 42 and 131; 42 and 132; 42 and 133; 42 and 134; 42 and 135; 42 and 136; 42 and 137; 42 and 138; 42 and 139; 42 and 140; 42 and 141; 42 and 142; 42 and 143; 42 and 144; 42 and 145; 42 and 146; 42 and 147; 42 and 148; 42 and 149; 42 and 150; 42 and 151; 42 and 152; 42 and 153; 42 and 154; 42 and 155; 42 and 156; 42 and 157; 42 and 158; 42 and 159; 42 and 160; 42 and 161; 42 and 162; 42 and 163; 42 and 164; 42 and 165; 42 and 166; 42 and 167; 43 and 67; 43 and 68; 43 and 69; 43 and 70; 43 and 71; 43 and 72; 43 and 73; 43 and 74; 43 and 75; 43 and 76; 43 and 77; 43 and 78; 43 and 79; 43 and 80; 43 and 81; 43 and 82; 43 and 83; 43 and 84; 43 and 85; 43 and 86; 43 and 87; 43 and 88; 43 and 89; 43 and 90; 43 and 91; 43 and 92; 43 and 93; 43 and 94; 43 and 95; 43 and 96; 43 and 97; 43 and 98; 43 and 99; 43 and 100; 43 and 101; 43 and 102; 43 and 103; 43 and 104; 43 and 105; 43 and 106; 43 and 107; 43 and 108; 43 and 109; 43 and 110; 43 and 111; 43 and 112; 43 and 113; 43 and 114; 43 and 115; 43 and 116; 43 and 117; 43 and 118; 43 and 119; 43 and 120; 43 and 121; 43 and 122; 43 and 123; 43 and 124; 43 and 125; 43 and 126; 43 and 127; 43 and 128; 43 and 129; 43 and 130; 43 and 131; 43 and 132; 43 and 133; 43 and 134; 43 and 135; 43 and 136; 43 and 137; 43 and 138; 43 and 139; 43 and 140; 43 and 141; 43 and 142; 43 and 143; 43 and 144; 43 and 145; 43 and 146; 43 and 147; 43 and 148; 43 and 149; 43 and 150; 43 and 151; 43 and 152; 43 and 153; 43 and 154; 43 and 155; 43 and 156; 43 and 157; 43 and 158; 43 and 159; 43 and 160; 43 and 161; 43 and 162; 43 and 163; 43 and 164; 43 and 165; 43 and 166; 43 and 167; 44 and 67; 44 and 68; 44 and 69; 44 and 70; 44 and 71; 44 and 72; 44 and 73; 44 and 74; 44 and 75; 44 and 76; 44 and 77; 44 and 78; 44 and 79; 44 and 80; 44 and 81; 44 and 82; 44 and 83; 44 and 84; 44 and 85; 44 and 86; 44 and 87; 44 and 88; 44 and 89; 44 and 90; 44 and 91; 44 and 92; 44 and 93; 44 and 94; 44 and 95; 44 and 96; 44 and 97; 44 and 98; 44 and 99; 44 and 100; 44 and 101; 44 and 102; 44 and 103; 44 and 104; 44 and 105; 44 and 106; 44 and 107; 44 and 108; 44 and 109; 44 and 110; 44 and 111; 44 and 112; 44 and 113; 44 and 114; 44 and 115; 44 and 116; 44 and 117; 44 and 118; 44 and 119; 44 and 120; 44 and 121; 44 and 122; 44 and 123; 44 and 124; 44 and 125; 44 and 126; 44 and 127; 44 and 128; 44 and 129; 44 and 130; 44 and 131; 44 and 132; 44 and 133; 44 and 134; 44 and 135; 44 and 136; 44 and 137; 44 and 138; 44 and 139; 44 and 140; 44 and 141; 44 and 142; 44 and 143; 44 and 144; 44 and 145; 44 and 146; 44 and 147; 44 and 148; 44 and 149; 44 and 150; 44 and 151; 44 and 152; 44 and 153; 44 and 154; 44 and 155; 44 and 156; 44 and 157; 44 and 158; 44 and 159; 44 and 160; 44 and 161; 44 and 162; 44 and 163; 44 and 164; 44 and 165; 44 and 166; 44 and 167; 45 and 67; 45 and 68; 45 and 69; 45 and 70; 45 and 71; 45 and 72; 45 and 73; 45 and 74; 45 and 75; 45 and 76; 45 and 77; 45 and 78; 45 and 79; 45 and 80; 45 and 81; 45 and 82; 45 and 83; 45 and 84; 45 and 85; 45 and 86; 45 and 87; 45 and 88; 45 and 89; 45 and 90; 45 and 91; 45 and 92; 45 and 93; 45 and 94; 45 and 95; 45 and 96; 45 and 97; 45 and 98; 45 and 99; 45 and 100; 45 and 101; 45 and 102; 45 and 103; 45 and 104; 45 and 105; 45 and 106; 45 and 107; 45 and 108; 45 and 109; 45 and 110; 45 and 111; 45 and 112; 45 and 113; 45 and 114; 45 and 115; 45 and 116; 45 and 117; 45 and 118; 45 and 119; 45 and 120; 45 and 121; 45 and 122; 45 and 123; 45 and 124; 45 and 125; 45 and 126; 45 and 127; 45 and 128; 45 and 129; 45 and 130; 45 and 131; 45 and 132; 45 and 133; 45 and 134; 45 and 135; 45 and 136; 45 and 137; 45 and 138; 45 and 139; 45 and 140; 45 and 141; 45 and 142; 45 and 143; 45 and 144; 45 and 145; 45 and 146; 45 and 147; 45 and 148; 45 and 149; 45 and 150; 45 and 151; 45 and 152; 45 and 153; 45 and 154; 45 and 155; 45 and 156; 45 and 157; 45 and 158; 45 and 159; 45 and 160; 45 and 161; 45 and 162; 45 and 163; 45 and 164; 45 and 165; 45 and 166; 45 and 167; 46 and 67; 46 and 68; 46 and 69; 46 and 70; 46 and 71; 46 and 72; 46 and 73; 46 and 74; 46 and 75; 46 and 76; 46 and 77; 46 and 78; 46 and 79; 46 and 80; 46 and 81; 46 and 82; 46 and 83; 46 and 84; 46 and 85; 46 and 86; 46 and 87; 46 and 88; 46 and 89; 46 and 90; 46 and 91; 46 and 92; 46 and 93; 46 and 94; 46 and 95; 46 and 96; 46 and 97; 46 and 98; 46 and 99; 46 and 100; 46 and 101; 46 and 102; 46 and 103; 46 and 104; 46 and 105; 46 and 106; 46 and 107; 46 and 108; 46 and 109; 46 and 110; 46 and 111; 46 and 112; 46 and 113; 46 and 114; 46 and 115; 46 and 116; 46 and 117; 46 and 118; 46 and 119; 46 and 120; 46 and 121; 46 and 122; 46 and 123; 46 and 124; 46 and 125; 46 and 126; 46 and 127; 46 and 128; 46 and 129; 46 and 130; 46 and 131; 46 and 132; 46 and 133; 46 and 134; 46 and 135; 46 and 136; 46 and 137; 46 and 138; 46 and 139; 46 and 140; 46 and 141; 46 and 142; 46 and 143; 46 and 144; 46 and 145; 46 and 146; 46 and 147; 46 and 148; 46 and 149; 46 and 150; 46 and 151; 46 and 152; 46 and 153; 46 and 154; 46 and 155; 46 and 156; 46 and 157; 46 and 158; 46 and 159; 46 and 160; 46 and 161; 46 and 162; 46 and 163; 46 and 164; 46 and 165; 46 and 166; 46 and 167; 47 and 67; 47 and 68; 47 and 69; 47 and 70; 47 and 71; 47 and 72; 47 and 73; 47 and 74; 47 and 75; 47 and 76; 47 and 77; 47 and 78; 47 and 79; 47 and 80; 47 and 81; 47 and 82; 47 and 83; 47 and 84; 47 and 85; 47 and 86; 47 and 87; 47 and 88; 47 and 89; 47 and 90; 47 and 91; 47 and 92; 47 and 93; 47 and 94; 47 and 95; 47 and 96; 47 and 97; 47 and 98; 47 and 99; 47 and 100; 47 and 101; 47 and 102; 47 and 103; 47 and 104; 47 and 105; 47 and 106; 47 and 107; 47 and 108; 47 and 109; 47 and 110; 47 and 111; 47 and 112; 47 and 113; 47 and 114; 47 and 115; 47 and 116; 47 and 117; 47 and 118; 47 and 119; 47 and 120; 47 and 121; 47 and 122; 47 and 123; 47 and 124; 47 and 125; 47 and 126; 47 and 127; 47 and 128; 47 and 129; 47 and 130; 47 and 131; 47 and 132; 47 and 133; 47 and 134; 47 and 135; 47 and 136; 47 and 137; 47 and 138; 47 and 139; 47 and 140; 47 and 141; 47 and 142; 47 and 143; 47 and 144; 47 and 145; 47 and 146; 47 and 147; 47 and 148; 47 and 149; 47 and 150; 47 and 151; 47 and 152; 47 and 153; 47 and 154; 47 and 155; 47 and 156; 47 and 157; 47 and 158; 47 and 159; 47 and 160; 47 and 161; 47 and 162; 47 and 163; 47 and 164; 47 and 165; 47 and 166; 47 and 167; 48 and 67; 48 and 68; 48 and 69; 48 and 70; 48 and 71; 48 and 72; 48 and 73; 48 and 74; 48 and 75; 48 and 76; 48 and 77; 48 and 78; 48 and 79; 48 and 80; 48 and 81; 48 and 82; 48 and 83; 48 and 84; 48 and 85; 48 and 86; 48 and 87; 48 and 88; 48 and 89; 48 and 90; 48 and 91; 48 and 92; 48 and 93; 48 and 94; 48 and 95; 48 and 96; 48 and 97; 48 and 98; 48 and 99; 48 and 100; 48 and 101; 48 and 102; 48 and 103; 48 and 104; 48 and 105; 48 and 106; 48 and 107; 48 and 108; 48 and 109; 48 and 110; 48 and 111; 48 and 112; 48 and 113; 48 and 114; 48 and 115; 48 and 116; 48 and 117; 48 and 118; 48 and 119; 48 and 120; 48 and 121; 48 and 122; 48 and 123; 48 and 124; 48 and 125; 48 and 126; 48 and 127; 48 and 128; 48 and 129; 48 and 130; 48 and 131; 48 and 132; 48 and 133; 48 and 134; 48 and 135; 48 and 136; 48 and 137; 48 and 138; 48 and 139; 48 and 140; 48 and 141; 48 and 142; 48 and 143; 48 and 144; 48 and 145; 48 and 146; 48 and 147; 48 and 148; 48 and 149; 48 and 150; 48 and 151; 48 and 152; 48 and 153; 48 and 154; 48 and 155; 48 and 156; 48 and 157; 48 and 158; 48 and 159; 48 and 160; 48 and 161; 48 and 162; 48 and 163; 48 and 164; 48 and 165; 48 and 166; 48 and 167; 49 and 67; 49 and 68; 49 and 69; 49 and 70; 49 and 71; 49 and 72; 49 and 73; 49 and 74; 49 and 75; 49 and 76; 49 and 77; 49 and 78; 49 and 79; 49 and 80; 49 and 81; 49 and 82; 49 and 83; 49 and 84; 49 and 85; 49 and 86; 49 and 87; 49 and 88; 49 and 89; 49 and 90; 49 and 91; 49 and 92; 49 and 93; 49 and 94; 49 and 95; 49 and 96; 49 and 97; 49 and 98; 49 and 99; 49 and 100; 49 and 101; 49 and 102; 49 and 103; 49 and 104; 49 and 105; 49 and 106; 49 and 107; 49 and 108; 49 and 109; 49 and 110; 49 and 111; 49 and 112; 49 and 113; 49 and 114; 49 and 115; 49 and 116; 49 and 117; 49 and 118; 49 and 119; 49 and 120; 49 and 121; 49 and 122; 49 and 123; 49 and 124; 49 and 125; 49 and 126; 49 and 127; 49 and 128; 49 and 129; 49 and 130; 49 and 131; 49 and 132; 49 and 133; 49 and 134; 49 and 135; 49 and 136; 49 and 137; 49 and 138; 49 and 139; 49 and 140; 49 and 141; 49 and 142; 49 and 143; 49 and 144; 49 and 145; 49 and 146; 49 and 147; 49 and 148; 49 and 149; 49 and 150; 49 and 151; 49 and 152; 49 and 153; 49 and 154; 49 and 155; 49 and 156; 49 and 157; 49 and 158; 49 and 159; 49 and 160; 49 and 161; 49 and 162; 49 and 163; 49 and 164; 49 and 165; 49 and 166; 49 and 167; 50 and 67; 50 and 68; 50 and 69; 50 and 70; 50 and 71; 50 and 72; 50 and 73; 50 and 74; 50 and 75; 50 and 76; 50 and 77; 50 and 78; 50 and 79; 50 and 80; 50 and 81; 50 and 82; 50 and 83; 50 and 84; 50 and 85; 50 and 86; 50 and 87; 50 and 88; 50 and 89; 50 and 90; 50 and 91; 50 and 92; 50 and 93; 50 and 94; 50 and 95; 50 and 96; 50 and 97; 50 and 98; 50 and 99; 50 and 100; 50 and 101; 50 and 102; 50 and 103; 50 and 104; 50 and 105; 50 and 106; 50 and 107; 50 and 108; 50 and 109; 50 and 110; 50 and 111; 50 and 112; 50 and 113; 50 and 114; 50 and 115; 50 and 116; 50 and 117; 50 and 118; 50 and 119; 50 and 120; 50 and 121; 50 and 122; 50 and 123; 50 and 124; 50 and 125; 50 and 126; 50 and 127; 50 and 128; 50 and 129; 50 and 130; 50 and 131; 50 and 132; 50 and 133; 50 and 134; 50 and 135; 50 and 136; 50 and 137; 50 and 138; 50 and 139; 50 and 140; 50 and 141; 50 and 142; 50 and 143; 50 and 144; 50 and 145; 50 and 146; 50 and 147; 50 and 148; 50 and 149; 50 and 150; 50 and 151; 50 and 152; 50 and 153; 50 and 154; 50 and 155; 50 and 156; 50 and 157; 50 and 158; 50 and 159; 50 and 160; 50 and 161; 50 and 162; 50 and 163; 50 and 164; 50 and 165; 50 and 166; 50 and 167; 51 and 67; 51 and 68; 51 and 69; 51 and 70; 51 and 71; 51 and 72; 51 and 73; 51 and 74; 51 and 75; 51 and 76; 51 and 77; 51 and 78; 51 and 79; 51 and 80; 51 and 81; 51 and 82; 51 and 83; 51 and 84; 51 and 85; 51 and 86; 51 and 87; 51 and 88; 51 and 89; 51 and 90; 51 and 91; 51 and 92; 51 and 93; 51 and 94; 51 and 95; 51 and 96; 51 and 97; 51 and 98; 51 and 99; 51 and 100; 51 and 101; 51 and 102; 51 and 103; 51 and 104; 51 and 105; 51 and 106; 51 and 107; 51 and 108; 51 and 109; 51 and 110; 51 and 111; 51 and 112; 51 and 113; 51 and 114; 51 and 115; 51 and 116; 51 and 117; 51 and 118; 51 and 119; 51 and 120; 51 and 121; 51 and 122; 51 and 123; 51 and 124; 51 and 125; 51 and 126; 51 and 127; 51 and 128; 51 and 129; 51 and 130; 51 and 131; 51 and 132; 51 and 133; 51 and 134; 51 and 135; 51 and 136; 51 and 137; 51 and 138; 51 and 139; 51 and 140; 51 and 141; 51 and 142; 51 and 143; 51 and 144; 51 and 145; 51 and 146; 51 and 147; 51 and 148; 51 and 149; 51 and 150; 51 and 151; 51 and 152; 51 and 153; 51 and 154; 51 and 155; 51 and 156; 51 and 157; 51 and 158; 51 and 159; 51 and 160; 51 and 161; 51 and 162; 51 and 163; 51 and 164; 51 and 165; 51 and 166; 51 and 167; 52 and 67; 52 and 68; 52 and 69; 52 and 70; 52 and 71; 52 and 72; 52 and 73; 52 and 74; 52 and 75; 52 and 76; 52 and 77; 52 and 78; 52 and 79; 52 and 80; 52 and 81; 52 and 82; 52 and 83; 52 and 84; 52 and 85; 52 and 86; 52 and 87; 52 and 88; 52 and 89; 52 and 90; 52 and 91; 52 and 92; 52 and 93; 52 and 94; 52 and 95; 52 and 96; 52 and 97; 52 and 98; 52 and 99; 52 and 100; 52 and 101; 52 and 102; 52 and 103; 52 and 104; 52 and 105; 52 and 106; 52 and 107; 52 and 108; 52 and 109; 52 and 110; 52 and 111; 52 and 112; 52 and 113; 52 and 114; 52 and 115; 52 and 116; 52 and 117; 52 and 118; 52 and 119; 52 and 120; 52 and 121; 52 and 122; 52 and 123; 52 and 124; 52 and 125; 52 and 126; 52 and 127; 52 and 128; 52 and 129; 52 and 130; 52 and 131; 52 and 132; 52 and 133; 52 and 134; 52 and 135; 52 and 136; 52 and 137; 52 and 138; 52 and 139; 52 and 140; 52 and 141; 52 and 142; 52 and 143; 52 and 144; 52 and 145; 52 and 146; 52 and 147; 52 and 148; 52 and 149; 52 and 150; 52 and 151; 52 and 152; 52 and 153; 52 and 154; 52 and 155; 52 and 156; 52 and 157; 52 and 158; 52 and 159; 52 and 160; 52 and 161; 52 and 162; 52 and 163; 52 and 164; 52 and 165; 52 and 166; 52 and 167; 53 and 67; 53 and 68; 53 and 69; 53 and 70; 53 and 71; 53 and 72; 53 and 73; 53 and 74; 53 and 75; 53 and 76; 53 and 77; 53 and 78; 53 and 79; 53 and 80; 53 and 81; 53 and 82; 53 and 83; 53 and 84; 53 and 85; 53 and 86; 53 and 87; 53 and 88; 53 and 89; 53 and 90; 53 and 91; 53 and 92; 53 and 93; 53 and 94; 53 and 95; 53 and 96; 53 and 97; 53 and 98; 53 and 99; 53 and 100; 53 and 101; 53 and 102; 53 and 103; 53 and 104; 53 and 105; 53 and 106; 53 and 107; 53 and 108; 53 and 109; 53 and 110; 53 and 111; 53 and 112; 53 and 113; 53 and 114; 53 and 115; 53 and 116; 53 and 117; 53 and 118; 53 and 119; 53 and 120; 53 and 121; 53 and 122; 53 and 123; 53 and 124; 53 and 125; 53 and 126; 53 and 127; 53 and 128; 53 and 129; 53 and 130; 53 and 131; 53 and 132; 53 and 133; 53 and 134; 53 and 135; 53 and 136; 53 and 137; 53 and 138; 53 and 139; 53 and 140; 53 and 141; 53 and 142; 53 and 143; 53 and 144; 53 and 145; 53 and 146; 53 and 147; 53 and 148; 53 and 149; 53 and 150; 53 and 151; 53 and 152; 53 and 153; 53 and 154; 53 and 155; 53 and 156; 53 and 157; 53 and 158; 53 and 159; 53 and 160; 53 and 161; 53 and 162; 53 and 163; 53 and 164; 53 and 165; 53 and 166; 53 and 167; 54 and 67; 54 and 68; 54 and 69; 54 and 70; 54 and 71; 54 and 72; 54 and 73; 54 and 74; 54 and 75; 54 and 76; 54 and 77; 54 and 78; 54 and 79; 54 and 80; 54 and 81; 54 and 82; 54 and 83; 54 and 84; 54 and 85; 54 and 86; 54 and 87; 54 and 88; 54 and 89; 54 and 90; 54 and 91; 54 and 92; 54 and 93; 54 and 94; 54 and 95; 54 and 96; 54 and 97; 54 and 98; 54 and 99; 54 and 100; 54 and 101; 54 and 102; 54 and 103; 54 and 104; 54 and 105; 54 and 106; 54 and 107; 54 and 108; 54 and 109; 54 and 110; 54 and 111; 54 and 112; 54 and 113; 54 and 114; 54 and 115; 54 and 116; 54 and 117; 54 and 118; 54 and 119; 54 and 120; 54 and 121; 54 and 122; 54 and 123; 54 and 124; 54 and 125; 54 and 126; 54 and 127; 54 and 128; 54 and 129; 54 and 130; 54 and 131; 54 and 132; 54 and 133; 54 and 134; 54 and 135; 54 and 136; 54 and 137; 54 and 138; 54 and 139; 54 and 140; 54 and 141; 54 and 142; 54 and 143; 54 and 144; 54 and 145; 54 and 146; 54 and 147; 54 and 148; 54 and 149; 54 and 150; 54 and 151; 54 and 152; 54 and 153; 54 and 154; 54 and 155; 54 and 156; 54 and 157; 54 and 158; 54 and 159; 54 and 160; 54 and 161; 54 and 162; 54 and 163; 54 and 164; 54 and 165; 54 and 166; 54 and 167; 55 and 67; 55 and 68; 55 and 69; 55 and 70; 55 and 71; 55 and 72; 55 and 73; 55 and 74; 55 and 75; 55 and 76; 55 and 77; 55 and 78; 55 and 79; 55 and 80; 55 and 81; 55 and 82; 55 and 83; 55 and 84; 55 and 85; 55 and 86; 55 and 87; 55 and 88; 55 and 89; 55 and 90; 55 and 91; 55 and 92; 55 and 93; 55 and 94; 55 and 95; 55 and 96; 55 and 97; 55 and 98; 55 and 99; 55 and 100; 55 and 101; 55 and 102; 55 and 103; 55 and 104; 55 and 105; 55 and 106; 55 and 107; 55 and 108; 55 and 109; 55 and 110; 55 and 111; 55 and 112; 55 and 113; 55 and 114; 55 and 115; 55 and 116; 55 and 117; 55 and 118; 55 and 119; 55 and 120; 55 and 121; 55 and 122; 55 and 123; 55 and 124; 55 and 125; 55 and 126; 55 and 127; 55 and 128; 55 and 129; 55 and 130; 55 and 131; 55 and 132; 55 and 133; 55 and 134; 55 and 135; 55 and 136; 55 and 137; 55 and 138; 55 and 139; 55 and 140; 55 and 141; 55 and 142; 55 and 143; 55 and 144; 55 and 145; 55 and 146; 55 and 147; 55 and 148; 55 and 149; 55 and 150; 55 and 151; 55 and 152; 55 and 153; 55 and 154; 55 and 155; 55 and 156; 55 and 157; 55 and 158; 55 and 159; 55 and 160; 55 and 161; 55 and 162; 55 and 163; 55 and 164; 55 and 165; 55 and 166; 55 and 167; 56 and 67; 56 and 68; 56 and 69; 56 and 70; 56 and 71; 56 and 72; 56 and 73; 56 and 74; 56 and 75; 56 and 76; 56 and 77; 56 and 78; 56 and 79; 56 and 80; 56 and 81; 56 and 82; 56 and 83; 56 and 84; 56 and 85; 56 and 86; 56 and 87; 56 and 88; 56 and 89; 56 and 90; 56 and 91; 56 and 92; 56 and 93; 56 and 94; 56 and 95; 56 and 96; 56 and 97; 56 and 98; 56 and 99; 56 and 100; 56 and 101; 56 and 102; 56 and 103; 56 and 104; 56 and 105; 56 and 106; 56 and 107; 56 and 108; 56 and 109; 56 and 110; 56 and 111; 56 and 112; 56 and 113; 56 and 114; 56 and 115; 56 and 116; 56 and 117; 56 and 118; 56 and 119; 56 and 120; 56 and 121; 56 and 122; 56 and 123; 56 and 124; 56 and 125; 56 and 126; 56 and 127; 56 and 128; 56 and 129; 56 and 130; 56 and 131; 56 and 132; 56 and 133; 56 and 134; 56 and 135; 56 and 136; 56 and 137; 56 and 138; 56 and 139; 56 and 140; 56 and 141; 56 and 142; 56 and 143; 56 and 144; 56 and 145; 56 and 146; 56 and 147; 56 and 148; 56 and 149; 56 and 150; 56 and 151; 56 and 152; 56 and 153; 56 and 154; 56 and 155; 56 and 156; 56 and 157; 56 and 158; 56 and 159; 56 and 160; 56 and 161; 56 and 162; 56 and 163; 56 and 164; 56 and 165; 56 and 166; 56 and 167; 57 and 67; 57 and 68; 57 and 69; 57 and 70; 57 and 71; 57 and 72; 57 and 73; 57 and 74; 57 and 75; 57 and 76; 57 and 77; 57 and 78; 57 and 79; 57 and 80; 57 and 81; 57 and 82; 57 and 83; 57 and 84; 57 and 85; 57 and 86; 57 and 87; 57 and 88; 57 and 89; 57 and 90; 57 and 91; 57 and 92; 57 and 93; 57 and 94; 57 and 95; 57 and 96; 57 and 97; 57 and 98; 57 and 99; 57 and 100; 57 and 101; 57 and 102; 57 and 103; 57 and 104; 57 and 105; 57 and 106; 57 and 107; 57 and 108; 57 and 109; 57 and 110; 57 and 111; 57 and 112; 57 and 113; 57 and 114; 57 and 115; 57 and 116; 57 and 117; 57 and 118; 57 and 119; 57 and 120; 57 and 121; 57 and 122; 57 and 123; 57 and 124; 57 and 125; 57 and 126; 57 and 127; 57 and 128; 57 and 129; 57 and 130; 57 and 131; 57 and 132; 57 and 133; 57 and 134; 57 and 135; 57 and 136; 57 and 137; 57 and 138; 57 and 139; 57 and 140; 57 and 141; 57 and 142; 57 and 143; 57 and 144; 57 and 145; 57 and 146; 57 and 147; 57 and 148; 57 and 149; 57 and 150; 57 and 151; 57 and 152; 57 and 153; 57 and 154; 57 and 155; 57 and 156; 57 and 157; 57 and 158; 57 and 159; 57 and 160; 57 and 161; 57 and 162; 57 and 163; 57 and 164; 57 and 165; 57 and 166; 57 and 167; 58 and 67; 58 and 68; 58 and 69; 58 and 70; 58 and 71; 58 and 72; 58 and 73; 58 and 74; 58 and 75; 58 and 76; 58 and 77; 58 and 78; 58 and 79; 58 and 80; 58 and 81; 58 and 82; 58 and 83; 58 and 84; 58 and 85; 58 and 86; 58 and 87; 58 and 88; 58 and 89; 58 and 90; 58 and 91; 58 and 92; 58 and 93; 58 and 94; 58 and 95; 58 and 96; 58 and 97; 58 and 98; 58 and 99; 58 and 100; 58 and 101; 58 and 102; 58 and 103; 58 and 104; 58 and 105; 58 and 106; 58 and 107; 58 and 108; 58 and 109; 58 and 110; 58 and 111; 58 and 112; 58 and 113; 58 and 114; 58 and 115; 58 and 116; 58 and 117; 58 and 118; 58 and 119; 58 and 120; 58 and 121; 58 and 122; 58 and 123; 58 and 124; 58 and 125; 58 and 126; 58 and 127; 58 and 128; 58 and 129; 58 and 130; 58 and 131; 58 and 132; 58 and 133; 58 and 134; 58 and 135; 58 and 136; 58 and 137; 58 and 138; 58 and 139; 58 and 140; 58 and 141; 58 and 142; 58 and 143; 58 and 144; 58 and 145; 58 and 146; 58 and 147; 58 and 148; 58 and 149; 58 and 150; 58 and 151; 58 and 152; 58 and 153; 58 and 154; 58 and 155; 58 and 156; 58 and 157; 58 and 158; 58 and 159; 58 and 160; 58 and 161; 58 and 162; 58 and 163; 58 and 164; 58 and 165; 58 and 166; 58 and 167; 59 and 67; 59 and 68; 59 and 69; 59 and 70; 59 and 71; 59 and 72; 59 and 73; 59 and 74; 59 and 75; 59 and 76; 59 and 77; 59 and 78; 59 and 79; 59 and 80; 59 and 81; 59 and 82; 59 and 83; 59 and 84; 59 and 85; 59 and 86; 59 and 87; 59 and 88; 59 and 89; 59 and 90; 59 and 91; 59 and 92; 59 and 93; 59 and 94; 59 and 95; 59 and 96; 59 and 97; 59 and 98; 59 and 99; 59 and 100; 59 and 101; 59 and 102; 59 and 103; 59 and 104; 59 and 105; 59 and 106; 59 and 107; 59 and 108; 59 and 109; 59 and 110; 59 and 111; 59 and 112; 59 and 113; 59 and 114; 59 and 115; 59 and 116; 59 and 117; 59 and 118; 59 and 119; 59 and 120; 59 and 121; 59 and 122; 59 and 123; 59 and 124; 59 and 125; 59 and 126; 59 and 127; 59 and 128; 59 and 129; 59 and 130; 59 and 131; 59 and 132; 59 and 133; 59 and 134; 59 and 135; 59 and 136; 59 and 137; 59 and 138; 59 and 139; 59 and 140; 59 and 141; 59 and 142; 59 and 143; 59 and 144; 59 and 145; 59 and 146; 59 and 147; 59 and 148; 59 and 149; 59 and 150; 59 and 151; 59 and 152; 59 and 153; 59 and 154; 59 and 155; 59 and 156; 59 and 157; 59 and 158; 59 and 159; 59 and 160; 59 and 161; 59 and 162; 59 and 163; 59 and 164; 59 and 165; 59 and 166; 59 and 167; 60 and 67; 60 and 68; 60 and 69; 60 and 70; 60 and 71; 60 and 72; 60 and 73; 60 and 74; 60 and 75; 60 and 76; 60 and 77; 60 and 78; 60 and 79; 60 and 80; 60 and 81; 60 and 82; 60 and 83; 60 and 84; 60 and 85; 60 and 86; 60 and 87; 60 and 88; 60 and 89; 60 and 90; 60 and 91; 60 and 92; 60 and 93; 60 and 94; 60 and 95; 60 and 96; 60 and 97; 60 and 98; 60 and 99; 60 and 100; 60 and 101; 60 and 102; 60 and 103; 60 and 104; 60 and 105; 60 and 106; 60 and 107; 60 and 108; 60 and 109; 60 and 110; 60 and 111; 60 and 112; 60 and 113; 60 and 114; 60 and 115; 60 and 116; 60 and 117; 60 and 118; 60 and 119; 60 and 120; 60 and 121; 60 and 122; 60 and 123; 60 and 124; 60 and 125; 60 and 126; 60 and 127; 60 and 128; 60 and 129; 60 and 130; 60 and 131; 60 and 132; 60 and 133; 60 and 134; 60 and 135; 60 and 136; 60 and 137; 60 and 138; 60 and 139; 60 and 140; 60 and 141; 60 and 142; 60 and 143; 60 and 144; 60 and 145; 60 and 146; 60 and 147; 60 and 148; 60 and 149; 60 and 150; 60 and 151; 60 and 152; 60 and 153; 60 and 154; 60 and 155; 60 and 156; 60 and 157; 60 and 158; 60 and 159; 60 and 160; 60 and 161; 60 and 162; 60 and 163; 60 and 164; 60 and 165; 60 and 166; 60 and 167; 61 and 67; 61 and 68; 61 and 69; 61 and 70; 61 and 71; 61 and 72; 61 and 73; 61 and 74; 61 and 75; 61 and 76; 61 and 77; 61 and 78; 61 and 79; 61 and 80; 61 and 81; 61 and 82; 61 and 83; 61 and 84; 61 and 85; 61 and 86; 61 and 87; 61 and 88; 61 and 89; 61 and 90; 61 and 91; 61 and 92; 61 and 93; 61 and 94; 61 and 95; 61 and 96; 61 and 97; 61 and 98; 61 and 99; 61 and 100; 61 and 101; 61 and 102; 61 and 103; 61 and 104; 61 and 105; 61 and 106; 61 and 107; 61 and 108; 61 and 109; 61 and 110; 61 and 111; 61 and 112; 61 and 113; 61 and 114; 61 and 115; 61 and 116; 61 and 117; 61 and 118; 61 and 119; 61 and 120; 61 and 121; 61 and 122; 61 and 123; 61 and 124; 61 and 125; 61 and 126; 61 and 127; 61 and 128; 61 and 129; 61 and 130; 61 and 131; 61 and 132; 61 and 133; 61 and 134; 61 and 135; 61 and 136; 61 and 137; 61 and 138; 61 and 139; 61 and 140; 61 and 141; 61 and 142; 61 and 143; 61 and 144; 61 and 145; 61 and 146; 61 and 147; 61 and 148; 61 and 149; 61 and 150; 61 and 151; 61 and 152; 61 and 153; 61 and 154; 61 and 155; 61 and 156; 61 and 157; 61 and 158; 61 and 159; 61 and 160; 61 and 161; 61 and 162; 61 and 163; 61 and 164; 61 and 165; 61 and 166; 61 and 167; 62 and 67; 62 and 68; 62 and 69; 62 and 70; 62 and 71; 62 and 72; 62 and 73; 62 and 74; 62 and 75; 62 and 76; 62 and 77; 62 and 78; 62 and 79; 62 and 80; 62 and 81; 62 and 82; 62 and 83; 62 and 84; 62 and 85; 62 and 86; 62 and 87; 62 and 88; 62 and 89; 62 and 90; 62 and 91; 62 and 92; 62 and 93; 62 and 94; 62 and 95; 62 and 96; 62 and 97; 62 and 98; 62 and 99; 62 and 100; 62 and 101; 62 and 102; 62 and 103; 62 and 104; 62 and 105; 62 and 106; 62 and 107; 62 and 108; 62 and 109; 62 and 110; 62 and 111; 62 and 112; 62 and 113; 62 and 114; 62 and 115; 62 and 116; 62 and 117; 62 and 118; 62 and 119; 62 and 120; 62 and 121; 62 and 122; 62 and 123; 62 and 124; 62 and 125; 62 and 126; 62 and 127; 62 and 128; 62 and 129; 62 and 130; 62 and 131; 62 and 132; 62 and 133; 62 and 134; 62 and 135; 62 and 136; 62 and 137; 62 and 138; 62 and 139; 62 and 140; 62 and 141; 62 and 142; 62 and 143; 62 and 144; 62 and 145; 62 and 146; 62 and 147; 62 and 148; 62 and 149; 62 and 150; 62 and 151; 62 and 152; 62 and 153; 62 and 154; 62 and 155; 62 and 156; 62 and 157; 62 and 158; 62 and 159; 62 and 160; 62 and 161; 62 and 162; 62 and 163; 62 and 164; 62 and 165; 62 and 166; 62 and 167; 63 and 67; 63 and 68; 63 and 69; 63 and 70; 63 and 71; 63 and 72; 63 and 73; 63 and 74; 63 and 75; 63 and 76; 63 and 77; 63 and 78; 63 and 79; 63 and 80; 63 and 81; 63 and 82; 63 and 83; 63 and 84; 63 and 85; 63 and 86; 63 and 87; 63 and 88; 63 and 89; 63 and 90; 63 and 91; 63 and 92; 63 and 93; 63 and 94; 63 and 95; 63 and 96; 63 and 97; 63 and 98; 63 and 99; 63 and 100; 63 and 101; 63 and 102; 63 and 103; 63 and 104; 63 and 105; 63 and 106; 63 and 107; 63 and 108; 63 and 109; 63 and 110; 63 and 111; 63 and 112; 63 and 113; 63 and 114; 63 and 115; 63 and 116; 63 and 117; 63 and 118; 63 and 119; 63 and 120; 63 and 121; 63 and 122; 63 and 123; 63 and 124; 63 and 125; 63 and 126; 63 and 127; 63 and 128; 63 and 129; 63 and 130; 63 and 131; 63 and 132; 63 and 133; 63 and 134; 63 and 135; 63 and 136; 63 and 137; 63 and 138; 63 and 139; 63 and 140; 63 and 141; 63 and 142; 63 and 143; 63 and 144; 63 and 145; 63 and 146; 63 and 147; 63 and 148; 63 and 149; 63 and 150; 63 and 151; 63 and 152; 63 and 153; 63 and 154; 63 and 155; 63 and 156; 63 and 157; 63 and 158; 63 and 159; 63 and 160; 63 and 161; 63 and 162; 63 and 163; 63 and 164; 63 and 165; 63 and 166; 63 and 167; 64 and 67; 64 and 68; 64 and 69; 64 and 70; 64 and 71; 64 and 72; 64 and 73; 64 and 74; 64 and 75; 64 and 76; 64 and 77; 64 and 78; 64 and 79; 64 and 80; 64 and 81; 64 and 82; 64 and 83; 64 and 84; 64 and 85; 64 and 86; 64 and 87; 64 and 88; 64 and 89; 64 and 90; 64 and 91; 64 and 92; 64 and 93; 64 and 94; 64 and 95; 64 and 96; 64 and 97; 64 and 98; 64 and 99; 64 and 100; 64 and 101; 64 and 102; 64 and 103; 64 and 104; 64 and 105; 64 and 106; 64 and 107; 64 and 108; 64 and 109; 64 and 110; 64 and 111; 64 and 112; 64 and 113; 64 and 114; 64 and 115; 64 and 116; 64 and 117; 64 and 118; 64 and 119; 64 and 120; 64 and 121; 64 and 122; 64 and 123; 64 and 124; 64 and 125; 64 and 126; 64 and 127; 64 and 128; 64 and 129; 64 and 130; 64 and 131; 64 and 132; 64 and 133; 64 and 134; 64 and 135; 64 and 136; 64 and 137; 64 and 138; 64 and 139; 64 and 140; 64 and 141; 64 and 142; 64 and 143; 64 and 144; 64 and 145; 64 and 146; 64 and 147; 64 and 148; 64 and 149; 64 and 150; 64 and 151; 64 and 152; 64 and 153; 64 and 154; 64 and 155; 64 and 156; 64 and 157; 64 and 158; 64 and 159; 64 and 160; 64 and 161; 64 and 162; 64 and 163; 64 and 164; 64 and 165; 64 and 166; 64 and 167; 65 and 67; 65 and 68; 65 and 69; 65 and 70; 65 and 71; 65 and 72; 65 and 73; 65 and 74; 65 and 75; 65 and 76; 65 and 77; 65 and 78; 65 and 79; 65 and 80; 65 and 81; 65 and 82; 65 and 83; 65 and 84; 65 and 85; 65 and 86; 65 and 87; 65 and 88; 65 and 89; 65 and 90; 65 and 91; 65 and 92; 65 and 93; 65 and 94; 65 and 95; 65 and 96; 65 and 97; 65 and 98; 65 and 99; 65 and 100; 65 and 101; 65 and 102; 65 and 103; 65 and 104; 65 and 105; 65 and 106; 65 and 107; 65 and 108; 65 and 109; 65 and 110; 65 and 111; 65 and 112; 65 and 113; 65 and 114; 65 and 115; 65 and 116; 65 and 117; 65 and 118; 65 and 119; 65 and 120; 65 and 121; 65 and 122; 65 and 123; 65 and 124; 65 and 125; 65 and 126; 65 and 127; 65 and 128; 65 and 129; 65 and 130; 65 and 131; 65 and 132; 65 and 133; 65 and 134; 65 and 135; 65 and 136; 65 and 137; 65 and 138; 65 and 139; 65 and 140; 65 and 141; 65 and 142; 65 and 143; 65 and 144; 65 and 145; 65 and 146; 65 and 147; 65 and 148; 65 and 149; 65 and 150; 65 and 151; 65 and 152; 65 and 153; 65 and 154; 65 and 155; 65 and 156; 65 and 157; 65 and 158; 65 and 159; 65 and 160; 65 and 161; 65 and 162; 65 and 163; 65 and 164; 65 and 165; 65 and 166; and 65 and 167.

In some embodiments, compositions, methods/uses, and systems are provided comprising a pair of guide RNAs comprising a first and second spacer, or one or more vectors encoding the pair of guide RNAs, wherein the first and second spacer sequences comprise any one of the following pairs of SEQ ID NOs: 6 and 72; 6 and 81; 6 and 84; 6 and 98; 6 and 100; 6 and 114; 6 and 122; 6 and 134; 6 and 139; 6 and 149; 6 and 166; 8 and 72; 8 and 72; 8 and 81; 8 and 84; 8 and 98; 8 and 100; 8 and 114; 8 and 122; 8 and 134; 8 and 139; 8 and 149; 8 and 166; 10 and 72; 10 and 81; 10 and 84; 10 and 98; 10 and 100; 10 and 114; 10 and 122; 10 and 134; 10 and 139; 10 and 149; 10 and 166; 21 and 72; 21 and 81; 21 and 84; 21 and 98; 21 and 100; 21 and 114; 21 and 122; 21 and 134; 21 and 139; 21 and 149; 21 and 166; 58 and 72; 58 and 81; 58 and 84; 58 and 98; 58 and 100; 58 and 114; 58 and 122; 58 and 134; 58 and 139; 58 and 149; 58 and 166; 62 and 72; 62 and 81; 62 and 84; 62 and 98; 62 and 100; 62 and 114; 62 and 122; 62 and 134; 62 and 139; 62 and 149; 62 and 166; 63 and 72; 63 and 81; 63 and 84; 63 and 98; 63 and 100; 63 and 114; 63 and 122; 63 and 134; 63 and 139; 63 and 149; 63 and 166; 64 and 72; 64 and 81; 64 and 84; 64 and 98; 64 and 100; 64 and 114; 64 and 122; 64 and 134; 64 and 139; 64 and 149; and 64 and 166.

In some embodiments, the methods comprise delivering to a cell a nucleic acid molecule encoding SluCas9, wherein the SluCas9 comprises the amino acid sequence of SEQ ID NO: 712. In some embodiments, the methods comprise delivering to a cell a nucleic acid molecule encoding SluCas9, wherein the SluCas9 is a variant of the amino acid sequence of SEQ ID NO: 712. In some embodiments, the methods comprise delivering to a cell a nucleic acid molecule encoding SluCas9, wherein the SluCas9 comprises an amino acid sequence selected from any one of SEQ ID NOs: 718-720.

In some embodiments, the subject is a mammal. In some embodiments, the subject is human.

### IV. DNA-PK Inhibitor

Where a DNA-PK inhibitor is used in a composition or method disclosed herein, it may be any DNA-PK inhibitor known in the art. DNA-PK inhibitors are discussed in detail, for example, in WO2014/159690; WO2013/163190; WO2018/013840; WO 2019/143675; WO 2019/143677; WO 2019/143678; US2014275059; US2013281431; US2020361877; US2020353101 and Robert et al., Genome Medicine (2015) 7:93, each of which are incorporated by reference herein. In some embodiments, the DNA-PK inhibitor is NU7441, KU-0060648, or any one of Compounds 1, 2, 3, 4, 5, or 6 (structures shown below), each of which is also described in at least one of the foregoing citations. In some embodiments, the DNA-PK inhibitor is Compound 1. In some embodiments, the DNA-PK inhibitor is Compound 2. In some embodiments, the DNA-PK inhibitor is Compound 6. In some embodiments, the DNA-PK inhibitor is Compound 3. Structures for exemplary DNA-PK inhibitors are as follows. Unless otherwise indicated, reference to a DNA-PK inhibitor by name or structure encompasses pharmaceutically acceptable salts thereof.

| DNA-PK Inhibitor | Structure |
|---|---|
| NU7441 | 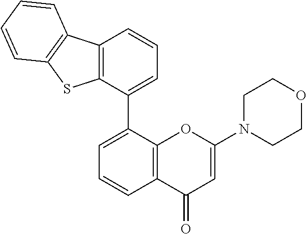 [SMILES: O=c1cc(N2CCOCC2)oc2c(-c3cccc4c3sc3ccccc34)cccc12] |
| KU-0060648 | 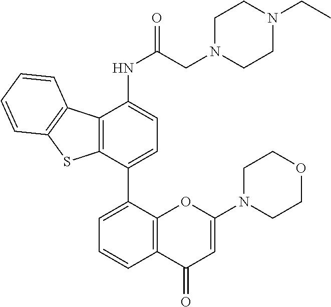 [SMILES: CCN1CCN(CC(=O)Nc2ccc(-c3cccc4c(=O)cc(N5CCOCC5)oc34)c3sc4ccccc4c23)CC1] |
| Compound 1 | 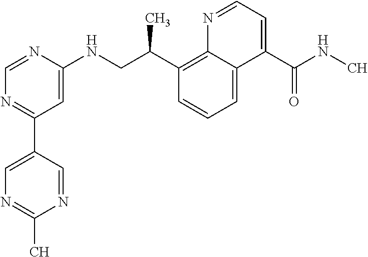 [SMILES: CNC(=O)c1ccnc2c([C@H](C)CNc3cc(-c4cnc(C)nc4)ncn3)cccc12] |
| Compound 2 | 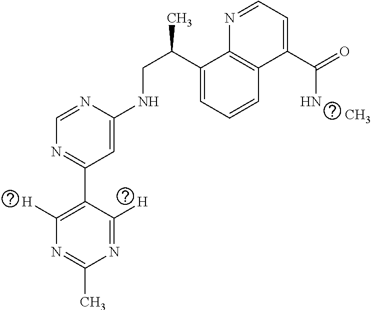 |
| Compound 3 | 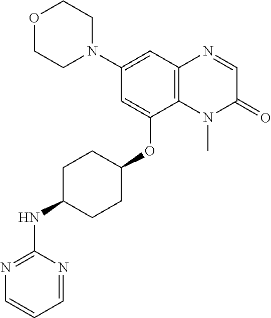 [SMILES: Cn1c(=O)cnc2cc(N3CCOCC3)cc(O[C@H]3CC[C@@H](Nc4ncccn4)CC3)c21] |
| Compound 4 | 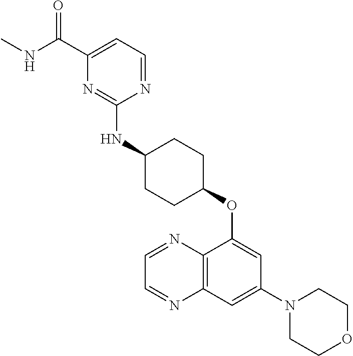 [SMILES: CNC(=O)c1ccnc(N[C@H]2CC[C@@H](Oc3cc(N4CCOCC4)cc4nccnc34)CC2)n1] |
| Compound 5 | 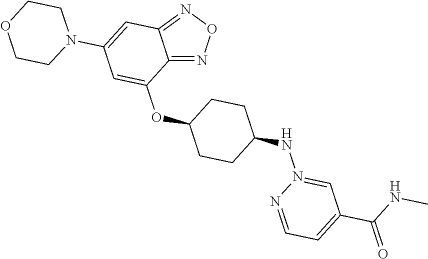 |
| Compound 6 | 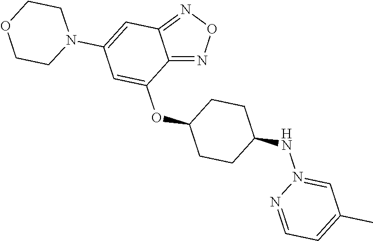 |

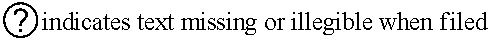

In any of the foregoing embodiments where a DNA-PK inhibitor is used, it may be used in combination with only one gRNA or vector encoding only one gRNA to promote excision, i.e., the method does not always involve providing two or more guides that promote cleavage near a CTG repeat.

In some embodiments where a DNA-PK inhibitor is used, it may be used in combination with a pair of gRNAs or vector encoding a pair of guide RNAs to promote excision. In some embodiments, the pair of gRNAs comprise gRNAs that are not the same. In particular embodiments, the pair of gRNAs together target sequences that flank a CTG repeat region in the genome of a cell.

### V. Combination Therapy

In some embodiments, the invention comprises combination therapies comprising any of the methods or uses described herein together with an additional therapy suitable for ameliorating DM1.

### VI. Delivery of Guide RNA Compositions

The methods and uses disclosed herein may use any suitable approach for delivering the guide RNAs and compositions described herein. Exemplary delivery approaches include vectors, such as viral vectors; lipid nanoparticles; transfection; and electroporation. In some embodiments, vectors or LNPs associated with the single-vector guide RNAs/Cas9's disclosed herein are for use in preparing a medicament for treating DM1.

Where a vector is used, it may be a viral vector, such as a non-integrating viral vector. In some embodiments, the viral vector is an adeno-associated virus vector, a lentiviral vector, an integrase-deficient lentiviral vector, an adenoviral vector, a vaccinia viral vector, an alphaviral vector, or a herpes simplex viral vector. In some embodiments, the viral vector is an adeno-associated virus (AAV) vector. In some embodiments, the AAV vector is an AAV1, AAV2, AAV3, AAV4, AAV5, AAV6, AAV7, AAV8, AAVrh10 (see, e.g., SEQ ID NO: 81 of U.S. Pat. No. 9,790,472, which is incorporated by reference herein in its entirety), AAVrh74 (see, e.g., SEQ ID NO: 1 of US 2015/0111955, which is incorporated by reference herein in its entirety), or AAV9 vector, wherein the number following AAV indicates the AAV serotype. Any variant of an AAV vector or serotype thereof, such as a self-complementary AAV (scAAV) vector, is encompassed within the general terms AAV vector, AAV1 vector, etc. See, e.g., McCarty et al., Gene Ther. 2001; 8:1248-54, Naso et al., BioDrugs 2017; 31:317-334, and references cited therein for detailed discussion of various AAV vectors.

In some embodiments, the vector (e.g., viral vector, such as an adeno-associated viral vector) comprises a tissue-specific (e.g., muscle-specific) promoter, e.g., which is operatively linked to a sequence encoding the guide RNA. In some embodiments, the muscle-specific promoter is a muscle creatine kinase promoter, a desmin promoter, an MHCK7 promoter, or an SPc5-12 promoter. In some embodiments, the muscle-specific promoter is a CK8 promoter. In some embodiments, the muscle-specific promoter is a CK8e promoter. Muscle-specific promoters are described in detail, e.g., in US2004/0175727 A1; Wang et al., Expert Opin Drug Deliv. (2014) 11, 345-364; Wang et al., Gene Therapy (2008) 15, 1489-1499. In some embodiments, the tissue-specific promoter is a neuron-specific promoter, such as an enolase promoter. See, e.g., Naso et al., BioDrugs 2017; 31:317-334; Dashkoff et al., Mol Ther Methods C/in Dev. 2016; 3:16081, and references cited therein for detailed discussion of tissue-specific promoters including neuron-specific promoters.

In some embodiments, in addition to guide RNA and Cas9 sequences, the vectors further comprise nucleic acids that do not encode guide RNAs. Nucleic acids that do not encode guide RNA and Cas9 include, but are not limited to, promoters, enhancers, and regulatory sequences. In some embodiments, the vector comprises one or more nucleotide sequence(s) encoding a crRNA, a trRNA, or a crRNA and trRNA.

Lipid nanoparticles (LNPs) are a known means for delivery of nucleotide and protein cargo, and may be used for delivery of the guide RNAs, compositions, or pharmaceutical formulations disclosed herein. In some embodiments, the LNPs deliver nucleic acid, protein, or nucleic acid together with protein.

Electroporation is a well-known means for delivery of cargo, and any electroporation methodology may be used for delivering the single vectors disclosed herein.

In some embodiments, the invention comprises a method for delivering any one of the single vectors disclosed herein to an ex vivo cell, wherein the guide RNA is encoded by a vector, associated with an LNP, or in aqueous solution. In some embodiments, the guide RNA/LNP or guide RNA is also associated with a Cas9 or sequence encoding Cas9 (e.g., in the same vector, LNP, or solution).

### EXAMPLES

The following examples are provided to illustrate certain disclosed embodiments and are not to be construed as limiting the scope of this disclosure in any way.

### Example 1: Evaluation of DM1 sgRNAs

A. Materials and Methods

1. sgRNA Selection

The 3′ UTR of the human DMPK gene was scanned for the SluCas9 PAM sequence NNGG on either the sense or antisense strand, and 172 sgRNA protospacer sequences (22-nucleotide in length) adjacent to the PAMs were identified (Table 1A). 166 sgRNAs were selected for evaluation in primary DM1 patient myoblasts based on in silico off-target assessment. Further exemplary guide sequences are shown in Table 1B.

TABLE 1A
SluCas9 sgRNAs with the SluCas9 PAM sequences in the 3′ UTR region of human DMPK gene

| | | | | | | | | | | Predicted |
|---|---|---|---|---|---|---|---|---|---|---|
| SluCas9 | SEQ | | | | | | | | Protospacer | off-target |
| sgRNA | ID | | Protospacer sequence | Protospacer | Protospacer | PAM | PAM | PAM | GC | site |
| name | NO | Strand | (22 bp) | start | end | sequence | start | end | content | number |
| SluU01 | 1 | − | AACCCTAGAACTGTCTTCGACT | 45770467 | 45770488 | CCGG | 45770463 | 45770466 | 45.45 | 1 |
| SluU02 | 2 | − | ACCCTAGAACTGTCTTCGACTC | 45770466 | 45770487 | CGGG | 45770462 | 45770465 | 50 | 10 |
| SluU03 | 3 | − | CCCTAGAACTGTCTTCGACTCC | 45770465 | 45770486 | GGGG | 45770461 | 45770464 | 54.55 | 1 |
| SluU04 | 4 | + | CCCCGGAGTCGAAGACAGTTCT | 45770461 | 45770482 | AGGG | 45770483 | 45770486 | 59.09 | 2 |
| SluU05 | 5 | + | GCCCCGGAGTCGAAGACAGTTC | 45770460 | 45770481 | TAGG | 45770482 | 45770485 | 63.64 | 5 |
| SluU06 | 6 | − | TGTCTTCGACTCCGGGGCCCCG | 45770456 | 45770477 | TTGG | 45770452 | 45770455 | 72.73 | 2 |
| SluU07 | 7 | + | CACTCAGTCTTCCAACGGGGCC | 45770441 | 45770462 | CCGG | 45770463 | 45770466 | 63.64 | 0 |
| SluU08 | 8 | − | GCCCCGTTGGAAGACTGAGTGC | 45770440 | 45770461 | CCGG | 45770436 | 45770439 | 63.64 | 2 |
| SluU09 | 9 | − | CCCCGTTGGAAGACTGAGTGCC | 45770439 | 45770460 | CGGG | 45770435 | 45770438 | 63.64 | 4 |
| SluU10 | 10 | − | CCCGTTGGAAGACTGAGTGCCC | 45770438 | 45770459 | GGGG | 45770434 | 45770437 | 63.64 | 3 |
| SluU11 | 11 | + | CCCGGGCACTCAGTCTTCCAAC | 45770435 | 45770456 | GGGG | 45770457 | 45770460 | 63.64 | 7 |
| SluU12 | 12 | + | CCCCGGGCACTCAGTCTTCCAA | 45770434 | 45770455 | CGGG | 45770456 | 45770459 | 63.64 | 4 |
| SluU13 | 13 | + | GCCCCGGGCACTCAGTCTTCCA | 45770433 | 45770454 | ACGG | 45770455 | 45770458 | 68.18 | 8 |
| SluU14 | 14 | − | TGGAAGACTGAGTGCCCGGGGC | 45770433 | 45770454 | ACGG | 45770429 | 45770432 | 68.18 | 12 |
| SluU15 | 15 | + | GCGCGGCTTCTGTGCCGTGCCC | 45770415 | 45770436 | CGGG | 45770437 | 45770440 | 77.27 | 5 |
| SluU16 | 16 | + | GGCGCGGCTTCTGTGCCGTGCC | 45770414 | 45770435 | CCGG | 45770436 | 45770439 | 77.27 | 1 |
| SluU17 | 17 | + | TGTGAACTGGCAGGCGGTGGGC | 45770395 | 45770416 | GCGG | 45770417 | 45770420 | 68.18 | 22 |
| SluU18 | 18 | + | GCGGTTGTGAACTGGCAGGCGG | 45770390 | 45770411 | TGGG | 45770412 | 45770415 | 68.18 | 3 |
| SluU19 | 19 | + | AGCGGTTGTGAACTGGCAGGCG | 45770389 | 45770410 | GTGG | 45770411 | 45770414 | 63.64 | 2 |
| SluU20 | 20 | + | CGGAGCGGTTGTGAACTGGCAG | 45770386 | 45770407 | GCGG | 45770408 | 45770411 | 63.64 | 4 |
| SluU21 | 21 | + | GCTCGGAGCGGTTGTGAACTGG | 45770383 | 45770404 | CAGG | 45770405 | 45770408 | 63.64 | 0 |
| SluU22 | 22 | − | CCAGTTCACAACCGCTCCGAGC | 45770383 | 45770404 | GTGG | 45770379 | 45770382 | 63.64 | 1 |
| SluU23 | 23 | − | CAGTTCACAACCGCTCCGAGCG | 45770382 | 45770403 | TGGG | 45770378 | 45770381 | 63.64 | 1 |
| SluU24 | 24 | + | CCACGCTCGGAGCGGTTGTGAA | 45770379 | 45770400 | CTGG | 45770401 | 45770404 | 63.64 | 0 |
| SluU25 | 25 | + | TGGGCGGAGACCCACGCTCGGA | 45770368 | 45770389 | GCGG | 45770390 | 45770393 | 72.73 | 1 |
| SluU26 | 26 | + | GGAGCTGGGCGGAGACCCACGC | 45770363 | 45770384 | TCGG | 45770385 | 45770388 | 77.27 | 4 |
| SluU27 | 27 | − | CGCCCAGCTCCAGTCCTGTGAT | 45770352 | 45770373 | CCGG | 45770348 | 45770351 | 63.64 | 6 |
| SluU28 | 28 | − | GCCCAGCTCCAGTCCTGTGATC | 45770351 | 45770372 | CGGG | 45770347 | 45770350 | 63.64 | 8 |
| SluU29 | 29 | + | CGGATCACAGGACTGGAGCTGG | 45770349 | 45770370 | GCGG | 45770371 | 45770374 | 63.64 | 13 |
| SluU30 | 30 | + | GCCCGGATCACAGGACTGGAGC | 45770346 | 45770367 | TGGG | 45770368 | 45770371 | 68.18 | 5 |
| SluU31 | 31 | + | GGCCCGGATCACAGGACTGGAG | 45770345 | 45770366 | CTGG | 45770367 | 45770370 | 68.18 | 6 |
| SluU32 | 32 | + | GGGGCGGGCCCGGATCACAGGA | 45770339 | 45770360 | CTGG | 45770361 | 45770364 | 77.27 | 3 |
| SluU33 | 33 | − | TGTGATCCGGGCCCGCCCCCTA | 45770336 | 45770357 | GCgg | 45770332 | 45770335 | 72.73 | 2 |
| SluU34 | 34 | + | GCTAGGGGGCGGGCCCGGATCA | 45770334 | 45770355 | CAGG | 45770356 | 45770359 | 77.27 | 1 |
| SluU35 | 35 | − | ATCCGGGCCCGCCCCCTAGCgg | 45770332 | 45770353 | ccgg | 45770328 | 45770331 | 81.82 | 3 |
| SluU36 | 36 | − | TCCGGGCCCGCCCCCTAGCggc | 45770331 | 45770352 | cggg | 45770327 | 45770330 | 86.36 | 7 |
| SluU37 | 37 | − | CCGGGCCCGCCCCCTAGCggcc | 45770330 | 45770351 | gggg | 45770326 | 45770329 | 90.91 | 10 |
| SluU38 | 38 | − | GGCCCGCCCCCTAGCggccggg | 45770327 | 45770348 | gagg | 45770323 | 45770326 | 90.91 | 10 |
| SluU39 | 39 | + | ccccggccGCTAGGGGGCGGGC | 45770326 | 45770347 | CCGG | 45770348 | 45770351 | 90.91 | 10 |
| SluU40 | 40 | − | GCCCGCCCCCTAGCggccgggg | 45770326 | 45770347 | aggg | 45770322 | 45770325 | 90.91 | 12 |
| SluU41 | 41 | − | CGCCCCCTAGCggccggggagg | 45770323 | 45770344 | gagg | 45770319 | 45770322 | 86.36 | 7 |
| SluU42 | 42 | − | GCCCCCTAGCggccggggaggg | 45770322 | 45770343 | aggg | 45770318 | 45770321 | 86.36 | 9 |
| SluU43 | 43 | + | tccctccccggccGCTAGGGGG | 45770321 | 45770342 | CGGG | 45770343 | 45770346 | 81.82 | 5 |
| SluU44 | 44 | − | CCCCCTAGCggccggggaggga | 45770321 | 45770342 | gggg | 45770317 | 45770320 | 81.82 | 8 |
| SluU45 | 45 | + | ctccctccccggccGCTAGGGG | 45770320 | 45770341 | GCGG | 45770342 | 45770345 | 81.82 | 6 |
| SluU46 | 46 | + | cccctccctccccggccGCTAG | 45770317 | 45770338 | GGGG | 45770339 | 45770342 | 81.82 | 11 |
| SluU47 | 47 | − | CTAGCggccggggagggagggg | 45770317 | 45770338 | ccgg | 45770313 | 45770316 | 81.82 | 33 |
| SluU48 | 48 | + | gcccctccctccccggccGCTA | 45770316 | 45770337 | GGGG | 45770338 | 45770341 | 81.82 | 11 |
| SluU49 | 49 | − | TAGCggccggggagggaggggc | 45770316 | 45770337 | cggg | 45770312 | 45770315 | 81.82 | 22 |
| SluU50 | 50 | + | ggcccctccctccccggccGCT | 45770315 | 45770336 | AGGG | 45770337 | 45770340 | 86.36 | 29 |
| SluU51 | 51 | + | cggcccctccctccccggccGC | 45770314 | 45770335 | TAGG | 45770336 | 45770339 | 90.91 | 28 |
| SluU52 | 52 | − | cggggagggaggggccgggtcc | 45770309 | 45770330 | gcgg | 45770305 | 45770308 | 86.36 | 66 |
| SluU53 | 53 | + | cgcggacccggcccctccctcc | 45770306 | 45770327 | ccgg | 45770328 | 45770331 | 86.36 | 21 |
| SluU54 | 54 | − | gagggaggggccgggtccgcgg | 45770305 | 45770326 | ccgg | 45770301 | 45770304 | 86.36 | 41 |
| SluU55 | 55 | − | gggccgggtccgcggccggcga | 45770298 | 45770319 | acgg | 45770294 | 45770297 | 90.91 | 41 |
| SluU56 | 56 | − | ggccgggtccgcggccggcgaa | 45770297 | 45770318 | cggg | 45770293 | 45770296 | 86.36 | 10 |
| SluU57 | 57 | − | gccgggtccgcggccggcgaac | 45770296 | 45770317 | gggg | 45770292 | 45770295 | 86.36 | 5 |
| SluU58 | 58 | + | gccccgttcgccggccgeggac | 45770291 | 45770312 | ccgg | 45770313 | 45770316 | 86.36 | 1 |
| SluU59 | 59 | − | cgcggccggcgaacggggcTCG | 45770288 | 45770309 | AAGG | 45770284 | 45770287 | 86.36 | 5 |
| SluU60 | 60 | − | gcggccggcgaacggggcTCGA | 45770287 | 45770308 | AGGG | 45770283 | 45770286 | 81.82 | 2 |
| SluU61 | 61 | − | CTTCGAgccccgttcgccggcc | 45770285 | 45770306 | gcgg | 45770307 | 45770310 | 77.27 | 1 |
| SluU62 | 62 | + | AGGACCCTTCGAgccccgttcg | 45770279 | 45770300 | ccgg | 45770301 | 45770304 | 68.18 | 0 |
| SluU63 | 63 | − | ggggcTCGAAGGGTCCTTGTAG | 45770274 | 45770295 | CCGG | 45770270 | 45770273 | 63.64 | 4 |
| SluU64 | 64 | − | gggcTCGAAGGGTCCTTGTAGC | 45770273 | 45770294 | CGGG | 45770269 | 45770272 | 63.64 | 1 |
| SluU65 | 65 | + | cagcagcagcaTTCCCGGCTAC | 45770256 | 45770277 | AAGG | 45770278 | 45770281 | 63.64 | 7 |
| SluU66* | 66 | + | agcagcagcagcagcagcaTTC | 45770248 | 45770269 | CCGG | 45770270 | 45770273 | 59.09 | 647 |
| SluD01 | 67 | − | GATCACAGACCATTTCTTTCTT | 45770179 | 45770200 | TCGG | 45770175 | 45770178 | 36.36 | 14 |
| SluD02 | 68 | − | CAGACCATTTCTTTCTTTCGGC | 45770174 | 45770195 | CAGG | 45770170 | 45770173 | 45.45 | 5 |
| SluD03 | 69 | − | ATTTCTTTCTTTCGGCCAGGCT | 45770168 | 45770189 | GAGG | 45770164 | 45770167 | 45.45 | 14 |
| SluD04 | 70 | + | TCAGCCTGGCCGAAAGAAAGAA | 45770166 | 45770187 | ATGG | 45770188 | 45770191 | 50 | 6 |
| SluD05 | 71 | − | TCGGCCAGGCTGAGGCCCTGAC | 45770157 | 45770178 | GTGG | 45770153 | 45770156 | 72.73 | 8 |
| SluD06 | 72 | − | CCAGGCTGAGGCCCTGACGTGG | 45770153 | 45770174 | ATGG | 45770149 | 45770152 | 72.73 | 12 |
| SluD07 | 73 | − | CAGGCTGAGGCCCTGACGTGGA | 45770152 | 45770173 | TGGG | 45770148 | 45770151 | 68.18 | 6 |
| SluD08 | 74 | + | CCATCCACGTCAGGGCCTCAGC | 45770149 | 45770170 | CTGG | 45770171 | 45770174 | 68.18 | 5 |
| SluD09 | 75 | − | CCTGACGTGGATGGGCAAACTG | 45770141 | 45770162 | CAGG | 45770137 | 45770140 | 59.09 | 2 |
| SluD10 | 76 | + | CTGCAGTTTGCCCATCCACGTC | 45770138 | 45770159 | AGGG | 45770160 | 45770163 | 59.09 | 3 |
| SluD11 | 77 | + | CCTGCAGTTTGCCCATCCACGT | 45770137 | 45770158 | CAGG | 45770159 | 45770162 | 59.09 | 3 |
| SluD12 | 78 | − | CGTGGATGGGCAAACTGCAGGC | 45770136 | 45770157 | CTGG | 45770132 | 45770135 | 63.64 | 7 |
| SluD13 | 79 | − | GTGGATGGGCAAACTGCAGGCC | 45770135 | 45770156 | TGGG | 45770131 | 45770134 | 63.64 | 31 |
| SluD16 | 80 | − | CAGGCCTGGGAAGGCAGCAAGC | 45770119 | 45770140 | CGGG | 45770115 | 45770118 | 68.18 | 40 |
| SluD14 | 81 | − | ATGGGCAAACTGCAGGCCTGGG | 45770131 | 45770152 | AAGG | 45770127 | 45770130 | 63.64 | 77 |
| SluD15 | 82 | − | GCAGGCCTGGGAAGGCAGCAAG | 45770120 | 45770141 | CCGG | 45770116 | 45770119 | 68.18 | 46 |
| SluD17 | 83 | + | ACGGCCCGGCTTGCTGCCTTCC | 45770111 | 45770132 | CAGG | 45770133 | 45770136 | 72.73 | 4 |
| SluD18 | 84 | + | TGGAGGATGGAACACGGACGGC | 45770094 | 45770115 | CCGG | 45770116 | 45770119 | 63.64 | 3 |
| SluD19 | 85 | + | GTGCGTGGAGGATGGAACACGG | 45770089 | 45770110 | ACGG | 45770111 | 45770114 | 63.64 | 0 |
| SluD20 | 86 | + | GGGGGTGCGTGGAGGATGGAAC | 45770085 | 45770106 | ACGG | 45770107 | 45770110 | 68.18 | 26 |
| SluD21 | 87 | + | GATAGGTGGGGGTGCGTGGAGG | 45770078 | 45770099 | ATGG | 45770100 | 45770103 | 68.18 | 29 |
| SluD22 | 88 | − | CTCCACGCACCCCCACCTATCG | 45770077 | 45770098 | TTGG | 45770073 | 45770076 | 68.18 | 1 |
| SluD23 | 89 | + | CAACGATAGGTGGGGGTGCGTG | 45770074 | 45770095 | GAGG | 45770096 | 45770099 | 63.64 | 0 |
| SluD24 | 90 | + | AACCAACGATAGGTGGGGGTGC | 45770071 | 45770092 | GTGG | 45770093 | 45770096 | 59.09 | 1 |
| SluD25 | 91 | + | CTTTGCGAACCAACGATAGGTG | 45770064 | 45770085 | GGGG | 45770086 | 45770089 | 50 | 1 |
| SluD26 | 92 | + | ACTTTGCGAACCAACGATAGGT | 45770063 | 45770084 | GGGG | 45770085 | 45770088 | 45.45 | 3 |
| SluD27 | 93 | + | CACTTTGCGAACCAACGATAGG | 45770062 | 45770083 | TGGG | 45770084 | 45770087 | 50 | 3 |
| SluD28 | 94 | + | GCACTTTGCGAACCAACGATAG | 45770061 | 45770082 | GTGG | 45770083 | 45770086 | 50 | 3 |
| SluD29 | 95 | + | TTTGCACTTTGCGAACCAACGA | 45770058 | 45770079 | TAGG | 45770080 | 45770083 | 45.45 | 2 |
| SluD30 | 96 | − | TTCTTGTGCATGACGCCCTGCT | 45770033 | 45770054 | CTGG | 45770029 | 45770032 | 54.55 | 5 |
| SluD31 | 97 | − | TCTTGTGCATGACGCCCTGCTC | 45770032 | 45770053 | TGGG | 45770028 | 45770031 | 59.09 | 3 |
| SluD32 | 98 | − | CTTGTGCATGACGCCCTGCTCT | 45770031 | 45770052 | GGGG | 45770027 | 45770030 | 59.09 | 6 |
| SluD33 | 99 | − | GACGCCCTGCTCTGGGGAGCGT | 45770022 | 45770043 | CTGG | 45770018 | 45770021 | 72.73 | 4 |
| SluD34 | 100 | + | CGCGCCAGACGCTCCCCAGAGC | 45770014 | 45770035 | AGGG | 45770036 | 45770039 | 77.27 | 7 |
| SluD35 | 101 | + | TCGCGCCAGACGCTCCCCAGAG | 45770013 | 45770034 | CAGG | 45770035 | 45770038 | 72.73 | 1 |
| SluD36 | 102 | − | GGCGCGATCTCTGCCTGCTTAC | 45769998 | 45770019 | TCGG | 45769994 | 45769997 | 63.64 | 1 |
| SluD37 | 103 | − | GCGCGATCTCTGCCTGCTTACT | 45769997 | 45770018 | CGGG | 45769993 | 45769996 | 59.09 | 1 |
| SluD38 | 104 | + | AAAAGCAAATTTCCCGAGTAAG | 45769981 | 45770002 | CAGG | 45770003 | 45770006 | 36.36 | 5 |
| SluD39 | 105 | − | TGCTTTTGCCAAACCCGCTTTT | 45769966 | 45769987 | TCGG | 45769962 | 45769965 | 45.45 | 2 |
| SluD40 | 106 | − | GCTTTTGCCAAACCCGCTTTTT | 45769965 | 45769986 | CGGG | 45769961 | 45769964 | 45.45 | 3 |
| SluD41 | 107 | − | CTTTTGCCAAACCCGCTTTTTC | 45769964 | 45769985 | GGGG | 45769960 | 45769963 | 45.45 | 2 |
| SluD42 | 108 | + | CGGGATCCCCGAAAAAGCGGGT | 45769954 | 45769975 | TTGG | 45769976 | 45769979 | 63.64 | 2 |
| SluD43 | 109 | + | GGGCGCGGGATCCCCGAAAAAG | 45769949 | 45769970 | CGGG | 45769971 | 45769974 | 68.18 | 1 |
| SluD44 | 110 | + | GGGGCGCGGGATCCCCGAAAAA | 45769948 | 45769969 | GCGG | 45769970 | 45769973 | 68.18 | 1 |
| SluD45 | 111 | + | AGCGCAAGTGAGGAGGGGGGCG | 45769932 | 45769953 | CGGG | 45769954 | 45769957 | 72.73 | 14 |
| SluD46 | 112 | + | CAGCGCAAGTGAGGAGGGGGGC | 45769931 | 45769952 | GCGG | 45769953 | 45769956 | 72.73 | 13 |
| SluD47 | 113 | − | CCCCTCCTCACTTGCGCTGCTC | 45769928 | 45769949 | TCGG | 45769924 | 45769927 | 68.18 | 8 |
| SluD48 | 114 | + | GAGAGCAGCGCAAGTGAGGAGG | 45769926 | 45769947 | GGGG | 45769948 | 45769951 | 63.64 | 37 |
| SluD49 | 115 | + | CGAGAGCAGCGCAAGTGAGGAG | 45769925 | 45769946 | GGGG | 45769947 | 45769950 | 63.64 | 5 |
| SluD50 | 116 | + | CCGAGAGCAGCGCAAGTGAGGA | 45769924 | 45769945 | GGGG | 45769946 | 45769949 | 63.64 | 5 |
| SluD51 | 117 | + | TCCGAGAGCAGCGCAAGTGAGG | 45769923 | 45769944 | AGGG | 45769945 | 45769948 | 63.64 | 4 |
| SluD52 | 118 | + | CTCCGAGAGCAGCGCAAGTGAG | 45769922 | 45769943 | GAGG | 45769944 | 45769947 | 63.64 | 3 |
| SluD53 | 119 | + | GGGCTCCGAGAGCAGCGCAAGT | 45769919 | 45769940 | GAGG | 45769941 | 45769944 | 68.18 | 6 |
| SluD54 | 120 | − | TGCGCTGCTCTCGGAGCCCCAG | 45769916 | 45769937 | CCGG | 45769912 | 45769915 | 72.73 | 7 |
| SluD55 | 121 | − | GCCCCAGCCGGCTCCGCCCGCT | 45769901 | 45769922 | TCGG | 45769897 | 45769900 | 86.36 | 7 |
| SluD56 | 122 | − | CCAGCCGGCTCCGCCCGCTTCG | 45769898 | 45769919 | GCGG | 45769894 | 45769897 | 81.82 | 4 |
| SluD57 | 123 | + | GCCGAAGCGGGCGGAGCCGGCT | 45769896 | 45769917 | GGGG | 45769918 | 45769921 | 81.82 | 12 |
| SluD58 | 124 | + | CGCCGAAGCGGGCGGAGCCGGC | 45769895 | 45769916 | TGGG | 45769917 | 45769920 | 86.36 | 9 |
| SluD59 | 125 | + | CCGCCGAAGCGGGCGGAGCCGG | 45769894 | 45769915 | CTGG | 45769916 | 45769919 | 86.36 | 6 |
| SluD60 | 126 | − | CGGCTCCGCCCGCTTCGGCGGT | 45769893 | 45769914 | TTGG | 45769889 | 45769892 | 81.82 | 3 |
| SluD61 | 127 | + | CAAACCGCCGAAGCGGGCGGAG | 45769890 | 45769911 | CCGG | 45769912 | 45769915 | 72.73 | 0 |
| SluD62 | 128 | + | AATATCCAAACCGCCGAAGCGG | 45769884 | 45769905 | GCGG | 45769906 | 45769909 | 54.55 | 0 |
| SluD63 | 129 | + | ATAAATATCCAAACCGCCGAAG | 45769881 | 45769902 | CGGG | 45769903 | 45769906 | 40.91 | 2 |
| SluD64 | 130 | + | AATAAATATCCAAACCGCCGAA | 45769880 | 45769901 | GCGG | 45769902 | 45769905 | 36.36 | 1 |
| SluD65 | 131 | − | ACCTCGTCCTCCGACTCGCTGA | 45769857 | 45769878 | CAGG | 45769853 | 45769856 | 63.64 | 0 |
| SluD66 | 132 | + | GCCTGTCAGCGAGTCGGAGGAC | 45769852 | 45769873 | GAGG | 45769874 | 45769877 | 68.18 | 45 |
| SluD67 | 133 | − | CCTCCGACTCGCTGACAGGCTA | 45769850 | 45769871 | CAGG | 45769846 | 45769849 | 63.64 | 4 |
| SluD68 | 134 | + | CCTGTAGCCTGTCAGCGAGTCG | 45769846 | 45769867 | GAGG | 45769868 | 45769871 | 63.64 | 2 |
| SluD69 | 135 | + | GGTCCTGTAGCCTGTCAGCGAG | 45769843 | 45769864 | TCGG | 45769865 | 45769868 | 63.64 | 1 |
| SluD70 | 136 | − | CCCAACAACCCCAATCCACGTT | 45769821 | 45769842 | TTGG | 45769817 | 45769820 | 54.55 | 1 |
| SluD71 | 137 | + | AAAACGTGGATTGGGGTTGTTG | 45769819 | 45769840 | GGGG | 45769841 | 45769844 | 45.45 | 5 |
| SluD72 | 138 | + | CAAAACGTGGATTGGGGTTGTT | 45769818 | 45769839 | GGGG | 45769840 | 45769843 | 45.45 | 5 |
| SluD73 | 139 | + | CCAAAACGTGGATTGGGGTTGT | 45769817 | 45769838 | TGGG | 45769839 | 45769842 | 50 | 6 |
| SluD74 | 140 | + | TCCAAAACGTGGATTGGGGTTG | 45769816 | 45769837 | TTGG | 45769838 | 45769841 | 50 | 7 |
| SluD75 | 141 | + | CAGTGCATCCAAAACGTGGATT | 45769809 | 45769830 | GGGG | 45769831 | 45769834 | 45.45 | 6 |
| SluD76 | 142 | + | TCAGTGCATCCAAAACGTGGAT | 45769808 | 45769829 | TGGG | 45769830 | 45769833 | 45.45 | 4 |
| SluD77 | 143 | + | CTCAGTGCATCCAAAACGTGGA | 45769807 | 45769828 | TTGG | 45769829 | 45769832 | 50 | 3 |
| SluD78 | 144 | + | GGGGTCTCAGTGCATCCAAAAC | 45769802 | 45769823 | GTGG | 45769824 | 45769827 | 54.55 | 3 |
| SluD79 | 145 | − | TGCACTGAGACCCCGACATTCC | 45769794 | 45769815 | TCGG | 45769790 | 45769793 | 59.09 | 4 |
| SluD80 | 146 | + | ACAATAAATACCGAGGAATGTC | 45769780 | 45769801 | GGGG | 45769802 | 45769805 | 36.36 | 2 |
| SluD81 | 147 | + | GACAATAAATACCGAGGAATGT | 45769779 | 45769800 | CGGG | 45769801 | 45769804 | 36.36 | 4 |
| SluD82 | 148 | + | AGACAATAAATACCGAGGAATG | 45769778 | 45769799 | TCGG | 45769800 | 45769803 | 36.36 | 12 |
| SluD83 | 149 | + | GTGGGGACAGACAATAAATACC | 45769770 | 45769791 | GAGG | 45769792 | 45769795 | 45.45 | 13 |
| SluD84 | 150 | − | GTATTTATTGTCTGTCCCCACC | 45769769 | 45769790 | TAGG | 45769765 | 45769768 | 45.45 | 6 |
| SluD85 | 151 | + | GTCGGGGGTGGGGGTCCTAGGT | 45769750 | 45769771 | GGGG | 45769772 | 45769775 | 72.73 | 89 |
| SluD86 | 152 | + | GGTCGGGGGTGGGGGTCCTAGG | 45769749 | 45769770 | TGGG | 45769771 | 45769774 | 77.27 | 37 |
| SluD87 | 153 | + | GGGTCGGGGGTGGGGGTCCTAG | 45769748 | 45769769 | GTGG | 45769770 | 45769773 | 77.27 | 44 |
| SluD88 | 154 | + | CGAGGGTCGGGGGTGGGGGTCC | 45769745 | 45769766 | TAGG | 45769767 | 45769770 | 81.82 | 56 |
| SluD89 | 155 | + | TTATTCGCGAGGGTCGGGGGTG | 45769738 | 45769759 | GGGG | 45769760 | 45769763 | 63.64 | 4 |
| SluD90 | 156 | − | CACCCCCGACCCTCGCGAATAA | 45769738 | 45769759 | AAGG | 45769734 | 45769737 | 63.64 | 0 |
| SluD91 | 157 | + | TTTATTCGCGAGGGTCGGGGGT | 45769737 | 45769758 | GGGG | 45769759 | 45769762 | 59.09 | 3 |
| SluD92 | 158 | + | TTTTATTCGCGAGGGTCGGGGG | 45769736 | 45769757 | TGGG | 45769758 | 45769761 | 59.09 | 5 |
| SluD93 | 159 | + | CTTTTATTCGCGAGGGTCGGGG | 45769735 | 45769756 | GTGG | 45769757 | 45769760 | 59.09 | 2 |
| SluD94 | 160 | + | GGCCTTTTATTCGCGAGGGTCG | 45769732 | 45769753 | GGGG | 45769754 | 45769757 | 59.09 | 1 |
| SluD95 | 161 | + | GGGCCTTTTATTCGCGAGGGTC | 45769731 | 45769752 | GGGG | 45769753 | 45769756 | 59.09 | 4 |
| SluD96 | 162 | + | AGGGCCTTTTATTCGCGAGGGT | 45769730 | 45769751 | CGGG | 45769752 | 45769755 | 54.55 | 2 |
| SluD97 | 163 | + | GAGGGCCTTTTATTCGCGAGGG | 45769729 | 45769750 | TCGG | 45769751 | 45769754 | 59.09 | 1 |
| SluD98 | 164 | + | GATGGAGGGCCTTTTATTCGCG | 45769725 | 45769746 | AGGG | 45769747 | 45769750 | 54.55 | 1 |
| SluD99 | 165 | + | AGATGGAGGGCCTTTTATTCGC | 45769724 | 45769745 | GAGG | 45769746 | 45769749 | 50 | 2 |
| SluD100 | 166 | + | GTCCAGAGCTTTGGGCAGATGG | 45769708 | 45769729 | AGGG | 45769730 | 45769733 | 59.09 | 1 |
| SluD101 | 167 | | AGTCCAGAGCTTTGGGCAGATG | 45769707 | 45769728 | GAGG | 45769729 | 45769722 | 54.55 | 10 |
| SluR1* | 168 | − | gctgctgctgctgctgctgctg | 45770208 | 45770229 | ctgG | 45770204 | 45770207 | 68.18 | 1932 |
| SluR2* | 169 | − | ctgctgctgctgctgctgctgc | 45770207 | 45770228 | tgGG | 45770203 | 45770206 | 68.18 | 1644 |
| SluR3* | 170 | − | tgctgctgctgctgctgctgct | 45770206 | 45770227 | gGGG | 45770202 | 45770205 | 63.64 | 1521 |
| SluR4* | 171 | − | gctgctgctgctgctgctgctg | 45770205 | 45770226 | GGGG | 45770201 | 45770204 | 68.18 | 1932 |
| SluR5* | 172 | − | ctgctgctgctgctgctgctgG | 45770204 | 45770225 | GGGG | 45770200 | 45770203 | 68.18 | 1352 |

*not selected for evaluation in primary DM1 patient myoblasts due to high number of predicted OFF-target sites

TABLE 1B
Exemplary SluCas9 sgRNAs with PAM sequences in
the 3′ UTR region of human DMPK gene

| SEQ ID NO | Strand | Guide Sequence |
|---|---|---|
| 1 | − | AACCCTAGAACTGTCTTCGACT |
| 201 | − | AACCCTAGAACTGTCTTCGAC |
| 202 | − | AACCCTAGAACTGTCTTCGA |
| 2 | − | ACCCTAGAACTGTCTTCGACTC |
| 203 | − | ACCCTAGAACTGTCTTCGACT |
| 204 | − | ACCCTAGAACTGTCTTCGAC |
| 3 | − | CCCTAGAACTGTCTTCGACTCC |
| 205 | − | CCCTAGAACTGTCTTCGACTC |
| 206 | − | CCCTAGAACTGTCTTCGACT |
| 4 | + | CCCCGGAGTCGAAGACAGTTCT |
| 207 | + | CCCGGAGTCGAAGACAGTTCT |
| 208 | + | CCGGAGTCGAAGACAGTTCT |
| 5 | + | GCCCCGGAGTCGAAGACAGTTC |
| 209 | + | CCCCGGAGTCGAAGACAGTTC |
| 210 | + | CCCGGAGTCGAAGACAGTTC |
| 6 | − | TGTCTTCGACTCCGGGGCCCCG |
| 211 | − | TGTCTTCGACTCCGGGGCCCC |
| 212 | − | TGTCTTCGACTCCGGGGCCC |
| 7 | + | CACTCAGTCTTCCAACGGGGCC |
| 213 | + | ACTCAGTCTTCCAACGGGGCC |
| 214 | + | CTCAGTCTTCCAACGGGGCC |
| 8 | − | GCCCCGTTGGAAGACTGAGTGC |
| 215 | − | GCCCCGTTGGAAGACTGAGTG |
| 216 | − | GCCCCGTTGGAAGACTGAGT |
| 9 | − | CCCCGTTGGAAGACTGAGTGCC |
| 217 | − | CCCCGTTGGAAGACTGAGTGC |
| 218 | − | CCCCGTTGGAAGACTGAGTG |
| 10 | − | CCCGTTGGAAGACTGAGTGCCC |
| 219 | − | CCCGTTGGAAGACTGAGTGCC |
| 220 | − | CCCGTTGGAAGACTGAGTGC |
| 11 | + | CCCGGGCACTCAGTCTTCCAAC |
| 221 | + | CCGGGCACTCAGTCTTCCAAC |
| 222 | + | CGGGCACTCAGTCTTCCAAC |
| 12 | + | CCCCGGGCACTCAGTCTTCCAA |
| 223 | + | CCCGGGCACTCAGTCTTCCAA |
| 224 | + | CCGGGCACTCAGTCTTCCAA |
| 13 | + | GCCCCGGGCACTCAGTCTTCCA |
| 225 | + | CCCCGGGCACTCAGTCTTCCA |
| 226 | + | CCCGGGCACTCAGTCTTCCA |
| 14 | − | TGGAAGACTGAGTGCCCGGGGC |
| 227 | − | TGGAAGACTGAGTGCCCGGGG |
| 228 | − | TGGAAGACTGAGTGCCCGGG |
| 15 | + | GCGCGGCTTCTGTGCCGTGCCC |
| 229 | + | CGCGGCTTCTGTGCCGTGCCC |
| 230 | + | GCGGCTTCTGTGCCGTGCCC |
| 16 | + | GGCGCGGCTTCTGTGCCGTGCC |
| 231 | + | GCGCGGCTTCTGTGCCGTGCC |
| 232 | + | CGCGGCTTCTGTGCCGTGCC |
| 17 | + | TGTGAACTGGCAGGCGGTGGGC |
| 233 | + | GTGAACTGGCAGGCGGTGGGC |
| 234 | + | TGAACTGGCAGGCGGTGGGC |
| 18 | + | GCGGTTGTGAACTGGCAGGCGG |
| 235 | + | CGGTTGTGAACTGGCAGGCGG |
| 236 | + | GGTTGTGAACTGGCAGGCGG |
| 19 | + | AGCGGTTGTGAACTGGCAGGCG |
| 237 | + | GCGGTTGTGAACTGGCAGGCG |
| 238 | + | CGGTTGTGAACTGGCAGGCG |
| 20 | + | CGGAGCGGTTGTGAACTGGCAG |
| 239 | + | GGAGCGGTTGTGAACTGGCAG |
| 240 | + | GAGCGGTTGTGAACTGGCAG |
| 21 | + | GCTCGGAGCGGTTGTGAACTGG |
| 241 | + | CTCGGAGCGGTTGTGAACTGG |
| 242 | + | TCGGAGCGGTTGTGAACTGG |
| 22 | − | CCAGTTCACAACCGCTCCGAGC |
| 243 | − | CCAGTTCACAACCGCTCCGAG |
| 244 | − | CCAGTTCACAACCGCTCCGA |
| 23 | − | CAGTTCACAACCGCTCCGAGCG |
| 245 | − | CAGTTCACAACCGCTCCGAGC |
| 246 | − | CAGTTCACAACCGCTCCGAG |
| 24 | + | CCACGCTCGGAGCGGTTGTGAA |
| 247 | + | CACGCTCGGAGCGGTTGTGAA |
| 248 | + | ACGCTCGGAGCGGTTGTGAA |
| 25 | + | TGGGCGGAGACCCACGCTCGGA |
| 249 | + | GGGCGGAGACCCACGCTCGGA |
| 250 | + | GGCGGAGACCCACGCTCGGA |
| 26 | + | GGAGCTGGGCGGAGACCCACGC |
| 251 | + | GAGCTGGGCGGAGACCCACGC |
| 252 | + | AGCTGGGCGGAGACCCACGC |
| 27 | − | CGCCCAGCTCCAGTCCTGTGAT |
| 253 | − | CGCCCAGCTCCAGTCCTGTGA |
| 254 | − | CGCCCAGCTCCAGTCCTGTG |
| 28 | − | GCCCAGCTCCAGTCCTGTGATC |
| 255 | − | GCCCAGCTCCAGTCCTGTGAT |
| 256 | − | GCCCAGCTCCAGTCCTGTGA |
| 29 | + | CGGATCACAGGACTGGAGCTGG |
| 257 | + | GGATCACAGGACTGGAGCTGG |
| 258 | + | GATCACAGGACTGGAGCTGG |
| 30 | + | GCCCGGATCACAGGACTGGAGC |
| 259 | + | CCCGGATCACAGGACTGGAGC |
| 260 | + | CCGGATCACAGGACTGGAGC |
| 31 | + | GGCCCGGATCACAGGACTGGAG |
| 261 | + | GCCCGGATCACAGGACTGGAG |
| 262 | + | CCCGGATCACAGGACTGGAG |
| 32 | + | GGGGCGGGCCCGGATCACAGGA |
| 263 | + | GGGCGGGCCCGGATCACAGGA |
| 264 | + | GGCGGGCCCGGATCACAGGA |
| 33 | − | TGTGATCCGGGCCCGCCCCCTA |
| 265 | − | TGTGATCCGGGCCCGCCCCCT |
| 266 | − | TGTGATCCGGGCCCGCCCCC |
| 34 | + | GCTAGGGGGCGGGCCCGGATCA |
| 267 | + | CTAGGGGGCGGGCCCGGATCA |
| 268 | + | TAGGGGGCGGGCCCGGATCA |
| 35 | − | ATCCGGGCCCGCCCCCTAGCgg |
| 269 | − | ATCCGGGCCCGCCCCCTAGCg |
| 270 | − | ATCCGGGCCCGCCCCCTAGC |
| 36 | − | TCCGGGCCCGCCCCCTAGCggc |
| 271 | − | TCCGGGCCCGCCCCCTAGCgg |
| 272 | − | TCCGGGCCCGCCCCCTAGCg |
| 37 | − | CCGGGCCCGCCCCCTAGCggcc |
| 273 | − | CCGGGCCCGCCCCCTAGCggc |
| 274 | − | CCGGGCCCGCCCCCTAGCgg |
| 38 | − | GGCCCGCCCCCTAGCggccggg |
| 275 | − | GGCCCGCCCCCTAGCggccgg |
| 276 | − | GGCCCGCCCCCTAGCggccg |
| 39 | + | ccccggccGCTAGGGGGCGGGC |
| 277 | + | cccggccGCTAGGGGGCGGGC |
| 278 | + | ccggccGCTAGGGGGCGGGC |
| 40 | − | GCCCGCCCCCTAGCggccgggg |
| 279 | − | GCCCGCCCCCTAGCggccggg |
| 280 | − | GCCCGCCCCCTAGCggccgg |
| 41 | − | CGCCCCCTAGCggccggggagg |
| 281 | − | CGCCCCCTAGCggccggggag |
| 282 | − | CGCCCCCTAGCggccgggga |
| 42 | − | GCCCCCTAGCggccggggaggg |
| 283 | − | GCCCCCTAGCggccggggagg |
| 284 | − | GCCCCCTAGCggccggggag |
| 43 | + | tccctccccggccGCTAGGGGG |
| 285 | + | ccctccccggccGCTAGGGGG |
| 286 | + | cctccccggccGCTAGGGGG |
| 44 | − | CCCCCTAGCggccggggaggga |
| 287 | − | CCCCCTAGCggccggggaggg |
| 288 | − | CCCCCTAGCggccggggagg |
| 45 | + | ctccctccccggccGCTAGGGG |
| 289 | + | tccctccccggccGCTAGGGG |
| 290 | + | ccctccccggccGCTAGGGG |
| 46 | + | cccctccctccccggccGCTAG |
| 291 | + | ccctccctccccggccGCTAG |
| 292 | + | cctccctccccggccGCTAG |
| 47 | − | CTAGCggccggggagggagggg |
| 293 | − | CTAGCggccggggagggaggg |
| 294 | − | CTAGCggccggggagggagg |
| 48 | + | gcccctccctccccggccGCTA |
| 295 | + | cccctccctccccggccGCTA |
| 296 | + | ccctccctccccggccGCTA |
| 49 | − | TAGCggccggggagggaggggc |
| 297 | − | TAGCggccggggagggagggg |
| 298 | − | TAGCggccggggagggaggg |
| 50 | + | ggcccctccctccccggccGCT |
| 299 | + | gcccctccctccccggccGCT |
| 300 | + | cccctccctccccggccGCT |
| 51 | + | cggcccctccctccccggccGC |
| 301 | + | ggcccctccctccccggccGC |
| 302 | + | gcccctccctccccggccGC |
| 52 | − | cggggagggaggggccgggtcc |
| 303 | − | cggggagggaggggccgggtc |
| 304 | − | cggggagggaggggccgggt |
| 53 | + | cgcggacccggcccctccctcc |
| 305 | + | geggacceggcccctccctcc |
| 306 | + | cggacccggcccctccctcc |
| 54 | − | gagggaggggccgggtccgcgg |
| 307 | − | gagggaggggccgggtccgcg |
| 308 | − | gagggaggggccgggtccgc |
| 55 | − | gggccgggtccgcggccggcga |
| 309 | − | gggccgggtccgcggccggcg |
| 310 | − | gggccgggtccgcggccggc |
| 56 | − | ggccgggtccgcggccggcgaa |
| 311 | − | ggccgggtccgcggccggcga |
| 312 | − | ggccgggtccgcggccggcg |
| 57 | − | gccgggtccgcggccggcgaac |
| 313 | − | gccgggtccgcggccggcgaa |
| 314 | − | gccgggtccgcggccggcga |
| 58 | + | gccccgttcgccggccgcggac |
| 315 | + | ccccgttcgccggccgcggac |
| 316 | + | cccgttcgccggccgcggac |
| 59 | − | cgcggccggcgaacggggcTCG |
| 317 | − | cgcggccggcgaacggggcTC |
| 318 | − | cgcggccggcgaacggggcT |
| 60 | − | gcggccggcgaacggggcTCGA |
| 319 | − | gcggccggcgaacggggcTCG |
| 320 | − | gcggccggcgaacggggcTC |
| 61 | + | CTTCGAgccccgttcgccggcc |
| 321 | + | TTCGAgccccgttcgccggcc |
| 322 | + | TCGAgccccgttcgccggcc |
| 62 | + | AGGACCCTTCGAgccccgttcg |
| 323 | + | GGACCCTTCGAgccccgttcg |
| 324 | + | GACCCTTCGAgccccgttcg |
| 63 | − | ggggcTCGAAGGGTCCTTGTAG |
| 325 | − | ggggcTCGAAGGGTCCTTGTA |
| 326 | − | ggggcTCGAAGGGTCCTTGT |
| 64 | − | gggcTCGAAGGGTCCTTGTAGC |
| 327 | − | gggcTCGAAGGGTCCTTGTAG |
| 328 | − | gggcTCGAAGGGTCCTTGTA |
| 65 | + | cagcagcagcaTTCCCGGCTAC |
| 329 | + | agcagcagcaTTCCCGGCTAC |
| 330 | + | gcagcagcaTTCCCGGCTAC |
| 66 | + | agcagcagcagcagcagcaTTC |
| 331 | + | gcagcagcagcagcagcaTTC |
| 332 | + | cagcagcagcagcagcaTTC |
| 67 | − | GATCACAGACCATTTCTTTCTT |
| 333 | − | GATCACAGACCATTTCTTTCT |
| 334 | − | GATCACAGACCATTTCTTTC |
| 68 | − | CAGACCATTTCTTTCTTTCGGC |
| 335 | − | CAGACCATTTCTTTCTTTCGG |
| 336 | − | CAGACCATTTCTTTCTTTCG |
| 69 | − | ATTTCTTTCTTTCGGCCAGGCT |
| 337 | − | ATTTCTTTCTTTCGGCCAGGC |
| 338 | − | ATTTCTTTCTTTCGGCCAGG |
| 70 | + | TCAGCCTGGCCGAAAGAAAGAA |
| 339 | + | CAGCCTGGCCGAAAGAAAGAA |
| 340 | + | AGCCTGGCCGAAAGAAAGAA |
| 71 | − | TCGGCCAGGCTGAGGCCCTGAC |
| 341 | − | TCGGCCAGGCTGAGGCCCTGA |
| 342 | − | TCGGCCAGGCTGAGGCCCTG |
| 72 | − | CCAGGCTGAGGCCCTGACGTGG |
| 343 | − | CCAGGCTGAGGCCCTGACGTG |
| 344 | − | CCAGGCTGAGGCCCTGACGT |
| 73 | − | CAGGCTGAGGCCCTGACGTGGA |
| 345 | − | CAGGCTGAGGCCCTGACGTGG |
| 346 | − | CAGGCTGAGGCCCTGACGTG |
| 74 | + | CCATCCACGTCAGGGCCTCAGC |
| 347 | + | CATCCACGTCAGGGCCTCAGC |
| 348 | + | ATCCACGTCAGGGCCTCAGC |
| 75 | − | CCTGACGTGGATGGGCAAACTG |
| 349 | − | CCTGACGTGGATGGGCAAACT |
| 350 | − | CCTGACGTGGATGGGCAAAC |
| 76 | + | CTGCAGTTTGCCCATCCACGTC |
| 351 | + | TGCAGTTTGCCCATCCACGTC |
| 173 | + | GCAGTTTGCCCATCCACGTC |
| 77 | + | CCTGCAGTTTGCCCATCCACGT |
| 352 | + | CTGCAGTTTGCCCATCCACGT |
| 353 | + | TGCAGTTTGCCCATCCACGT |
| 78 | − | CGTGGATGGGCAAACTGCAGGC |
| 354 | − | CGTGGATGGGCAAACTGCAGG |
| 355 | − | CGTGGATGGGCAAACTGCAG |
| 79 | − | GTGGATGGGCAAACTGCAGGCC |
| 356 | − | GTGGATGGGCAAACTGCAGGC |
| 357 | − | GTGGATGGGCAAACTGCAGG |
| 80 | − | CAGGCCTGGGAAGGCAGCAAGC |
| 358 | − | CAGGCCTGGGAAGGCAGCAAG |
| 359 | − | CAGGCCTGGGAAGGCAGCAA |
| 81 | − | ATGGGCAAACTGCAGGCCTGGG |
| 360 | − | ATGGGCAAACTGCAGGCCTGG |
| 361 | − | ATGGGCAAACTGCAGGCCTG |
| 82 | − | GCAGGCCTGGGAAGGCAGCAAG |
| 362 | − | GCAGGCCTGGGAAGGCAGCAA |
| 363 | − | GCAGGCCTGGGAAGGCAGCA |
| 83 | + | ACGGCCCGGCTTGCTGCCTTCC |
| 364 | + | CGGCCCGGCTTGCTGCCTTCC |
| 365 | + | GGCCCGGCTTGCTGCCTTCC |
| 84 | + | TGGAGGATGGAACACGGACGGC |
| 366 | + | GGAGGATGGAACACGGACGGC |
| 367 | + | GAGGATGGAACACGGACGGC |
| 85 | + | GTGCGTGGAGGATGGAACACGG |
| 368 | + | TGCGTGGAGGATGGAACACGG |
| 369 | + | GCGTGGAGGATGGAACACGG |
| 86 | + | GGGGGTGCGTGGAGGATGGAAC |
| 370 | + | GGGGTGCGTGGAGGATGGAAC |
| 371 | + | GGGTGCGTGGAGGATGGAAC |
| 87 | + | GATAGGTGGGGGTGCGTGGAGG |
| 372 | + | ATAGGTGGGGGTGCGTGGAGG |
| 373 | + | TAGGTGGGGGTGCGTGGAGG |
| 88 | − | CTCCACGCACCCCCACCTATCG |
| 374 | − | CTCCACGCACCCCCACCTATC |
| 375 | − | CTCCACGCACCCCCACCTAT |
| 89 | + | CAACGATAGGTGGGGGTGCGTG |
| 376 | + | AACGATAGGTGGGGGTGCGTG |
| 377 | + | ACGATAGGTGGGGGTGCGTG |
| 90 | + | AACCAACGATAGGTGGGGGTGC |
| 378 | + | ACCAACGATAGGTGGGGGTGC |
| 379 | + | CCAACGATAGGTGGGGGTGC |
| 91 | + | CTTTGCGAACCAACGATAGGTG |
| 380 | + | TTTGCGAACCAACGATAGGTG |
| 381 | + | TTGCGAACCAACGATAGGTG |
| 92 | + | ACTTTGCGAACCAACGATAGGT |
| 382 | + | CTTTGCGAACCAACGATAGGT |
| 383 | + | TTTGCGAACCAACGATAGGT |
| 93 | + | CACTTTGCGAACCAACGATAGG |
| 384 | + | ACTTTGCGAACCAACGATAGG |
| 385 | + | CTTTGCGAACCAACGATAGG |
| 94 | + | GCACTTTGCGAACCAACGATAG |
| 386 | + | CACTTTGCGAACCAACGATAG |
| 387 | + | ACTTTGCGAACCAACGATAG |
| 95 | + | TTTGCACTTTGCGAACCAACGA |
| 388 | + | TTGCACTTTGCGAACCAACGA |
| 389 | + | TGCACTTTGCGAACCAACGA |
| 96 | − | TTCTTGTGCATGACGCCCTGCT |
| 390 | − | TTCTTGTGCATGACGCCCTGC |
| 391 | − | TTCTTGTGCATGACGCCCTG |
| 97 | − | TCTTGTGCATGACGCCCTGCTC |
| 392 | − | TCTTGTGCATGACGCCCTGCT |
| 393 | − | TCTTGTGCATGACGCCCTGC |
| 98 | − | CTTGTGCATGACGCCCTGCTCT |
| 394 | − | CTTGTGCATGACGCCCTGCTC |
| 395 | − | CTTGTGCATGACGCCCTGCT |
| 99 | − | GACGCCCTGCTCTGGGGAGCGT |
| 396 | − | GACGCCCTGCTCTGGGGAGCG |
| 397 | − | GACGCCCTGCTCTGGGGAGC |
| 100 | + | CGCGCCAGACGCTCCCCAGAGC |
| 398 | + | GCGCCAGACGCTCCCCAGAGC |
| 399 | + | CGCCAGACGCTCCCCAGAGC |
| 101 | + | TCGCGCCAGACGCTCCCCAGAG |
| 400 | + | CGCGCCAGACGCTCCCCAGAG |
| 401 | + | GCGCCAGACGCTCCCCAGAG |
| 102 | − | GGCGCGATCTCTGCCTGCTTAC |
| 402 | − | GGCGCGATCTCTGCCTGCTTA |
| 403 | − | GGCGCGATCTCTGCCTGCTT |
| 103 | − | GCGCGATCTCTGCCTGCTTACT |
| 404 | − | GCGCGATCTCTGCCTGCTTAC |
| 405 | − | GCGCGATCTCTGCCTGCTTA |
| 104 | + | AAAAGCAAATTTCCCGAGTAAG |
| 406 | + | AAAGCAAATTTCCCGAGTAAG |
| 407 | + | AAGCAAATTTCCCGAGTAAG |
| 105 | − | TGCTTTTGCCAAACCCGCTTTT |
| 408 | − | TGCTTTTGCCAAACCCGCTTT |
| 409 | − | TGCTTTTGCCAAACCCGCTT |
| 106 | − | GCTTTTGCCAAACCCGCTTTTT |
| 410 | − | GCTTTTGCCAAACCCGCTTTT |
| 411 | − | GCTTTTGCCAAACCCGCTTT |
| 107 | − | CTTTTGCCAAACCCGCTTTTTC |
| 412 | − | CTTTTGCCAAACCCGCTTTTT |
| 413 | − | CTTTTGCCAAACCCGCTTTT |
| 108 | + | CGGGATCCCCGAAAAAGCGGGT |
| 414 | + | GGGATCCCCGAAAAAGCGGGT |
| 415 | + | GGATCCCCGAAAAAGCGGGT |
| 109 | + | GGGCGCGGGATCCCCGAAAAAG |
| 416 | + | GGCGCGGGATCCCCGAAAAAG |
| 417 | + | GCGCGGGATCCCCGAAAAAG |
| 110 | + | GGGGCGCGGGATCCCCGAAAAA |
| 418 | + | GGGCGCGGGATCCCCGAAAAA |
| 419 | + | GGCGCGGGATCCCCGAAAAA |
| 111 | + | AGCGCAAGTGAGGAGGGGGGCG |
| 420 | + | GCGCAAGTGAGGAGGGGGGCG |
| 421 | + | CGCAAGTGAGGAGGGGGGCG |
| 112 | + | CAGCGCAAGTGAGGAGGGGGGC |
| 422 | + | AGCGCAAGTGAGGAGGGGGGC |
| 423 | + | GCGCAAGTGAGGAGGGGGGC |
| 113 | − | CCCCTCCTCACTTGCGCTGCTC |
| 424 | − | CCCCTCCTCACTTGCGCTGCT |
| 425 | − | CCCCTCCTCACTTGCGCTGC |
| 114 | + | GAGAGCAGCGCAAGTGAGGAGG |
| 426 | + | AGAGCAGCGCAAGTGAGGAGG |
| 427 | + | GAGCAGCGCAAGTGAGGAGG |
| 115 | + | CGAGAGCAGCGCAAGTGAGGAG |
| 428 | + | GAGAGCAGCGCAAGTGAGGAG |
| 429 | + | AGAGCAGCGCAAGTGAGGAG |
| 116 | + | CCGAGAGCAGCGCAAGTGAGGA |
| 430 | + | CGAGAGCAGCGCAAGTGAGGA |
| 431 | + | GAGAGCAGCGCAAGTGAGGA |
| 117 | + | TCCGAGAGCAGCGCAAGTGAGG |
| 432 | + | CCGAGAGCAGCGCAAGTGAGG |
| 433 | + | CGAGAGCAGCGCAAGTGAGG |
| 118 | + | CTCCGAGAGCAGCGCAAGTGAG |
| 434 | + | TCCGAGAGCAGCGCAAGTGAG |
| 435 | + | CCGAGAGCAGCGCAAGTGAG |
| 119 | + | GGGCTCCGAGAGCAGCGCAAGT |
| 436 | + | GGCTCCGAGAGCAGCGCAAGT |
| 437 | + | GCTCCGAGAGCAGCGCAAGT |
| 120 | − | TGCGCTGCTCTCGGAGCCCCAG |
| 438 | − | TGCGCTGCTCTCGGAGCCCCA |
| 439 | − | TGCGCTGCTCTCGGAGCCCC |
| 121 | − | GCCCCAGCCGGCTCCGCCCGCT |
| 440 | − | GCCCCAGCCGGCTCCGCCCGC |
| 441 | − | GCCCCAGCCGGCTCCGCCCG |
| 122 | − | CCAGCCGGCTCCGCCCGCTTCG |
| 442 | − | CCAGCCGGCTCCGCCCGCTTC |
| 443 | − | CCAGCCGGCTCCGCCCGCTT |
| 123 | + | GCCGAAGCGGGCGGAGCCGGCT |
| 444 | + | CCGAAGCGGGCGGAGCCGGCT |
| 445 | + | CGAAGCGGGCGGAGCCGGCT |
| 124 | + | CGCCGAAGCGGGCGGAGCCGGC |
| 446 | + | GCCGAAGCGGGCGGAGCCGGC |
| 447 | + | CCGAAGCGGGCGGAGCCGGC |
| 125 | + | CCGCCGAAGCGGGCGGAGCCGG |
| 448 | + | CGCCGAAGCGGGCGGAGCCGG |
| 449 | + | GCCGAAGCGGGCGGAGCCGG |
| 126 | − | CGGCTCCGCCCGCTTCGGCGGT |
| 450 | − | CGGCTCCGCCCGCTTCGGCGG |
| 451 | − | CGGCTCCGCCCGCTTCGGCG |
| 127 | + | CAAACCGCCGAAGCGGGCGGAG |
| 452 | + | AAACCGCCGAAGCGGGCGGAG |
| 453 | + | AACCGCCGAAGCGGGCGGAG |
| 128 | + | AATATCCAAACCGCCGAAGCGG |
| 454 | + | ATATCCAAACCGCCGAAGCGG |
| 455 | + | TATCCAAACCGCCGAAGCGG |
| 129 | + | ATAAATATCCAAACCGCCGAAG |
| 456 | + | TAAATATCCAAACCGCCGAAG |
| 457 | + | AAATATCCAAACCGCCGAAG |
| 130 | + | AATAAATATCCAAACCGCCGAA |
| 458 | + | ATAAATATCCAAACCGCCGAA |
| 459 | + | TAAATATCCAAACCGCCGAA |
| 131 | − | ACCTCGTCCTCCGACTCGCTGA |
| 460 | − | ACCTCGTCCTCCGACTCGCTG |
| 461 | − | ACCTCGTCCTCCGACTCGCT |
| 132 | + | GCCTGTCAGCGAGTCGGAGGAC |
| 462 | + | CCTGTCAGCGAGTCGGAGGAC |
| 463 | + | CTGTCAGCGAGTCGGAGGAC |
| 133 | − | CCTCCGACTCGCTGACAGGCTA |
| 464 | − | CCTCCGACTCGCTGACAGGCT |
| 465 | − | CCTCCGACTCGCTGACAGGC |
| 134 | + | CCTGTAGCCTGTCAGCGAGTCG |
| 466 | + | CTGTAGCCTGTCAGCGAGTCG |
| 467 | + | TGTAGCCTGTCAGCGAGTCG |
| 135 | + | GGTCCTGTAGCCTGTCAGCGAG |
| 468 | + | GTCCTGTAGCCTGTCAGCGAG |
| 469 | + | TCCTGTAGCCTGTCAGCGAG |
| 136 | − | CCCAACAACCCCAATCCACGTT |
| 470 | − | CCCAACAACCCCAATCCACGT |
| 471 | − | CCCAACAACCCCAATCCACG |
| 137 | + | AAAACGTGGATTGGGGTTGTTG |
| 472 | + | AAACGTGGATTGGGGTTGTTG |
| 473 | + | AACGTGGATTGGGGTTGTTG |
| 138 | + | CAAAACGTGGATTGGGGTTGTT |
| 474 | + | AAAACGTGGATTGGGGTTGTT |
| 475 | + | AAACGTGGATTGGGGTTGTT |
| 139 | + | CCAAAACGTGGATTGGGGTTGT |
| 476 | + | CAAAACGTGGATTGGGGTTGT |
| 477 | + | AAAACGTGGATTGGGGTTGT |
| 140 | + | TCCAAAACGTGGATTGGGGTTG |
| 478 | + | CCAAAACGTGGATTGGGGTTG |
| 479 | + | CAAAACGTGGATTGGGGTTG |
| 141 | + | CAGTGCATCCAAAACGTGGATT |
| 480 | + | AGTGCATCCAAAACGTGGATT |
| 481 | + | GTGCATCCAAAACGTGGATT |
| 142 | + | TCAGTGCATCCAAAACGTGGAT |
| 482 | + | CAGTGCATCCAAAACGTGGAT |
| 483 | + | AGTGCATCCAAAACGTGGAT |
| 143 | + | CTCAGTGCATCCAAAACGTGGA |
| 484 | + | TCAGTGCATCCAAAACGTGGA |
| 485 | + | CAGTGCATCCAAAACGTGGA |
| 144 | + | GGGGTCTCAGTGCATCCAAAAC |
| 486 | + | GGGTCTCAGTGCATCCAAAAC |
| 487 | + | GGTCTCAGTGCATCCAAAAC |
| 145 | − | TGCACTGAGACCCCGACATTCC |
| 488 | − | TGCACTGAGACCCCGACATTC |
| 489 | − | TGCACTGAGACCCCGACATT |
| 146 | + | ACAATAAATACCGAGGAATGTC |
| 490 | + | CAATAAATACCGAGGAATGTC |
| 491 | + | AATAAATACCGAGGAATGTC |
| 147 | + | GACAATAAATACCGAGGAATGT |
| 492 | + | ACAATAAATACCGAGGAATGT |
| 493 | + | CAATAAATACCGAGGAATGT |
| 148 | + | AGACAATAAATACCGAGGAATG |
| 494 | + | GACAATAAATACCGAGGAATG |
| 495 | + | ACAATAAATACCGAGGAATG |
| 149 | + | GTGGGGACAGACAATAAATACC |
| 496 | + | TGGGGACAGACAATAAATACC |
| 497 | + | GGGGACAGACAATAAATACC |
| 150 | − | GTATTTATTGTCTGTCCCCACC |
| 498 | − | GTATTTATTGTCTGTCCCCAC |
| 499 | − | GTATTTATTGTCTGTCCCCA |
| 151 | + | GTCGGGGGTGGGGGTCCTAGGT |
| 500 | + | TCGGGGGTGGGGGTCCTAGGT |
| 501 | + | CGGGGGTGGGGGTCCTAGGT |
| 152 | + | GGTCGGGGGTGGGGGTCCTAGG |
| 502 | + | GTCGGGGGTGGGGGTCCTAGG |
| 503 | + | TCGGGGGTGGGGGTCCTAGG |
| 153 | + | GGGTCGGGGGTGGGGGTCCTAG |
| 504 | + | GGTCGGGGGTGGGGGTCCTAG |
| 505 | + | GTCGGGGGTGGGGGTCCTAG |
| 154 | + | CGAGGGTCGGGGGTGGGGGTCC |
| 506 | + | GAGGGTCGGGGGTGGGGGTCC |
| 507 | + | AGGGTCGGGGGTGGGGGTCC |
| 155 | + | TTATTCGCGAGGGTCGGGGGTG |
| 508 | + | TATTCGCGAGGGTCGGGGGTG |
| 509 | + | ATTCGCGAGGGTCGGGGGTG |
| 156 | − | CACCCCCGACCCTCGCGAATAA |
| 510 | − | CACCCCCGACCCTCGCGAATA |
| 511 | − | CACCCCCGACCCTCGCGAAT |
| 157 | + | TTTATTCGCGAGGGTCGGGGGT |
| 512 | + | TTATTCGCGAGGGTCGGGGGT |
| 513 | + | TATTCGCGAGGGTCGGGGGT |
| 158 | + | TTTTATTCGCGAGGGTCGGGGG |
| 514 | + | TTTATTCGCGAGGGTCGGGGG |
| 515 | + | TTATTCGCGAGGGTCGGGGG |
| 159 | + | CTTTTATTCGCGAGGGTCGGGG |
| 516 | + | TTTTATTCGCGAGGGTCGGGG |
| 517 | + | TTTATTCGCGAGGGTCGGGG |
| 160 | + | GGCCTTTTATTCGCGAGGGTCG |
| 518 | + | GCCTTTTATTCGCGAGGGTCG |
| 519 | + | CCTTTTATTCGCGAGGGTCG |
| 161 | + | GGGCCTTTTATTCGCGAGGGTC |
| 520 | + | GGCCTTTTATTCGCGAGGGTC |
| 521 | + | GCCTTTTATTCGCGAGGGTC |
| 162 | + | AGGGCCTTTTATTCGCGAGGGT |
| 522 | + | GGGCCTTTTATTCGCGAGGGT |
| 523 | + | GGCCTTTTATTCGCGAGGGT |
| 163 | + | GAGGGCCTTTTATTCGCGAGGG |
| 524 | + | AGGGCCTTTTATTCGCGAGGG |
| 525 | + | GGGCCTTTTATTCGCGAGGG |
| 164 | + | GATGGAGGGCCTTTTATTCGCG |
| 526 | + | ATGGAGGGCCTTTTATTCGCG |
| 527 | + | TGGAGGGCCTTTTATTCGCG |
| 165 | + | AGATGGAGGGCCTTTTATTCGC |
| 528 | + | GATGGAGGGCCTTTTATTCGC |
| 529 | + | ATGGAGGGCCTTTTATTCGC |
| 166 | + | GTCCAGAGCTTTGGGCAGATGG |
| 530 | + | TCCAGAGCTTTGGGCAGATGG |
| 531 | + | CCAGAGCTTTGGGCAGATGG |

2. In Silico Off-Target Assessment

Off-target sites were computationally predicted for each sgRNA based on sequence similarity to the hg38 human reference genome (Table 1A), specifically, any site that was identified to have a PAM sequence and have up to 3 mismatches, or up to 2 mismatches and 1 DNA/RNA bulge, relative to the protospacer sequence.

3. Genomic DNA Extraction, PCR Amplification and TapeStation

Genomic DNA of DM1 myoblasts was isolated with the Kingfisher Flex purification system (Thermal Fisher) in 96-well format following the manufacturer's instruction. The DMPK 3′ UTR region was amplified using GoTaq Green Master Mix (Promega) and PCR primers flanking the 3′ UTR region. In some embodiments, a forward primer sequence that may be used is CGCTAGGAAGCAGCCAATGA (SEQ ID NO: 532), and a reverse primer sequence that may be used is TAGCTCCTCCCAGACCTTCG (SEQ ID NO: 533). Amplification was conducted using the following cycling parameters: 1 cycle at 95° C. for 2 min; 40 cycles of 95° C. for 30 sec, 63° C. for 30 sec, and 72° C. for 90 sec; 1 cycle at 72° C. for 5 min. Only the wild type allele is amplified by the PCR reaction. The PCR products were analyzed on the TapeStation system with High Sensitivity D5000 ScreenTape (Agilent Technologies).

4. Sanger Sequencing and ICE Analysis

PCR products were purified and sequenced by Sanger sequencing. In some embodiments, sequencing primer UTRsF3 (AATGACGAGTTCGGACGG; (SEQ ID NO: 534)) may be used for sequencing upstream sgRNAs, and the reverse PCR primer (TAGCTCCTCCCAGACCTTCG; (SEQ ID NO: 533)) may be used for sequencing downstream sgRNAs. Indel values were estimated using the ICE analysis algorithm (Synthego) with the chromatogram files obtained from Sanger sequencing.

5. Primary Myoblast Culture

Primary healthy myoblasts (P01431-18F) and DM1 patient myoblasts (03001-32F) were obtained from Cook MyoSite. Myoblasts were cultured in myoblast growth medium consisting of Myotonic Basal Medium (Cook MyoSite, MB-2222) and MyoTonic Growth Supplement (Cook MyoSite, MS-3333). Three days before nucleofection, primary human myoblasts were further purified with EasySep Human CD56 Positive Selection Kit II (StemCell Tech, 17855) following the manufacturer's instruction, and then maintained in myoblast growth medium until nucleofection.

6. Preparation of RNPs

RNPs were assembled with recombinant SluCas9 protein and chemically modified sgRNAs at a ratio of 1:3 (protein:sgRNA). For SINGLE-cut screening, RNP complexes were assembled with 30 pmol of SluCas9 and 90 pmol of sgRNA in P5 Primary Cell Nucleofector Solution (Lonza). After incubation at room temperature for 20 minutes, 10 μL of RNP complex were mixed with two hundred thousand of primary myoblasts resuspended in 10 μL of P5 Nucleofector Solution. For DOUBLE-cut screening, RNP complexes were first assembled for individual sgRNAs with 20 pmol of SluCas9 protein and 60 pmol of sgRNAs in 5 μL of P5 Nucleofector Solution. After incubation at room temperature for 20 minutes, the two RNP complexes (one for upstream sgRNA and one for downstream sgRNA) were mixed at 1:1 ratio and then further mixed with two hundred thousand of primary myoblasts resuspended in 10 μL of P5 Nucleofector Solution.

7. Nucleofection of RNPs into Primary DM1 Myoblasts

The Nucleofector 96-well Shuttle System (Lonza) was used to deliver the SluCa9/sgRNA RNPs into primary DM1 patient myoblasts using the nucleofection program CM138. Following nucleofection, myoblasts from each well of nucleofection shuttle were split into six wells of the 96-well cell culture plate (Greiner, 655090) coated with matrigel. The first three wells were treated with DMSO for 48 hrs before changing to fresh myoblast growth medium, and the other three wells were treated with 3 μM of DNA-PKi Compound 6 for 48 hrs before changing to fresh myoblast growth medium. 72 hrs post nucleofection, two wells of DMSO-treated myoblasts and two wells of DNA-PKi-treated myoblasts from each nucleofection were harvested for genomic DNA extraction using the Kingfisher Flex purification system (Thermal Fisher), whereas one well of DMSO-treated myoblasts and one well of DNA-PKi-treated myoblasts were stained for RNA foci by FISH staining.

8. ddPCR

The primers and probes of ddPCR are designed using the online primer design software Primer3Plus (http://www.bioinformatics.nl/cgi-bin/primer3plus/primer3plus.cgi). In some embodiments, two target primers/probe sets were used to detect CTG repeat excision, and a reference primers/probe set were used to amplify a region located in Exon 1 of human DMPK gene and to serve as a reference control for the target sets. Examples of possible ddPCR primer and probe sequences are listed in Table 2. The 24 μL of ddPCR reaction consists of 12 μL of Supermix for Probes (no dUTP) (Bio-Rad Laboratories), 1 μL of Reference primers mix (21.6 μM), 1 μL of Reference probe (6 μM), 1 μL of Target primers mix (21.6 μM), 1 μL of Target probe (6 μM), and 8 μL of sample genomic DNA. Droplets were generated using probe oil with the QX200 Droplet Generator (Bio-Rad Laboratories). Droplets were transferred to a 96-well PCR plate, sealed and cycled in a C1000 deep well Thermocycler (Bio-Rad Laboratories) under the following cycling protocol: 1 cycle at 95° C. for 10 min; 40 cycles of 94° C. for 30 see, and 58° C. for 1 min; 1 cycle at 98° C. for 10 min (for enzyme inactivation). The cycled plate was then transferred and read in the FAM and HEX channels using the Bio-Rad QX200 Droplet Reader (Bio-Rad Laboratories). ddPCR analysis is performed with the Bio-Rad QuantaSoft Pro Software.

TABLE 2
Primer and probe sequences for loss-of-signal ddPCR assays

| ddPCR set | Oligo type | Name | Sequence (5′ à 3′) |
|---|---|---|---|
| Target_Downstream | Forward | UTRF1 | GGGGATCACAGACCATTTCT (SEQ |
| | Primer | | ID NO: 535) |
| | Reverse | UTRR14 | TGGAGGATGGAACACGGAC (SEQ |
| | Primer | | ID NO: 536) |
| | Probe | UTRP2-FAM | TTCTTTCGGCCAGGCTGAGGCCCT |
| | | | (SEQ ID NO: 537) |
| Target_Upstream | Forward | UpExcisionF | CTAGCGGCCGGGGAG (SEQ ID NO: |
| | Primer | | 538) |
| | Reverse | UpExcisionR | AGCAGCATTCCCGGCTA (SEQ ID |
| | Primer | | NO: 539) |
| | Probe | UpExcisionP- | CGAACGGGGCTCGAAGGGTCCTTG |
| | | FAM | (SEQ ID NO: 540) |
| Reference | Forward | DMPKF8 | GGATATGTGACCATGCTACC (SEQ |
| | Primer | | ID NO: 541) |
| | Reverse | DMPKR7 | GGGTTGTATCCAGTACCTCT (SEQ |
| | Primer | | ID NO: 542) |
| | Probe | DMPKP6- | TGTCCTGTTCCTTCCCCCAGCCCCA |
| | | HEX | (SEQ ID NO: 543) |

9. FISH Staining of RNA Foci

Primary myoblasts were fixed for 15 min with 4% paraformaldehyde (PFA) and washed five times with 1×PBS for 10 min each at room temperature. Before staining, cells were permeabilized with 0.5% triton X-100 in 1×PBS for 5 min at room temperature, and then washed with 30% formamide and 2× saline-sodium citrate (SSC) mixture for 10 min at room temperature. Cells were then stained with 1 ng/μL of Cy3-PNA(CAG)₅ probe (PNA Bio, F5001) diluted in 30% formamide, 2×SSC, 2 μg/mL BSA, 66 μg/mL yeast tRNA, and 2 mM vanadyl complex for 15 min at 80° C. Following probe staining, cells were then washed in 30% formamide and 2×SSC mixture for 30 min at 42° C., then washed in 30% formamide and 2×SSC mixture for 30 min at 37° C., and then washed in 1×SSC solution for 10 min at room temperature, and finally washed in 1×PBS for 10 min at room temperature. Cells were next stained with anti-MBNL1 antibody (Santa Cruz, 3A4) diluted in 1% bovine serum albumin (BSA) for overnight at 4° C., and washed twice with 1×PBS for 10 min each at room temperature. Cells were then incubated with the secondary antibody goat anti-rabbit Alexa 647 (Thermo Fisher, A32728) diluted in 1% BSA for 1 hr at room temperature, and washed twice with 1×PBS for 10 min each at room temperature. Next, cells were stained with Hoechst solution (Thermo Fisher, H3569) at 0.1 mg/ml for 5 min, and washed once with 1×PBS for 5 min. PBS was aspirated and fresh 100 μl of fresh PBS is added to each well. High-throughput acquisition of images was completed with the ImageXpress Micro Confocal High-Content Imaging System (Molecular Devices). RNA foci quantifications were accomplished with a customized analysis module of the MetaXpress program (Molecular Devices).

B. Single Cut Screening

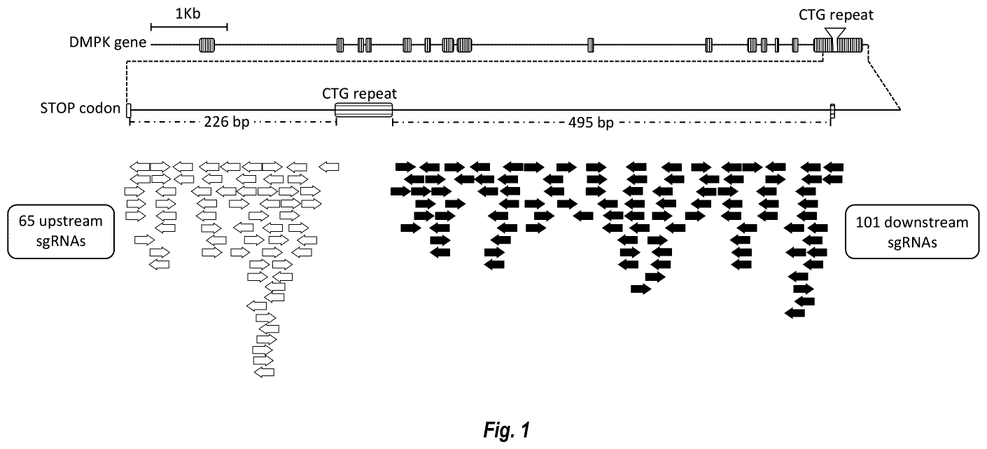

One hundred and seventy-two (172) SluCas9 sgRNAs with the canonical NNGG Protospacer Adjacent Motif (PAM) motif were identified targeting the 3′ UTR of the human DMPK gene, where a double-strand break (DSB) point would be made between the stop codon and the end of the last exon of DMPKgene, so that CRISPR-induced gene editing would not interfere with the DMPK coding sequence and mRNA maturation (Table 1A). Six (6) sgRNAs (SluU66, SluR1, SluR2, SluR3, SluR4, and SluR5) were excluded from further evaluation due to high number of predicted off-target sites (Table 1A). Among the remaining 166 sgRNAs, 65 sgRNAs (SluU01-SluU65) are located upstream of the CTG repeat expansion (between the stop codon and the CTG repeat expansion), and 101 sgRNAs (SluD01-SluD101) are located downstream of the CTG repeat expansion (between the CTG repeat expansion and the end of the last exon of DMPK gene) (FIG. 1).

To assess the efficiency of individual SluCas9 sgRNAs for inducing indel editing and CTG repeat excision, single-cut screening was performed in which individual SluCas9 sgRNAs and recombinant SluCas9 protein were assembled into ribonucleoprotein (RNP) and delivered into primary DM1 patient myoblasts. Nucleofected myoblasts were treated with either DMSO (vehicle) or 3 μM of DNA-dependent Protein Kinase Inhibitor (DNA-PKi) Compound 6 for 48 hrs. Seventy-two (72) hrs post nucleofection, myoblasts were subjected to either genomic DNA isolation or RNA foci staining by fluorescence in situ hybridization (FISH).

A 1174 bp sequence covering the CTG repeat expansion and the sgRNAs targeting region in the wild-type allele was amplified by PCR from the extracted genomic DNA. Sanger sequencing and ICE analysis were then performed to quantify the frequency of indels induced by individual sgRNAs. It is of note that only the vehicle-treated samples were used for ICE analysis.

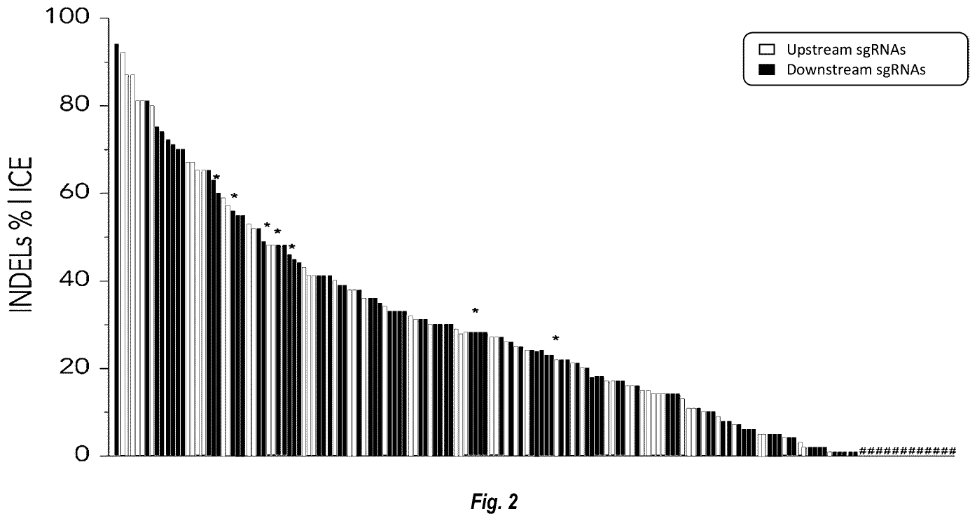

Among the 166 sgRNAs evaluated, 8 sgRNAs induced indel efficiency greater than 80%, 21 sgRNAs induced indel efficiency greater than 60%, and 44 sgRNAs induced indel efficiency greater than 40% (FIG. 2 and Table 3). Editing efficiency was assessed by Sanger sequencing and ICE analysis. The sgRNAs were ordered from the highest efficiency to the lowest efficiency in FIG. 2. The * indicates the sgRNAs with R2<0.9 in the ICE analysis (in-house QC standard for reliable ICE analysis).

Eleven (11) sgRNAs failed ICE analysis (denoted as “#” in FIG. 2) since the ICE algorithm could not align their sanger sequencing chromatograms with the control sequencing chromatogram.

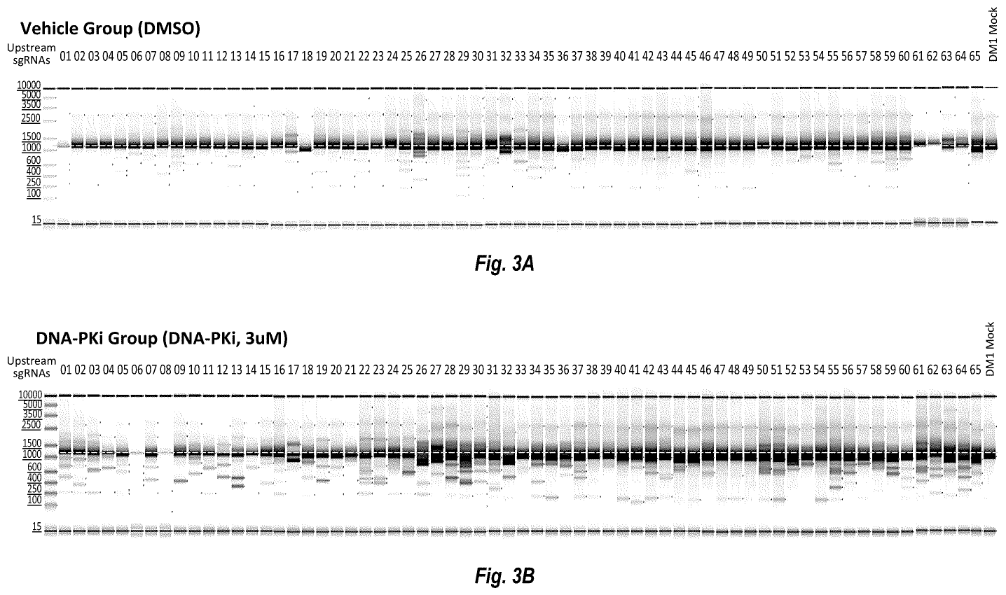

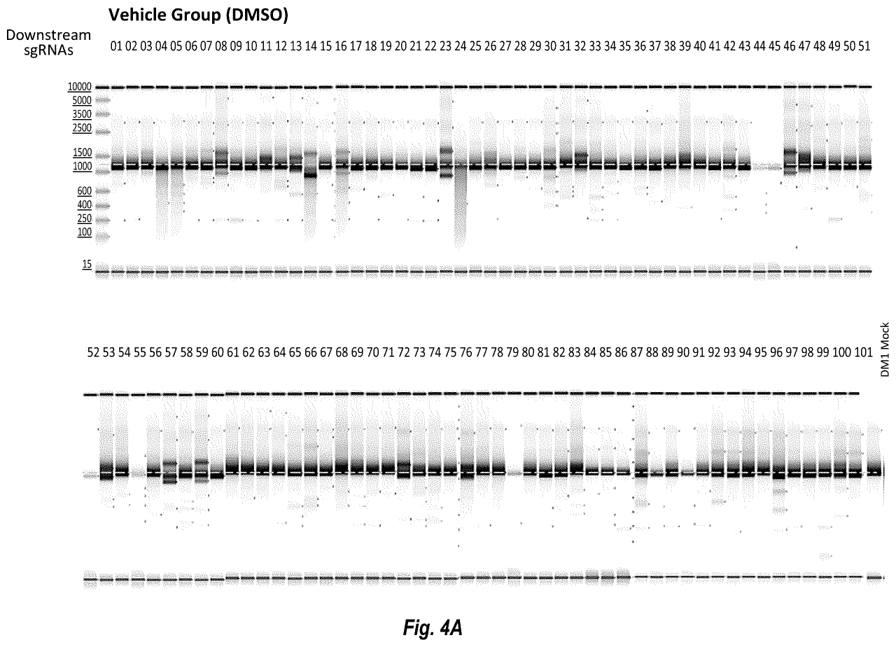

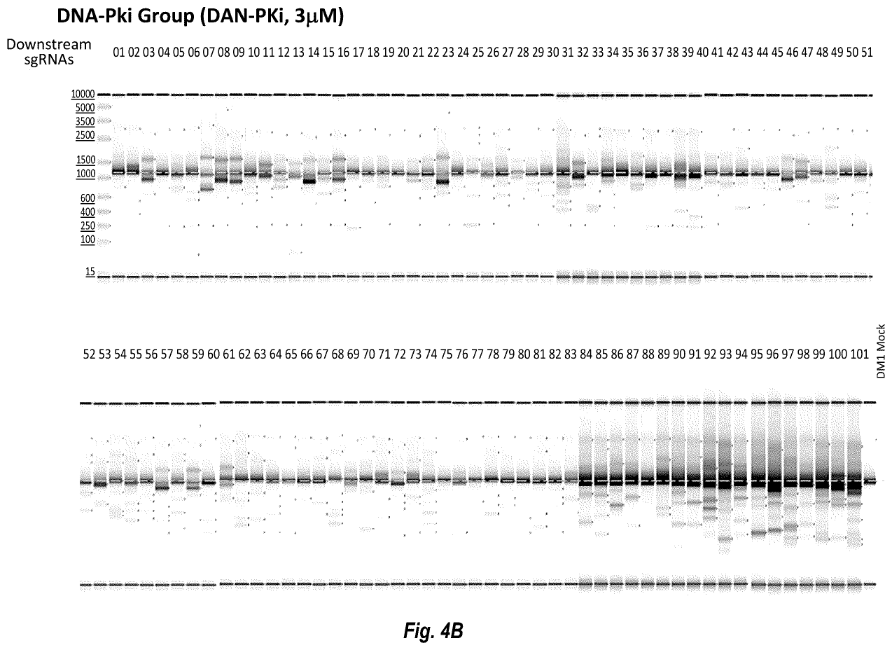

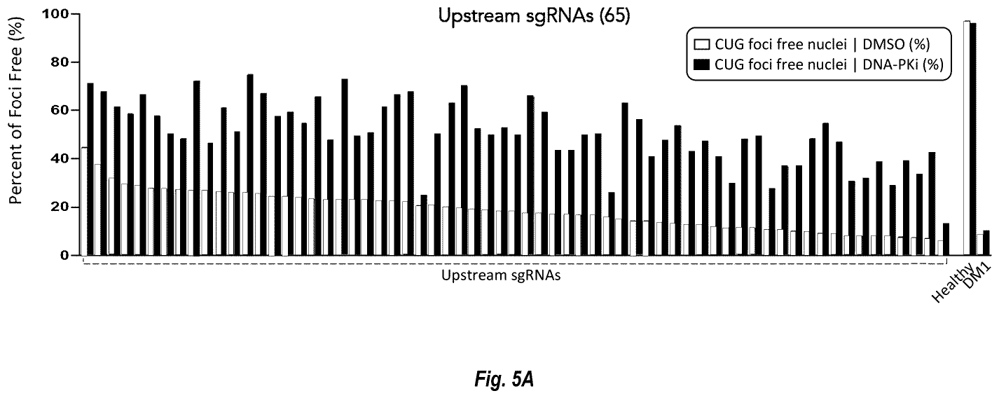

TapeStation analysis was used to assess the large indel (>30 bp) prof ile induced by individual SluCas9 sgRNAs (FIG. 3A-B for upstream sgRNAs and FIG. 4A-B for downstream sgRNAs). The top bands appearing at approximately size 10,000 are the upper standards, and the bottom bands appearing at approximately size 15 are the lower standards. The dashed lines indicate the 1174 bp PCR products amplified from non-edited wild-type allele or wild-type allele with small indels (<30 bp). Several SluCas9 sgRNAs induced large deletions of various sizes, represented by the PCR bands located below the 1174 bp PCR band (>30 bp). Compared to the vehicle group (A), the DNA-PKi group (B) showed more abundant large deletions. DM1 Mock is the DM1 patient myoblasts that were nucleofected with SluCas9 protein but not sgRNA.

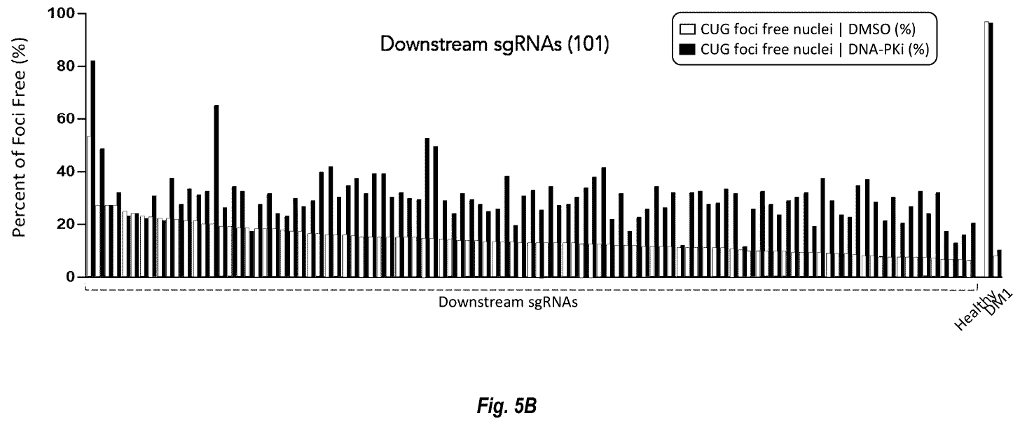

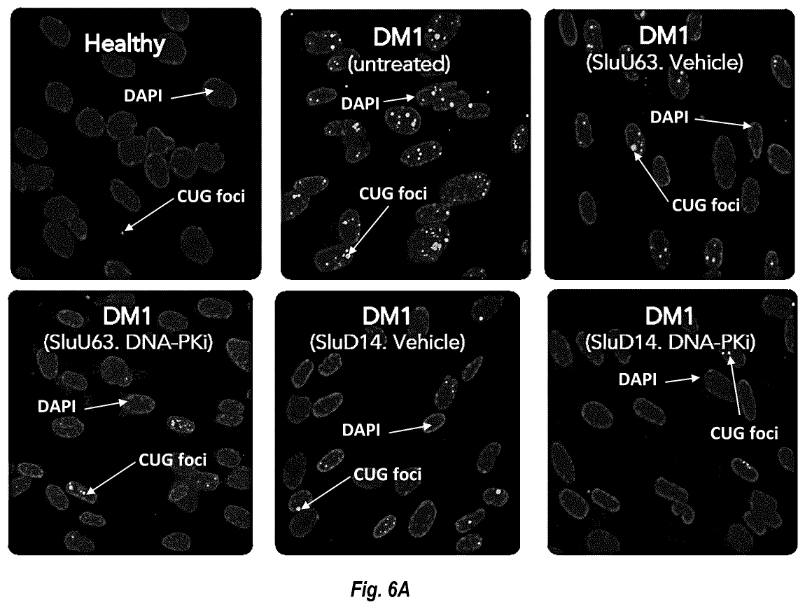

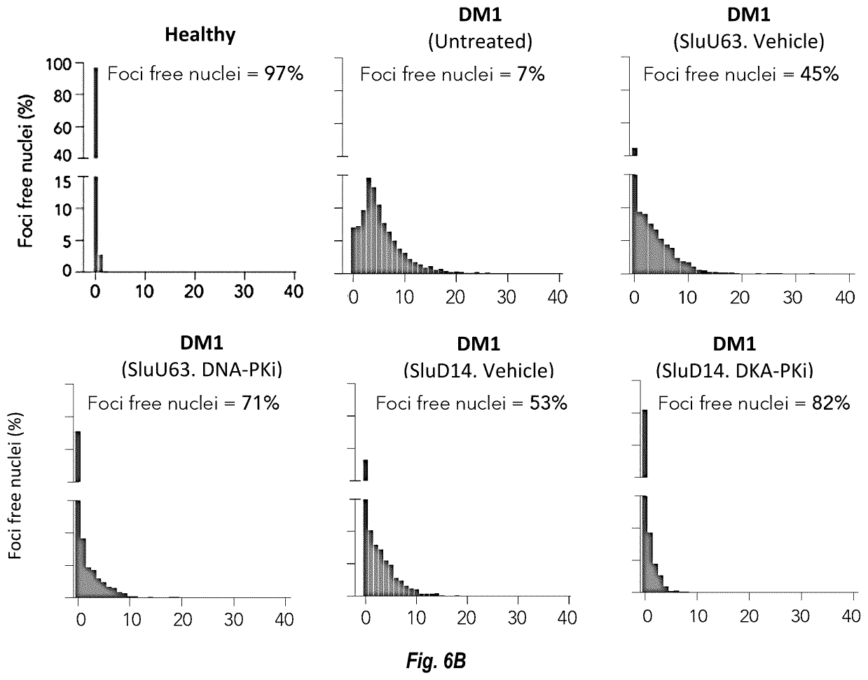

FISH staining of RNA foci showed reduction of CUG foci (formed by the CUG repeat expansion in the DMPK mRNA) in DM1 patient myoblasts by individual sgRNAs (FIG. 5A (upstream guides) and FIG. 5B (downstream guides) and Table 3). Shown are the percentage of CUG foci free nuclei in vehicle (white bars) or with DNA-PKi (black bars) treated myoblasts. The sgRNAs were ordered from the highest efficiency to the lowest efficiency in the vehicle group. The healthy myoblasts (Healthy) served as a positive control, and the DM1 patient myoblasts that were nucleofected with SluCas9 protein but not sgRNA (DM1) served as a negative control. Among the 166 sgRNAs evaluated, 4 sgRNAs (SluU08, SluU63, SluU64 and SluD14) completely abolished CUG RNA foci in more than 40% of myoblast nuclei with vehicle treatment. As the most efficient sgRNA, SluD14 abolished CUG RNA foci in 53% of myoblast nuclei with vehicle treatment, and in 81.82% of myoblast nuclei with DNA-PKi treatment. RNA foci distribution analysis showed that SluU63 and SluD14 not only eliminated the CUG foci in a large fraction of myoblast nuclei, but also reduced the frequency of myoblast nuclei that contain more than three CUG foci (FIG. 6A-B). SluU63 and SluD14 not only increased the frequency of CUG foci free myoblast nuclei (foci number per nucleus=0), but also reduced the frequency of myoblast nuclei that contain more than three CUG foci. Compared to vehicle treatment, DNA-PKi treatment abolished CUG foci in more myoblast nuclei and reduced the frequency of myoblast nuclei that contain more than three CUG foci. FIG. 6A-B and Table 3.

In addition to CUG foci staining, CAG foci staining was also performed, which is formed either by antisense transcript emanating from the downstream SIX5 gene or by the inversion of the CTG repeat sequence induced by individual SuCas9 sgRNAs. The vast majority of SuCas9 sgRNAs induced low level of CAG foci (Table 3).

TABLE 3
Single-Cut Data Summary

| SluCas9 | SEQ | | % of CUG foci | % of CUG foci | % of CAG foci | % of CAG foci |
|---|---|---|---|---|---|---|
| sgRNA | ID | Indel | free nuclei | free nuclei | positive nuclei | positive nuclei |
| name | NO. | efficiency | (Vehicle) | (DNA-PKi) | (Vehicle) | (DNA-PKi) |
| SluD01 | 67 | 0 | 18.45 | 17.29 | 0.79 | 1.34 |
| SluD02 | 68 | 0 | 8.76 | 22.29 | 1.13 | 1.08 |
| SluD03 | 69 | 2 | 14.38 | 49.43 | 1.19 | 2.14 |
| SluD04 | 70 | 1 | 13.29 | 19.38 | 1.17 | 1.94 |
| SluD05 | 71 | 22 | 26.95 | 26.99 | 1.42 | 1.1 |
| SluD06 | 72 | 55 | 24.51 | 23.09 | 3.12 | 1.35 |
| SluD07 | 73 | 28 | 12.52 | 29.79 | 2.37 | 0.82 |
| SluD08 | 74 | 45 | 27.09 | 48.28 | 5 | 1.22 |
| SluD09 | 75 | 5 | 12.5 | 33.83 | 1.16 | 1.48 |
| SluD10 | 76 | 18 | 13.43 | 27.41 | 2.84 | 1.26 |
| SluD11 | 77 | 16 | 14.54 | 52.55 | 1.7 | 2.22 |
| SluD12 | 78 | 44 | 17.76 | 23.77 | 4.65 | 1.21 |
| SluD13 | 79 | 55 | 15.93 | 42.02 | 4.07 | 2.77 |
| SluD14 | 81 | NA | 53.12 | 81.82 | 6.7 | 3.96 |
| SluD15 | 82 | 49 | 11.01 | 27.46 | 2.44 | 0.64 |
| SluD16 | 80 | 31 | 15.2 | 38.89 | 2.11 | 1.74 |
| SluD17 | 83 | 33 | 12.25 | 21.7 | 2.07 | 1.47 |
| SluD18 | 84 | 75 | 15.38 | 31.47 | 2.71 | 1.55 |
| SluD19 | 85 | 24 | 12.59 | 27.72 | 2.32 | 1.68 |
| SluD20 | 86 | 24 | 13.32 | 24.59 | 1.81 | 1.1 |
| SluD21 | 87 | 36 | 9.96 | 32.15 | 1.95 | 1.77 |
| SluD22 | 88 | 21 | 7.86 | 20.98 | 0.76 | 1.54 |
| SluD23 | 89 | 48 | 19.61 | 64.98 | 3.33 | 4.37 |
| SluD24 | 90 | 27 | 17.65 | 22.99 | 2.75 | 1.68 |
| SluD25 | 91 | 41 | 26.87 | 32.03 | 2.62 | 1.35 |
| SluD26 | 92 | NA | 19.92 | 32.32 | 3.44 | 2.09 |
| SluD27 | 93 | 8 | 7.6 | 20.28 | 0.93 | 1.4 |
| SluD28 | 94 | 48 | 11.31 | 31.88 | 1.23 | 1.29 |
| SluD29 | 95 | 35 | 7.97 | 28.37 | 1.72 | 2.38 |
| SluD30 | 96 | NA | 15.1 | 30.01 | 2.66 | 2.1 |
| SluD31 | 97 | 63 | 14.17 | 23.95 | 3.06 | 3.21 |
| SluD32 | 98 | 94 | 19.22 | 26.23 | 1.43 | 1.31 |
| SluD33 | 99 | 74 | 12.72 | 33.92 | 4.78 | 1.76 |
| SluD34 | 100 | 71 | 15 | 31.92 | 3.9 | 2.01 |
| SluD35 | 101 | 0 | 6.61 | 12.47 | 1.04 | 1.13 |
| SluD36 | 102 | 52 | 13.52 | 29.02 | 3.24 | 2.32 |
| SluD37 | 103 | 25 | 6.89 | 31.69 | 1.76 | 2.15 |
| SluD38 | 104 | 1 | 6.59 | 15.53 | 0.93 | 1.96 |
| SluD39 | 105 | 39 | 9.15 | 31.63 | 1.95 | 1.68 |
| SluD40 | 106 | 5 | 8.9 | 23.44 | 1.68 | 1.46 |
| SluD41 | 107 | 6 | 8.97 | 37.15 | 1.33 | 1.44 |
| SluD42 | 108 | NA | 13.3 | 38.26 | 4.25 | 2.85 |
| SluD43 | 109 | 1 | 6.41 | 20.27 | 1.31 | 0.82 |
| SluD44 | 110 | 24 | 8.05 | 36.44 | 1.49 | 2.29 |
| SluD45 | 111 | 8 | 7.51 | 32.39 | 1.17 | 1.16 |
| SluD46 | 112 | 46 | 8.24 | 34.71 | 1.69 | 1.16 |
| SluD47 | 113 | 18 | 8.95 | 28.63 | 1 | 1.11 |
| SluD48 | 114 | 60 | 13.31 | 25.41 | 2.3 | 2.91 |
| SluD49 | 115 | 41 | 12.39 | 37.77 | 2.83 | 2.3 |
| SluD50 | 116 | 4 | 7.64 | 30.13 | 1.81 | 2.2 |
| SluD51 | 117 | 33 | 14.34 | 28.86 | 2.15 | 2.13 |
| SluD52 | 118 | 6 | 7.59 | 26.8 | 1.26 | 1.57 |
| SluD53 | 119 | NA | 12.28 | 41.45 | 1.94 | 1.6 |
| SluD54 | 120 | 26 | 10.7 | 33.12 | 2.11 | 2.85 |
| SluD55 | 121 | 41 | 15.71 | 34.69 | 3.01 | 2.92 |
| SluD56 | 122 | 70 | 13.88 | 31.7 | 1.04 | 2.91 |
| SluD57 | 123 | NA | 15.61 | 37.23 | 2.09 | 1.02 |
| SluD58 | 124 | 33 | 18.03 | 31.45 | 1.37 | 1.96 |
| SluD59 | 125 | NA | 9.79 | 27.36 | 1.18 | 1.29 |
| SluD60 | 126 | 0 | 6.71 | 16.87 | 1.02 | 1.16 |
| SluD61 | 127 | 2 | 17.04 | 26.76 | 1.27 | 1.94 |
| SluD62 | 128 | 14 | 11.48 | 34.48 | 1.59 | 2.45 |
| SluD63 | 129 | 6 | 9.09 | 19.22 | 0.85 | 2.95 |
| SluD64 | 130 | 23 | 11.02 | 32.65 | 2.64 | 2.38 |
| SluD65 | 131 | 36 | 18.5 | 32.37 | 1.33 | 0.92 |
| SluD66 | 132 | 28 | 13.03 | 30.48 | 1.41 | 1.94 |
| SluD67 | 133 | 39 | 10.06 | 25.54 | 0.69 | 1.8 |
| SluD68 | 134 | 72 | 12.88 | 25.13 | 2.3 | 1.94 |
| SluD69 | 135 | 14 | 10.88 | 27.98 | 1.3 | 2.13 |
| SluD70 | 136 | 14 | 22.75 | 30.47 | 2.33 | 2.45 |
| SluD71 | 137 | 23 | 9.75 | 23.51 | 1.38 | 2.27 |
| SluD72 | 138 | 5 | 16.2 | 28.82 | 2.62 | 2.33 |
| SluD73 | 139 | 65 | 12.95 | 32.52 | 2.52 | 2.33 |
| SluD74 | 140 | 56 | 10.6 | 31.37 | 2.27 | 1.55 |
| SluD75 | 141 | 17 | 11.9 | 22.6 | 1.56 | 1.9 |
| SluD76 | 142 | 20 | 15.78 | 29.87 | 1.73 | 2.16 |
| SluD77 | 143 | 17 | 14.94 | 28.94 | 1.8 | 3.13 |
| SluD78 | 144 | 4 | 12.61 | 27 | 2.31 | 1.36 |
| SluD79 | 145 | 2 | 11.48 | 25.61 | 0.82 | 1.79 |
| SluD80 | 146 | 1 | 11.92 | 17.34 | 1.98 | 1.49 |
| SluD81 | 147 | 10 | 9.64 | 28.66 | 1.54 | 1.75 |
| SluD82 | 148 | 22 | 14.98 | 29.51 | 1.39 | 2.02 |
| SluD83 | 149 | 70 | 19.15 | 34 | 1.64 | 1.51 |
| SluD84 | 150 | 10 | 9.15 | 30.25 | 1.28 | 3.03 |
| SluD85 | 151 | 28 | 17.4 | 29.51 | 2.51 | 2.82 |
| SluD86 | 152 | 30 | 11.98 | 31.62 | 1.98 | 3.95 |
| SluD87 | 153 | 30 | 15.16 | 39.12 | 1.3 | 2.19 |
| SluD88 | 154 | 7 | 11.11 | 31.95 | 1.4 | 2.42 |
| SluD89 | 155 | 0 | 10.23 | 11.37 | 0.61 | 2.54 |
| SluD90 | 156 | 2 | 7.35 | 23.81 | 0.86 | 1.27 |
| SluD91 | 157 | 33 | 22.42 | 21.28 | 1 | 4.23 |
| SluD92 | 158 | 30 | 24.08 | 24.03 | 1.4 | 3.02 |
| SluD93 | 159 | 18 | 11.34 | 26.14 | 1.63 | 3.58 |
| SluD94 | 160 | 30 | 18.18 | 27.23 | 1 | 1.32 |
| SluD95 | 161 | 28 | 21.81 | 27.23 | 1.3 | 2.19 |
| SluD96 | 162 | 31 | 21 | 31.02 | 2.21 | 2.73 |
| SluD97 | 163 | 11 | 16.07 | 39.95 | 1.19 | 2.01 |
| SluD98 | 164 | 0 | 11.21 | 11.71 | 0.84 | 1.47 |
| SluD99 | 165 | 1 | 22.87 | 22.16 | 0.73 | 2.23 |
| SluD100 | 166 | 81 | 22.06 | 37.39 | 2.15 | 3.35 |
| SluD101 | 167 | 38 | 21.35 | 33.61 | 2.09 | 2.93 |
| SluU01 | 1 | 38 | 22.92 | 51.01 | 1.71 | 1.15 |
| SluU02 | 2 | 16 | 9.09 | 54.94 | 1.38 | 1.26 |
| SluU03 | 3 | 9 | 11.12 | 49.49 | 1.05 | 2.04 |
| SluU04 | 4 | NA | 17.21 | 65.78 | 0.72 | 1.21 |
| SluU05 | 5 | 65 | 26.37 | 61.16 | 1.53 | 1.64 |
| SluU06 | 6 | 81 | 25.49 | 67.15 | 1.01 | 1.1 |
| SluU07 | 7 | 57 | 26.22 | 51.08 | 2.36 | 1.27 |
| SluU08 | 8 | 87 | 31.42 | 61.56 | 2.21 | 1.61 |
| SluU09 | 9 | 65 | 26.05 | 74.97 | 1.48 | 1.22 |
| SluU10 | 10 | 81 | 27.04 | 72.18 | 2 | 0.66 |
| SluU11 | 11 | 21 | 19.12 | 52.22 | 1.44 | 1.27 |
| SluU12 | 12 | 20 | 16.44 | 50.23 | 1.29 | 1.38 |
| SluU13 | 13 | 27 | 17.02 | 43.12 | 1.11 | 1.07 |
| SluU14 | 14 | 52 | 29.12 | 58.84 | 1.02 | 1.63 |
| SluU15 | 15 | 4 | 7.97 | 31.71 | 0.75 | 1.28 |
| SluU16 | 16 | 7 | 10.73 | 27.5 | 1.28 | 1.73 |
| SluU17 | 17 | 32 | 21.96 | 67.55 | 1.5 | 1.28 |
| SluU18 | 18 | 14 | 9.74 | 48.57 | 1.1 | 1.24 |
| SluU19 | 19 | 48 | 19.3 | 69.93 | 1.54 | 0.83 |
| SluU20 | 20 | 30 | 18.45 | 49.95 | 1.15 | 1.43 |
| SluU21 | 21 | 80 | 23.65 | 54.83 | 0.68 | 0.66 |
| SluU22 | 22 | 41 | 27.56 | 50.21 | 0.96 | 1.28 |
| SluU23 | 23 | 5 | 11.23 | 29.8 | 1 | 1.53 |
| SluU24 | 24 | 28 | 20.62 | 50.23 | 1 | 1.56 |
| SluU25 | 25 | 38 | 23.15 | 47.96 | 0.92 | 1.29 |
| SluU26 | 26 | NA | 28.68 | 66.19 | 1.56 | 2.39 |
| SluU27 | 27 | 1 | 7.71 | 38.24 | 1.64 | 1.94 |
| SluU28 | 28 | 17 | 10.14 | 36.78 | 1.15 | 1.64 |
| SluU29 | 29 | 67 | 27.28 | 48.49 | 1.18 | 1.82 |
| SluU30 | 30 | 48 | 26.92 | 46.16 | 0.67 | 2.14 |
| SluU31 | 31 | 29 | 14.2 | 40.74 | 0.94 | 1.62 |
| SluU32 | 32 | 43 | 24.5 | 59.19 | 2.19 | 6.56 |
| SluU33 | 33 | 26 | 24.67 | 57.35 | 1.14 | 1.3 |
| SluU34 | 34 | 53 | 19.95 | 63.08 | 0.78 | 1.03 |
| SluU35 | 35 | 25 | 14.23 | 55.71 | 0.69 | 1.42 |
| SluU36 | 36 | 24 | 11.72 | 40.62 | 1.05 | 1.96 |
| SluU37 | 37 | 15 | 8.09 | 30.58 | 0.72 | 2.28 |
| SluU38 | 38 | 2 | 7.24 | 32.86 | 0.68 | 1.56 |
| SluU39 | 39 | 11 | 10.53 | 36.78 | 0.71 | 2.5 |
| SluU40 | 40 | 3 | 15.29 | 25.77 | 1 | 2.01 |
| SluU41 | 41 | 13 | 13.34 | 47.52 | 0.74 | 1.08 |
| SluU42 | 42 | 15 | 7.05 | 42.7 | 0.86 | 1.62 |
| SluU43 | 43 | 5 | 7.64 | 28.61 | 1.02 | 1.76 |
| SluU44 | 44 | 16 | 12.6 | 47 | 0.7 | 1.77 |
| SluU45 | 45 | 14 | 12.63 | 43.15 | 1.11 | 2.36 |
| SluU46 | 46 | 41 | 14.46 | 62.89 | 0.74 | 1.5 |
| SluU47 | 47 | 0 | 5.91 | 12.71 | 1 | 2.4 |
| SluU48 | 48 | 11 | 20.76 | 24.81 | 1.1 | 1.56 |
| SluU49 | 49 | 0 | 7.53 | 38.7 | 1.03 | 1.44 |
| SluU50 | 50 | 22 | 11.21 | 48.15 | 0.75 | 1.59 |
| SluU51 | 51 | 28 | 8.62 | 46.88 | 0.72 | 1.6 |
| SluU52 | 52 | 34 | 23.34 | 65.25 | 0.74 | 2.65 |
| SluU53 | 53 | 31 | 22.52 | 61.52 | 1.44 | 2.9 |
| SluU54 | 54 | 10 | 13.12 | 53.64 | 1.14 | 1.28 |
| SluU55 | 55 | 67 | 27.94 | 57.91 | 0.99 | 1.34 |
| SluU56 | 56 | 27 | 16.76 | 42.84 | 0.94 | 1.83 |
| SluU57 | 57 | 17 | 18.03 | 52.65 | 1.54 | 1.77 |
| SluU58 | 58 | 36 | 16.57 | 49.37 | 1.35 | 1.51 |
| SluU59 | 59 | 92 | 23.05 | 72.56 | 1.35 | 1.4 |
| SluU60 | 60 | 40 | 22.98 | 49.06 | 1.23 | 2.17 |
| SluU61 | 61 | 14 | 17.78 | 49.57 | 0.9 | 1.46 |
| SluU62 | 62 | 87 | 17.18 | 59.07 | 1 | 1.67 |
| SluU63 | 63 | NA | 44.78 | 70.99 | 3.29 | 5.49 |
| SluU64 | 64 | 59 | 37.58 | 67.39 | 1.23 | 2.58 |
| SluU65 | 65 | NA | 22.41 | 66 | 1.29 | 2.09 |
| DM | 1000 | N/A | 7.97 | 10.2 | 0.75 | 1.26 |
| control | | | | | | |
| Healthy | 1001 | N/A | 97.16 | 96.18 | 0.42 | 0.22 |
| control | | | | | | |

C. Double Cut Screening

Double-cut screening was performed to assess the efficiency of paired sgRNAs-induced CTG repeat excision and RNA foci reduction.

Eighty-eight (88) SluCas9 sgRNA pairs in the 3′ UTR of human DMPK gene were nominated for Dual-cut screening based on the Single-Cut screening results of 166 sluCas9 sgRNAs in primary DM1 patient myoblasts, as described above in Example B (See, e.g., FIG. 2 and Table 3).

To assess the efficiency of SluCas9 sgRNA pairs for inducing CTG repeat excision, Dual-cut screening was performed in which individual SluCas9 sgRNAs and recombinant SluCas9 protein were assembled into ribonucleoprotein (RNP) and delivered into primary DM1 patient myoblasts. Nucleofected myoblasts were treated with either DMSO (vehicle) or 3 μM of DNA-dependent Protein Kinase Inhibitor (DNA-PKi) Compound 6 for 48 hours. 72 hours post-nucleofection, myoblasts were subjected to either genomic DNA isolation or RNA foci staining by fluorescence in situ hybridization (FISH).

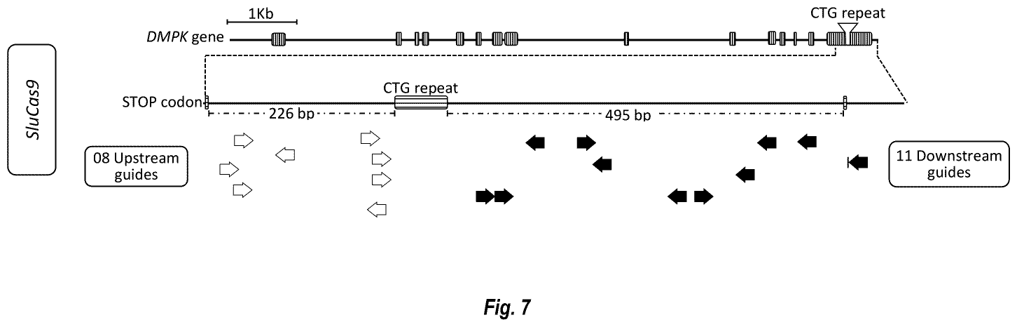

By combining the editing efficiency from ICE analysis and large indel prof ile from Tape Station analysis, eight (8) sgRNAs (SluU06, SluU08, SluU10, SluU21, SluU59, SluU62, SluU63, and SuU64) (SEQ ID NOs: 6, 8, 10, 21, 59, 62, 63, and 64, respectively) located upstream of the CTG repeat expansion (between the stop codon and the CTG repeat expansion), and 11 sgRNAs located downstream of the CTG repeat expansion (between the CTG repeat expansion and the end of the last exon of DMPK gene) (D06, D14, D18, D32, D34, D48, D56, D68, D73, D83, and D100) (SEQ ID NOs: 72, 81, 84, 98, 100, 114, 122, 134, 139, 149, and 166, respectively) were included for further evaluation in Dual-cut screening due to their high editing IN4DEL efficiency in SINGLE-cut screening of 166 sluCas9 sgRiNAs (Table 4 and FIG. 7). Each of the eight upstream sgRNAs was paired with each of the 11 downstream sgRiNAs to reach 88 tested pairs.

TABLE 4
sgRNAs for Dual-Cut

| | | | | | | | | Number of |
|---|---|---|---|---|---|---|---|---|
| | | Proto- | | | | | | predicted |
| SluCas9 | SEQ | spacer | Proto- | Proto- | | | | off-target |
| sgRNA | ID | sequence | spacer_ | spacer_ | PAM | PAM_ | PAM_ | site |
| name | NO | (22 bp) | start | end | Sequence | start | end | (22 mer) |
| U06 | 6 | TGTCTTC | 45770455 | 45770477 | TTGG | 457704 | 457704 | 2 |
| | | GACTCCG | | | | | | |
| | | GGGCCCC | | | | | | |
| | | G | | | | | | |
| U08 | 8 | GCCCCGT | 45770439 | 45770461 | CCGG | 457704 | 457704 | 2 |
| | | TGGAAGA | | | | | | |
| | | CTGAGTG | | | | | | |
| | | C | | | | | | |
| U10 | 10 | CCCGTTG | 45770437 | 45770459 | GGGG | 457704 | 457704 | 3 |
| | | GAAGACT | | | | | | |
| | | GAGTGCC | | | | | | |
| | | C | | | | | | |
| U21 | 21 | GCTCGGA | 45770382 | 45770404 | CAGG | 457704 | 457704 | 0 |
| | | GCGGTTG | | | | | | |
| | | TGAACTG | | | | | | |
| | | G | | | | | | |
| U58 | 58 | gccccgt | 45770290 | 45770312 | ccgg | 457703 | 457703 | 1 |
| | | tcgccgg | | | | | | |
| | | ccgcgga | | | | | | |
| | | C | | | | | | |
| U62 | 62 | AGGACCC | 45770278 | 45770300 | ccgg | 457703 | 457703 | 0 |
| | | TTCGAgc | | | | | | |
| | | cccgttc | | | | | | |
| | | g | | | | | | |
| U63 | 63 | ggggcTC | 45770273 | 45770295 | CCGG | 457702 | 457702 | 4 |
| | | GAAGGGT | | | | | | |
| | | CCTTGTA | | | | | | |
| | | G | | | | | | |
| U64 | 64 | gggcTCG | 45770272 | 45770294 | CGGG | 457702 | 457702 | 1 |
| | | AAGGGTC | | | | | | |
| | | CTTGTAG | | | | | | |
| | | C | | | | | | |
| D06 | 72 | CCAGGCT | 45770152 | 45770174 | ATGG | 457701 | 457701 | 12 |
| | | GAGGCCC | | | | | | |
| | | TGACGTG | | | | | | |
| | | G | | | | | | |
| D14 | 81 | ATGGGCA | 45770130 | 45770152 | AAGG | 457701 | 457701 | 77 |
| | | AACTGCA | | | | | | |
| | | GGCCTGG | | | | | | |
| | | G | | | | | | |
| D18 | 84 | TGGAGGA | 45770093 | 45770115 | CCGG | 457701 | 457701 | 3 |
| | | TGGAACA | | | | | | |
| | | CGGACGG | | | | | | |
| | | C | | | | | | |
| D32 | 98 | CTTGTGC | 45770030 | 45770052 | GGGG | 457700 | 457700 | 6 |
| | | ATGACGC | | | | | | |
| | | CCTGCTC | | | | | | |
| | | T | | | | | | |
| D34 | 100 | CGCGCCA | 45770013 | 45770035 | AGGG | 457700 | 457700 | 7 |
| | | GACGCTC | | | | | | |
| | | CCCAGAG | | | | | | |
| | | C | | | | | | |
| D48 | 114 | GAGAGCA | 45769925 | 45769947 | GGGG | 457699 | 457699 | 37 |
| | | GCGCAAG | | | | | | |
| | | TGAGGAG | | | | | | |
| | | G | | | | | | |
| D56 | 122 | CCAGCCG | 45769897 | 45769919 | GCGG | 457698 | 457698 | 4 |
| | | GCTCCGC | | | | | | |
| | | CCGCTTC | | | | | | |
| | | G | | | | | | |
| D68 | 134 | CCTGTAG | 45769845 | 45769867 | GAGG | 457698 | 457698 | 2 |
| | | CCTGTCA | | | | | | |
| | | GCGAGTC | | | | | | |
| | | G | | | | | | |
| D73 | 139 | CCAAAAC | 45769816 | 45769838 | TGGG | 457698 | 457698 | 6 |
| | | GTGGATT | | | | | | |
| | | GGGGTTG | | | | | | |
| | | T | | | | | | |
| D83 | 149 | GTGGGGA | 45769769 | 45769791 | GAGG | 457697 | 457697 | 13 |
| | | CAGACAA | | | | | | |
| | | TAAATAC | | | | | | |
| | | C | | | | | | |
| D100 | 166 | GTCCAGA | 45769708 | 45769729 | AGGG | 457697 | 457697 | 1 |
| | | GCTTTGG | | | | | | |
| | | GCAGATG | | | | | | |
| | | G | | | | | | |

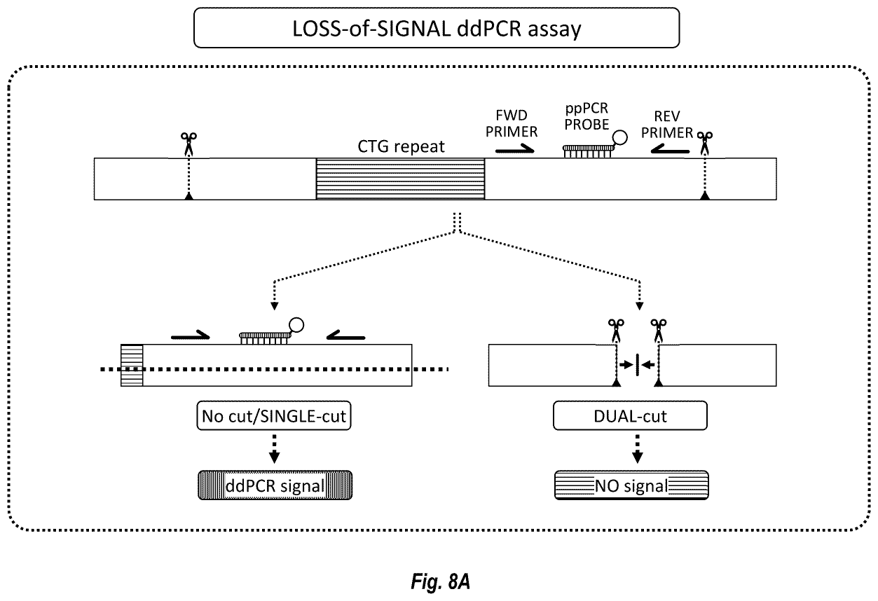

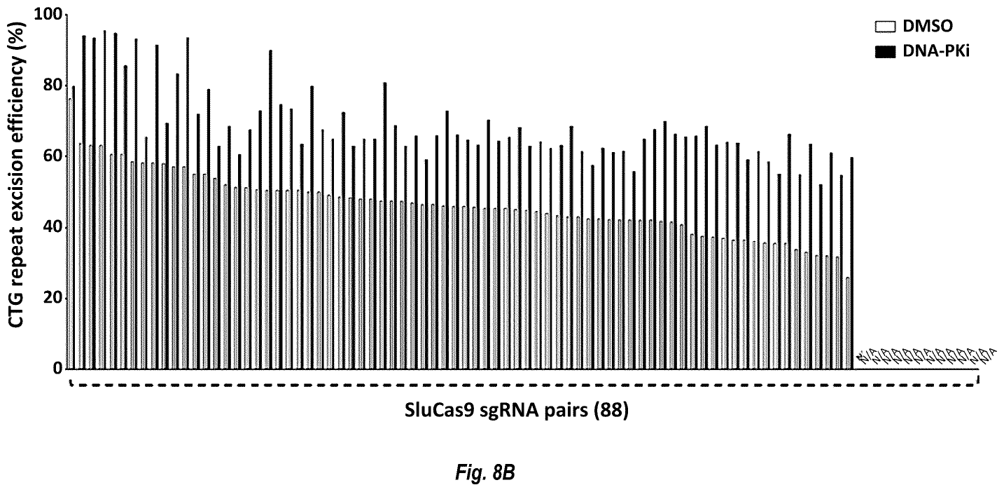

CRISPR repeat excision efficiency was assessed for each of the 88 pairs (FIG. 8A-B and Table 5). CTG repeat excision efficiency percentages are shown for vehicle (DMSO; white bars) and with DNA-PKi (black bars) (FIG. 8B and Table 5).

TABLE 5
Double-Cut Data Summary

| | SluCas9 | Excision | | % of CUG | % of CUG |
|---|---|---|---|---|---|
| SluCas9 | sgRNA pairs | efficiency | Excision | foci free | foci free |
| sgRNA pair | (SEQ ID | by ddPCR | efficiency | nuclei | nuclei |
| name | NOs.) | (Vehicle) | (by ddPCR) | (Vehicle) | (DNA-PKi) |
| SluU64 + SluD34 | 64 + 100 | 76.3522013 | 79.9065421 | 83.684029 | 91.4510166 |
| SluU10 + SluD34 | 10 + 100 | 52.1204065 | 68.5949082 | 80.8986227 | 88.6290558 |
| SluU06 + SluD34 | 6 + 100 | 51.3656895 | 67.5717048 | 78.5632184 | 90.6636304 |
| SluU08 + SluD34 | 8 + 100 | 58.1102534 | 69.4661848 | 77.9269202 | 91.1452514 |
| SluU63 + SluD34 | 63 + 100 | 63.2704403 | 93.5046729 | 77.8499278 | 87.2456726 |
| SluU64 + SluD32 | 64 + 98 | 60.754717 | 85.5607477 | 77.5994651 | 89.9914821 |
| SluU10 + SluD32 | 10 + 98 | 48.6201616 | 72.5162286 | 77.2907438 | 90.0761124 |
| SluU64 + SluD83 | 64 + 149 | 55.2201258 | 78.9719626 | 76.5463918 | 90.6284454 |
| SluU59 + SluD34 | 59 + 100 | 55.2201258 | 71.9626168 | 76.4747191 | 89.0050876 |
| SluU64 + SluD48 | 64 + 114 | 47.672956 | 80.8411215 | 75.4465736 | 88.4432945 |
| SluU63 + SluD32 | 63 + 98 | 60.754717 | 94.8130841 | 75.2569926 | 89.0378314 |
| SluU64 + SluD68 | 64 + 134 | 46.163522 | 72.8971963 | 74.9421488 | 89.558883 |
| SluU10 + SluD83 | 10 + 149 | 47.6269329 | 68.8032319 | 74.1650763 | 89.4985809 |
| SluU63 + SluD83 | 63 + 149 | 58.7421384 | 93.271028 | 74.1052632 | 89.7999436 |
| SluU08 + SluD32 | 8 + 98 | 50.8668983 | 73.0215 | 74.104352 | 90.9117909 |
| SluU63 + SluD48 | 63 + 114 | 63.7735849 | 94.2523364 | 73.7275449 | 90.0299401 |
| SluU63 + SluD100 | 63 + 166 | 58.2389937 | 91.4953271 | 73.1483715 | 89.9731423 |
| SluU64 + SluD18 | 64 + 84 | N/A | N/A | 72.5132626 | 89.744663 |
| SluU63 + SluD68 | 63 + 134 | 57.2327044 | 93.6448598 | 71.7386285 | 90.625 |
| SluU10 + SluD18 | 10 + 84 | 51.5451174 | 60.591133 | 71.691974 | 89.0786894 |
| SluU64 + SluD73 | 64 + 139 | 50.6918239 | 74.7663551 | 70.1931649 | 91.0221531 |
| SluU63 + SluD73 | 63 + 139 | 63.2704403 | 95.5140187 | 70.0337512 | 90.5766944 |
| SluU10 + SluD73 | 10 + 139 | 46.126206 | 64.7679665 | 69.9696191 | 88.4638022 |
| SluU64 + SluD100 | 64 + 166 | 45.1572327 | 68.2242991 | 69.881202 | 85.8852662 |
| SluU06 + SluD18 | 6 + 84 | 45.6118665 | 64.5320197 | 69.247197 | 87.7938808 |
| SluU59 + SluD32 | 59 + 98 | 57.2327044 | 83.317757 | 69.2344612 | 91.1886458 |
| SluU10 + SluD48 | 10 + 114 | 44.135395 | 62.2922518 | 69.1129401 | 88.0196937 |
| SluU63 + SluD18 | 63 + 84 | N/A | N/A | 68.6201261 | 88.1469115 |
| SluU10 + SluD68 | 10 + 134 | 42.3743888 | 61.2943695 | 68.1479579 | 89.5024272 |
| SluU08 + SluD83 | 8 + 149 | 45.636122 | 70.464067 | 68.1400438 | 90.3820465 |
| SluU10 + SluD100 | 10 + 166 | 44.6472468 | 64.2500345 | 67.8052158 | 88.2369615 |
| SluU06 + SluD32 | 6 + 98 | 50.6327402 | 73.5141108 | 67.7698574 | 89.8445596 |
| SluU63 + SluD14 | 63 + 81 | N/A | N/A | 67.3458725 | 85.9981372 |
| SluU64 + SluD06 | 64 + 72 | N/A | N/A | 67.2061329 | 86.5919064 |
| SluU08 + SluD18 | 8 + 84 | 50.5562423 | 63.546798 | 67.0639468 | 90.4390526 |
| SluU64 + SluD14 | 64 + 81 | N/A | N/A | 67.0583627 | 86.0696517 |
| SluU06 + SluD48 | 6 + 114 | 45.8920478 | 63.3281157 | 66.882309 | 89.4649934 |
| SluU63 + SluD06 | 63 + 72 | N/A | N/A | 66.8191057 | 88.4507042 |
| SluU06 + SluD14 | 6 + 81 | 48.0840544 | 65.0246305 | 66.7242869 | 83.3656331 |
| SluU08 + SluD100 | 8 + 166 | 41.6545001 | 66.3781594 | 66.3115278 | 88.5915112 |
| SluU06 + SluD83 | 6 + 149 | 46.1305596 | 66.1824962 | 66.0344397 | 89.4458172 |
| SluU59 + SluD18 | 59 + 84 | N/A | N/A | 65.7246213 | 88.2099828 |
| SluU08 + SluD73 | 8 + 139 | 50.1208885 | 67.6096865 | 65.4225352 | 89.9372784 |
| SluU06 + SluD73 | 6 + 139 | 43.4024458 | 63.290134 | 65.152325 | 88.3744171 |
| SluU08 + SluD68 | 8 + 134 | 42.6303147 | 62.5638783 | 65.1006711 | 88.2368082 |
| SluU06 + SluD100 | 6 + 166 | 40.8910761 | 65.6772248 | 64.9460709 | 87.3691099 |
| SluU08 + SluD48 | 8 + 114 | 42.1445842 | 55.9205378 | 64.9313772 | 87.6255088 |
| SluU62 + SluD34 | 62 + 100 | 48.1761006 | 64.953271 | 64.7101981 | 88.2910425 |
| SluU59 + SluD83 | 59 + 149 | 49.1823899 | 64.953271 | 64.6134347 | 90.4929577 |
| SluU06 + SluD68 | 6 + 134 | 47.3840676 | 62.9931864 | 64.0101523 | 89.3964655 |
| SluU10 + SluD14 | 10 + 81 | 43.1396786 | 61.5763547 | 62.9525032 | 84.0366972 |
| SluU59 + SluD68 | 59 + 134 | 58.2389937 | 65.4205607 | 62.8125 | 89.368216 |
| SluU06 + SluD06 | 6 + 72 | 42.645241 | 57.635468 | 62.8060523 | 87.0221328 |
| SluU59 + SluD100 | 59 + 166 | 37.6100629 | 68.6915888 | 62.4540938 | 90.0859753 |
| SluU59 + SluD48 | 59 + 114 | 47.1698113 | 65.8878505 | 62.1517771 | 87.2830725 |
| SluU10 + SluD06 | 10 + 72 | 48.578492 | 63.0541872 | 61.3699907 | 86.3519313 |
| SluU21 + SluD14 | 21 + 81 | 54.0173053 | 63.0541872 | 61.2514758 | 89.6 |
| SluU63 + SluD56 | 63 + 122 | 50.6918239 | 90.046729 | 60.9140859 | 88.1204685 |
| SluU64 + SluD56 | 64 + 122 | 42.1383648 | 67.7570093 | 60.3561047 | 87.6001687 |
| SluU62 + SluD14 | 62 + 81 | N/A | N/A | 59.5964691 | 85.6952317 |
| SluU59 + SluD14 | 59 + 81 | N/A | N/A | 59.2913386 | 84.8609355 |
| SluU59 + SluD73 | 59 + 139 | 42.1383648 | 64.953271 | 59.2228571 | 87.854129 |
| SluU08 + SluD06 | 8 + 72 | 46.6007417 | 59.1133005 | 59.2214112 | 86.6882163 |
| SluU62 + SluD18 | 62 + 84 | N/A | N/A | 57.1738188 | 89.3198263 |
| SluU62 + SluD83 | 62 + 149 | 43.1446541 | 68.6915888 | 56.6520468 | 88.9364129 |
| SluU08 + SluD14 | 8 + 81 | 45.1174289 | 63.0541872 | 56.3689997 | 82.0916503 |
| SluU21 + SluD34 | 21 + 100 | 42.1619983 | 61.7489987 | 56.0138249 | 88.4267119 |
| SluU62 + SluD32 | 62 + 98 | 50.1886792 | 79.9065421 | 55.6309362 | 88.7545685 |
| SluU21 + SluD18 | 21 + 84 | 45.6118665 | 65.5172414 | 54.2579625 | 89.213198 |
| SluU62 + SluD48 | 62 + 114 | 35.5974843 | 66.3551402 | 54.2204996 | 88.0831778 |
| SluU62 + SluD100 | 62 + 166 | 37.1069182 | 64.0186916 | 53.4463018 | 88.3484474 |
| SluU62 + SluD73 | 62 + 139 | 38.1132075 | 65.8878505 | 52.8724895 | 88.1341108 |
| SluU21 + SluD32 | 21 + 98 | 41.9409007 | 70.091156 | 52.7119042 | 88.7643521 |
| SluU59 + SluD06 | 59 + 72 | N/A | N/A | 51.9063707 | 87.4718196 |
| SluU21 + SluD48 | 21 + 114 | 36.6578819 | 59.1915658 | 51.7634636 | 86.3590772 |
| SluU21 + SluD83 | 21 + 149 | 37.421306 | 63.4351549 | 51.6537181 | 89.2267593 |
| SluU21 + SluD68 | 21 + 134 | 35.6690067 | 58.698955 | 51.496515 | 87.5733855 |
| SluU21 + SluD73 | 21 + 139 | 36.1721514 | 61.5659961 | 51.1208883 | 88.1798002 |
| SluU10 + SluD56 | 10 + 122 | 33.8862327 | 54.9226555 | 49.8346477 | 83.6832633 |
| SluU62 + SluD06 | 62 + 72 | N/A | N/A | 49.1497006 | 86.0702492 |
| SluU62 + SluD68 | 62 + 134 | 33.081761 | 63.5514019 | 49.0617228 | 87.7113867 |
| SluU21 + SluD100 | 21 + 166 | 36.675296 | 63.9910686 | 48.331221 | 87.6951574 |
| SluU21 + SluD06 | 21 + 72 | 46.6007417 | 66.0098522 | 48.2716352 | 88.4660081 |
| SluU06 + SluD56 | 6 + 122 | 31.6525565 | 54.909995 | 47.0844423 | 85.1293103 |
| SluU59 + SluD56 | 59 + 122 | 35.5974843 | 55.1401869 | 46.3956511 | 84.4827586 |
| SluU08 + SluD56 | 8 + 122 | 32.1644083 | 52.2639381 | 46.21121 | 83.7387264 |
| SluU62 + SluD56 | 62 + 122 | 32.0754717 | 61.2149533 | 36.419214 | 84.2970677 |
| SluU21 + SluD56 | 21 + 122 | 25.9404032 | 59.9684637 | 31.9021039 | 85.3877315 |
| DM control | | N/A | N/A | 1.955 | 1.813814075 |
| Healthy control | | N/A | N/A | 96.9184349 | 96.76620245 |

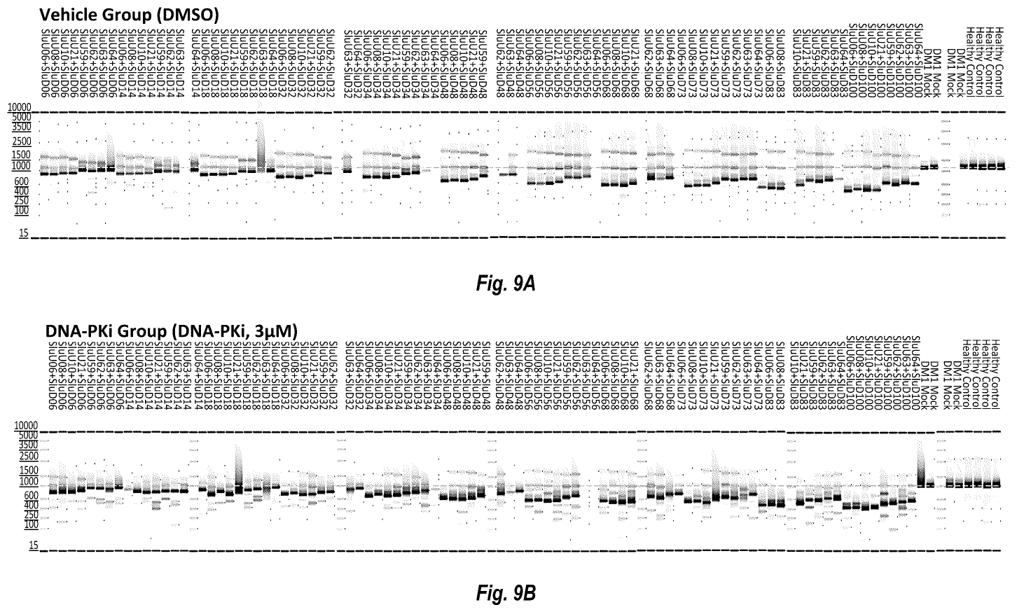

TapeStation analysis was used to assess the large indel (>30 bp) prof ile induced by the SluCas9+sgRNAs pairs (FIG. 9A (vehicle DMSO) and FIG. 9B (with DNA-PKi). The top bands appearing at approximately size 10,000 are the upper standards, and the bottom bands appearing at approximately size 15 are the lower standards. The dashed lines indicate the 1174 bp PCR products amplified from non-edited wild-type allele or wild-type allele with small indels (<30 bp). Several SluCas9 sgRNAs pairs induced large deletions of various sizes, represented by the PCR bands located below the 1174 bp PCR band (>30 bp). Compared to the vehicle group (A), the DNA-PKI group (B) showed more abundant large deletions. DM 1 Mock is the DM 1 patient myoblasts that were nucleofected with SluCas9 protein but not sgRNA.

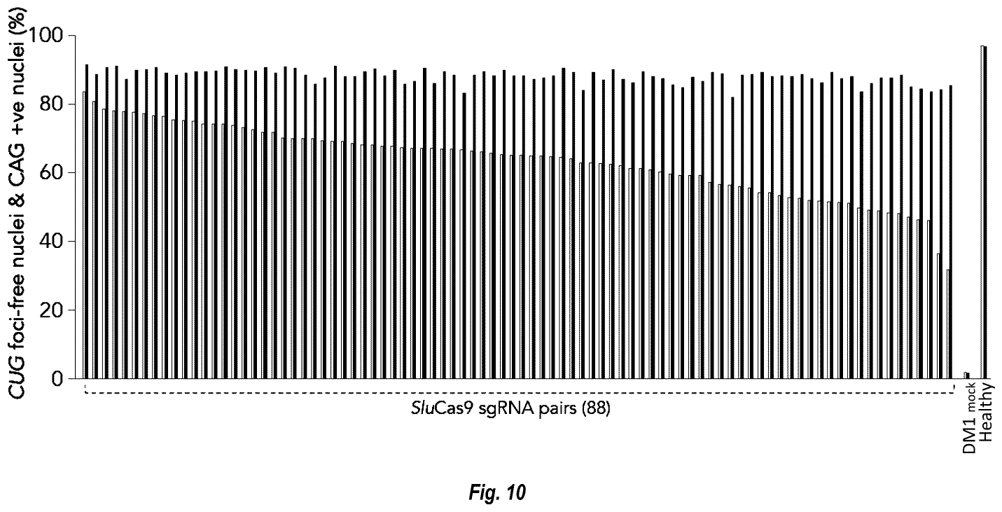

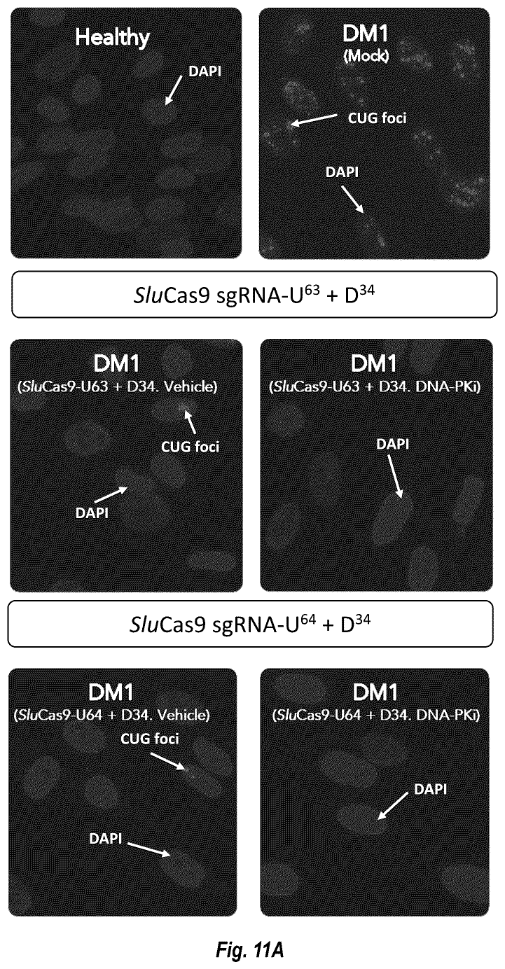

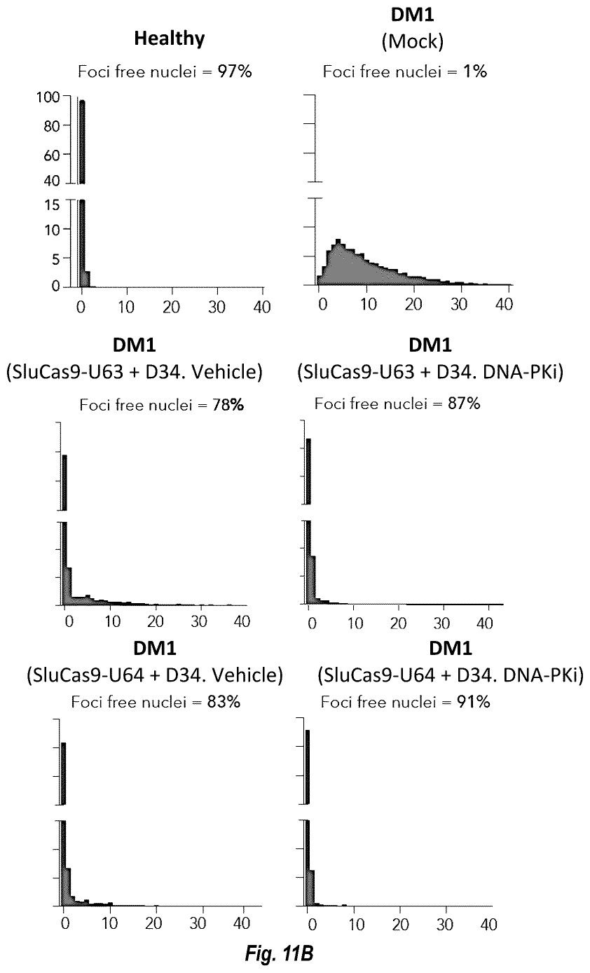

FISH staining of RNA foci showed reduction of CUG foci (formed by the CUG repeat expansion in the DMPKmRNA) in DM1 patient myoblasts nucleofected with the 88 SluCas9 sgRNA pairs (FIG. 10). The percentage of CUG foci free nuclei in vehicle (white bars) or DNA-PKI (black bars) treated myoblasts are shown. The sgRNA pairs were ordered from the highest efficiency to the lowest efficiency in the vehicle group. The healthy myoblasts (Healthy) served as a positive control, and the DM1 patient myoblasts that were nucleofected with SluCas9 protein but not sgRNA (DM1) served as a negative control. Additionally, two pairs of SluCas9 guides (U63+D34) (SEQ ID NOs: 63+100, respectively) and U64+D34 (SEQ ID NOs: 64+100, respectively) successfully reduced RNA foci with and without DNA-PKi (FIG. 11A-B). Four (4) sgRNAs showed exceptionally high RNA foci reduction efficiency (SluU08 (SEQ ID NO: 8), SluU63 (SEQ ID NO: 63), SluU64 (SEQ ID NO: 64) and SluD14 (SEQ ID NO: 81)), which indicates that they may excise the CTG repeat expansion either by working with their sgRNA partner(s) or by working themselves alone.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

1. A composition comprising a single nucleic acid molecule encoding one or more guide RNAs and a Cas9, wherein the single nucleic acid molecule comprises:
a. a first nucleic acid encoding one or more spacer sequence selected from any one of SEQ ID NOs: 1-65, 67-167, and 201-531 and a second nucleic acid encoding a Staphylococcus lugdunensis Cas9 (SluCas9);
b. a first nucleic acid encoding one or more spacer sequence comprising at least 20, or 21 contiguous nucleotides of a spacer sequence selected from any one of SEQ ID NOs: 1-65, 67-167, and 201-531 and a second nucleic acid encoding a Staphylococcus lugdunensis Cas9 (SluCas9);
c. a first nucleic acid encoding one or more spacer sequence that is at least 90% identical to any one of SEQ ID NOs: 1-65, 67-167, and 201-531 and a second nucleic acid encoding a Staphylococcus lugdunensis Cas9 (SluCas9);
d. a first nucleic acid encoding 2 spacer sequences selected from any one of SEQ ID NOs: 63 and 100, and 64 and 100, and a second nucleic acid encoding a Staphylococcus lugdunensis Cas9 (SluCas9); or
e. a first nucleic acid encoding one or more spacer sequence selected from any one of SEQ ID NOs: 8, 63, 64, and 81 and a second nucleic acid encoding a Staphylococcus lugdunensis Cas9 (SluCas9).

2. The composition of claim 1, further comprising a DNA-PK inhibitor.

3. The composition of claim 2, wherein the DNA-PK inhibitor is Compound 6, Compound 1, or Compound 2.

4. (canceled)

5. (canceled)

6. The composition of claim 1, wherein the guide RNA is an sgRNA.

7. The composition of claim 1, wherein the guide RNA is a modified guide RNA.

8.-11. (canceled)

12. The composition of claim 1, wherein the single nucleic acid molecule is associated with a lipid nanoparticle (LNP), or wherein the single nucleic acid molecule is associated with a viral vector.

13. (canceled)

14. The composition of claim 12, wherein the single nucleic acid molecule is associated with a viral vector, and wherein the viral vector is an adeno-associated virus vector, a lentiviral vector, an integrase-deficient lentiviral vector, an adenoviral vector, a vaccinia viral vector, an alphaviral vector, or a herpes simplex viral vector.

15.-22. (canceled)

23. The composition of claim 1, comprising a nucleic acid encoding SluCas9, wherein the SluCas9 comprises the amino acid sequence of any one of SEQ ID NOs: 712 or 718-720.

24.-31. (canceled)

32. A method of treating Myotonic Dystrophy Type 1 (DM1), the method comprising delivering to a cell a single nucleic acid molecule comprising a nucleic acid encoding a Staphylococcus lugdunensis Cas9 (SluCas9); and:
i) a nucleic acid encoding a guide RNA, wherein the guide RNA comprises:
a. one or more spacer sequence selected from any one of SEQ ID NOs: 1-65, 67-167, and 201-531;
b. one or more spacer sequence comprising at least 20, or 21 contiguous nucleotides of a spacer sequence selected from any one of SEQ ID NOs: 1-65, 67-167, and 201-531; or
c. one or more spacer sequence that is at least 90% identical to any one of SEQ ID NOs: 1-65, 67-167, and 201-531; or
ii) a nucleic acid encoding a pair of guide RNAs comprising:
a. a first spacer sequence selected from any one of SEQ ID NOs: 1-65 and a second spacer sequence selected from any one of SEQ ID NOs: 67-167;
b. a first and second spacer sequence comprising at least 20, or 21 contiguous nucleotides of any of the first and second spacer sequences of i) a.;
c. a first and second spacer sequence that is at least 90% identical to any of the first and second spacer sequences of i) a. or i) b.;
d. a first and second spacer, or one or more vectors encoding the pair of guide RNAs, wherein the first and second spacer sequences comprise any one of the following pairs of SEQ ID NOs; 6 and 72; 6 and 81; 6 and 84; 6 and 98; 6 and 100; 6 and 114; 6 and 122; 6 and 134; 6 and 139; 6 and 149; 6 and 166; 8 and 72; 8 and 72; 8 and 81; 8 and 84; 8 and 98; 8 and 100; 8 and 114; 8 and 122; 8 and 134; 8 and 139; 8 and 149; 8 and 166; 10 and 72; 10 and 81; 10 and 84; 10 and 98; 10 and 100; 10 and 114; 10 and 122; 10 and 134; 10 and 139; 10 and 149; 10 and 166; 21 and 72; 21 and 81; 21 and 84; 21 and 98; 21 and 100; 21 and 114; 21 and 122; 21 and 134; 21 and 139; 21 and 149; 21 and 166; 58 and 72; 58 and 81; 58 and 84; 58 and 98; 58 and 100; 58 and 114; 58 and 122; 58 and 134; 58 and 139; 58 and 149; 58 and 166; 62 and 72; 62 and 81; 62 and 84; 62 and 98; 62 and 100; 62 and 114; 62 and 122; 62 and 134; 62 and 139; 62 and 149; 62 and 166; 63 and 72; 63 and 81; 63 and 84; 63 and 98; 63 and 100; 63 and 114; 63 and 122; 63 and 134; 63 and 139; 63 and 149; 63 and 166; 64 and 72; 64 and 81; 64 and 84; 64 and 98; 64 and 100; 64 and 114; 64 and 122; 64 and 134; 64 and 139; 64 and 149; and 64 and 166; or
e. a first and second spacer, or one or more vectors encoding the pair of guide RNAs, wherein the first and second spacer sequences comprise SEQ ID NOs: 63 and 100 or SEQ ID NOs: 64 and 100.

33. (canceled)

34. (canceled)

35. A method of excising a CTG repeat in the 3′ UTR of the DMPK gene, the method comprising delivering to a cell a single nucleic acid molecule comprising a nucleic acid encoding a Staphylococcus lugdunensis Cas9 (SluCas9); and:
i) a nucleic acid encoding a guide RNA, wherein the guide RNA comprises:
a. one or more spacer sequence selected from any one of SEQ ID NOs: 1-65, 67-167, and 201-531;
b. one or more spacer sequence selected from any one of SEQ ID NOs: 8, 63, 64, and 81;
c. one or more spacer sequence comprising at least 20, or 21 contiguous nucleotides of a spacer sequence selected from any one of SEQ ID NOs: 1-65, 67-167, and 201-531; or
d. one or more spacer sequence that is at least 90% identical to any one of SEQ ID NOs: 1-65, 67-167, and 201-531; or
ii) a nucleic acid encoding a pair of guide RNAs comprising:
a. a first spacer sequence selected from any one of SEQ ID NOs: 1-65 and a second spacer sequence selected from any one of SEQ ID NOs: 67-167;
b. a first and second spacer sequence comprising at least 20, or 21 contiguous nucleotides of any of the first and second spacer sequences of i) and;
c. a first and second spacer sequence that is at least 90% identical to any of the first and second spacer sequences of i) a. or i) b.; or
d. a first and second spacer sequence selected from any one of SEQ ID NOs: 63 and 100, and SEQ ID NOs: 64 and 100.

36. (canceled)

37. (canceled)

38. The method of claim 32, comprising administering a DNA-PK inhibitor.

39. The method of claim 38, wherein the DNA-PK inhibitor is Compound 6, Compound 1, or Compound 2.

40. (canceled)

41. (canceled)

42. The method of claim 32, wherein the SluCas9 comprises the amino acid sequence of any one of SEQ ID NOs: 712 or 718-720.

43. (canceled)

44. (canceled)

45. The composition of claim 1, wherein the nucleic acid encoding the gRNA or the nucleic acid encoding the pair of gRNAs comprises a sequence selected from any one of SEQ ID NOs: 600-601, or 900-917.

46. (canceled)

47. The composition of claim 1, wherein the nucleic acid molecule encodes at least a first guide RNA and a second guide RNA, and wherein the nucleic acid molecule further encodes a spacer sequence for the first guide RNA, a scaffold sequence for the first guide RNA, a spacer sequence for the second RNA, and a scaffold sequence for the second guide RNA.

48.-52. (canceled)

53. The composition of claim 48, wherein the scaffold sequence for the first guide RNA comprises a sequence selected from the group consisting of SEQ ID NOs: 901-916, and wherein the scaffold sequence for the second guide RNA comprises a different sequence selected from the group consisting of SEQ ID NOs: 901-916.

54.-61. (canceled)

62. A composition comprising a first nucleic acid molecule and a second nucleic acid molecule, wherein the first nucleic acid molecule encodes a Staphylococcus lugdunensis Cas9 (SluCas9) and the second nucleic acid molecule encodes one or more guide RNAs comprising:
a. one or more spacer sequence selected from any one of SEQ ID NOs: 1-65, 67-167, and 201-531;
b. one or more spacer sequence comprising at least 20, or 21 contiguous nucleotides of a spacer sequence selected from any one of SEQ ID NOs: 1-65, 67-167, and 201-531; or
c. one or more spacer sequence that is at least 90% identical to any one of SEQ ID NOs: 1-65, 67-167, and 201-531.

63. The composition of claim 62, wherein the first nucleic acid molecule does not encode a guide RNA.

64. The composition of claim 62, wherein the first nucleic acid molecule encodes:
a. one or more spacer sequence selected from any one of SEQ ID NOs: 1-65, 67-167, and 201-531;
b. one or more spacer sequence comprising at least 20, or 21 contiguous nucleotides of a spacer sequence selected from any one of SEQ ID NOs: 1-65, 67-167, and 201-531; or
c. one or more spacer sequence that is at least 90% identical to any one of SEQ ID NOs: 1-65, 67-167, and 201-531.

65.-87. (canceled)


SEQUENCE LISTING (943 sequences)

SEQ ID NO: 1 - DNA, 22 bp, synthetic construct
  misc_feature 1..22: note = Synthetic
  source 1..22: mol_type = other DNA
aaccctagaactgtcttcgact

SEQ ID NO: 2 - DNA, 22 bp, synthetic construct
  misc_feature 1..22: note = Synthetic
  source 1..22: mol_type = other DNA
accctagaactgtcttcgactc

SEQ ID NO: 3 - DNA, 22 bp, synthetic construct
  misc_feature 1..22: note = Synthetic
  source 1..22: mol_type = other DNA
ccctagaactgtcttcgactcc

SEQ ID NO: 4 - DNA, 22 bp, synthetic construct
  misc_feature 1..22: note = Synthetic
  source 1..22: mol_type = other DNA
ccccggagtcgaagacagttct

SEQ ID NO: 5 - DNA, 22 bp, synthetic construct
  misc_feature 1..22: note = Synthetic
  source 1..22: mol_type = other DNA
gccccggagtcgaagacagttc

SEQ ID NO: 6 - DNA, 22 bp, synthetic construct
  misc_feature 1..22: note = Synthetic
  source 1..22: mol_type = other DNA
tgtcttcgactccggggccccg

SEQ ID NO: 7 - DNA, 22 bp, synthetic construct
  misc_feature 1..22: note = Synthetic
  source 1..22: mol_type = other DNA
cactcagtcttccaacggggcc

SEQ ID NO: 8 - DNA, 22 bp, synthetic construct
  misc_feature 1..22: note = Synthetic
  source 1..22: mol_type = other DNA
gccccgttggaagactgagtgc

SEQ ID NO: 9 - DNA, 22 bp, synthetic construct
  misc_feature 1..22: note = Synthetic
  source 1..22: mol_type = other DNA
ccccgttggaagactgagtgcc

SEQ ID NO: 10 - DNA, 22 bp, synthetic construct
  misc_feature 1..22: note = Synthetic
  source 1..22: mol_type = other DNA
cccgttggaagactgagtgccc

SEQ ID NO: 11 - DNA, 22 bp, synthetic construct
  misc_feature 1..22: note = Synthetic
  source 1..22: mol_type = other DNA
cccgggcactcagtcttccaac

SEQ ID NO: 12 - DNA, 22 bp, synthetic construct
  misc_feature 1..22: note = Synthetic
  source 1..22: mol_type = other DNA
ccccgggcactcagtcttccaa

SEQ ID NO: 13 - DNA, 22 bp, synthetic construct
  misc_feature 1..22: note = Synthetic
  source 1..22: mol_type = other DNA
gccccgggcactcagtcttcca

SEQ ID NO: 14 - DNA, 22 bp, synthetic construct
  misc_feature 1..22: note = Synthetic
  source 1..22: mol_type = other DNA
tggaagactgagtgcccggggc

SEQ ID NO: 15 - DNA, 22 bp, synthetic construct
  misc_feature 1..22: note = Synthetic
  source 1..22: mol_type = other DNA
gcgcggcttctgtgccgtgccc

SEQ ID NO: 16 - DNA, 22 bp, synthetic construct
  misc_feature 1..22: note = Synthetic
  source 1..22: mol_type = other DNA
ggcgcggcttctgtgccgtgcc

SEQ ID NO: 17 - DNA, 22 bp, synthetic construct
  misc_feature 1..22: note = Synthetic
  source 1..22: mol_type = other DNA
tgtgaactggcaggcggtgggc

SEQ ID NO: 18 - DNA, 22 bp, synthetic construct
  misc_feature 1..22: note = Synthetic
  source 1..22: mol_type = other DNA
gcggttgtgaactggcaggcgg

SEQ ID NO: 19 - DNA, 22 bp, synthetic construct
  misc_feature 1..22: note = Synthetic
  source 1..22: mol_type = other DNA
agcggttgtgaactggcaggcg

SEQ ID NO: 20 - DNA, 22 bp, synthetic construct
  misc_feature 1..22: note = Synthetic
  source 1..22: mol_type = other DNA
cggagcggttgtgaactggcag

SEQ ID NO: 21 - DNA, 22 bp, synthetic construct
  misc_feature 1..22: note = Synthetic
  source 1..22: mol_type = other DNA
gctcggagcggttgtgaactgg

SEQ ID NO: 22 - DNA, 22 bp, synthetic construct
  misc_feature 1..22: note = Synthetic
  source 1..22: mol_type = other DNA
ccagttcacaaccgctccgagc

SEQ ID NO: 23 - DNA, 22 bp, synthetic construct
  misc_feature 1..22: note = Synthetic
  source 1..22: mol_type = other DNA
cagttcacaaccgctccgagcg

SEQ ID NO: 24 - DNA, 22 bp, synthetic construct
  misc_feature 1..22: note = Synthetic
  source 1..22: mol_type = other DNA
ccacgctcggagcggttgtgaa

SEQ ID NO: 25 - DNA, 22 bp, synthetic construct
  misc_feature 1..22: note = Synthetic
  source 1..22: mol_type = other DNA
tgggcggagacccacgctcgga

SEQ ID NO: 26 - DNA, 22 bp, synthetic construct
  misc_feature 1..22: note = Synthetic
  source 1..22: mol_type = other DNA
ggagctgggcggagacccacgc

SEQ ID NO: 27 - DNA, 22 bp, synthetic construct
  misc_feature 1..22: note = Synthetic
  source 1..22: mol_type = other DNA
cgcccagctccagtcctgtgat

SEQ ID NO: 28 - DNA, 22 bp, synthetic construct
  misc_feature 1..22: note = Synthetic
  source 1..22: mol_type = other DNA
gcccagctccagtcctgtgatc

SEQ ID NO: 29 - DNA, 22 bp, synthetic construct
  misc_feature 1..22: note = Synthetic
  source 1..22: mol_type = other DNA
cggatcacaggactggagctgg

SEQ ID NO: 30 - DNA, 22 bp, synthetic construct
  misc_feature 1..22: note = Synthetic
  source 1..22: mol_type = other DNA
gcccggatcacaggactggagc

SEQ ID NO: 31 - DNA, 22 bp, synthetic construct
  misc_feature 1..22: note = Synthetic
  source 1..22: mol_type = other DNA
ggcccggatcacaggactggag

SEQ ID NO: 32 - DNA, 22 bp, synthetic construct
  misc_feature 1..22: note = Synthetic
  source 1..22: mol_type = other DNA
ggggcgggcccggatcacagga

SEQ ID NO: 33 - DNA, 22 bp, synthetic construct
  misc_feature 1..22: note = Synthetic
  source 1..22: mol_type = other DNA
tgtgatccgggcccgcccccta

SEQ ID NO: 34 - DNA, 22 bp, synthetic construct
  misc_feature 1..22: note = Synthetic
  source 1..22: mol_type = other DNA
gctagggggcgggcccggatca

SEQ ID NO: 35 - DNA, 22 bp, synthetic construct
  misc_feature 1..22: note = Synthetic
  source 1..22: mol_type = other DNA
atccgggcccgccccctagcgg

SEQ ID NO: 36 - DNA, 22 bp, synthetic construct
  misc_feature 1..22: note = Synthetic
  source 1..22: mol_type = other DNA
tccgggcccgccccctagcggc

SEQ ID NO: 37 - DNA, 22 bp, synthetic construct
  misc_feature 1..22: note = Synthetic
  source 1..22: mol_type = other DNA
ccgggcccgccccctagcggcc

SEQ ID NO: 38 - DNA, 22 bp, synthetic construct
  misc_feature 1..22: note = Synthetic
  source 1..22: mol_type = other DNA
ggcccgccccctagcggccggg

SEQ ID NO: 39 - DNA, 22 bp, synthetic construct
  misc_feature 1..22: note = Synthetic
  source 1..22: mol_type = other DNA
ccccggccgctagggggcgggc

SEQ ID NO: 40 - DNA, 22 bp, synthetic construct
  misc_feature 1..22: note = Synthetic
  source 1..22: mol_type = other DNA
gcccgccccctagcggccgggg

SEQ ID NO: 41 - DNA, 22 bp, synthetic construct
  misc_feature 1..22: note = Synthetic
  source 1..22: mol_type = other DNA
cgccccctagcggccggggagg

SEQ ID NO: 42 - DNA, 22 bp, synthetic construct
  misc_feature 1..22: note = Synthetic
  source 1..22: mol_type = other DNA
gccccctagcggccggggaggg

SEQ ID NO: 43 - DNA, 22 bp, synthetic construct
  misc_feature 1..22: note = Synthetic
  source 1..22: mol_type = other DNA
tccctccccggccgctaggggg

SEQ ID NO: 44 - DNA, 22 bp, synthetic construct
  misc_feature 1..22: note = Synthetic
  source 1..22: mol_type = other DNA
ccccctagcggccggggaggga

SEQ ID NO: 45 - DNA, 22 bp, synthetic construct
  misc_feature 1..22: note = Synthetic
  source 1..22: mol_type = other DNA
ctccctccccggccgctagggg

SEQ ID NO: 46 - DNA, 22 bp, synthetic construct
  misc_feature 1..22: note = Synthetic
  source 1..22: mol_type = other DNA
cccctccctccccggccgctag

SEQ ID NO: 47 - DNA, 22 bp, synthetic construct
  misc_feature 1..22: note = Synthetic
  source 1..22: mol_type = other DNA
ctagcggccggggagggagggg

SEQ ID NO: 48 - DNA, 22 bp, synthetic construct
  misc_feature 1..22: note = Synthetic
  source 1..22: mol_type = other DNA
gcccctccctccccggccgcta

SEQ ID NO: 49 - DNA, 22 bp, synthetic construct
  misc_feature 1..22: note = Synthetic
  source 1..22: mol_type = other DNA
tagcggccggggagggaggggc

SEQ ID NO: 50 - DNA, 22 bp, synthetic construct
  misc_feature 1..22: note = Synthetic
  source 1..22: mol_type = other DNA
ggcccctccctccccggccgct

SEQ ID NO: 51 - DNA, 22 bp, synthetic construct
  misc_feature 1..22: note = Synthetic
  source 1..22: mol_type = other DNA
cggcccctccctccccggccgc

SEQ ID NO: 52 - DNA, 22 bp, synthetic construct
  misc_feature 1..22: note = Synthetic
  source 1..22: mol_type = other DNA
cggggagggaggggccgggtcc

SEQ ID NO: 53 - DNA, 22 bp, synthetic construct
  misc_feature 1..22: note = Synthetic
  source 1..22: mol_type = other DNA
cgcggacccggcccctccctcc

SEQ ID NO: 54 - DNA, 22 bp, synthetic construct
  misc_feature 1..22: note = Synthetic
  source 1..22: mol_type = other DNA
gagggaggggccgggtccgcgg

SEQ ID NO: 55 - DNA, 22 bp, synthetic construct
  misc_feature 1..22: note = Synthetic
  source 1..22: mol_type = other DNA
gggccgggtccgcggccggcga

SEQ ID NO: 56 - DNA, 22 bp, synthetic construct
  misc_feature 1..22: note = Synthetic
  source 1..22: mol_type = other DNA
ggccgggtccgcggccggcgaa

SEQ ID NO: 57 - DNA, 22 bp, synthetic construct
  misc_feature 1..22: note = Synthetic
  source 1..22: mol_type = other DNA
gccgggtccgcggccggcgaac

SEQ ID NO: 58 - DNA, 22 bp, synthetic construct
  misc_feature 1..22: note = Synthetic
  source 1..22: mol_type = other DNA
gccccgttcgccggccgcggac

SEQ ID NO: 59 - DNA, 22 bp, synthetic construct
  misc_feature 1..22: note = Synthetic
  source 1..22: mol_type = other DNA
cgcggccggcgaacggggctcg

SEQ ID NO: 60 - DNA, 22 bp, synthetic construct
  misc_feature 1..22: note = Synthetic
  source 1..22: mol_type = other DNA
gcggccggcgaacggggctcga

SEQ ID NO: 61 - DNA, 22 bp, synthetic construct
  misc_feature 1..22: note = Synthetic
  source 1..22: mol_type = other DNA
cttcgagccccgttcgccggcc

SEQ ID NO: 62 - DNA, 22 bp, synthetic construct
  misc_feature 1..22: note = Synthetic
  source 1..22: mol_type = other DNA
aggacccttcgagccccgttcg

SEQ ID NO: 63 - DNA, 22 bp, synthetic construct
  misc_feature 1..22: note = Synthetic
  source 1..22: mol_type = other DNA
ggggctcgaagggtccttgtag

SEQ ID NO: 64 - DNA, 22 bp, synthetic construct
  misc_feature 1..22: note = Synthetic
  source 1..22: mol_type = other DNA
gggctcgaagggtccttgtagc

SEQ ID NO: 65 - DNA, 22 bp, synthetic construct
  misc_feature 1..22: note = Synthetic
  source 1..22: mol_type = other DNA
cagcagcagcattcccggctac

SEQ ID NO: 66 - DNA, 22 bp, synthetic construct
  misc_feature 1..22: note = Synthetic
  source 1..22: mol_type = other DNA
agcagcagcagcagcagcattc

SEQ ID NO: 67 - DNA, 22 bp, synthetic construct
  misc_feature 1..22: note = Synthetic
  source 1..22: mol_type = other DNA
gatcacagaccatttctttctt

SEQ ID NO: 68 - DNA, 22 bp, synthetic construct
  misc_feature 1..22: note = Synthetic
  source 1..22: mol_type = other DNA
cagaccatttctttctttcggc

SEQ ID NO: 69 - DNA, 22 bp, synthetic construct
  misc_feature 1..22: note = Synthetic
  source 1..22: mol_type = other DNA
atttctttctttcggccaggct

SEQ ID NO: 70 - DNA, 22 bp, synthetic construct
  misc_feature 1..22: note = Synthetic
  source 1..22: mol_type = other DNA
tcagcctggccgaaagaaagaa

SEQ ID NO: 71 - DNA, 22 bp, synthetic construct
  misc_feature 1..22: note = Synthetic
  source 1..22: mol_type = other DNA
tcggccaggctgaggccctgac

SEQ ID NO: 72 - DNA, 22 bp, synthetic construct
  misc_feature 1..22: note = Synthetic
  source 1..22: mol_type = other DNA
ccaggctgaggccctgacgtgg

SEQ ID NO: 73 - DNA, 22 bp, synthetic construct
  misc_feature 1..22: note = Synthetic
  source 1..22: mol_type = other DNA
caggctgaggccctgacgtgga

SEQ ID NO: 74 - DNA, 22 bp, synthetic construct
  misc_feature 1..22: note = Synthetic
  source 1..22: mol_type = other DNA
ccatccacgtcagggcctcagc

SEQ ID NO: 75 - DNA, 22 bp, synthetic construct
  misc_feature 1..22: note = Synthetic
  source 1..22: mol_type = other DNA
cctgacgtggatgggcaaactg

SEQ ID NO: 76 - DNA, 22 bp, synthetic construct
  misc_feature 1..22: note = Synthetic
  source 1..22: mol_type = other DNA
ctgcagtttgcccatccacgtc

SEQ ID NO: 77 - DNA, 22 bp, synthetic construct
  misc_feature 1..22: note = Synthetic
  source 1..22: mol_type = other DNA
cctgcagtttgcccatccacgt

SEQ ID NO: 78 - DNA, 22 bp, synthetic construct
  misc_feature 1..22: note = Synthetic
  source 1..22: mol_type = other DNA
cgtggatgggcaaactgcaggc

SEQ ID NO: 79 - DNA, 22 bp, synthetic construct
  misc_feature 1..22: note = Synthetic
  source 1..22: mol_type = other DNA
gtggatgggcaaactgcaggcc

SEQ ID NO: 80 - DNA, 22 bp, synthetic construct
  misc_feature 1..22: note = Synthetic
  source 1..22: mol_type = other DNA
caggcctgggaaggcagcaagc

SEQ ID NO: 81 - DNA, 22 bp, synthetic construct
  misc_feature 1..22: note = Synthetic
  source 1..22: mol_type = other DNA
atgggcaaactgcaggcctggg

SEQ ID NO: 82 - DNA, 22 bp, synthetic construct
  misc_feature 1..22: note = Synthetic
  source 1..22: mol_type = other DNA
gcaggcctgggaaggcagcaag

SEQ ID NO: 83 - DNA, 22 bp, synthetic construct
  misc_feature 1..22: note = Synthetic
  source 1..22: mol_type = other DNA
acggcccggcttgctgccttcc

SEQ ID NO: 84 - DNA, 22 bp, synthetic construct
  misc_feature 1..22: note = Synthetic
  source 1..22: mol_type = other DNA
tggaggatggaacacggacggc

SEQ ID NO: 85 - DNA, 22 bp, synthetic construct
  misc_feature 1..22: note = Synthetic
  source 1..22: mol_type = other DNA
gtgcgtggaggatggaacacgg

SEQ ID NO: 86 - DNA, 22 bp, synthetic construct
  misc_feature 1..22: note = Synthetic
  source 1..22: mol_type = other DNA
gggggtgcgtggaggatggaac

SEQ ID NO: 87 - DNA, 22 bp, synthetic construct
  misc_feature 1..22: note = Synthetic
  source 1..22: mol_type = other DNA
gataggtgggggtgcgtggagg

SEQ ID NO: 88 - DNA, 22 bp, synthetic construct
  misc_feature 1..22: note = Synthetic
  source 1..22: mol_type = other DNA
ctccacgcacccccacctatcg

SEQ ID NO: 89 - DNA, 22 bp, synthetic construct
  misc_feature 1..22: note = Synthetic
  source 1..22: mol_type = other DNA
caacgataggtgggggtgcgtg

SEQ ID NO: 90 - DNA, 22 bp, synthetic construct
  misc_feature 1..22: note = Synthetic
  source 1..22: mol_type = other DNA
aaccaacgataggtgggggtgc

SEQ ID NO: 91 - DNA, 22 bp, synthetic construct
  misc_feature 1..22: note = Synthetic
  source 1..22: mol_type = other DNA
ctttgcgaaccaacgataggtg

SEQ ID NO: 92 - DNA, 22 bp, synthetic construct
  misc_feature 1..22: note = Synthetic
  source 1..22: mol_type = other DNA
actttgcgaaccaacgataggt

SEQ ID NO: 93 - DNA, 22 bp, synthetic construct
  misc_feature 1..22: note = Synthetic
  source 1..22: mol_type = other DNA
cactttgcgaaccaacgatagg

SEQ ID NO: 94 - DNA, 22 bp, synthetic construct
  misc_feature 1..22: note = Synthetic
  source 1..22: mol_type = other DNA
gcactttgcgaaccaacgatag

SEQ ID NO: 95 - DNA, 22 bp, synthetic construct
  misc_feature 1..22: note = Synthetic
  source 1..22: mol_type = other DNA
tttgcactttgcgaaccaacga

SEQ ID NO: 96 - DNA, 22 bp, synthetic construct
  misc_feature 1..22: note = Synthetic
  source 1..22: mol_type = other DNA
ttcttgtgcatgacgccctgct

SEQ ID NO: 97 - DNA, 22 bp, synthetic construct
  misc_feature 1..22: note = Synthetic
  source 1..22: mol_type = other DNA
tcttgtgcatgacgccctgctc

SEQ ID NO: 98 - DNA, 22 bp, synthetic construct
  misc_feature 1..22: note = Synthetic
  source 1..22: mol_type = other DNA
cttgtgcatgacgccctgctct

SEQ ID NO: 99 - DNA, 22 bp, synthetic construct
  misc_feature 1..22: note = Synthetic
  source 1..22: mol_type = other DNA
gacgccctgctctggggagcgt

SEQ ID NO: 100 - DNA, 22 bp, synthetic construct
  misc_feature 1..22: note = Synthetic
  source 1..22: mol_type = other DNA
cgcgccagacgctccccagagc

SEQ ID NO: 101 - DNA, 22 bp, synthetic construct
  misc_feature 1..22: note = Synthetic
  source 1..22: mol_type = other DNA
tcgcgccagacgctccccagag

SEQ ID NO: 102 - DNA, 22 bp, synthetic construct
  misc_feature 1..22: note = Synthetic
  source 1..22: mol_type = other DNA
ggcgcgatctctgcctgcttac

SEQ ID NO: 103 - DNA, 22 bp, synthetic construct
  misc_feature 1..22: note = Synthetic
  source 1..22: mol_type = other DNA
gcgcgatctctgcctgcttact

SEQ ID NO: 104 - DNA, 22 bp, synthetic construct
  misc_feature 1..22: note = Synthetic
  source 1..22: mol_type = other DNA
aaaagcaaatttcccgagtaag

SEQ ID NO: 105 - DNA, 22 bp, synthetic construct
  misc_feature 1..22: note = Synthetic
  source 1..22: mol_type = other DNA
tgcttttgccaaacccgctttt

SEQ ID NO: 106 - DNA, 22 bp, synthetic construct
  misc_feature 1..22: note = Synthetic
  source 1..22: mol_type = other DNA
gcttttgccaaacccgcttttt

SEQ ID NO: 107 - DNA, 22 bp, synthetic construct
  misc_feature 1..22: note = Synthetic
  source 1..22: mol_type = other DNA
cttttgccaaacccgctttttc

SEQ ID NO: 108 - DNA, 22 bp, synthetic construct
  misc_feature 1..22: note = Synthetic
  source 1..22: mol_type = other DNA
cgggatccccgaaaaagcgggt

SEQ ID NO: 109 - DNA, 22 bp, synthetic construct
  misc_feature 1..22: note = Synthetic
  source 1..22: mol_type = other DNA
gggcgcgggatccccgaaaaag

SEQ ID NO: 110 - DNA, 22 bp, synthetic construct
  misc_feature 1..22: note = Synthetic
  source 1..22: mol_type = other DNA
ggggcgcgggatccccgaaaaa

SEQ ID NO: 111 - DNA, 22 bp, synthetic construct
  misc_feature 1..22: note = Synthetic
  source 1..22: mol_type = other DNA
agcgcaagtgaggaggggggcg

SEQ ID NO: 112 - DNA, 22 bp, synthetic construct
  misc_feature 1..22: note = Synthetic
  source 1..22: mol_type = other DNA
cagcgcaagtgaggaggggggc

SEQ ID NO: 113 - DNA, 22 bp, synthetic construct
  misc_feature 1..22: note = Synthetic
  source 1..22: mol_type = other DNA
cccctcctcacttgcgctgctc

SEQ ID NO: 114 - DNA, 22 bp, synthetic construct
  misc_feature 1..22: note = Synthetic
  source 1..22: mol_type = other DNA
gagagcagcgcaagtgaggagg

SEQ ID NO: 115 - DNA, 22 bp, synthetic construct
  misc_feature 1..22: note = Synthetic
  source 1..22: mol_type = other DNA
cgagagcagcgcaagtgaggag

SEQ ID NO: 116 - DNA, 22 bp, synthetic construct
  misc_feature 1..22: note = Synthetic
  source 1..22: mol_type = other DNA
ccgagagcagcgcaagtgagga

SEQ ID NO: 117 - DNA, 22 bp, synthetic construct
  misc_feature 1..22: note = Synthetic
  source 1..22: mol_type = other DNA
tccgagagcagcgcaagtgagg

SEQ ID NO: 118 - DNA, 22 bp, synthetic construct
  misc_feature 1..22: note = Synthetic
  source 1..22: mol_type = other DNA
ctccgagagcagcgcaagtgag

SEQ ID NO: 119 - DNA, 22 bp, synthetic construct
  misc_feature 1..22: note = Synthetic
  source 1..22: mol_type = other DNA
gggctccgagagcagcgcaagt

SEQ ID NO: 120 - DNA, 22 bp, synthetic construct
  misc_feature 1..22: note = Synthetic
  source 1..22: mol_type = other DNA
tgcgctgctctcggagccccag

SEQ ID NO: 121 - DNA, 22 bp, synthetic construct
  misc_feature 1..22: note = Synthetic
  source 1..22: mol_type = other DNA
gccccagccggctccgcccgct

SEQ ID NO: 122 - DNA, 22 bp, synthetic construct
  misc_feature 1..22: note = Synthetic
  source 1..22: mol_type = other DNA
ccagccggctccgcccgcttcg

SEQ ID NO: 123 - DNA, 22 bp, synthetic construct
  misc_feature 1..22: note = Synthetic
  source 1..22: mol_type = other DNA
gccgaagcgggcggagccggct

SEQ ID NO: 124 - DNA, 22 bp, synthetic construct
  misc_feature 1..22: note = Synthetic
  source 1..22: mol_type = other DNA
cgccgaagcgggcggagccggc

SEQ ID NO: 125 - DNA, 22 bp, synthetic construct
  misc_feature 1..22: note = Synthetic
  source 1..22: mol_type = other DNA
ccgccgaagcgggcggagccgg

SEQ ID NO: 126 - DNA, 22 bp, synthetic construct
  misc_feature 1..22: note = Synthetic
  source 1..22: mol_type = other DNA
cggctccgcccgcttcggcggt

SEQ ID NO: 127 - DNA, 22 bp, synthetic construct
  misc_feature 1..22: note = Synthetic
  source 1..22: mol_type = other DNA
caaaccgccgaagcgggcggag

SEQ ID NO: 128 - DNA, 22 bp, synthetic construct
  misc_feature 1..22: note = Synthetic
  source 1..22: mol_type = other DNA
aatatccaaaccgccgaagcgg

SEQ ID NO: 129 - DNA, 22 bp, synthetic construct
  misc_feature 1..22: note = Synthetic
  source 1..22: mol_type = other DNA
ataaatatccaaaccgccgaag

SEQ ID NO: 130 - DNA, 22 bp, synthetic construct
  misc_feature 1..22: note = Synthetic
  source 1..22: mol_type = other DNA
aataaatatccaaaccgccgaa

SEQ ID NO: 131 - DNA, 22 bp, synthetic construct
  misc_feature 1..22: note = Synthetic
  source 1..22: mol_type = other DNA
acctcgtcctccgactcgctga

SEQ ID NO: 132 - DNA, 22 bp, synthetic construct
  misc_feature 1..22: note = Synthetic
  source 1..22: mol_type = other DNA
gcctgtcagcgagtcggaggac

SEQ ID NO: 133 - DNA, 22 bp, synthetic construct
  misc_feature 1..22: note = Synthetic
  source 1..22: mol_type = other DNA
cctccgactcgctgacaggcta

SEQ ID NO: 134 - DNA, 22 bp, synthetic construct
  misc_feature 1..22: note = Synthetic
  source 1..22: mol_type = other DNA
cctgtagcctgtcagcgagtcg

SEQ ID NO: 135 - DNA, 22 bp, synthetic construct
  misc_feature 1..22: note = Synthetic
  source 1..22: mol_type = other DNA
ggtcctgtagcctgtcagcgag

SEQ ID NO: 136 - DNA, 22 bp, synthetic construct
  misc_feature 1..22: note = Synthetic
  source 1..22: mol_type = other DNA
cccaacaaccccaatccacgtt

SEQ ID NO: 137 - DNA, 22 bp, synthetic construct
  misc_feature 1..22: note = Synthetic
  source 1..22: mol_type = other DNA
aaaacgtggattggggttgttg

SEQ ID NO: 138 - DNA, 22 bp, synthetic construct
  misc_feature 1..22: note = Synthetic
  source 1..22: mol_type = other DNA
caaaacgtggattggggttgtt

SEQ ID NO: 139 - DNA, 22 bp, synthetic construct
  misc_feature 1..22: note = Synthetic
  source 1..22: mol_type = other DNA
ccaaaacgtggattggggttgt

SEQ ID NO: 140 - DNA, 22 bp, synthetic construct
  misc_feature 1..22: note = Synthetic
  source 1..22: mol_type = other DNA
tccaaaacgtggattggggttg

SEQ ID NO: 141 - DNA, 22 bp, synthetic construct
  misc_feature 1..22: note = Synthetic
  source 1..22: mol_type = other DNA
cagtgcatccaaaacgtggatt

SEQ ID NO: 142 - DNA, 22 bp, synthetic construct
  misc_feature 1..22: note = Synthetic
  source 1..22: mol_type = other DNA
tcagtgcatccaaaacgtggat

SEQ ID NO: 143 - DNA, 22 bp, synthetic construct
  misc_feature 1..22: note = Synthetic
  source 1..22: mol_type = other DNA
ctcagtgcatccaaaacgtgga

SEQ ID NO: 144 - DNA, 22 bp, synthetic construct
  misc_feature 1..22: note = Synthetic
  source 1..22: mol_type = other DNA
ggggtctcagtgcatccaaaac

SEQ ID NO: 145 - DNA, 22 bp, synthetic construct
  misc_feature 1..22: note = Synthetic
  source 1..22: mol_type = other DNA
tgcactgagaccccgacattcc

SEQ ID NO: 146 - DNA, 22 bp, synthetic construct
  misc_feature 1..22: note = Synthetic
  source 1..22: mol_type = other DNA
acaataaataccgaggaatgtc

SEQ ID NO: 147 - DNA, 22 bp, synthetic construct
  misc_feature 1..22: note = Synthetic
  source 1..22: mol_type = other DNA
gacaataaataccgaggaatgt

SEQ ID NO: 148 - DNA, 22 bp, synthetic construct
  misc_feature 1..22: note = Synthetic
  source 1..22: mol_type = other DNA
agacaataaataccgaggaatg

SEQ ID NO: 149 - DNA, 22 bp, synthetic construct
  misc_feature 1..22: note = Synthetic
  source 1..22: mol_type = other DNA
gtggggacagacaataaatacc

SEQ ID NO: 150 - DNA, 22 bp, synthetic construct
  misc_feature 1..22: note = Synthetic
  source 1..22: mol_type = other DNA
gtatttattgtctgtccccacc

SEQ ID NO: 151 - DNA, 22 bp, synthetic construct
  misc_feature 1..22: note = Synthetic
  source 1..22: mol_type = other DNA
gtcgggggtgggggtcctaggt

SEQ ID NO: 152 - DNA, 22 bp, synthetic construct
  misc_feature 1..22: note = Synthetic
  source 1..22: mol_type = other DNA
ggtcgggggtgggggtcctagg

SEQ ID NO: 153 - DNA, 22 bp, synthetic construct
  misc_feature 1..22: note = Synthetic
  source 1..22: mol_type = other DNA
gggtcgggggtgggggtcctag

SEQ ID NO: 154 - DNA, 22 bp, synthetic construct
  misc_feature 1..22: note = Synthetic
  source 1..22: mol_type = other DNA
cgagggtcgggggtgggggtcc

SEQ ID NO: 155 - DNA, 22 bp, synthetic construct
  misc_feature 1..22: note = Synthetic
  source 1..22: mol_type = other DNA
ttattcgcgagggtcgggggtg

SEQ ID NO: 156 - DNA, 22 bp, synthetic construct
  misc_feature 1..22: note = Synthetic
  source 1..22: mol_type = other DNA
cacccccgaccctcgcgaataa

SEQ ID NO: 157 - DNA, 22 bp, synthetic construct
  misc_feature 1..22: note = Synthetic
  source 1..22: mol_type = other DNA
tttattcgcgagggtcgggggt

SEQ ID NO: 158 - DNA, 22 bp, synthetic construct
  misc_feature 1..22: note = Synthetic
  source 1..22: mol_type = other DNA
ttttattcgcgagggtcggggg

SEQ ID NO: 159 - DNA, 22 bp, synthetic construct
  misc_feature 1..22: note = Synthetic
  source 1..22: mol_type = other DNA
cttttattcgcgagggtcgggg

SEQ ID NO: 160 - DNA, 22 bp, synthetic construct
  misc_feature 1..22: note = Synthetic
  source 1..22: mol_type = other DNA
ggccttttattcgcgagggtcg

SEQ ID NO: 161 - DNA, 22 bp, synthetic construct
  misc_feature 1..22: note = Synthetic
  source 1..22: mol_type = other DNA
gggccttttattcgcgagggtc

SEQ ID NO: 162 - DNA, 22 bp, synthetic construct
  misc_feature 1..22: note = Synthetic
  source 1..22: mol_type = other DNA
agggccttttattcgcgagggt

SEQ ID NO: 163 - DNA, 22 bp, synthetic construct
  misc_feature 1..22: note = Synthetic
  source 1..22: mol_type = other DNA
gagggccttttattcgcgaggg

SEQ ID NO: 164 - DNA, 22 bp, synthetic construct
  misc_feature 1..22: note = Synthetic
  source 1..22: mol_type = other DNA
gatggagggccttttattcgcg

SEQ ID NO: 165 - DNA, 22 bp, synthetic construct
  misc_feature 1..22: note = Synthetic
  source 1..22: mol_type = other DNA
agatggagggccttttattcgc

SEQ ID NO: 166 - DNA, 22 bp, synthetic construct
  misc_feature 1..22: note = Synthetic
  source 1..22: mol_type = other DNA
gtccagagctttgggcagatgg

SEQ ID NO: 167 - DNA, 22 bp, synthetic construct
  misc_feature 1..22: note = Synthetic
  source 1..22: mol_type = other DNA
agtccagagctttgggcagatg

SEQ ID NO: 168 - DNA, 22 bp, synthetic construct
  misc_feature 1..22: note = Synthetic
  source 1..22: mol_type = other DNA
gctgctgctgctgctgctgctg

SEQ ID NO: 169 - DNA, 22 bp, synthetic construct
  misc_feature 1..22: note = Synthetic
  source 1..22: mol_type = other DNA
ctgctgctgctgctgctgctgc

SEQ ID NO: 170 - DNA, 22 bp, synthetic construct
  misc_feature 1..22: note = Synthetic
  source 1..22: mol_type = other DNA
tgctgctgctgctgctgctgct

SEQ ID NO: 171 - DNA, 22 bp, synthetic construct
  misc_feature 1..22: note = Synthetic
  source 1..22: mol_type = other DNA
gctgctgctgctgctgctgctg

SEQ ID NO: 172 - DNA, 22 bp, synthetic construct
  misc_feature 1..22: note = Synthetic
  source 1..22: mol_type = other DNA
ctgctgctgctgctgctgctgg

SEQ ID NO: 173 - DNA, 20 bp, synthetic construct
  misc_feature 1..20: note = Synthetic
  source 1..20: mol_type = other DNA
gcagtttgcccatccacgtc

SEQ ID NO: 174-200 - skipped (27 entries)

SEQ ID NO: 201 - DNA, 21 bp, synthetic construct
  misc_feature 1..21: note = Synthetic
  source 1..21: mol_type = other DNA
aaccctagaactgtcttcgac

SEQ ID NO: 202 - DNA, 20 bp, synthetic construct
  misc_feature 1..20: note = Synthetic
  source 1..20: mol_type = other DNA
aaccctagaactgtcttcga

SEQ ID NO: 203 - DNA, 21 bp, synthetic construct
  misc_feature 1..21: note = Synthetic
  source 1..21: mol_type = other DNA
accctagaactgtcttcgact

SEQ ID NO: 204 - DNA, 20 bp, synthetic construct
  misc_feature 1..20: note = Synthetic
  source 1..20: mol_type = other DNA
accctagaactgtcttcgac

SEQ ID NO: 205 - DNA, 21 bp, synthetic construct
  misc_feature 1..21: note = Synthetic
  source 1..21: mol_type = other DNA
ccctagaactgtcttcgactc

SEQ ID NO: 206 - DNA, 20 bp, synthetic construct
  misc_feature 1..20: note = Synthetic
  source 1..20: mol_type = other DNA
ccctagaactgtcttcgact

SEQ ID NO: 207 - DNA, 21 bp, synthetic construct
  misc_feature 1..21: note = Synthetic
  source 1..21: mol_type = other DNA
cccggagtcgaagacagttct

SEQ ID NO: 208 - DNA, 20 bp, synthetic construct
  misc_feature 1..20: note = Synthetic
  source 1..20: mol_type = other DNA
ccggagtcgaagacagttct

SEQ ID NO: 209 - DNA, 21 bp, synthetic construct
  misc_feature 1..21: note = Synthetic
  source 1..21: mol_type = other DNA
ccccggagtcgaagacagttc

SEQ ID NO: 210 - DNA, 20 bp, synthetic construct
  misc_feature 1..20: note = Synthetic
  source 1..20: mol_type = other DNA
cccggagtcgaagacagttc

SEQ ID NO: 211 - DNA, 21 bp, synthetic construct
  misc_feature 1..21: note = Synthetic
  source 1..21: mol_type = other DNA
tgtcttcgactccggggcccc

SEQ ID NO: 212 - DNA, 20 bp, synthetic construct
  misc_feature 1..20: note = Synthetic
  source 1..20: mol_type = other DNA
tgtcttcgactccggggccc

SEQ ID NO: 213 - DNA, 21 bp, synthetic construct
  misc_feature 1..21: note = Synthetic
  source 1..21: mol_type = other DNA
actcagtcttccaacggggcc

SEQ ID NO: 214 - DNA, 20 bp, synthetic construct
  misc_feature 1..20: note = Synthetic
  source 1..20: mol_type = other DNA
ctcagtcttccaacggggcc

SEQ ID NO: 215 - DNA, 21 bp, synthetic construct
  misc_feature 1..21: note = Synthetic
  source 1..21: mol_type = other DNA
gccccgttggaagactgagtg

SEQ ID NO: 216 - DNA, 20 bp, synthetic construct
  misc_feature 1..20: note = Synthetic
  source 1..20: mol_type = other DNA
gccccgttggaagactgagt

SEQ ID NO: 217 - DNA, 21 bp, synthetic construct
  misc_feature 1..21: note = Synthetic
  source 1..21: mol_type = other DNA
ccccgttggaagactgagtgc

SEQ ID NO: 218 - DNA, 20 bp, synthetic construct
  misc_feature 1..20: note = Synthetic
  source 1..20: mol_type = other DNA
ccccgttggaagactgagtg

SEQ ID NO: 219 - DNA, 21 bp, synthetic construct
  misc_feature 1..21: note = Synthetic
  source 1..21: mol_type = other DNA
cccgttggaagactgagtgcc

SEQ ID NO: 220 - DNA, 20 bp, synthetic construct
  misc_feature 1..20: note = Synthetic
  source 1..20: mol_type = other DNA
cccgttggaagactgagtgc

SEQ ID NO: 221 - DNA, 21 bp, synthetic construct
  misc_feature 1..21: note = Synthetic
  source 1..21: mol_type = other DNA
ccgggcactcagtcttccaac

SEQ ID NO: 222 - DNA, 20 bp, synthetic construct
  misc_feature 1..20: note = Synthetic
  source 1..20: mol_type = other DNA
cgggcactcagtcttccaac

SEQ ID NO: 223 - DNA, 21 bp, synthetic construct
  misc_feature 1..21: note = Synthetic
  source 1..21: mol_type = other DNA
cccgggcactcagtcttccaa

SEQ ID NO: 224 - DNA, 20 bp, synthetic construct
  misc_feature 1..20: note = Synthetic
  source 1..20: mol_type = other DNA
ccgggcactcagtcttccaa

SEQ ID NO: 225 - DNA, 21 bp, synthetic construct
  misc_feature 1..21: note = Synthetic
  source 1..21: mol_type = other DNA
ccccgggcactcagtcttcca

SEQ ID NO: 226 - DNA, 20 bp, synthetic construct
  misc_feature 1..20: note = Synthetic
  source 1..20: mol_type = other DNA
cccgggcactcagtcttcca

SEQ ID NO: 227 - DNA, 21 bp, synthetic construct
  misc_feature 1..21: note = Synthetic
  source 1..21: mol_type = other DNA
tggaagactgagtgcccgggg

SEQ ID NO: 228 - DNA, 20 bp, synthetic construct
  misc_feature 1..20: note = Synthetic
  source 1..20: mol_type = other DNA
tggaagactgagtgcccggg

SEQ ID NO: 229 - DNA, 21 bp, synthetic construct
  misc_feature 1..21: note = Synthetic
  source 1..21: mol_type = other DNA
cgcggcttctgtgccgtgccc

SEQ ID NO: 230 - DNA, 20 bp, synthetic construct
  misc_feature 1..20: note = Synthetic
  source 1..20: mol_type = other DNA
gcggcttctgtgccgtgccc

SEQ ID NO: 231 - DNA, 21 bp, synthetic construct
  misc_feature 1..21: note = Synthetic
  source 1..21: mol_type = other DNA
gcgcggcttctgtgccgtgcc

SEQ ID NO: 232 - DNA, 20 bp, synthetic construct
  misc_feature 1..20: note = Synthetic
  source 1..20: mol_type = other DNA
cgcggcttctgtgccgtgcc

SEQ ID NO: 233 - DNA, 21 bp, synthetic construct
  misc_feature 1..21: note = Synthetic
  source 1..21: mol_type = other DNA
gtgaactggcaggcggtgggc

SEQ ID NO: 234 - DNA, 20 bp, synthetic construct
  misc_feature 1..20: note = Synthetic
  source 1..20: mol_type = other DNA
tgaactggcaggcggtgggc

SEQ ID NO: 235 - DNA, 21 bp, synthetic construct
  misc_feature 1..21: note = Synthetic
  source 1..21: mol_type = other DNA
cggttgtgaactggcaggcgg

SEQ ID NO: 236 - DNA, 20 bp, synthetic construct
  misc_feature 1..20: note = Synthetic
  source 1..20: mol_type = other DNA
ggttgtgaactggcaggcgg

SEQ ID NO: 237 - DNA, 21 bp, synthetic construct
  misc_feature 1..21: note = Synthetic
  source 1..21: mol_type = other DNA
gcggttgtgaactggcaggcg

SEQ ID NO: 238 - DNA, 20 bp, synthetic construct
  misc_feature 1..20: note = Synthetic
  source 1..20: mol_type = other DNA
cggttgtgaactggcaggcg

SEQ ID NO: 239 - DNA, 21 bp, synthetic construct
  misc_feature 1..21: note = Synthetic
  source 1..21: mol_type = other DNA
ggagcggttgtgaactggcag

SEQ ID NO: 240 - DNA, 20 bp, synthetic construct
  misc_feature 1..20: note = Synthetic
  source 1..20: mol_type = other DNA
gagcggttgtgaactggcag

SEQ ID NO: 241 - DNA, 21 bp, synthetic construct
  misc_feature 1..21: note = Synthetic
  source 1..21: mol_type = other DNA
ctcggagcggttgtgaactgg

SEQ ID NO: 242 - DNA, 20 bp, synthetic construct
  misc_feature 1..20: note = Synthetic
  source 1..20: mol_type = other DNA
tcggagcggttgtgaactgg

SEQ ID NO: 243 - DNA, 21 bp, synthetic construct
  misc_feature 1..21: note = Synthetic
  source 1..21: mol_type = other DNA
ccagttcacaaccgctccgag

SEQ ID NO: 244 - DNA, 20 bp, synthetic construct
  misc_feature 1..20: note = Synthetic
  source 1..20: mol_type = other DNA
ccagttcacaaccgctccga

SEQ ID NO: 245 - DNA, 21 bp, synthetic construct
  misc_feature 1..21: note = Synthetic
  source 1..21: mol_type = other DNA
cagttcacaaccgctccgagc

SEQ ID NO: 246 - DNA, 20 bp, synthetic construct
  misc_feature 1..20: note = Synthetic
  source 1..20: mol_type = other DNA
cagttcacaaccgctccgag

SEQ ID NO: 247 - DNA, 21 bp, synthetic construct
  misc_feature 1..21: note = Synthetic
  source 1..21: mol_type = other DNA
cacgctcggagcggttgtgaa

SEQ ID NO: 248 - DNA, 20 bp, synthetic construct
  misc_feature 1..20: note = Synthetic
  source 1..20: mol_type = other DNA
acgctcggagcggttgtgaa

SEQ ID NO: 249 - DNA, 21 bp, synthetic construct
  misc_feature 1..21: note = Synthetic
  source 1..21: mol_type = other DNA
gggcggagacccacgctcgga

SEQ ID NO: 250 - DNA, 20 bp, synthetic construct
  misc_feature 1..20: note = Synthetic
  source 1..20: mol_type = other DNA
ggcggagacccacgctcgga

SEQ ID NO: 251 - DNA, 21 bp, synthetic construct
  misc_feature 1..21: note = Synthetic
  source 1..21: mol_type = other DNA
gagctgggcggagacccacgc

SEQ ID NO: 252 - DNA, 20 bp, synthetic construct
  misc_feature 1..20: note = Synthetic
  source 1..20: mol_type = other DNA
agctgggcggagacccacgc

SEQ ID NO: 253 - DNA, 21 bp, synthetic construct
  misc_feature 1..21: note = Synthetic
  source 1..21: mol_type = other DNA
cgcccagctccagtcctgtga

SEQ ID NO: 254 - DNA, 20 bp, synthetic construct
  misc_feature 1..20: note = Synthetic
  source 1..20: mol_type = other DNA
cgcccagctccagtcctgtg

SEQ ID NO: 255 - DNA, 21 bp, synthetic construct
  misc_feature 1..21: note = Synthetic
  source 1..21: mol_type = other DNA
gcccagctccagtcctgtgat

SEQ ID NO: 256 - DNA, 20 bp, synthetic construct
  misc_feature 1..20: note = Synthetic
  source 1..20: mol_type = other DNA
gcccagctccagtcctgtga

SEQ ID NO: 257 - DNA, 21 bp, synthetic construct
  misc_feature 1..21: note = Synthetic
  source 1..21: mol_type = other DNA
ggatcacaggactggagctgg

SEQ ID NO: 258 - DNA, 20 bp, synthetic construct
  misc_feature 1..20: note = Synthetic
  source 1..20: mol_type = other DNA
gatcacaggactggagctgg

SEQ ID NO: 259 - DNA, 21 bp, synthetic construct
  misc_feature 1..21: note = Synthetic
  source 1..21: mol_type = other DNA
cccggatcacaggactggagc

SEQ ID NO: 260 - DNA, 20 bp, synthetic construct
  misc_feature 1..20: note = Synthetic
  source 1..20: mol_type = other DNA
ccggatcacaggactggagc

SEQ ID NO: 261 - DNA, 21 bp, synthetic construct
  misc_feature 1..21: note = Synthetic
  source 1..21: mol_type = other DNA
gcccggatcacaggactggag

SEQ ID NO: 262 - DNA, 20 bp, synthetic construct
  misc_feature 1..20: note = Synthetic
  source 1..20: mol_type = other DNA
cccggatcacaggactggag

SEQ ID NO: 263 - DNA, 21 bp, synthetic construct
  misc_feature 1..21: note = Synthetic
  source 1..21: mol_type = other DNA
gggcgggcccggatcacagga

SEQ ID NO: 264 - DNA, 20 bp, synthetic construct
  misc_feature 1..20: note = Synthetic
  source 1..20: mol_type = other DNA
ggcgggcccggatcacagga

SEQ ID NO: 265 - DNA, 21 bp, synthetic construct
  misc_feature 1..21: note = Synthetic
  source 1..21: mol_type = other DNA
tgtgatccgggcccgccccct

SEQ ID NO: 266 - DNA, 20 bp, synthetic construct
  misc_feature 1..20: note = Synthetic
  source 1..20: mol_type = other DNA
tgtgatccgggcccgccccc

SEQ ID NO: 267 - DNA, 21 bp, synthetic construct
  misc_feature 1..21: note = Synthetic
  source 1..21: mol_type = other DNA
ctagggggcgggcccggatca

SEQ ID NO: 268 - DNA, 20 bp, synthetic construct
  misc_feature 1..20: note = Synthetic
  source 1..20: mol_type = other DNA
tagggggcgggcccggatca

SEQ ID NO: 269 - DNA, 21 bp, synthetic construct
  misc_feature 1..21: note = Synthetic
  source 1..21: mol_type = other DNA
atccgggcccgccccctagcg

SEQ ID NO: 270 - DNA, 20 bp, synthetic construct
  misc_feature 1..20: note = Synthetic
  source 1..20: mol_type = other DNA
atccgggcccgccccctagc

SEQ ID NO: 271 - DNA, 21 bp, synthetic construct
  misc_feature 1..21: note = Synthetic
  source 1..21: mol_type = other DNA
tccgggcccgccccctagcgg

SEQ ID NO: 272 - DNA, 20 bp, synthetic construct
  misc_feature 1..20: note = Synthetic
  source 1..20: mol_type = other DNA
tccgggcccgccccctagcg

SEQ ID NO: 273 - DNA, 21 bp, synthetic construct
  misc_feature 1..21: note = Synthetic
  source 1..21: mol_type = other DNA
ccgggcccgccccctagcggc

SEQ ID NO: 274 - DNA, 20 bp, synthetic construct
  misc_feature 1..20: note = Synthetic
  source 1..20: mol_type = other DNA
ccgggcccgccccctagcgg

SEQ ID NO: 275 - DNA, 21 bp, synthetic construct
  misc_feature 1..21: note = Synthetic
  source 1..21: mol_type = other DNA
ggcccgccccctagcggccgg

SEQ ID NO: 276 - DNA, 20 bp, synthetic construct
  misc_feature 1..20: note = Synthetic
  source 1..20: mol_type = other DNA
ggcccgccccctagcggccg

SEQ ID NO: 277 - DNA, 21 bp, synthetic construct
  misc_feature 1..21: note = Synthetic
  source 1..21: mol_type = other DNA
cccggccgctagggggcgggc

SEQ ID NO: 278 - DNA, 20 bp, synthetic construct
  misc_feature 1..20: note = Synthetic
  source 1..20: mol_type = other DNA
ccggccgctagggggcgggc

SEQ ID NO: 279 - DNA, 21 bp, synthetic construct
  misc_feature 1..21: note = Synthetic
  source 1..21: mol_type = other DNA
gcccgccccctagcggccggg

SEQ ID NO: 280 - DNA, 20 bp, synthetic construct
  misc_feature 1..20: note = Synthetic
  source 1..20: mol_type = other DNA
gcccgccccctagcggccgg

SEQ ID NO: 281 - DNA, 21 bp, synthetic construct
  misc_feature 1..21: note = Synthetic
  source 1..21: mol_type = other DNA
cgccccctagcggccggggag

SEQ ID NO: 282 - DNA, 20 bp, synthetic construct
  misc_feature 1..20: note = Synthetic
  source 1..20: mol_type = other DNA
cgccccctagcggccgggga

SEQ ID NO: 283 - DNA, 21 bp, synthetic construct
  misc_feature 1..21: note = Synthetic
  source 1..21: mol_type = other DNA
gccccctagcggccggggagg

SEQ ID NO: 284 - DNA, 20 bp, synthetic construct
  misc_feature 1..20: note = Synthetic
  source 1..20: mol_type = other DNA
gccccctagcggccggggag

SEQ ID NO: 285 - DNA, 21 bp, synthetic construct
  misc_feature 1..21: note = Synthetic
  source 1..21: mol_type = other DNA
ccctccccggccgctaggggg

SEQ ID NO: 286 - DNA, 20 bp, synthetic construct
  misc_feature 1..20: note = Synthetic
  source 1..20: mol_type = other DNA
cctccccggccgctaggggg

SEQ ID NO: 287 - DNA, 21 bp, synthetic construct
  misc_feature 1..21: note = Synthetic
  source 1..21: mol_type = other DNA
ccccctagcggccggggaggg

SEQ ID NO: 288 - DNA, 20 bp, synthetic construct
  misc_feature 1..20: note = Synthetic
  source 1..20: mol_type = other DNA
ccccctagcggccggggagg

SEQ ID NO: 289 - DNA, 21 bp, synthetic construct
  misc_feature 1..21: note = Synthetic
  source 1..21: mol_type = other DNA
tccctccccggccgctagggg

SEQ ID NO: 290 - DNA, 20 bp, synthetic construct
  misc_feature 1..20: note = Synthetic
  source 1..20: mol_type = other DNA
ccctccccggccgctagggg

SEQ ID NO: 291 - DNA, 21 bp, synthetic construct
  misc_feature 1..21: note = Synthetic
  source 1..21: mol_type = other DNA
ccctccctccccggccgctag

SEQ ID NO: 292 - DNA, 20 bp, synthetic construct
  misc_feature 1..20: note = Synthetic
  source 1..20: mol_type = other DNA
cctccctccccggccgctag

SEQ ID NO: 293 - DNA, 21 bp, synthetic construct
  misc_feature 1..21: note = Synthetic
  source 1..21: mol_type = other DNA
ctagcggccggggagggaggg

SEQ ID NO: 294 - DNA, 20 bp, synthetic construct
  misc_feature 1..20: note = Synthetic
  source 1..20: mol_type = other DNA
ctagcggccggggagggagg

SEQ ID NO: 295 - DNA, 21 bp, synthetic construct
  misc_feature 1..21: note = Synthetic
  source 1..21: mol_type = other DNA
cccctccctccccggccgcta

SEQ ID NO: 296 - DNA, 20 bp, synthetic construct
  misc_feature 1..20: note = Synthetic
  source 1..20: mol_type = other DNA
ccctccctccccggccgcta

SEQ ID NO: 297 - DNA, 21 bp, synthetic construct
  misc_feature 1..21: note = Synthetic
  source 1..21: mol_type = other DNA
tagcggccggggagggagggg

SEQ ID NO: 298 - DNA, 20 bp, synthetic construct
  misc_feature 1..20: note = Synthetic
  source 1..20: mol_type = other DNA
tagcggccggggagggaggg

SEQ ID NO: 299 - DNA, 21 bp, synthetic construct
  misc_feature 1..21: note = Synthetic
  source 1..21: mol_type = other DNA
gcccctccctccccggccgct

SEQ ID NO: 300 - DNA, 20 bp, synthetic construct
  misc_feature 1..20: note = Synthetic
  source 1..20: mol_type = other DNA
cccctccctccccggccgct

SEQ ID NO: 301 - DNA, 21 bp, synthetic construct
  misc_feature 1..21: note = Synthetic
  source 1..21: mol_type = other DNA
ggcccctccctccccggccgc

SEQ ID NO: 302 - DNA, 20 bp, synthetic construct
  misc_feature 1..20: note = Synthetic
  source 1..20: mol_type = other DNA
gcccctccctccccggccgc

SEQ ID NO: 303 - DNA, 21 bp, synthetic construct
  misc_feature 1..21: note = Synthetic
  source 1..21: mol_type = other DNA
cggggagggaggggccgggtc

SEQ ID NO: 304 - DNA, 20 bp, synthetic construct
  misc_feature 1..20: note = Synthetic
  source 1..20: mol_type = other DNA
cggggagggaggggccgggt

SEQ ID NO: 305 - DNA, 21 bp, synthetic construct
  misc_feature 1..21: note = Synthetic
  source 1..21: mol_type = other DNA
gcggacccggcccctccctcc

SEQ ID NO: 306 - DNA, 20 bp, synthetic construct
  misc_feature 1..20: note = Synthetic
  source 1..20: mol_type = other DNA
cggacccggcccctccctcc

SEQ ID NO: 307 - DNA, 21 bp, synthetic construct
  misc_feature 1..21: note = Synthetic
  source 1..21: mol_type = other DNA
gagggaggggccgggtccgcg

SEQ ID NO: 308 - DNA, 20 bp, synthetic construct
  misc_feature 1..20: note = Synthetic
  source 1..20: mol_type = other DNA
gagggaggggccgggtccgc

SEQ ID NO: 309 - DNA, 21 bp, synthetic construct
  misc_feature 1..21: note = Synthetic
  source 1..21: mol_type = other DNA
gggccgggtccgcggccggcg

SEQ ID NO: 310 - DNA, 20 bp, synthetic construct
  misc_feature 1..20: note = Synthetic
  source 1..20: mol_type = other DNA
gggccgggtccgcggccggc

SEQ ID NO: 311 - DNA, 21 bp, synthetic construct
  misc_feature 1..21: note = Synthetic
  source 1..21: mol_type = other DNA
ggccgggtccgcggccggcga

SEQ ID NO: 312 - DNA, 20 bp, synthetic construct
  misc_feature 1..20: note = Synthetic
  source 1..20: mol_type = other DNA
ggccgggtccgcggccggcg

SEQ ID NO: 313 - DNA, 21 bp, synthetic construct
  misc_feature 1..21: note = Synthetic
  source 1..21: mol_type = other DNA
gccgggtccgcggccggcgaa

SEQ ID NO: 314 - DNA, 20 bp, synthetic construct
  misc_feature 1..20: note = Synthetic
  source 1..20: mol_type = other DNA
gccgggtccgcggccggcga

SEQ ID NO: 315 - DNA, 21 bp, synthetic construct
  misc_feature 1..21: note = Synthetic
  source 1..21: mol_type = other DNA
ccccgttcgccggccgcggac

SEQ ID NO: 316 - DNA, 20 bp, synthetic construct
  misc_feature 1..20: note = Synthetic
  source 1..20: mol_type = other DNA
cccgttcgccggccgcggac

SEQ ID NO: 317 - DNA, 21 bp, synthetic construct
  misc_feature 1..21: note = Synthetic
  source 1..21: mol_type = other DNA
cgcggccggcgaacggggctc

SEQ ID NO: 318 - DNA, 20 bp, synthetic construct
  misc_feature 1..20: note = Synthetic
  source 1..20: mol_type = other DNA
cgcggccggcgaacggggct

SEQ ID NO: 319 - DNA, 21 bp, synthetic construct
  misc_feature 1..21: note = Synthetic
  source 1..21: mol_type = other DNA
gcggccggcgaacggggctcg

SEQ ID NO: 320 - DNA, 20 bp, synthetic construct
  misc_feature 1..20: note = Synthetic
  source 1..20: mol_type = other DNA
gcggccggcgaacggggctc

SEQ ID NO: 321 - DNA, 21 bp, synthetic construct
  misc_feature 1..21: note = Synthetic
  source 1..21: mol_type = other DNA
ttcgagccccgttcgccggcc

SEQ ID NO: 322 - DNA, 20 bp, synthetic construct
  misc_feature 1..20: note = Synthetic
  source 1..20: mol_type = other DNA
tcgagccccgttcgccggcc

SEQ ID NO: 323 - DNA, 21 bp, synthetic construct
  misc_feature 1..21: note = Synthetic
  source 1..21: mol_type = other DNA
ggacccttcgagccccgttcg

SEQ ID NO: 324 - DNA, 20 bp, synthetic construct
  misc_feature 1..20: note = Synthetic
  source 1..20: mol_type = other DNA
gacccttcgagccccgttcg

SEQ ID NO: 325 - DNA, 21 bp, synthetic construct
  misc_feature 1..21: note = Synthetic
  source 1..21: mol_type = other DNA
ggggctcgaagggtccttgta

SEQ ID NO: 326 - DNA, 20 bp, synthetic construct
  misc_feature 1..20: note = Synthetic
  source 1..20: mol_type = other DNA
ggggctcgaagggtccttgt

SEQ ID NO: 327 - DNA, 21 bp, synthetic construct
  misc_feature 1..21: note = Synthetic
  source 1..21: mol_type = other DNA
gggctcgaagggtccttgtag

SEQ ID NO: 328 - DNA, 20 bp, synthetic construct
  misc_feature 1..20: note = Synthetic
  source 1..20: mol_type = other DNA
gggctcgaagggtccttgta

SEQ ID NO: 329 - DNA, 21 bp, synthetic construct
  misc_feature 1..21: note = Synthetic
  source 1..21: mol_type = other DNA
agcagcagcattcccggctac

SEQ ID NO: 330 - DNA, 20 bp, synthetic construct
  misc_feature 1..20: note = Synthetic
  source 1..20: mol_type = other DNA
gcagcagcattcccggctac

SEQ ID NO: 331 - DNA, 21 bp, synthetic construct
  misc_feature 1..21: note = Synthetic
  source 1..21: mol_type = other DNA
gcagcagcagcagcagcattc

SEQ ID NO: 332 - DNA, 20 bp, synthetic construct
  misc_feature 1..20: note = Synthetic
  source 1..20: mol_type = other DNA
cagcagcagcagcagcattc

SEQ ID NO: 333 - DNA, 21 bp, synthetic construct
  misc_feature 1..21: note = Synthetic
  source 1..21: mol_type = other DNA
gatcacagaccatttctttct

SEQ ID NO: 334 - DNA, 20 bp, synthetic construct
  misc_feature 1..20: note = Synthetic
  source 1..20: mol_type = other DNA
gatcacagaccatttctttc

SEQ ID NO: 335 - DNA, 21 bp, synthetic construct
  misc_feature 1..21: note = Synthetic
  source 1..21: mol_type = other DNA
cagaccatttctttctttcgg

SEQ ID NO: 336 - DNA, 20 bp, synthetic construct
  misc_feature 1..20: note = Synthetic
  source 1..20: mol_type = other DNA
cagaccatttctttctttcg

SEQ ID NO: 337 - DNA, 21 bp, synthetic construct
  misc_feature 1..21: note = Synthetic
  source 1..21: mol_type = other DNA
atttctttctttcggccaggc

SEQ ID NO: 338 - DNA, 20 bp, synthetic construct
  misc_feature 1..20: note = Synthetic
  source 1..20: mol_type = other DNA
atttctttctttcggccagg

SEQ ID NO: 339 - DNA, 21 bp, synthetic construct
  misc_feature 1..21: note = Synthetic
  source 1..21: mol_type = other DNA
cagcctggccgaaagaaagaa

SEQ ID NO: 340 - DNA, 20 bp, synthetic construct
  misc_feature 1..20: note = Synthetic
  source 1..20: mol_type = other DNA
agcctggccgaaagaaagaa

SEQ ID NO: 341 - DNA, 21 bp, synthetic construct
  misc_feature 1..21: note = Synthetic
  source 1..21: mol_type = other DNA
tcggccaggctgaggccctga

SEQ ID NO: 342 - DNA, 20 bp, synthetic construct
  misc_feature 1..20: note = Synthetic
  source 1..20: mol_type = other DNA
tcggccaggctgaggccctg

SEQ ID NO: 343 - DNA, 21 bp, synthetic construct
  misc_feature 1..21: note = Synthetic
  source 1..21: mol_type = other DNA
ccaggctgaggccctgacgtg

SEQ ID NO: 344 - DNA, 20 bp, synthetic construct
  misc_feature 1..20: note = Synthetic
  source 1..20: mol_type = other DNA
ccaggctgaggccctgacgt

SEQ ID NO: 345 - DNA, 21 bp, synthetic construct
  misc_feature 1..21: note = Synthetic
  source 1..21: mol_type = other DNA
caggctgaggccctgacgtgg

SEQ ID NO: 346 - DNA, 20 bp, synthetic construct
  misc_feature 1..20: note = Synthetic
  source 1..20: mol_type = other DNA
caggctgaggccctgacgtg

SEQ ID NO: 347 - DNA, 21 bp, synthetic construct
  misc_feature 1..21: note = Synthetic
  source 1..21: mol_type = other DNA
catccacgtcagggcctcagc

SEQ ID NO: 348 - DNA, 20 bp, synthetic construct
  misc_feature 1..20: note = Synthetic
  source 1..20: mol_type = other DNA
atccacgtcagggcctcagc

SEQ ID NO: 349 - DNA, 21 bp, synthetic construct
  misc_feature 1..21: note = Synthetic
  source 1..21: mol_type = other DNA
cctgacgtggatgggcaaact

SEQ ID NO: 350 - DNA, 20 bp, synthetic construct
  misc_feature 1..20: note = Synthetic
  source 1..20: mol_type = other DNA
cctgacgtggatgggcaaac

SEQ ID NO: 351 - DNA, 21 bp, synthetic construct
  misc_feature 1..21: note = Synthetic
  source 1..21: mol_type = other DNA
tgcagtttgcccatccacgtc

SEQ ID NO: 352 - DNA, 21 bp, synthetic construct
  misc_feature 1..21: note = Synthetic
  source 1..21: mol_type = other DNA
ctgcagtttgcccatccacgt

SEQ ID NO: 353 - DNA, 20 bp, synthetic construct
  misc_feature 1..20: note = Synthetic
  source 1..20: mol_type = other DNA
tgcagtttgcccatccacgt

SEQ ID NO: 354 - DNA, 21 bp, synthetic construct
  misc_feature 1..21: note = Synthetic
  source 1..21: mol_type = other DNA
cgtggatgggcaaactgcagg

SEQ ID NO: 355 - DNA, 20 bp, synthetic construct
  misc_feature 1..20: note = Synthetic
  source 1..20: mol_type = other DNA
cgtggatgggcaaactgcag

SEQ ID NO: 356 - DNA, 21 bp, synthetic construct
  misc_feature 1..21: note = Synthetic
  source 1..21: mol_type = other DNA
gtggatgggcaaactgcaggc

SEQ ID NO: 357 - DNA, 20 bp, synthetic construct
  misc_feature 1..20: note = Synthetic
  source 1..20: mol_type = other DNA
gtggatgggcaaactgcagg

SEQ ID NO: 358 - DNA, 21 bp, synthetic construct
  misc_feature 1..21: note = Synthetic
  source 1..21: mol_type = other DNA
caggcctgggaaggcagcaag

SEQ ID NO: 359 - DNA, 20 bp, synthetic construct
  misc_feature 1..20: note = Synthetic
  source 1..20: mol_type = other DNA
caggcctgggaaggcagcaa

SEQ ID NO: 360 - DNA, 21 bp, synthetic construct
  misc_feature 1..21: note = Synthetic
  source 1..21: mol_type = other DNA
atgggcaaactgcaggcctgg

SEQ ID NO: 361 - DNA, 20 bp, synthetic construct
  misc_feature 1..20: note = Synthetic
  source 1..20: mol_type = other DNA
atgggcaaactgcaggcctg

SEQ ID NO: 362 - DNA, 21 bp, synthetic construct
  misc_feature 1..21: note = Synthetic
  source 1..21: mol_type = other DNA
gcaggcctgggaaggcagcaa

SEQ ID NO: 363 - DNA, 20 bp, synthetic construct
  misc_feature 1..20: note = Synthetic
  source 1..20: mol_type = other DNA
gcaggcctgggaaggcagca

SEQ ID NO: 364 - DNA, 21 bp, synthetic construct
  misc_feature 1..21: note = Synthetic
  source 1..21: mol_type = other DNA
cggcccggcttgctgccttcc

SEQ ID NO: 365 - DNA, 20 bp, synthetic construct
  misc_feature 1..20: note = Synthetic
  source 1..20: mol_type = other DNA
ggcccggcttgctgccttcc

SEQ ID NO: 366 - DNA, 21 bp, synthetic construct
  misc_feature 1..21: note = Synthetic
  source 1..21: mol_type = other DNA
ggaggatggaacacggacggc

SEQ ID NO: 367 - DNA, 20 bp, synthetic construct
  misc_feature 1..20: note = Synthetic
  source 1..20: mol_type = other DNA
gaggatggaacacggacggc

SEQ ID NO: 368 - DNA, 21 bp, synthetic construct
  misc_feature 1..21: note = Synthetic
  source 1..21: mol_type = other DNA
tgcgtggaggatggaacacgg

SEQ ID NO: 369 - DNA, 20 bp, synthetic construct
  misc_feature 1..20: note = Synthetic
  source 1..20: mol_type = other DNA
gcgtggaggatggaacacgg

SEQ ID NO: 370 - DNA, 21 bp, synthetic construct
  misc_feature 1..21: note = Synthetic
  source 1..21: mol_type = other DNA
ggggtgcgtggaggatggaac

SEQ ID NO: 371 - DNA, 20 bp, synthetic construct
  misc_feature 1..20: note = Synthetic
  source 1..20: mol_type = other DNA
gggtgcgtggaggatggaac

SEQ ID NO: 372 - DNA, 21 bp, synthetic construct
  misc_feature 1..21: note = Synthetic
  source 1..21: mol_type = other DNA
ataggtgggggtgcgtggagg

SEQ ID NO: 373 - DNA, 20 bp, synthetic construct
  misc_feature 1..20: note = Synthetic
  source 1..20: mol_type = other DNA
taggtgggggtgcgtggagg

SEQ ID NO: 374 - DNA, 21 bp, synthetic construct
  misc_feature 1..21: note = Synthetic
  source 1..21: mol_type = other DNA
ctccacgcacccccacctatc

SEQ ID NO: 375 - DNA, 20 bp, synthetic construct
  misc_feature 1..20: note = Synthetic
  source 1..20: mol_type = other DNA
ctccacgcacccccacctat

SEQ ID NO: 376 - DNA, 21 bp, synthetic construct
  misc_feature 1..21: note = Synthetic
  source 1..21: mol_type = other DNA
aacgataggtgggggtgcgtg

SEQ ID NO: 377 - DNA, 20 bp, synthetic construct
  misc_feature 1..20: note = Synthetic
  source 1..20: mol_type = other DNA
acgataggtgggggtgcgtg

SEQ ID NO: 378 - DNA, 21 bp, synthetic construct
  misc_feature 1..21: note = Synthetic
  source 1..21: mol_type = other DNA
accaacgataggtgggggtgc

SEQ ID NO: 379 - DNA, 20 bp, synthetic construct
  misc_feature 1..20: note = Synthetic
  source 1..20: mol_type = other DNA
ccaacgataggtgggggtgc

SEQ ID NO: 380 - DNA, 21 bp, synthetic construct
  misc_feature 1..21: note = Synthetic
  source 1..21: mol_type = other DNA
tttgcgaaccaacgataggtg

SEQ ID NO: 381 - DNA, 20 bp, synthetic construct
  misc_feature 1..20: note = Synthetic
  source 1..20: mol_type = other DNA
ttgcgaaccaacgataggtg

SEQ ID NO: 382 - DNA, 21 bp, synthetic construct
  misc_feature 1..21: note = Synthetic
  source 1..21: mol_type = other DNA
ctttgcgaaccaacgataggt

SEQ ID NO: 383 - DNA, 20 bp, synthetic construct
  misc_feature 1..20: note = Synthetic
  source 1..20: mol_type = other DNA
tttgcgaaccaacgataggt

SEQ ID NO: 384 - DNA, 21 bp, synthetic construct
  misc_feature 1..21: note = Synthetic
  source 1..21: mol_type = other DNA
actttgcgaaccaacgatagg

SEQ ID NO: 385 - DNA, 20 bp, synthetic construct
  misc_feature 1..20: note = Synthetic
  source 1..20: mol_type = other DNA
ctttgcgaaccaacgatagg

SEQ ID NO: 386 - DNA, 21 bp, synthetic construct
  misc_feature 1..21: note = Synthetic
  source 1..21: mol_type = other DNA
cactttgcgaaccaacgatag

SEQ ID NO: 387 - DNA, 20 bp, synthetic construct
  misc_feature 1..20: note = Synthetic
  source 1..20: mol_type = other DNA
actttgcgaaccaacgatag

SEQ ID NO: 388 - DNA, 21 bp, synthetic construct
  misc_feature 1..21: note = Synthetic
  source 1..21: mol_type = other DNA
ttgcactttgcgaaccaacga

SEQ ID NO: 389 - DNA, 20 bp, synthetic construct
  misc_feature 1..20: note = Synthetic
  source 1..20: mol_type = other DNA
tgcactttgcgaaccaacga

SEQ ID NO: 390 - DNA, 21 bp, synthetic construct
  misc_feature 1..21: note = Synthetic
  source 1..21: mol_type = other DNA
ttcttgtgcatgacgccctgc

SEQ ID NO: 391 - DNA, 20 bp, synthetic construct
  misc_feature 1..20: note = Synthetic
  source 1..20: mol_type = other DNA
ttcttgtgcatgacgccctg

SEQ ID NO: 392 - DNA, 21 bp, synthetic construct
  misc_feature 1..21: note = Synthetic
  source 1..21: mol_type = other DNA
tcttgtgcatgacgccctgct

SEQ ID NO: 393 - DNA, 20 bp, synthetic construct
  misc_feature 1..20: note = Synthetic
  source 1..20: mol_type = other DNA
tcttgtgcatgacgccctgc

SEQ ID NO: 394 - DNA, 21 bp, synthetic construct
  misc_feature 1..21: note = Synthetic
  source 1..21: mol_type = other DNA
cttgtgcatgacgccctgctc

SEQ ID NO: 395 - DNA, 20 bp, synthetic construct
  misc_feature 1..20: note = Synthetic
  source 1..20: mol_type = other DNA
cttgtgcatgacgccctgct

SEQ ID NO: 396 - DNA, 21 bp, synthetic construct
  misc_feature 1..21: note = Synthetic
  source 1..21: mol_type = other DNA
gacgccctgctctggggagcg

SEQ ID NO: 397 - DNA, 20 bp, synthetic construct
  misc_feature 1..20: note = Synthetic
  source 1..20: mol_type = other DNA
gacgccctgctctggggagc

SEQ ID NO: 398 - DNA, 21 bp, synthetic construct
  misc_feature 1..21: note = Synthetic
  source 1..21: mol_type = other DNA
gcgccagacgctccccagagc

SEQ ID NO: 399 - DNA, 20 bp, synthetic construct
  misc_feature 1..20: note = Synthetic
  source 1..20: mol_type = other DNA
cgccagacgctccccagagc

SEQ ID NO: 400 - DNA, 21 bp, synthetic construct
  misc_feature 1..21: note = Synthetic
  source 1..21: mol_type = other DNA
cgcgccagacgctccccagag

SEQ ID NO: 401 - DNA, 20 bp, synthetic construct
  misc_feature 1..20: note = Synthetic
  source 1..20: mol_type = other DNA
gcgccagacgctccccagag

SEQ ID NO: 402 - DNA, 21 bp, synthetic construct
  misc_feature 1..21: note = Synthetic
  source 1..21: mol_type = other DNA
ggcgcgatctctgcctgctta

SEQ ID NO: 403 - DNA, 20 bp, synthetic construct
  misc_feature 1..20: note = Synthetic
  source 1..20: mol_type = other DNA
ggcgcgatctctgcctgctt

SEQ ID NO: 404 - DNA, 21 bp, synthetic construct
  misc_feature 1..21: note = Synthetic
  source 1..21: mol_type = other DNA
gcgcgatctctgcctgcttac

SEQ ID NO: 405 - DNA, 20 bp, synthetic construct
  misc_feature 1..20: note = Synthetic
  source 1..20: mol_type = other DNA
gcgcgatctctgcctgctta

SEQ ID NO: 406 - DNA, 21 bp, synthetic construct
  misc_feature 1..21: note = Synthetic
  source 1..21: mol_type = other DNA
aaagcaaatttcccgagtaag

SEQ ID NO: 407 - DNA, 20 bp, synthetic construct
  misc_feature 1..20: note = Synthetic
  source 1..20: mol_type = other DNA
aagcaaatttcccgagtaag

SEQ ID NO: 408 - DNA, 21 bp, synthetic construct
  misc_feature 1..21: note = Synthetic
  source 1..21: mol_type = other DNA
tgcttttgccaaacccgcttt

SEQ ID NO: 409 - DNA, 20 bp, synthetic construct
  misc_feature 1..20: note = Synthetic
  source 1..20: mol_type = other DNA
tgcttttgccaaacccgctt

SEQ ID NO: 410 - DNA, 21 bp, synthetic construct
  misc_feature 1..21: note = Synthetic
  source 1..21: mol_type = other DNA
gcttttgccaaacccgctttt

SEQ ID NO: 411 - DNA, 20 bp, synthetic construct
  misc_feature 1..20: note = Synthetic
  source 1..20: mol_type = other DNA
gcttttgccaaacccgcttt

SEQ ID NO: 412 - DNA, 21 bp, synthetic construct
  misc_feature 1..21: note = Synthetic
  source 1..21: mol_type = other DNA
cttttgccaaacccgcttttt

SEQ ID NO: 413 - DNA, 20 bp, synthetic construct
  misc_feature 1..20: note = Synthetic
  source 1..20: mol_type = other DNA
cttttgccaaacccgctttt

SEQ ID NO: 414 - DNA, 21 bp, synthetic construct
  misc_feature 1..21: note = Synthetic
  source 1..21: mol_type = other DNA
gggatccccgaaaaagcgggt

SEQ ID NO: 415 - DNA, 20 bp, synthetic construct
  misc_feature 1..20: note = Synthetic
  source 1..20: mol_type = other DNA
ggatccccgaaaaagcgggt

SEQ ID NO: 416 - DNA, 21 bp, synthetic construct
  misc_feature 1..21: note = Synthetic
  source 1..21: mol_type = other DNA
ggcgcgggatccccgaaaaag

SEQ ID NO: 417 - DNA, 20 bp, synthetic construct
  misc_feature 1..20: note = Synthetic
  source 1..20: mol_type = other DNA
gcgcgggatccccgaaaaag

SEQ ID NO: 418 - DNA, 21 bp, synthetic construct
  misc_feature 1..21: note = Synthetic
  source 1..21: mol_type = other DNA
gggcgcgggatccccgaaaaa

SEQ ID NO: 419 - DNA, 20 bp, synthetic construct
  misc_feature 1..20: note = Synthetic
  source 1..20: mol_type = other DNA
ggcgcgggatccccgaaaaa

SEQ ID NO: 420 - DNA, 21 bp, synthetic construct
  misc_feature 1..21: note = Synthetic
  source 1..21: mol_type = other DNA
gcgcaagtgaggaggggggcg

SEQ ID NO: 421 - DNA, 20 bp, synthetic construct
  misc_feature 1..20: note = Synthetic
  source 1..20: mol_type = other DNA
cgcaagtgaggaggggggcg

SEQ ID NO: 422 - DNA, 21 bp, synthetic construct
  misc_feature 1..21: note = Synthetic
  source 1..21: mol_type = other DNA
agcgcaagtgaggaggggggc

SEQ ID NO: 423 - DNA, 20 bp, synthetic construct
  misc_feature 1..20: note = Synthetic
  source 1..20: mol_type = other DNA
gcgcaagtgaggaggggggc

SEQ ID NO: 424 - DNA, 21 bp, synthetic construct
  misc_feature 1..21: note = Synthetic
  source 1..21: mol_type = other DNA
cccctcctcacttgcgctgct

SEQ ID NO: 425 - DNA, 20 bp, synthetic construct
  misc_feature 1..20: note = Synthetic
  source 1..20: mol_type = other DNA
cccctcctcacttgcgctgc

SEQ ID NO: 426 - DNA, 21 bp, synthetic construct
  misc_feature 1..21: note = Synthetic
  source 1..21: mol_type = other DNA
agagcagcgcaagtgaggagg

SEQ ID NO: 427 - DNA, 20 bp, synthetic construct
  misc_feature 1..20: note = Synthetic
  source 1..20: mol_type = other DNA
gagcagcgcaagtgaggagg

SEQ ID NO: 428 - DNA, 21 bp, synthetic construct
  misc_feature 1..21: note = Synthetic
  source 1..21: mol_type = other DNA
gagagcagcgcaagtgaggag

SEQ ID NO: 429 - DNA, 20 bp, synthetic construct
  misc_feature 1..20: note = Synthetic
  source 1..20: mol_type = other DNA
agagcagcgcaagtgaggag

SEQ ID NO: 430 - DNA, 21 bp, synthetic construct
  misc_feature 1..21: note = Synthetic
  source 1..21: mol_type = other DNA
cgagagcagcgcaagtgagga

SEQ ID NO: 431 - DNA, 20 bp, synthetic construct
  misc_feature 1..20: note = Synthetic
  source 1..20: mol_type = other DNA
gagagcagcgcaagtgagga

SEQ ID NO: 432 - DNA, 21 bp, synthetic construct
  misc_feature 1..21: note = Synthetic
  source 1..21: mol_type = other DNA
ccgagagcagcgcaagtgagg

SEQ ID NO: 433 - DNA, 20 bp, synthetic construct
  misc_feature 1..20: note = Synthetic
  source 1..20: mol_type = other DNA
cgagagcagcgcaagtgagg

SEQ ID NO: 434 - DNA, 21 bp, synthetic construct
  misc_feature 1..21: note = Synthetic
  source 1..21: mol_type = other DNA
tccgagagcagcgcaagtgag

SEQ ID NO: 435 - DNA, 20 bp, synthetic construct
  misc_feature 1..20: note = Synthetic
  source 1..20: mol_type = other DNA
ccgagagcagcgcaagtgag

SEQ ID NO: 436 - DNA, 21 bp, synthetic construct
  misc_feature 1..21: note = Synthetic
  source 1..21: mol_type = other DNA
ggctccgagagcagcgcaagt

SEQ ID NO: 437 - DNA, 20 bp, synthetic construct
  misc_feature 1..20: note = Synthetic
  source 1..20: mol_type = other DNA
gctccgagagcagcgcaagt

SEQ ID NO: 438 - DNA, 21 bp, synthetic construct
  misc_feature 1..21: note = Synthetic
  source 1..21: mol_type = other DNA
tgcgctgctctcggagcccca

SEQ ID NO: 439 - DNA, 20 bp, synthetic construct
  misc_feature 1..20: note = Synthetic
  source 1..20: mol_type = other DNA
tgcgctgctctcggagcccc

SEQ ID NO: 440 - DNA, 21 bp, synthetic construct
  misc_feature 1..21: note = Synthetic
  source 1..21: mol_type = other DNA
gccccagccggctccgcccgc

SEQ ID NO: 441 - DNA, 20 bp, synthetic construct
  misc_feature 1..20: note = Synthetic
  source 1..20: mol_type = other DNA
gccccagccggctccgcccg

SEQ ID NO: 442 - DNA, 21 bp, synthetic construct
  misc_feature 1..21: note = Synthetic
  source 1..21: mol_type = other DNA
ccagccggctccgcccgcttc

SEQ ID NO: 443 - DNA, 20 bp, synthetic construct
  misc_feature 1..20: note = Synthetic
  source 1..20: mol_type = other DNA
ccagccggctccgcccgctt

SEQ ID NO: 444 - DNA, 21 bp, synthetic construct
  misc_feature 1..21: note = Synthetic
  source 1..21: mol_type = other DNA
ccgaagcgggcggagccggct

SEQ ID NO: 445 - DNA, 20 bp, synthetic construct
  misc_feature 1..20: note = Synthetic
  source 1..20: mol_type = other DNA
cgaagcgggcggagccggct

SEQ ID NO: 446 - DNA, 21 bp, synthetic construct
  misc_feature 1..21: note = Synthetic
  source 1..21: mol_type = other DNA
gccgaagcgggcggagccggc

SEQ ID NO: 447 - DNA, 20 bp, synthetic construct
  misc_feature 1..20: note = Synthetic
  source 1..20: mol_type = other DNA
ccgaagcgggcggagccggc

SEQ ID NO: 448 - DNA, 21 bp, synthetic construct
  misc_feature 1..21: note = Synthetic
  source 1..21: mol_type = other DNA
cgccgaagcgggcggagccgg

SEQ ID NO: 449 - DNA, 20 bp, synthetic construct
  misc_feature 1..20: note = Synthetic
  source 1..20: mol_type = other DNA
gccgaagcgggcggagccgg

SEQ ID NO: 450 - DNA, 21 bp, synthetic construct
  misc_feature 1..21: note = Synthetic
  source 1..21: mol_type = other DNA
cggctccgcccgcttcggcgg

SEQ ID NO: 451 - DNA, 20 bp, synthetic construct
  misc_feature 1..20: note = Synthetic
  source 1..20: mol_type = other DNA
cggctccgcccgcttcggcg

SEQ ID NO: 452 - DNA, 21 bp, synthetic construct
  misc_feature 1..21: note = Synthetic
  source 1..21: mol_type = other DNA
aaaccgccgaagcgggcggag

SEQ ID NO: 453 - DNA, 20 bp, synthetic construct
  misc_feature 1..20: note = Synthetic
  source 1..20: mol_type = other DNA
aaccgccgaagcgggcggag

SEQ ID NO: 454 - DNA, 21 bp, synthetic construct
  misc_feature 1..21: note = Synthetic
  source 1..21: mol_type = other DNA
atatccaaaccgccgaagcgg

SEQ ID NO: 455 - DNA, 20 bp, synthetic construct
  misc_feature 1..20: note = Synthetic
  source 1..20: mol_type = other DNA
tatccaaaccgccgaagcgg

SEQ ID NO: 456 - DNA, 21 bp, synthetic construct
  misc_feature 1..21: note = Synthetic
  source 1..21: mol_type = other DNA
taaatatccaaaccgccgaag

SEQ ID NO: 457 - DNA, 20 bp, synthetic construct
  misc_feature 1..20: note = Synthetic
  source 1..20: mol_type = other DNA
aaatatccaaaccgccgaag

SEQ ID NO: 458 - DNA, 21 bp, synthetic construct
  misc_feature 1..21: note = Synthetic
  source 1..21: mol_type = other DNA
ataaatatccaaaccgccgaa

SEQ ID NO: 459 - DNA, 20 bp, synthetic construct
  misc_feature 1..20: note = Synthetic
  source 1..20: mol_type = other DNA
taaatatccaaaccgccgaa

SEQ ID NO: 460 - DNA, 21 bp, synthetic construct
  misc_feature 1..21: note = Synthetic
  source 1..21: mol_type = other DNA
acctcgtcctccgactcgctg

SEQ ID NO: 461 - DNA, 20 bp, synthetic construct
  misc_feature 1..20: note = Synthetic
  source 1..20: mol_type = other DNA
acctcgtcctccgactcgct

SEQ ID NO: 462 - DNA, 21 bp, synthetic construct
  misc_feature 1..21: note = Synthetic
  source 1..21: mol_type = other DNA
cctgtcagcgagtcggaggac

SEQ ID NO: 463 - DNA, 20 bp, synthetic construct
  misc_feature 1..20: note = Synthetic
  source 1..20: mol_type = other DNA
ctgtcagcgagtcggaggac

SEQ ID NO: 464 - DNA, 21 bp, synthetic construct
  misc_feature 1..21: note = Synthetic
  source 1..21: mol_type = other DNA
cctccgactcgctgacaggct

SEQ ID NO: 465 - DNA, 20 bp, synthetic construct
  misc_feature 1..20: note = Synthetic
  source 1..20: mol_type = other DNA
cctccgactcgctgacaggc

SEQ ID NO: 466 - DNA, 21 bp, synthetic construct
  misc_feature 1..21: note = Synthetic
  source 1..21: mol_type = other DNA
ctgtagcctgtcagcgagtcg

SEQ ID NO: 467 - DNA, 20 bp, synthetic construct
  misc_feature 1..20: note = Synthetic
  source 1..20: mol_type = other DNA
tgtagcctgtcagcgagtcg

SEQ ID NO: 468 - DNA, 21 bp, synthetic construct
  misc_feature 1..21: note = Synthetic
  source 1..21: mol_type = other DNA
gtcctgtagcctgtcagcgag

SEQ ID NO: 469 - DNA, 20 bp, synthetic construct
  misc_feature 1..20: note = Synthetic
  source 1..20: mol_type = other DNA
tcctgtagcctgtcagcgag

SEQ ID NO: 470 - DNA, 21 bp, synthetic construct
  misc_feature 1..21: note = Synthetic
  source 1..21: mol_type = other DNA
cccaacaaccccaatccacgt

SEQ ID NO: 471 - DNA, 20 bp, synthetic construct
  misc_feature 1..20: note = Synthetic
  source 1..20: mol_type = other DNA
cccaacaaccccaatccacg

SEQ ID NO: 472 - DNA, 21 bp, synthetic construct
  misc_feature 1..21: note = Synthetic
  source 1..21: mol_type = other DNA
aaacgtggattggggttgttg

SEQ ID NO: 473 - DNA, 20 bp, synthetic construct
  misc_feature 1..20: note = Synthetic
  source 1..20: mol_type = other DNA
aacgtggattggggttgttg

SEQ ID NO: 474 - DNA, 21 bp, synthetic construct
  misc_feature 1..21: note = Synthetic
  source 1..21: mol_type = other DNA
aaaacgtggattggggttgtt

SEQ ID NO: 475 - DNA, 20 bp, synthetic construct
  misc_feature 1..20: note = Synthetic
  source 1..20: mol_type = other DNA
aaacgtggattggggttgtt

SEQ ID NO: 476 - DNA, 21 bp, synthetic construct
  misc_feature 1..21: note = Synthetic
  source 1..21: mol_type = other DNA
caaaacgtggattggggttgt

SEQ ID NO: 477 - DNA, 20 bp, synthetic construct
  misc_feature 1..20: note = Synthetic
  source 1..20: mol_type = other DNA
aaaacgtggattggggttgt

SEQ ID NO: 478 - DNA, 21 bp, synthetic construct
  misc_feature 1..21: note = Synthetic
  source 1..21: mol_type = other DNA
ccaaaacgtggattggggttg

SEQ ID NO: 479 - DNA, 20 bp, synthetic construct
  misc_feature 1..20: note = Synthetic
  source 1..20: mol_type = other DNA
caaaacgtggattggggttg

SEQ ID NO: 480 - DNA, 21 bp, synthetic construct
  misc_feature 1..21: note = Synthetic
  source 1..21: mol_type = other DNA
agtgcatccaaaacgtggatt

SEQ ID NO: 481 - DNA, 20 bp, synthetic construct
  misc_feature 1..20: note = Synthetic
  source 1..20: mol_type = other DNA
gtgcatccaaaacgtggatt

SEQ ID NO: 482 - DNA, 21 bp, synthetic construct
  misc_feature 1..21: note = Synthetic
  source 1..21: mol_type = other DNA
cagtgcatccaaaacgtggat

SEQ ID NO: 483 - DNA, 20 bp, synthetic construct
  misc_feature 1..20: note = Synthetic
  source 1..20: mol_type = other DNA
agtgcatccaaaacgtggat

SEQ ID NO: 484 - DNA, 21 bp, synthetic construct
  misc_feature 1..21: note = Synthetic
  source 1..21: mol_type = other DNA
tcagtgcatccaaaacgtgga

SEQ ID NO: 485 - DNA, 20 bp, synthetic construct
  misc_feature 1..20: note = Synthetic
  source 1..20: mol_type = other DNA
cagtgcatccaaaacgtgga

SEQ ID NO: 486 - DNA, 21 bp, synthetic construct
  misc_feature 1..21: note = Synthetic
  source 1..21: mol_type = other DNA
gggtctcagtgcatccaaaac

SEQ ID NO: 487 - DNA, 20 bp, synthetic construct
  misc_feature 1..20: note = Synthetic
  source 1..20: mol_type = other DNA
ggtctcagtgcatccaaaac

SEQ ID NO: 488 - DNA, 21 bp, synthetic construct
  misc_feature 1..21: note = Synthetic
  source 1..21: mol_type = other DNA
tgcactgagaccccgacattc

SEQ ID NO: 489 - DNA, 20 bp, synthetic construct
  misc_feature 1..20: note = Synthetic
  source 1..20: mol_type = other DNA
tgcactgagaccccgacatt

SEQ ID NO: 490 - DNA, 21 bp, synthetic construct
  misc_feature 1..21: note = Synthetic
  source 1..21: mol_type = other DNA
caataaataccgaggaatgtc

SEQ ID NO: 491 - DNA, 20 bp, synthetic construct
  misc_feature 1..20: note = Synthetic
  source 1..20: mol_type = other DNA
aataaataccgaggaatgtc

SEQ ID NO: 492 - DNA, 21 bp, synthetic construct
  misc_feature 1..21: note = Synthetic
  source 1..21: mol_type = other DNA
acaataaataccgaggaatgt

SEQ ID NO: 493 - DNA, 20 bp, synthetic construct
  misc_feature 1..20: note = Synthetic
  source 1..20: mol_type = other DNA
caataaataccgaggaatgt

SEQ ID NO: 494 - DNA, 21 bp, synthetic construct
  misc_feature 1..21: note = Synthetic
  source 1..21: mol_type = other DNA
gacaataaataccgaggaatg

SEQ ID NO: 495 - DNA, 20 bp, synthetic construct
  misc_feature 1..20: note = Synthetic
  source 1..20: mol_type = other DNA
acaataaataccgaggaatg

SEQ ID NO: 496 - DNA, 21 bp, synthetic construct
  misc_feature 1..21: note = Synthetic
  source 1..21: mol_type = other DNA
tggggacagacaataaatacc

SEQ ID NO: 497 - DNA, 20 bp, synthetic construct
  misc_feature 1..20: note = Synthetic
  source 1..20: mol_type = other DNA
ggggacagacaataaatacc

SEQ ID NO: 498 - DNA, 21 bp, synthetic construct
  misc_feature 1..21: note = Synthetic
  source 1..21: mol_type = other DNA
gtatttattgtctgtccccac

SEQ ID NO: 499 - DNA, 20 bp, synthetic construct
  misc_feature 1..20: note = Synthetic
  source 1..20: mol_type = other DNA
gtatttattgtctgtcccca

SEQ ID NO: 500 - DNA, 21 bp, synthetic construct
  misc_feature 1..21: note = Synthetic
  source 1..21: mol_type = other DNA
tcgggggtgggggtcctaggt

SEQ ID NO: 501 - DNA, 20 bp, synthetic construct
  misc_feature 1..20: note = Synthetic
  source 1..20: mol_type = other DNA
cgggggtgggggtcctaggt

SEQ ID NO: 502 - DNA, 21 bp, synthetic construct
  misc_feature 1..21: note = Synthetic
  source 1..21: mol_type = other DNA
gtcgggggtgggggtcctagg

SEQ ID NO: 503 - DNA, 20 bp, synthetic construct
  misc_feature 1..20: note = Synthetic
  source 1..20: mol_type = other DNA
tcgggggtgggggtcctagg

SEQ ID NO: 504 - DNA, 21 bp, synthetic construct
  misc_feature 1..21: note = Synthetic
  source 1..21: mol_type = other DNA
ggtcgggggtgggggtcctag

SEQ ID NO: 505 - DNA, 20 bp, synthetic construct
  misc_feature 1..20: note = Synthetic
  source 1..20: mol_type = other DNA
gtcgggggtgggggtcctag

SEQ ID NO: 506 - DNA, 21 bp, synthetic construct
  misc_feature 1..21: note = Synthetic
  source 1..21: mol_type = other DNA
gagggtcgggggtgggggtcc

SEQ ID NO: 507 - DNA, 20 bp, synthetic construct
  misc_feature 1..20: note = Synthetic
  source 1..20: mol_type = other DNA
agggtcgggggtgggggtcc

SEQ ID NO: 508 - DNA, 21 bp, synthetic construct
  misc_feature 1..21: note = Synthetic
  source 1..21: mol_type = other DNA
tattcgcgagggtcgggggtg

SEQ ID NO: 509 - DNA, 20 bp, synthetic construct
  misc_feature 1..20: note = Synthetic
  source 1..20: mol_type = other DNA
attcgcgagggtcgggggtg

SEQ ID NO: 510 - DNA, 21 bp, synthetic construct
  misc_feature 1..21: note = Synthetic
  source 1..21: mol_type = other DNA
cacccccgaccctcgcgaata

SEQ ID NO: 511 - DNA, 20 bp, synthetic construct
  misc_feature 1..20: note = Synthetic
  source 1..20: mol_type = other DNA
cacccccgaccctcgcgaat

SEQ ID NO: 512 - DNA, 21 bp, synthetic construct
  misc_feature 1..21: note = Synthetic
  source 1..21: mol_type = other DNA
ttattcgcgagggtcgggggt

SEQ ID NO: 513 - DNA, 20 bp, synthetic construct
  misc_feature 1..20: note = Synthetic
  source 1..20: mol_type = other DNA
tattcgcgagggtcgggggt

SEQ ID NO: 514 - DNA, 21 bp, synthetic construct
  misc_feature 1..21: note = Synthetic
  source 1..21: mol_type = other DNA
tttattcgcgagggtcggggg

SEQ ID NO: 515 - DNA, 20 bp, synthetic construct
  misc_feature 1..20: note = Synthetic
  source 1..20: mol_type = other DNA
ttattcgcgagggtcggggg

SEQ ID NO: 516 - DNA, 21 bp, synthetic construct
  misc_feature 1..21: note = Synthetic
  source 1..21: mol_type = other DNA
ttttattcgcgagggtcgggg

SEQ ID NO: 517 - DNA, 20 bp, synthetic construct
  misc_feature 1..20: note = Synthetic
  source 1..20: mol_type = other DNA
tttattcgcgagggtcgggg

SEQ ID NO: 518 - DNA, 21 bp, synthetic construct
  misc_feature 1..21: note = Synthetic
  source 1..21: mol_type = other DNA
gccttttattcgcgagggtcg

SEQ ID NO: 519 - DNA, 20 bp, synthetic construct
  misc_feature 1..20: note = Synthetic
  source 1..20: mol_type = other DNA
ccttttattcgcgagggtcg

SEQ ID NO: 520 - DNA, 21 bp, synthetic construct
  misc_feature 1..21: note = Synthetic
  source 1..21: mol_type = other DNA
ggccttttattcgcgagggtc

SEQ ID NO: 521 - DNA, 20 bp, synthetic construct
  misc_feature 1..20: note = Synthetic
  source 1..20: mol_type = other DNA
gccttttattcgcgagggtc

SEQ ID NO: 522 - DNA, 21 bp, synthetic construct
  misc_feature 1..21: note = Synthetic
  source 1..21: mol_type = other DNA
gggccttttattcgcgagggt

SEQ ID NO: 523 - DNA, 20 bp, synthetic construct
  misc_feature 1..20: note = Synthetic
  source 1..20: mol_type = other DNA
ggccttttattcgcgagggt

SEQ ID NO: 524 - DNA, 21 bp, synthetic construct
  misc_feature 1..21: note = Synthetic
  source 1..21: mol_type = other DNA
agggccttttattcgcgaggg

SEQ ID NO: 525 - DNA, 20 bp, synthetic construct
  misc_feature 1..20: note = Synthetic
  source 1..20: mol_type = other DNA
gggccttttattcgcgaggg

SEQ ID NO: 526 - DNA, 21 bp, synthetic construct
  misc_feature 1..21: note = Synthetic
  source 1..21: mol_type = other DNA
atggagggccttttattcgcg

SEQ ID NO: 527 - DNA, 20 bp, synthetic construct
  misc_feature 1..20: note = Synthetic
  source 1..20: mol_type = other DNA
tggagggccttttattcgcg

SEQ ID NO: 528 - DNA, 21 bp, synthetic construct
  misc_feature 1..21: note = Synthetic
  source 1..21: mol_type = other DNA
gatggagggccttttattcgc

SEQ ID NO: 529 - DNA, 20 bp, synthetic construct
  misc_feature 1..20: note = Synthetic
  source 1..20: mol_type = other DNA
atggagggccttttattcgc

SEQ ID NO: 530 - DNA, 21 bp, synthetic construct
  misc_feature 1..21: note = Synthetic
  source 1..21: mol_type = other DNA
tccagagctttgggcagatgg

SEQ ID NO: 531 - DNA, 20 bp, synthetic construct
  misc_feature 1..20: note = Synthetic
  source 1..20: mol_type = other DNA
ccagagctttgggcagatgg

SEQ ID NO: 532 - DNA, 20 bp, synthetic construct
  misc_feature 1..20: note = Synthetic
  source 1..20: mol_type = other DNA
cgctaggaagcagccaatga

SEQ ID NO: 533 - DNA, 20 bp, synthetic construct
  misc_feature 1..20: note = Synthetic
  source 1..20: mol_type = other DNA
tagctcctcccagaccttcg

SEQ ID NO: 534 - DNA, 18 bp, synthetic construct
  misc_feature 1..18: note = Synthetic
  source 1..18: mol_type = other DNA
aatgacgagttcggacgg

SEQ ID NO: 535 - DNA, 20 bp, synthetic construct
  misc_feature 1..20: note = Synthetic
  source 1..20: mol_type = other DNA
ggggatcacagaccatttct

SEQ ID NO: 536 - DNA, 19 bp, synthetic construct
  misc_feature 1..19: note = Synthetic
  source 1..19: mol_type = other DNA
tggaggatggaacacggac

SEQ ID NO: 537 - DNA, 24 bp, synthetic construct
  misc_feature 1..24: note = Synthetic
  source 1..24: mol_type = other DNA
ttctttcggccaggctgaggccct

SEQ ID NO: 538 - DNA, 15 bp, synthetic construct
  misc_feature 1..15: note = Synthetic
  source 1..15: mol_type = other DNA
ctagcggccggggag

SEQ ID NO: 539 - DNA, 17 bp, synthetic construct
  misc_feature 1..17: note = Synthetic
  source 1..17: mol_type = other DNA
agcagcattcccggcta

SEQ ID NO: 540 - DNA, 24 bp, synthetic construct
  misc_feature 1..24: note = Synthetic
  source 1..24: mol_type = other DNA
cgaacggggctcgaagggtccttg

SEQ ID NO: 541 - DNA, 20 bp, synthetic construct
  misc_feature 1..20: note = Synthetic
  source 1..20: mol_type = other DNA
ggatatgtgaccatgctacc

SEQ ID NO: 542 - DNA, 20 bp, synthetic construct
  misc_feature 1..20: note = Synthetic
  source 1..20: mol_type = other DNA
gggttgtatccagtacctct

SEQ ID NO: 543 - DNA, 25 bp, synthetic construct
  misc_feature 1..25: note = Synthetic
  source 1..25: mol_type = other DNA
tgtcctgttccttcccccagcccca

SEQ ID NO: 544-599 - skipped (56 entries)

SEQ ID NO: 600 - DNA, 76 bp, synthetic construct
  misc_feature 1..76: note = Synthetic
  source 1..76: mol_type = other DNA
gttttagtactctggaaacagaatctactgaaacaagacaatatgtcgtgtttatcccatcaatttattggtggga

SEQ ID NO: 601 - DNA, 86 bp, synthetic construct
  misc_feature 1..86: note = Synthetic
  source 1..86: mol_type = other DNA
gtttaagtactctgtgctggaaacagcacagaatctactgaaacaagacaatatgtcgtgtttatcccatcaatttattggtggga

SEQ ID NO: 602-699 - skipped (98 entries)

SEQ ID NO: 700 - DNA, 450 bp, synthetic construct
  misc_feature 1..450: note = Synthetic
  source 1..450: mol_type = other DNA
ctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 701 - DNA, 436 bp, synthetic construct
  misc_feature 1..436: note = Synthetic
  source 1..436: mol_type = other DNA
tgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 702 - DNA, 472 bp, synthetic construct
  misc_feature 1..472: note = Synthetic
  source 1..472: mol_type = other DNA
cgagtccaacacccgtgggaatcccatgggcaccatggcccctcgctccaaaaatgctttcgcgtcgcgcagacactgctcggtagtttcggggatcagcgtttgagtaagagcccgcgtctgaaccctccgcgccgccccggccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaa

SEQ ID NO: 703 - DNA, 108 bp, synthetic construct
  misc_feature 1..108: note = Synthetic
  source 1..108: mol_type = other DNA
gctcggcgcgcccatatttgcatgtcgctatgtgttctgggaaatcaccataaacgtgaaatgtctttggatttgggaatcttataagttctgtatgagaccacggta

SEQ ID NO: 704 - DNA, 251 bp, synthetic construct
  misc_feature 1..251: note = Synthetic
  source 1..251: mol_type = other DNA
tgacggcgcgccctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggta

SEQ ID NO: 705 - DNA, 249 bp, synthetic construct
  misc_feature 1..249: note = Synthetic
  source 1..249: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 706 - DNA, 243 bp, synthetic construct
  misc_feature 1..243: note = Synthetic
  source 1..243: mol_type = other DNA
ctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctc

SEQ ID NO: 707 - DNA, 100 bp, synthetic construct
  misc_feature 1..100: note = Synthetic
  source 1..100: mol_type = other DNA
aatatttgcatgtcgctatgtgttctgggaaatcaccataaacgtgaaatgtctttggatttgggaatcttataagttctgtatgagaccactctttccc

SEQ ID NO: 708 - skipped

SEQ ID NO: 709 - DNA, 143 bp, synthetic construct
  misc_feature 1..143: note = Synthetic
  source 1..143: mol_type = other DNA
ggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 710 - DNA, 137 bp, synthetic construct
  misc_feature 1..137: note = Synthetic
  source 1..137: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagaggga

SEQ ID NO: 711 - skipped

SEQ ID NO: 712 - protein, 1053 aa, synthetic construct
  REGION 1..1053: note = Synthetic
NQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 713 - protein, 7 aa, synthetic construct
  REGION 1..7: note = Synthetic
PKKKRKV

SEQ ID NO: 714 - protein, 16 aa, synthetic construct
  REGION 1..16: note = Synthetic
KRPAATKKAGQAKKKK

SEQ ID NO: 715 - skipped

SEQ ID NO: 716 - protein, 1057 aa, synthetic construct
  REGION 1..1057: note = Synthetic
MNQKFILGLDIGITSVGYGLIDYETKNIIDAGVRLFPEANVENNEGRRSKRGSRRLKRRRIHRLDRVKHLLAEYDLLDLTNIPKSTNPYQTRVKGLNEKLSKDELVIALLHIAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGKGSPFGWEGNIKKWFEQMMGHCTYFPEELRSVKYSYSAELFNALNDLNNLVITRDEDAKLNYGEKFQIIENVFKQKKTPNLKQIAIEIGVHETEIKGYRVNKSGTPEFTEFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVKRTFIQSINVINKVIEKYGIPEDIIIELARENNSDDRKKFINNLQKKNEATRKRINEIIGQTGNQNAKRIVEKIRLHDQQEGKCLYSLKDIPLEDLLRNPNNYDIDHIIPRSVSFDDSMHNKVLVRREQNAKKNNQTPYQYLTSGYADIKYSVFKQHVLNLAENKDRMTKKKREYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 717 - protein, 1053 aa, synthetic construct
  REGION 1..1053: note = Synthetic
MNQKFILGLDIGITSVGYGLIDYETKNIIDAGVRLFPEANVENNEGRRSKRGSRRLKRRRIHRLERVKSLLSEYKIISGLAPTNNQPYNIRVKGLTEQLTKDELAVALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTEFKLYHDLKSVLFDQSILENEDVLDQIAEILTIYQDKDSIKSKLTELDILLNEEDKENIAQLTGYNGTHRLSLKCIRLVLEEQWYSSRNQMEIFTHLNIKPKKINLTAANKIPKAMIDEFILSPVVKRTFIQSINVINKVIEKYGIPEDIIIELARENNSDDRKKFINNLQKKNEATRKRINEIIGQTGNQNAKRIVEKIRLHDQQEGKCLYSLESIALMDLLNNPQNYEVDHIIPRSVAFDNSIHNKVLVKQIENSKKGNRTPYQYLNSSDAKLSYNQFKQHILNLSKSKDRISKKKKDYLLEERDINKFEVQKEFINRNLVDTRYATRELTSYLKAYFSANNMDVKVKTINGSFTNHLRKVWRFDKYRNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 718 - protein, 1053 aa, synthetic construct
  REGION 1..1053: note = Synthetic
NQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRKLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPHIKKTIGKKVNSIEKLTTDVLGNVFTNTQYTKPQLLFKRGN

SEQ ID NO: 719 - protein, 1053 aa, synthetic construct
  REGION 1..1053: note = Synthetic
NQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELASVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRAQMEIFAHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATAELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 720 - protein, 1053 aa, synthetic construct
  REGION 1..1053: note = Synthetic
NQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELASVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRAQMEIFAHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATAELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRKLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPHIKKTIGKKVNSIEKLTTDVLGNVFTNTQYTKPQLLFKRGN

SEQ ID NO: 721 - protein, 1057 aa, synthetic construct
  REGION 1..1057: note = Synthetic
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 722 - protein, 1054 aa, synthetic construct
  REGION 1..1054: note = Synthetic
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHNINVSSEDEDASNELSTKEQINRNNKLLKDKYVCEVQLQRLKEGQIRGEKNRFKTTDILKEIDQLLKVQKDYHNLDIDFINQYKEIVETRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 723 - protein, 1057 aa, synthetic construct
  REGION 1..1057: note = Synthetic
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 724 - protein, 1057 aa, synthetic construct
  REGION 1..1057: note = Synthetic
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 725 - protein, 1057 aa, synthetic construct
  REGION 1..1057: note = Synthetic
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 726-899 - skipped (174 entries)

SEQ ID NO: 900 - DNA, 77 bp, synthetic construct
  misc_feature 1..77: note = Synthetic
  source 1..77: mol_type = other DNA
gttttagtactctggaaacagaatctactgaaacaagacaatatgtcgtgtttatcccatcaatttattggtgggat

SEQ ID NO: 901 - DNA, 77 bp, synthetic construct
  misc_feature 1..77: note = Synthetic
  source 1..77: mol_type = other DNA
gtttcagtactctggaaacagaatctactgaaacaagacaatatgtcgtgtttatcccatcaatttattggtgggat

SEQ ID NO: 902 - DNA, 77 bp, synthetic construct
  misc_feature 1..77: note = Synthetic
  source 1..77: mol_type = other DNA
gtttggtaacctaggaaactagatcttaccaaacaagacaatatgtcgtgtttatcccatcaatttattggtgggat

SEQ ID NO: 903 - DNA, 77 bp, synthetic construct
  misc_feature 1..77: note = Synthetic
  source 1..77: mol_type = other DNA
gtttcagtactctggaaacagaatctactgaaacaaggcaaaatgccgtgtttatcccatcaatttattggtgggat

SEQ ID NO: 904 - DNA, 77 bp, synthetic construct
  misc_feature 1..77: note = Synthetic
  source 1..77: mol_type = other DNA
gtttcagtactctggaaacagaatctactgaaacaagacaatatgtcgcgcccatcccatcaatttattggtgggat

SEQ ID NO: 905 - DNA, 77 bp, synthetic construct
  misc_feature 1..77: note = Synthetic
  source 1..77: mol_type = other DNA
gtttcagtactctggaaacagaatctactgaaacaagacaatatgtcgtgtttatgggttgaatttattcgacccat

SEQ ID NO: 906 - DNA, 77 bp, synthetic construct
  misc_feature 1..77: note = Synthetic
  source 1..77: mol_type = other DNA
gtttggtaacctaggaaactagatcttaccaaacaaggcaaaatgccgtgtttatcccatcaatttattggtgggat

SEQ ID NO: 907 - DNA, 77 bp, synthetic construct
  misc_feature 1..77: note = Synthetic
  source 1..77: mol_type = other DNA
gtttggtaacctaggaaactagatcttaccaaacaagacaatatgtcgcgcccatcccatcaatttattggtgggat

SEQ ID NO: 908 - DNA, 77 bp, synthetic construct
  misc_feature 1..77: note = Synthetic
  source 1..77: mol_type = other DNA
gtttggtaacctaggaaactagatcttaccaaacaagacaatatgtcgtgtttatgggttgaatttattcgacccat

SEQ ID NO: 909 - DNA, 77 bp, synthetic construct
  misc_feature 1..77: note = Synthetic
  source 1..77: mol_type = other DNA
gtttcagtactctggaaacagaatctactgaaacaaggcaaaatgccgcgcccatcccatcaatttattggtgggat

SEQ ID NO: 910 - DNA, 77 bp, synthetic construct
  misc_feature 1..77: note = Synthetic
  source 1..77: mol_type = other DNA
gtttcagtactctggaaacagaatctactgaaacaaggcaaaatgccgtgtttatgggttgaatttattcgacccat

SEQ ID NO: 911 - DNA, 77 bp, synthetic construct
  misc_feature 1..77: note = Synthetic
  source 1..77: mol_type = other DNA
gtttcagtactctggaaacagaatctactgaaacaagacaatatgtcgcgcccatgggttgaatttattcgacccat

SEQ ID NO: 912 - DNA, 77 bp, synthetic construct
  misc_feature 1..77: note = Synthetic
  source 1..77: mol_type = other DNA
gtttggtaacctaggaaactagatcttaccaaacaaggcaaaatgccgcgcccatcccatcaatttattggtgggat

SEQ ID NO: 913 - DNA, 77 bp, synthetic construct
  misc_feature 1..77: note = Synthetic
  source 1..77: mol_type = other DNA
gtttggtaacctaggaaactagatcttaccaaacaaggcaaaatgccgtgtttatgggttgaatttattcgacccat

SEQ ID NO: 914 - DNA, 77 bp, synthetic construct
  misc_feature 1..77: note = Synthetic
  source 1..77: mol_type = other DNA
gtttggtaacctaggaaactagatcttaccaaacaagacaatatgtcgcgcccatgggttgaatttattcgacccat

SEQ ID NO: 915 - DNA, 77 bp, synthetic construct
  misc_feature 1..77: note = Synthetic
  source 1..77: mol_type = other DNA
gtttcagtactctggaaacagaatctactgaaacaaggcaaaatgccgcgcccatgggttgaatttattcgacccat

SEQ ID NO: 916 - DNA, 77 bp, synthetic construct
  misc_feature 1..77: note = Synthetic
  source 1..77: mol_type = other DNA
gtttggtaacctaggaaactagatcttaccaaacaaggcaaaatgccgcgcccatgggttgaatttattcgacccat

SEQ ID NO: 917 - DNA, 87 bp, synthetic construct
  misc_feature 1..87: note = Synthetic
  source 1..87: mol_type = other DNA
gtttcagtactctgtgctggaaacagcacagaatctactgaaacaagacaatatgtcgtgtttatcccatcaatttattggtgggat

SEQ ID NO: 918-939 - skipped (22 entries)

SEQ ID NO: 940 - protein, 4 aa, synthetic construct
  REGION 1..4: note = Synthetic
GSVD

SEQ ID NO: 941 - protein, 4 aa, synthetic construct
  REGION 1..4: note = Synthetic
GSGS

SEQ ID NO: 942 - protein, 9 aa, synthetic construct
  REGION 1..9: note = Synthetic
PAAKKKKLD

SEQ ID NO: 943 - DNA, 27 bp, synthetic construct
  misc_feature 1..27: note = Synthetic
  source 1..27: mol_type = other DNA
ccggcagctaagaaaaagaaactggat